(19) United States
(12) Patent Application Publication
(10) Pub. No.: US 2023/0373941 A1
(43) Pub. Date: Nov. 23, 2023

(54) COMPOUNDS FOR ELECTRONIC DEVICES

(71) Applicant: Merck Patent GmbH, Darmstadt (DE)

(72) Inventors: Elvira MONTENEGRO, Darmstadt (DE); Teresa MUJICA-FERNAUD, Darmstadt (DE); Rachel TUFFIN, Darmstadt (DE)

(21) Appl. No.: 18/030,512

(22) PCT Filed: Aug. 20, 2021
(86) PCT No.: PCT/EP2021/073110

§ 371 Date: Apr. 6, 2023

(30) Foreign Application Priority Data
Oct. 9, 2020 (EP) 20201069.0
May 7, 2021 (EP) 21172857.1

(51) Int. Cl. C07D 307/91; C07C 211/60; C07D 209/70; H10K 85/60; H10K 50/15

(52) U.S. Cl. CPC C07D 307/91; C07C 211/60; C07D 209/70; H10K 85/6574; H10K 85/633; H10K 85/615; H10K 85/6572; H10K 50/15; C07C 2603/18

(57) ABSTRACT

The present application relates to compounds of the formula (1), to processes for preparing these compounds, to the use of these compounds in electronic devices and to electronic devices containing at least one of these compounds.

The present application relates to spirobifluoreneamines comprising particular aromatic or heteroaromatic ring systems on the amine nitrogen atom. The compounds are suitable for use in electronic devices.

Electronic devices in the context of this application are understood to mean what are called organic electronic devices, which comprise organic semiconductor materials as functional materials. More particularly, these are understood to mean OLEDs (organic electroluminescent devices). The term OLEDs is understood to mean electronic devices which have one or more layers comprising organic compounds and emit light on application of electrical voltage. The construction and general principle of function of OLEDs are known to those skilled in the art.

In electronic devices, especially OLEDs, there is great interest in an improvement in the performance data. In these aspects, it has not yet been possible to find any entirely satisfactory solution.

A great influence on the performance data of electronic devices is possessed by emission layers and layers having a hole-transporting function. Novel compounds are also being sought for use in these layers, especially hole-transporting compounds and compounds that can serve as hole-transporting matrix material, especially for phosphorescent emitters, in an emitting layer. For this purpose, there is a search especially for compounds that have a high glass transition temperature, high stability, and high conductivity for holes. A high stability of the compound is a prerequisite for achieving a long lifetime of the electronic device. There is moreover a need to find compounds whose use in electronic devices results in improvement of the performance data of the devices, especially in high efficiency, long lifetime and low operating voltage.

In the prior art, triarylamine compounds such as spirobifluoreneamines and fluoreneamines in particular are known as hole transport materials and hole-transporting matrix materials for electronic devices. However, there remains room for improvement in respect of the abovementioned properties.

It has now been found that spirobifluoreneamines according to the following formula (I) which are characterized in that they comprise certain aromatic or heteroaromatic ring systems at the amine nitrogen atom are exceptionally suitable for use in electronic devices. They are especially suitable for use in OLEDs, and even more particularly therein for use as hole transport materials and for use as hole-transporting matrix materials, especially for phosphorescent emitters. The compounds lead to high lifetime, high efficiency and low operating voltage of the devices. Further preferably, the compounds found have a high glass transition temperature, high stability and high conductivity for holes.

The present application thus provides compounds of a formula (I)

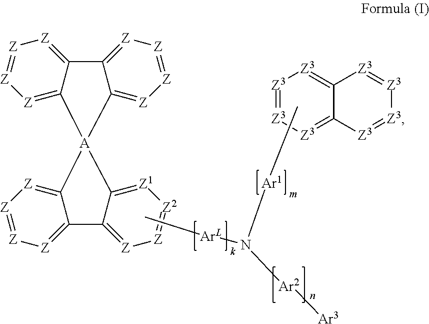

wherein the amino group shown with a variable bond position at the spirobifluorene in formula (I) is bonded either to Z¹ or to Z²; and
wherein the following applies for the groups and indices that occur:
A is C or Si;
Ar^{L} is the same or different at each instance and is selected from aromatic ring systems which have 6 to 40 aromatic ring atoms and are each substituted by R² radicals, and heteroaromatic ring systems which have 5 to 40 aromatic ring atoms and are each substituted by R² radicals;
Ar¹ is the same or different at each instance and is selected from aromatic ring systems which have 6 to 24 aromatic ring atoms and are substituted by R³ radicals, and heteroaromatic ring systems which have 5 to 24 aromatic ring atoms and are substituted by R³ radicals;
Ar² is the same or different at each instance and is selected from phenyl substituted by R⁵ radicals, naphthyl substituted by R⁵ radicals and heteroaromatic ring systems which have 6 to 10 aromatic ring atoms and are each substituted by R⁵ radicals;
Ar³ is selected from groups of the formulae (Ar³-1) to (Ar³-7) which are bonded to the rest of the formula (I) via the bond marked with *

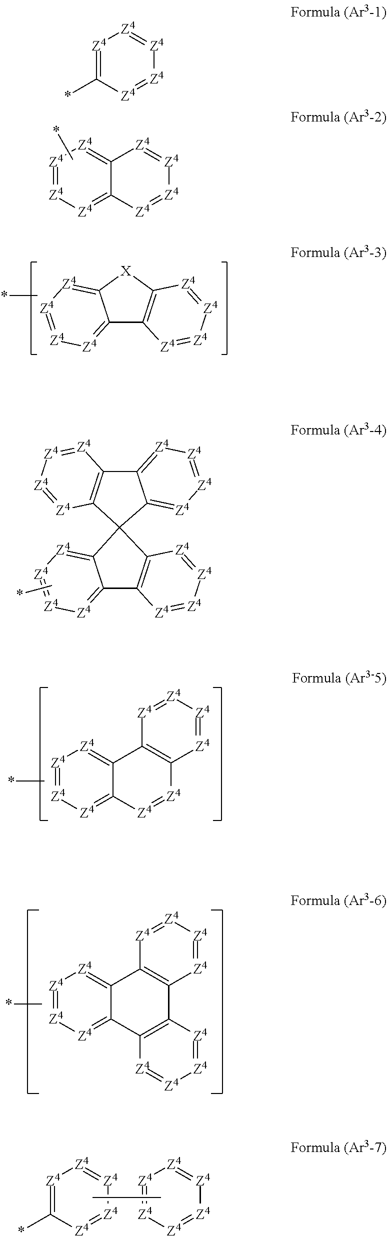

wherein Z⁴ is the same or different at each instance and is selected from CR⁶ and N and wherein in formulae (Ar³-2) to (Ar³-7) the group Z⁴ which bonds to a freely drawn bond is C; wherein in formula (Ar³-3) X representing C(R⁶)₂ may also be bonded to the rest of formula (I), wherein a group R⁶ in this group X is then replaced by the bond to the rest of formula (I);
and wherein X is selected from O, S, NR⁶ and C(R⁶)₂; and wherein in formula (Ar³-3) one or more units Z⁴═Z⁴ may be replaced by a unit selected from the units

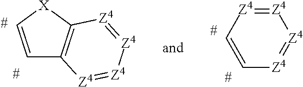

wherein the atoms marked with # in each case replace the atoms Z⁴;
and wherein in formula (Ar³-3) one or more units Z⁴—Z⁴ may be replaced by a unit selected from the units

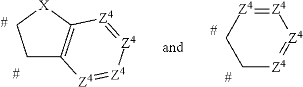

wherein the atoms marked with # in each case replace the atoms Z⁴;
Z is the same or different at each instance and is selected from CR¹ and N;
Z¹ and Z² are each C when the amino group shown with a variable bond position is bonded thereto and otherwise selected from CR¹ and N;
Z³ is the same or different at each instance and is selected from CR⁴ and N;
R¹ is the same or different at each instance and is selected from H, D, F, Cl, Br, I, C(═O)R⁷, CN, Si(R⁷)₃, N(R⁷)₂, —(Ar^{L})ₖ—N(Ar⁴)₂, P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals R¹, R², R³, R⁴, R⁵ and R⁶ may be joined to one another and may form a ring; where the alkyl, alkoxy, alkenyl and alkynyl groups mentioned and the aromatic ring systems and heteroaromatic ring systems mentioned are each substituted by R⁷ radicals; and where one or more CH₂ groups in the alkyl, alkoxy, alkenyl and alkynyl groups mentioned may be replaced by —R⁷C═CR⁷—, —C≡C—, Si(R⁷)₂, C═O, C═NR⁷, —C(═O)O—, —C(═O)NR⁷—, NR⁷, P(═O)(R⁷), —O—, —S—, SO or SO₂;
R², R³, R⁴, R⁵ and R⁶ are each the same or different at each instance and are selected from H, D, F, Cl, Br, I, C(═O)R⁷, CN, Si(R⁷)₃, N(R⁷)₂, P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals R², R³, R⁴, R⁵ and R⁶ may be joined to one another and may form a ring; where the alkyl, alkoxy, alkenyl and alkynyl groups mentioned and the aromatic ring systems and heteroaromatic ring systems mentioned are each substituted by R⁷ radicals; and where one or more CH₂ groups in the alkyl, alkoxy, alkenyl and alkynyl groups mentioned may be replaced by —R⁷C═CR⁷—, —C≡C—, Si(R⁷)₂, C═O, C═NR⁷, —C(═O)O—, —C(═O)NR⁷—, NR⁷, P(═O)(R⁷), —O—, —S—, SO or SO₂;
Ar⁴ is the same or different at each instance and is selected from aromatic ring systems which have 6 to 40 aromatic ring atoms and are substituted by one or more R⁷ radicals, and heteroaromatic ring systems which have 5 to 40 aromatic ring atoms and are substituted by one or more R⁷ radicals;
R⁷ is the same or different at each instance and is selected from H, D, F, Cl, Br, I, C(═O)R⁸, CN, Si(R⁸)₃, N(R⁸)₂, P(═O)(R⁸)₂, OR⁸, S(═O)R⁸, S(═O)₂R⁸, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹, R², R³, R⁴, R⁵ and R⁶ radicals may be joined to one another and may form a ring; where the alkyl, alkoxy, alkenyl and alkynyl groups mentioned and the aromatic ring systems and heteroaromatic ring systems mentioned are each substituted by R⁸ radicals; and where one or more CH₂ groups in the alkyl, alkoxy, alkenyl and alkynyl groups mentioned may be replaced by −R⁸C═CR⁸—, —C≡C—, Si(R⁸)₂, C═O, C═NR⁸, —C(═O)O—, —C(═O)NR⁸—, NR⁸, P(═O)(R⁸), —O—, —S—, SO or SO₂;
R⁸ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁸ radicals may be joined to one another and may form a ring; and where the alkyl, alkoxy, alkenyl and alkynyl groups, aromatic ring systems and heteroaromatic ring systems mentioned may be substituted by one or more radicals selected from F and CN;
k is 0, 1, 2, 3 or 4;
m is 0, 1, 2, 3 or 4;
n is 1, 2, 3 or 4.

The units in square brackets in formulae (Ar³-3) and (Ar³-5) may be bonded at any of their positions to the bond marked with *.

The definitions which follow are applicable to the chemical groups that are used in the present application. They are applicable unless any more specific definitions are given.

An aryl group in the context of this invention is understood to mean either a single aromatic cycle, i.e. benzene, or a fused aromatic polycycle, for example naphthalene, phenanthrene or anthracene. A fused aromatic polycycle in the context of the present application consists of two or more single aromatic cycles fused to one another. Fusion between cycles is understood here to mean that the cycles share at least one edge with one another. An aryl group in the context of this invention contains 6 to 40 aromatic ring atoms. In addition, an aryl group does not contain any heteroatom as aromatic ring atom, but only carbon atoms.

A heteroaryl group in the context of this invention is understood to mean either a single heteroaromatic cycle, for example pyridine, pyrimidine or thiophene, or a fused heteroaromatic polycycle, for example quinoline or carbazole. A fused heteroaromatic polycycle in the context of the present application consists of two or more single aromatic or heteroaromatic cycles that are fused to one another, where at least one of the aromatic and heteroaromatic cycles is a heteroaromatic cycle. Fusion between cycles is understood here to mean that the cycles share at least one edge with one another. A heteroaryl group in the context of this invention contains 5 to 40 aromatic ring atoms of which at least one is a heteroatom. The heteroatoms of the heteroaryl group are preferably selected from N, O and S.

An aryl or heteroaryl group, each of which may be substituted by the abovementioned radicals, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo[1,2-a]benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention is a system which does not necessarily contain solely aryl groups, but which may additionally contain one or more nonaromatic rings fused to at least one aryl group. These nonaromatic rings contain exclusively carbon atoms as ring atoms. Examples of groups covered by this definition are tetrahydronaphthalene, fluorene and spirobifluorene. In addition, the term “aromatic ring system” includes systems that consist of two or more aromatic ring systems joined to one another via single bonds, for example biphenyl, terphenyl, 7-phenyl-2-fluorenyl, quaterphenyl and 3,5-diphenyl-1-phenyl. An aromatic ring system in the context of this invention contains 6 to 40 carbon atoms and no heteroatoms in the ring system. The definition of “aromatic ring system” does not include heteroaryl groups.

A heteroaromatic ring system conforms to the abovementioned definition of an aromatic ring system, except that it must contain at least one heteroatom as ring atom. As is the case for the aromatic ring system, the heteroaromatic ring system need not contain exclusively aryl groups and heteroaryl groups, but may additionally contain one or more nonaromatic rings fused to at least one aryl or heteroaryl group. The nonaromatic rings may contain exclusively carbon atoms as ring atoms, or they may additionally contain one or more heteroatoms, where the heteroatoms are preferably selected from N, O and S. One example of such a heteroaromatic ring system is benzopyranyl. In addition, the term “heteroaromatic ring system” is understood to mean systems that consist of two or more aromatic or heteroaromatic ring systems that are bonded to one another via single bonds, for example 4,6-diphenyl-2-triazinyl. A heteroaromatic ring system in the context of this invention contains 5 to 40 ring atoms selected from carbon and heteroatoms, where at least one of the ring atoms is a heteroatom. The heteroatoms of the heteroaromatic ring system are preferably selected from N, O and S.

The terms “heteroaromatic ring system” and “aromatic ring system” as defined in the present application thus differ from one another in that an aromatic ring system cannot have a heteroatom as ring atom, whereas a heteroaromatic ring system must have at least one heteroatom as ring atom. This heteroatom may be present as a ring atom of a nonaromatic heterocyclic ring or as a ring atom of an aromatic heterocyclic ring.

In accordance with the above definitions, any aryl group is covered by the term “aromatic ring system”, and any heteroaryl group is covered by the term “heteroaromatic ring system”.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be substituted by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical binds to the position to which the hydrogen atom was bonded, forming a ring. Preferably, group A is C.

Preferably, Z¹ and Z² are each C when the amino group shown with a variable bond position is bonded thereto and otherwise are CR¹.

Preferably, Z³ is CR⁴.

Preferably, Z is CR¹.

Preferably, Z⁴ is CR⁶.

It is particularly preferable when Z¹ and Z² are each C when the amino group shown with a variable bond position is bonded thereto and otherwise are CR¹, Z is CR¹, Z³ is CR⁴ and Z⁴ is CR⁶.

Preferably, R¹ is the same or different at each instance and is selected from H, D, F, CN, Si(R⁷)₃, N(R⁷)₂, —[Ar^{L}]ₖ—N(Ar⁴)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁷ radicals; and where one or more CH₂ groups in the alkyl or alkoxy groups mentioned may be replaced by —C≡C—, —R⁷C═CR⁷—, Si(R⁷)₂, C═O, C═NR⁷, —NR⁷—, —O—, —S—, —C(═O)O— or —C(═O)NR⁷—. It is particularly preferable when R¹ is the same or different at each instance and is selected from H, —[Ar^{L}]ₖ—N(Ar⁴)₂, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where said alkyl groups, said aromatic ring systems and said heteroaromatic ring systems are each substituted by R⁷ radicals. It is yet more preferable when R¹ is H.

Preferred groups R¹ in the compound of formula (I) are selected from the following groups:

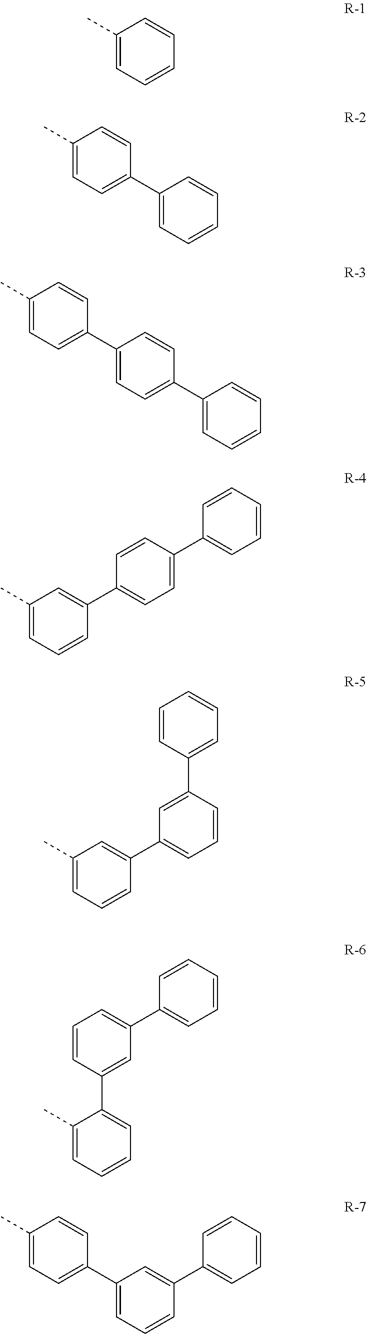

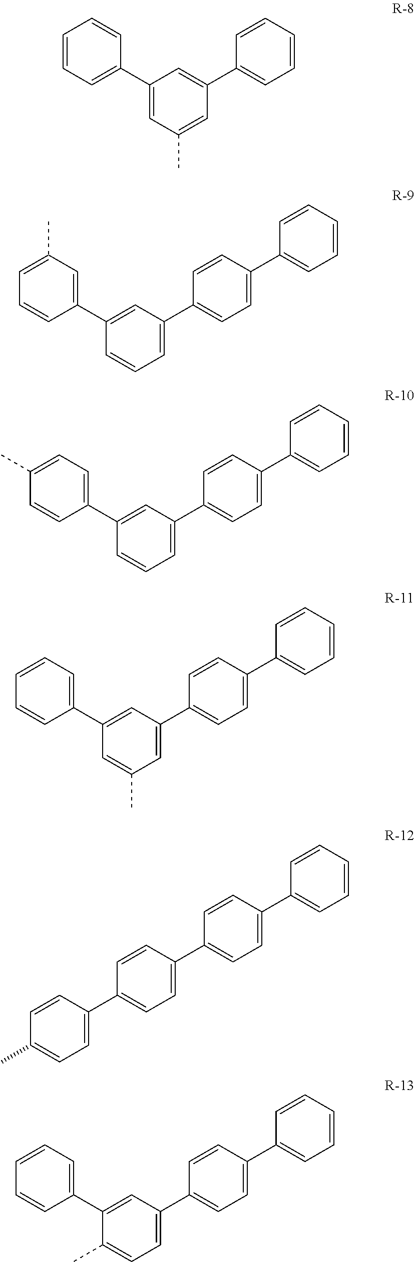

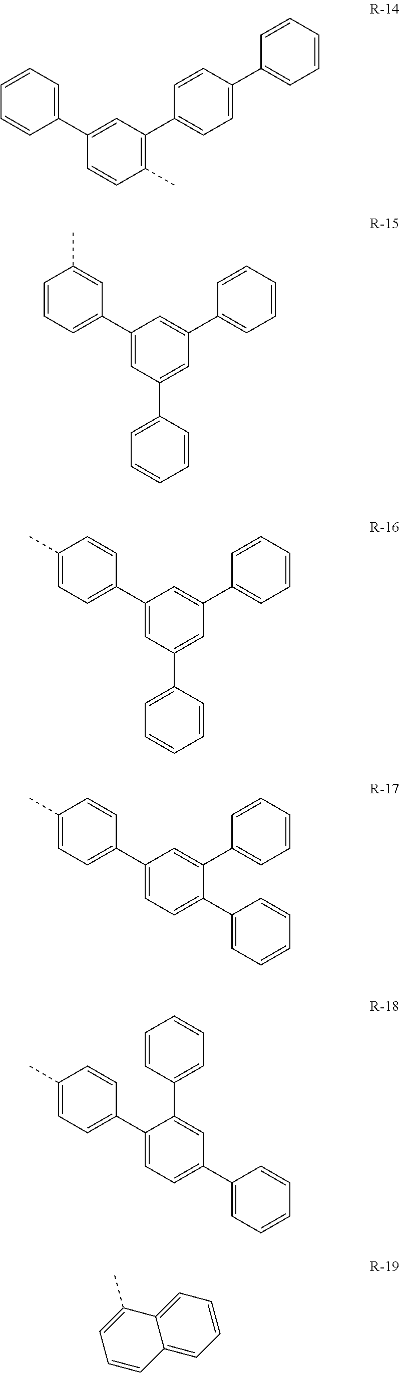

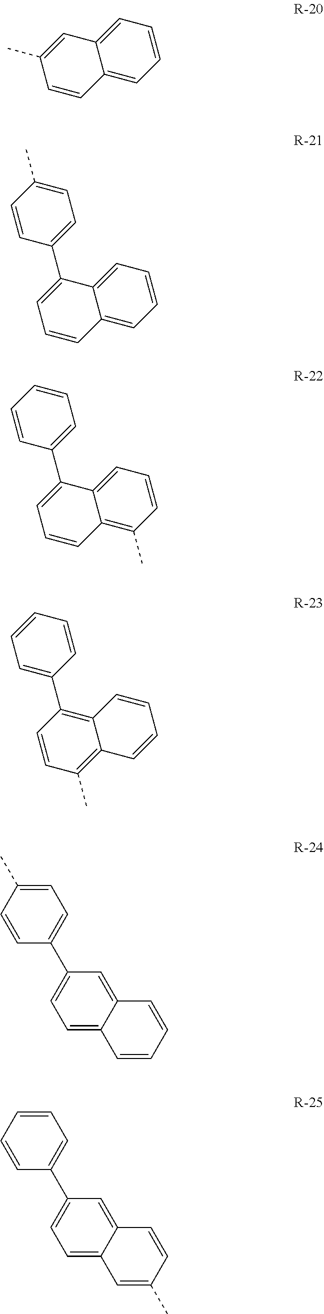

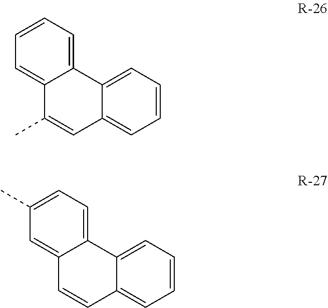

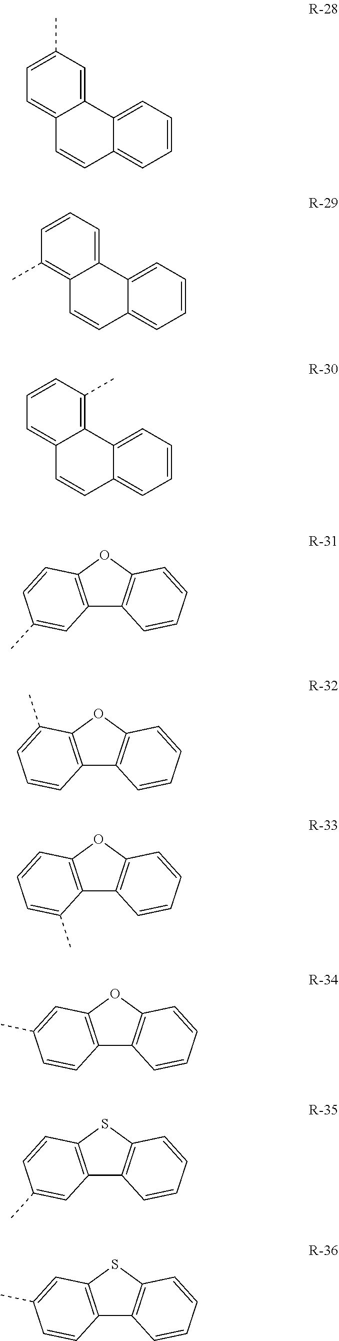

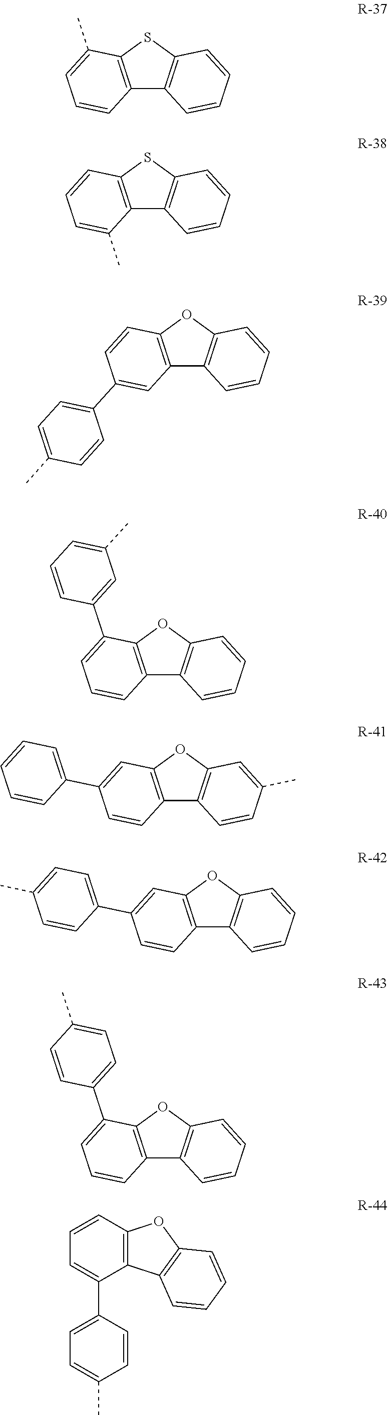

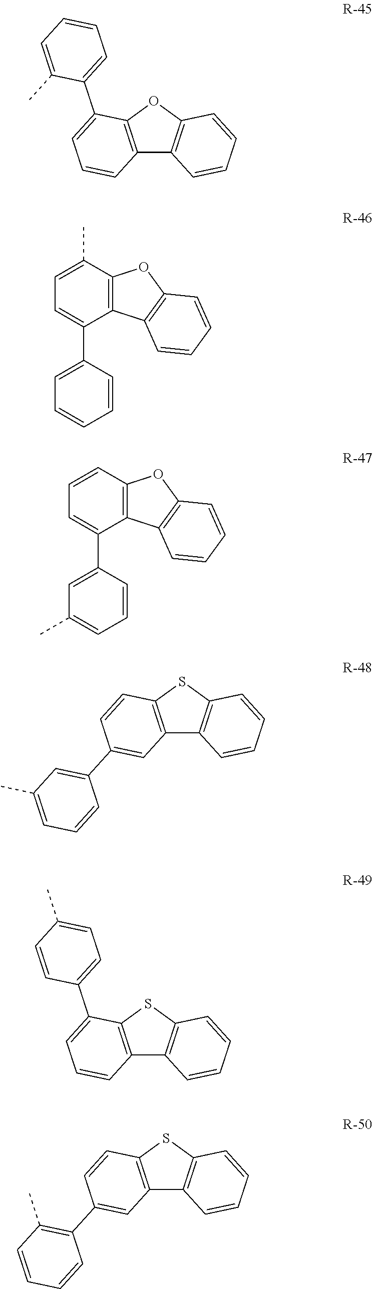

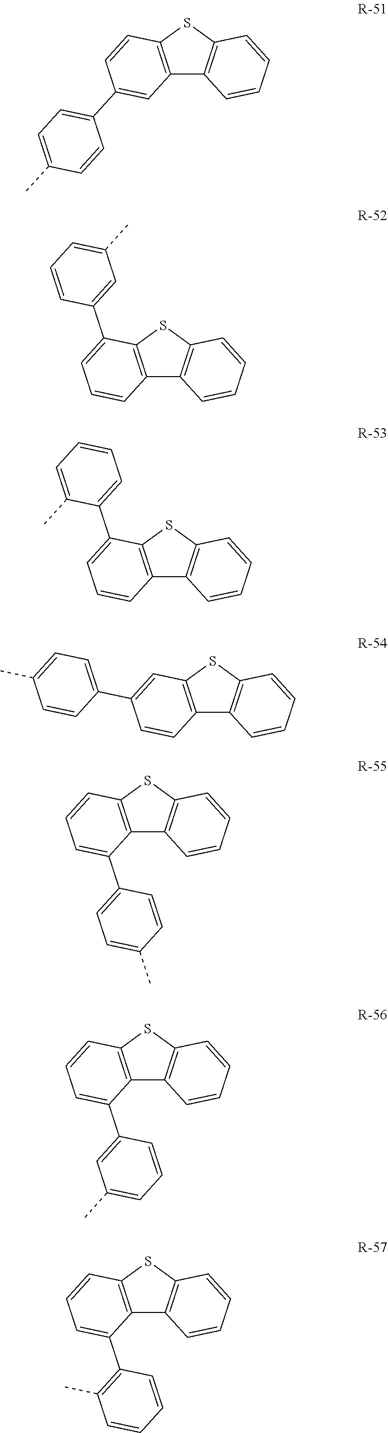

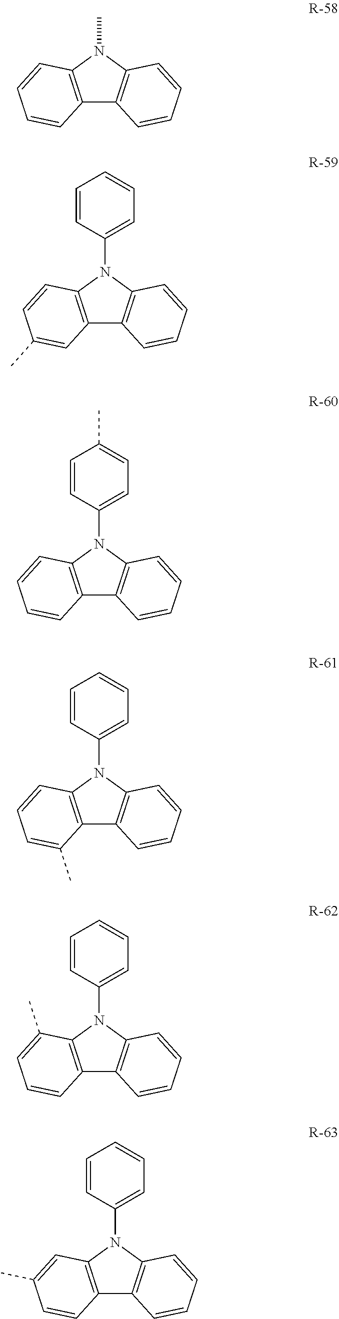

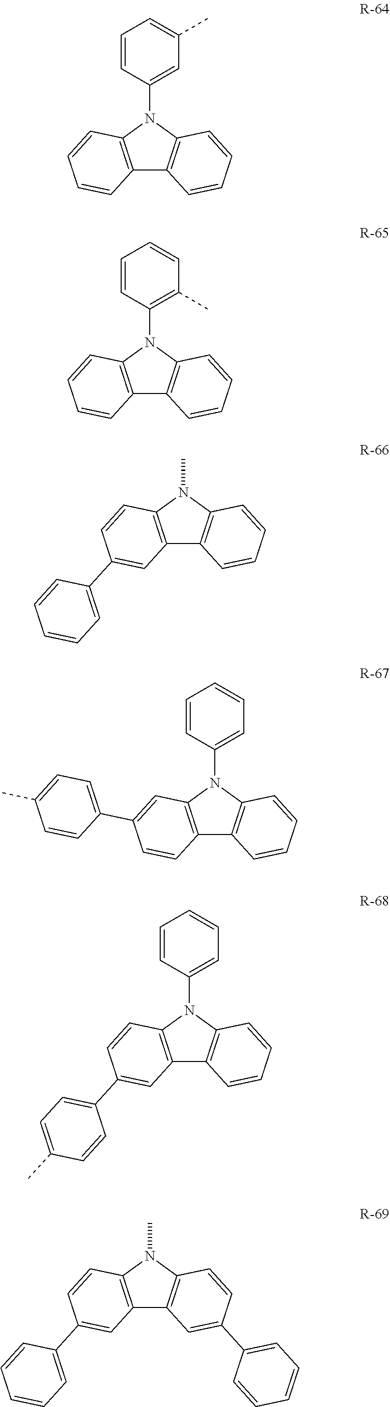

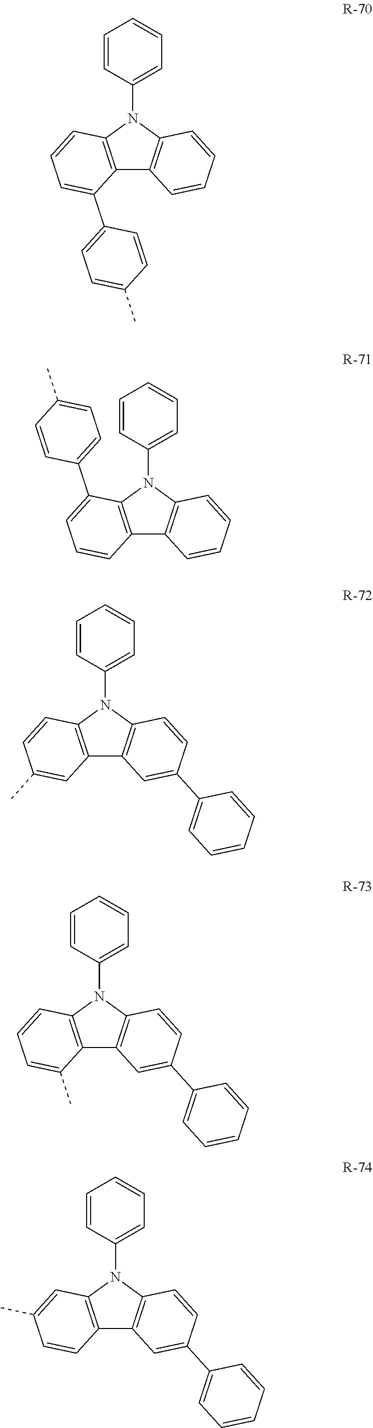

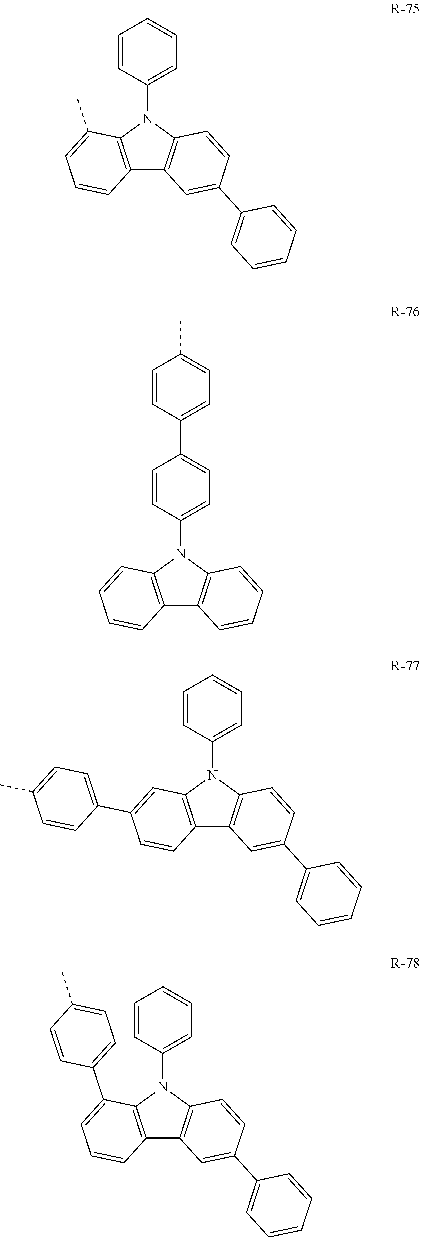

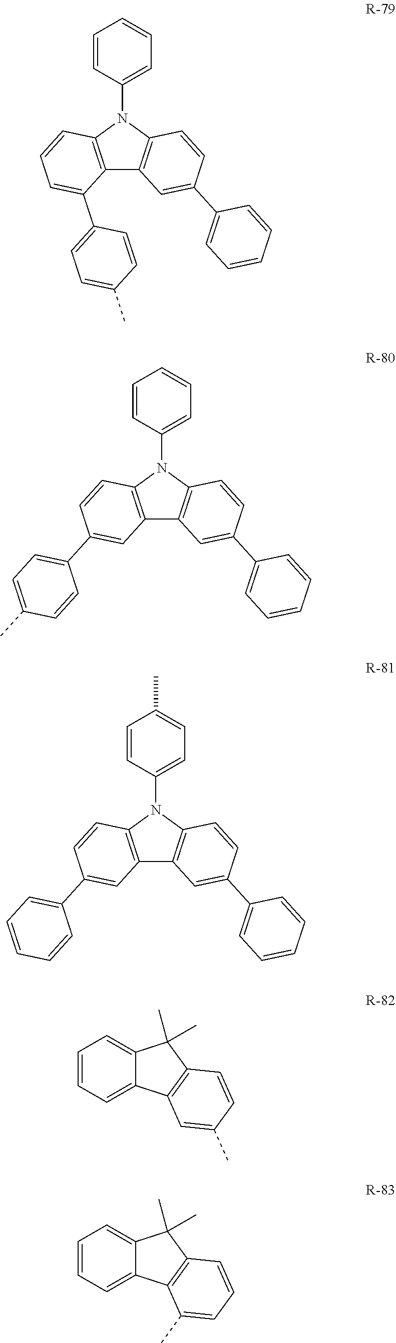

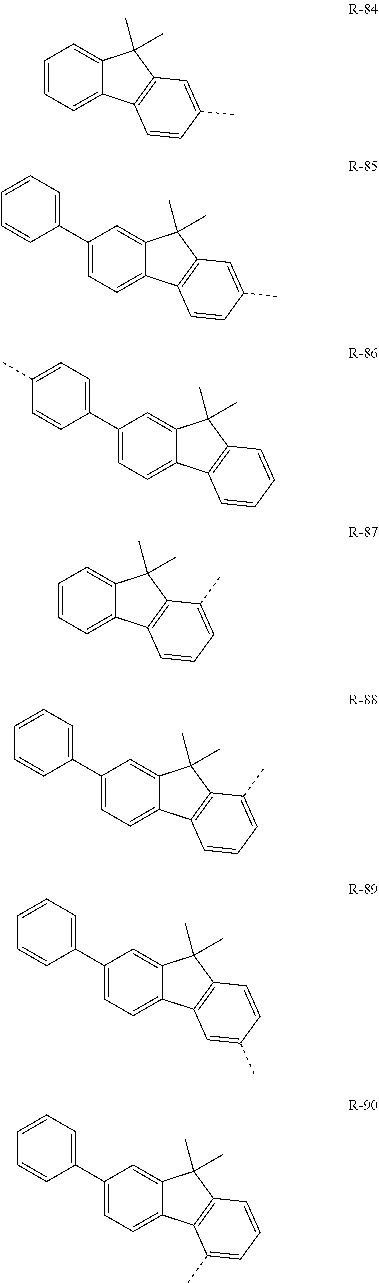

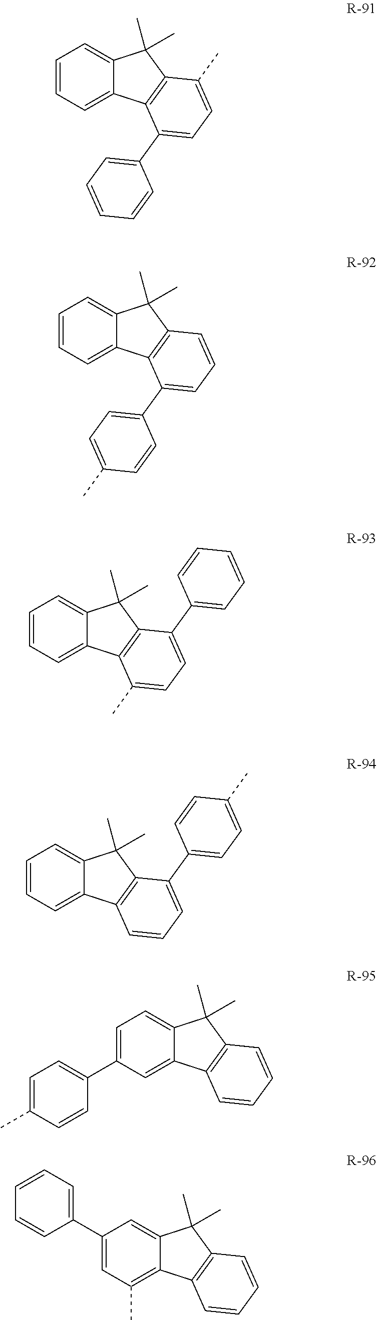

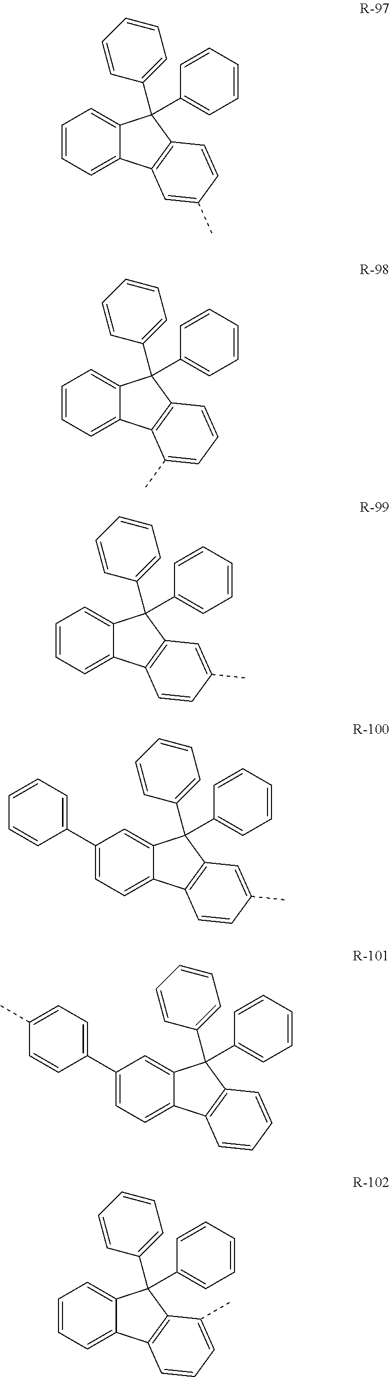

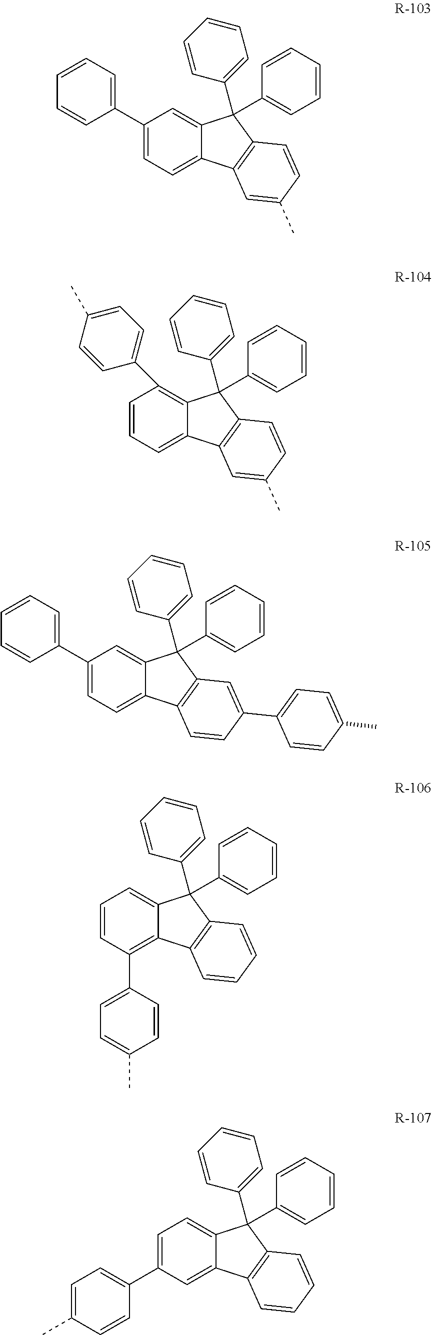

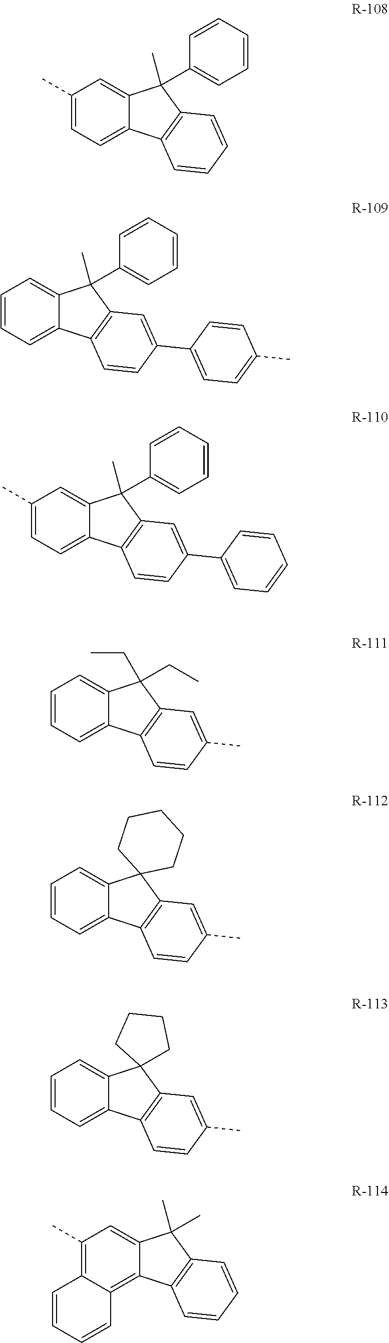

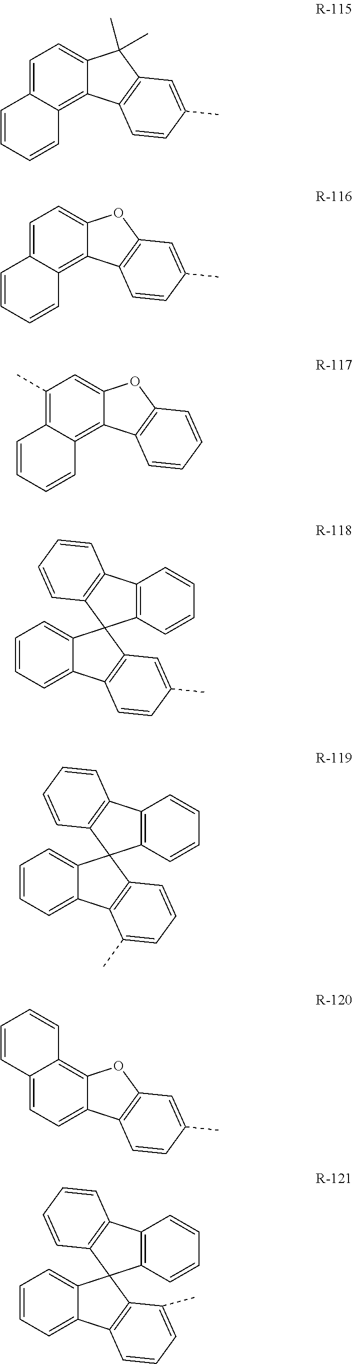

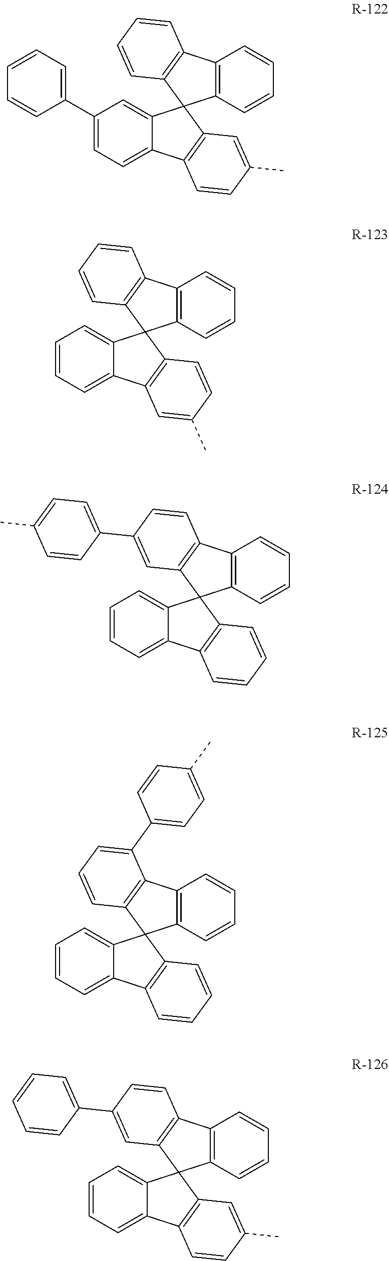

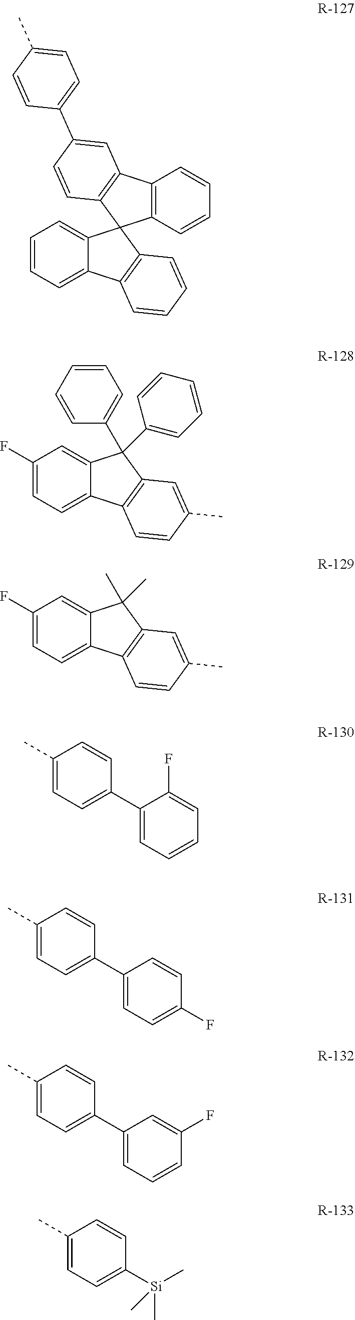

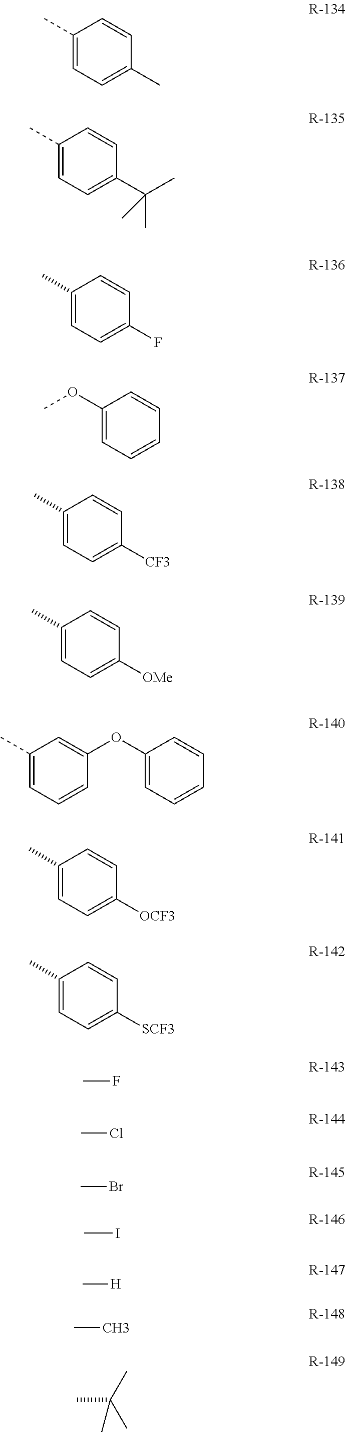

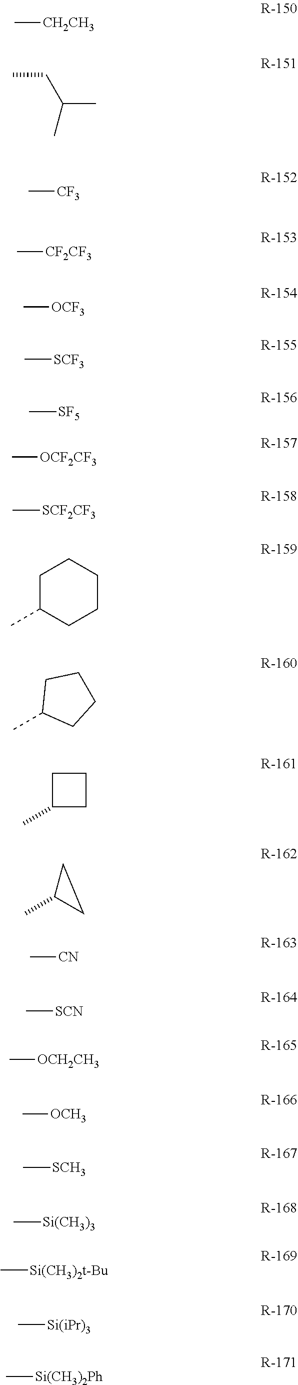

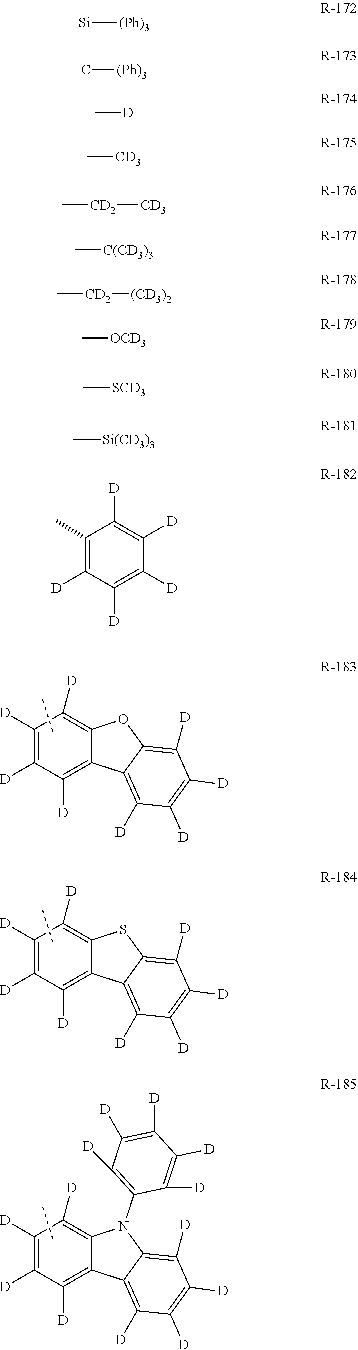

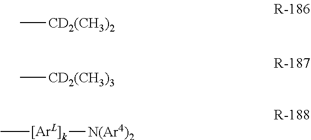

Among these particular preference is given to the formulae R-1, R-2, R-143, R-147, R-148, R-149, R-154.

Preferably, R², R³, R⁴, R⁵ and R⁶ are the same or different at each instance and are selected from H, D, F, CN, Si(R⁷)₃, N(R⁷)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁷ radicals; and where one or more CH₂ groups in the alkyl or alkoxy groups mentioned may be replaced by —C≡C—, —R⁷C═CR⁷—, Si(R⁷)₂, C═O, C═NR⁷, —NR⁷—, —O—, —S—, —C(═O)O— or —C(═O)NR⁷—.

In a preferred embodiment the compound contains at least one, preferably one, group R⁴ which is selected from aromatic ring systems having 6 to 40 aromatic ring atoms and heteroaromatic ring systems having 5 to 40 aromatic ring atoms, each of which are substituted by R⁷ radicals. Particular preference is given to phenyl, biphenyl, terphenyl, naphthyl, phenanthrenyl, fluorenyl, carbazolyl, dibenzofuranyl, dibenzothiophenyl, each of which are substituted by R⁷ radicals.

Preferably, R⁷ is the same or different at each instance and is selected from H, D, F, CN, Si(R⁸)₃, N(R⁸)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁸ radicals; and where one or more CH₂ groups in the alkyl or alkoxy groups mentioned may be replaced by —C≡C—, —R⁸C═CR⁸—, Si(R⁸)₂, C═O, C═NR⁸, —NR⁸—, —O—, —S—, —C(═O)O— or —C(═O)NR⁸—.

Index k is preferably selected from 0, 1 and 2, particularly preferably selected from 0 and 1, very particularly preferably index k is 0.

Ar^{L} is preferably the same or different at each instance and is selected from aromatic or heteroaromatic rings having 6 aromatic ring atoms and aromatic or heteroaromatic ring systems having 10 aromatic ring atoms, particularly preferably selected from phenyl, biphenyl, naphthyl and fluorenyl, each of which are substituted by R² radicals; very particularly preferably selected from phenyl substituted by R² radicals.

Preferably, Ar^{L} is the same or different at each instance and is selected from groups of the following formulae:

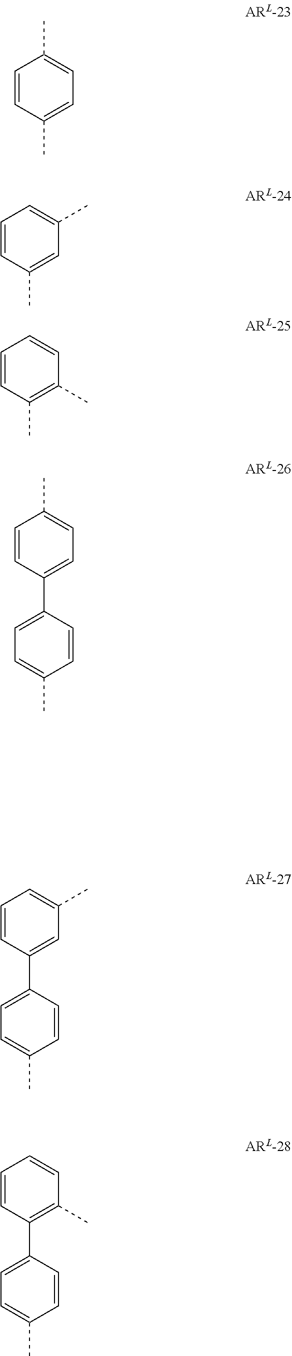

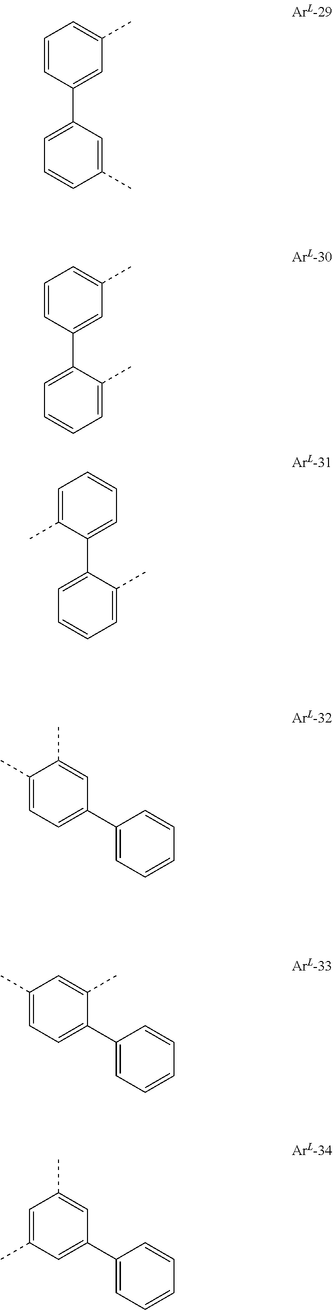

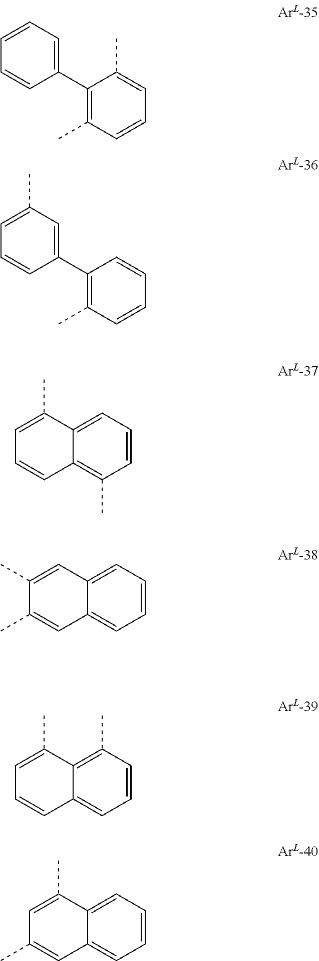

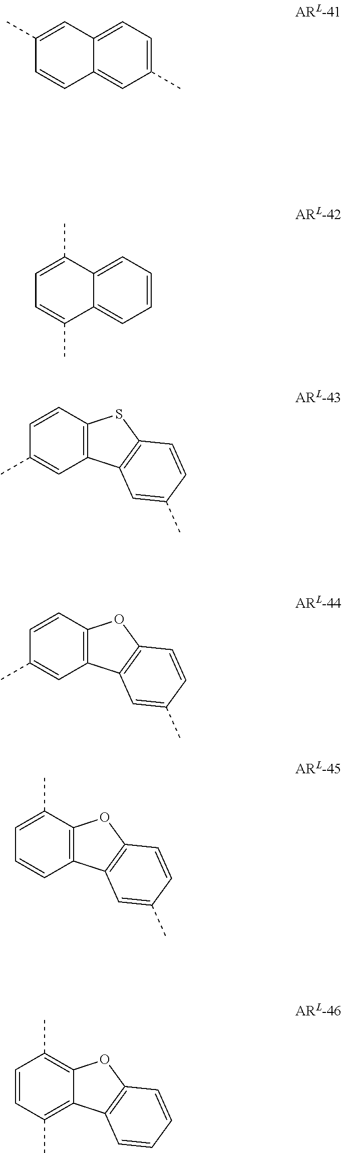

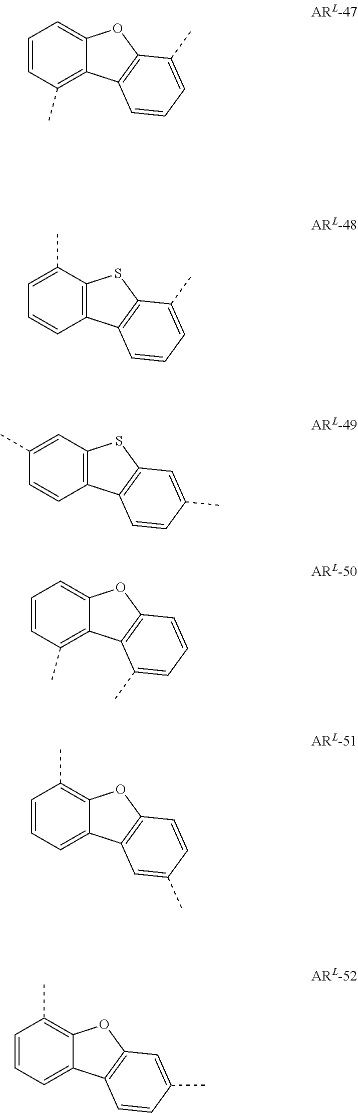

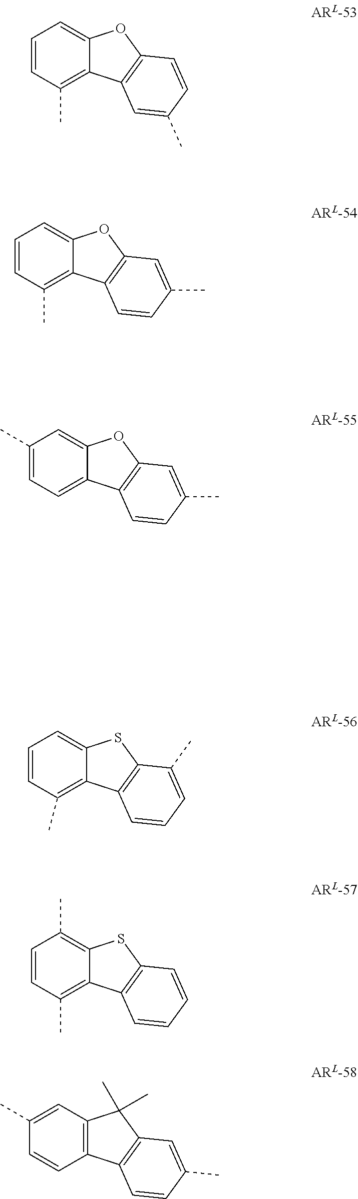

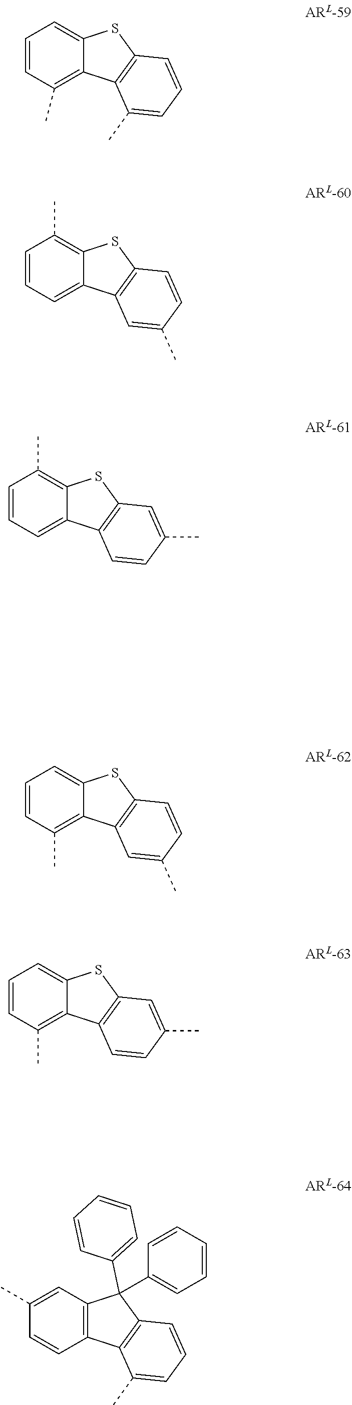

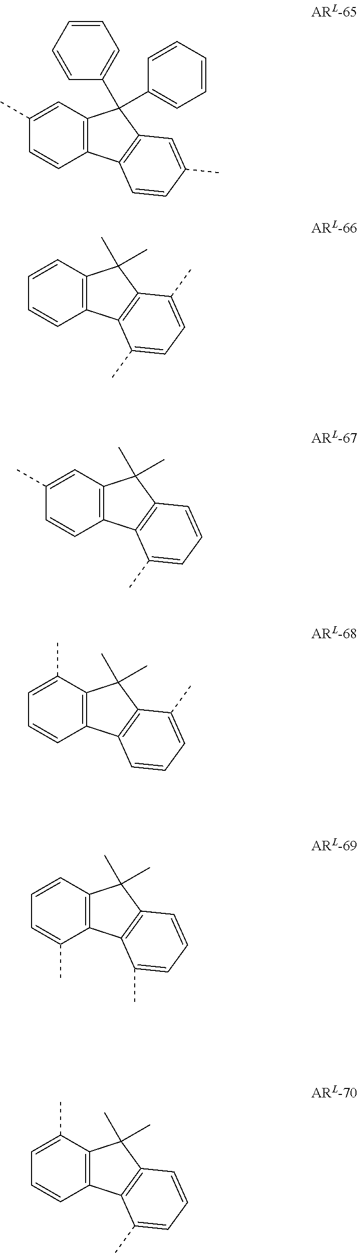

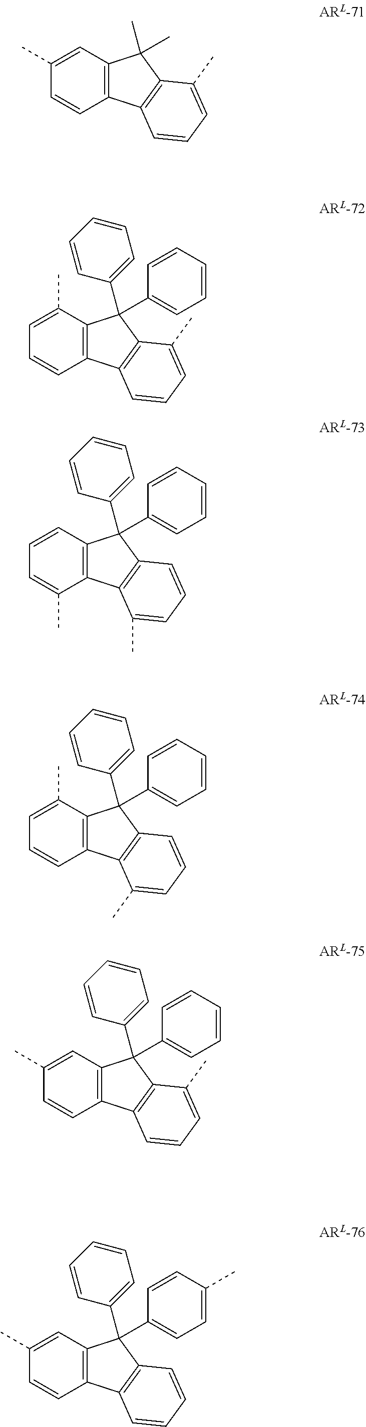

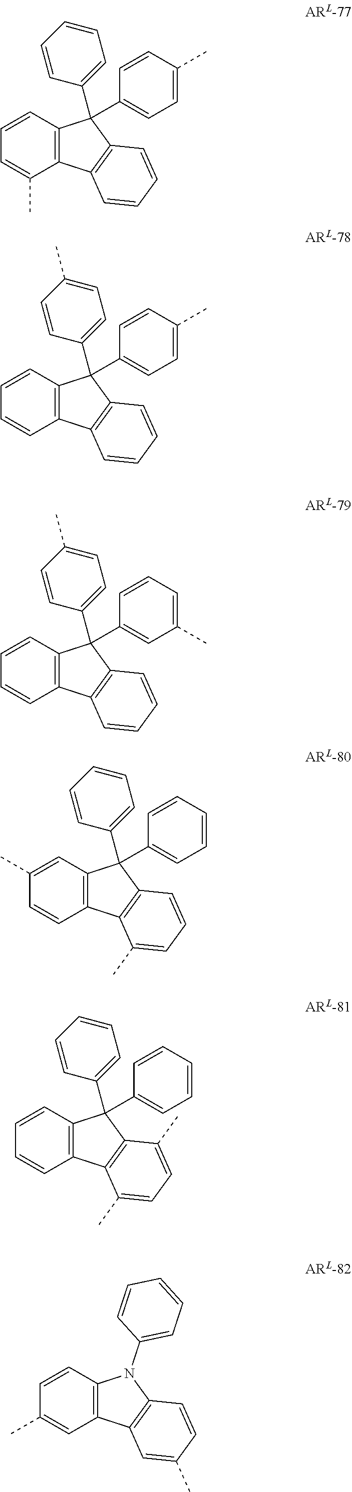

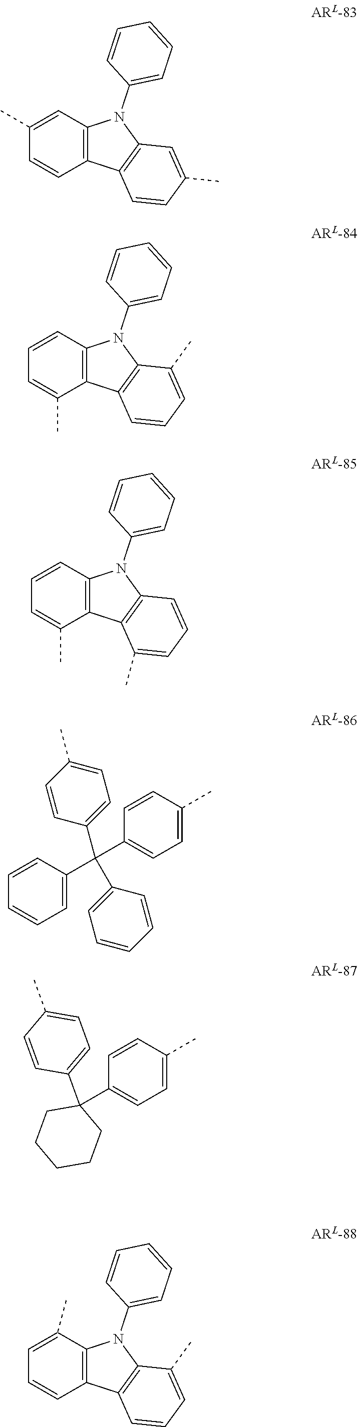

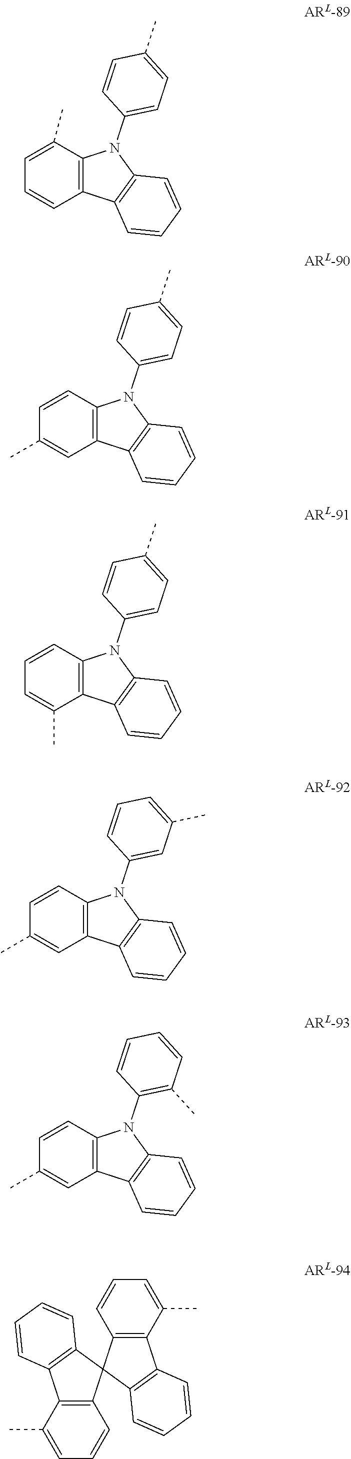

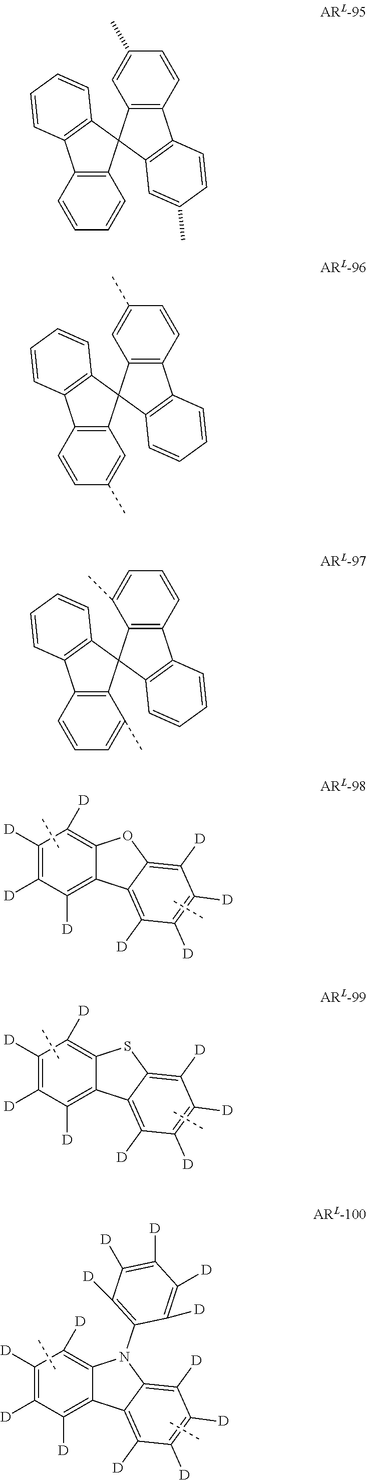

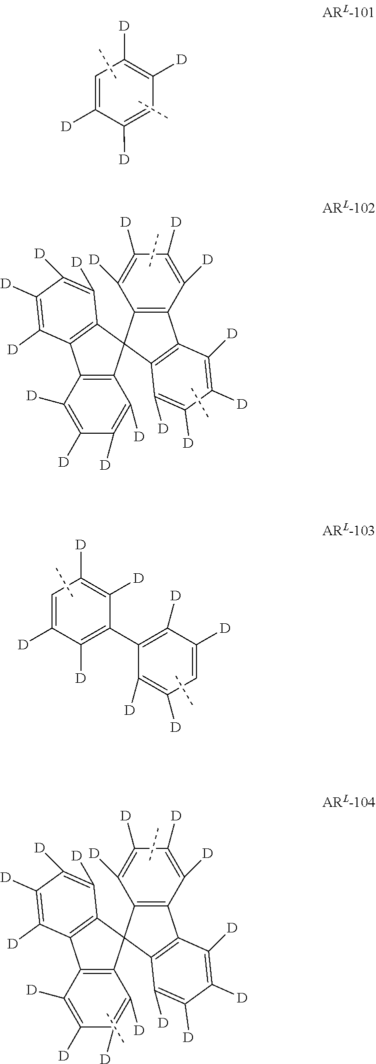

where the dotted lines represent the bonds to the rest of the formula. Among the abovementioned formulae, particular preference is given to the formulae Ar^{L}-23, Ar^{L}-24, Ar^{L}-25, Ar^{L}-26, Ar^{L}-37, Ar^{L}-42, Ar^{L}-47, Ar^{L}-58.

Index m is preferably 0 or 1. In an alternative preferred embodiment index m is 2 or 3, more preferably 2.

Preferably, Ar¹ is the same or different at each instance and is selected from aromatic or heteroaromatic ring systems which have 6 aromatic ring atoms and are substituted by R³ radicals, from aromatic or heteroaromatic ring systems which have 10 aromatic ring atoms and are substituted by R³ radicals and from fluorenyl substituted by R³ radicals. It is particularly preferable when Ar¹ is the same or different at each instance and is selected from phenyl, naphthyl, fluorenyl and biphenyl, each substituted by R³ radicals. It is very particularly preferable when Ar³ is phenyl substituted by R³ radicals.

The unit

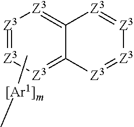

is preferably a unit

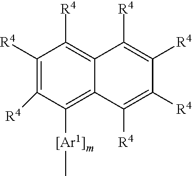

so that the compounds of the formula (I) correspond to a preferred formula (I-sub):

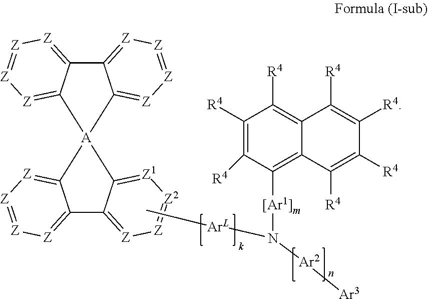

The unit

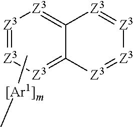

is particularly preferably selected from units of the formula

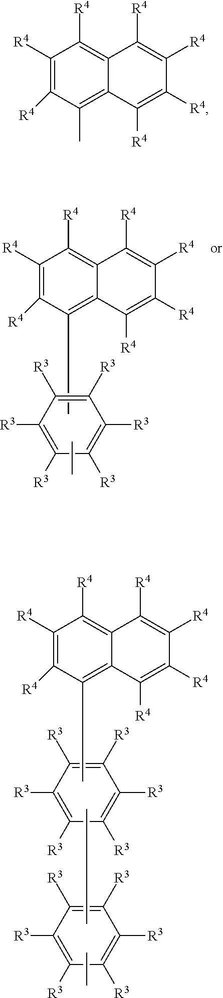

so that the compounds of the formula (I) correspond to a preferred formula (I-sub-1), (I-sub-2) or (I-sub-3):

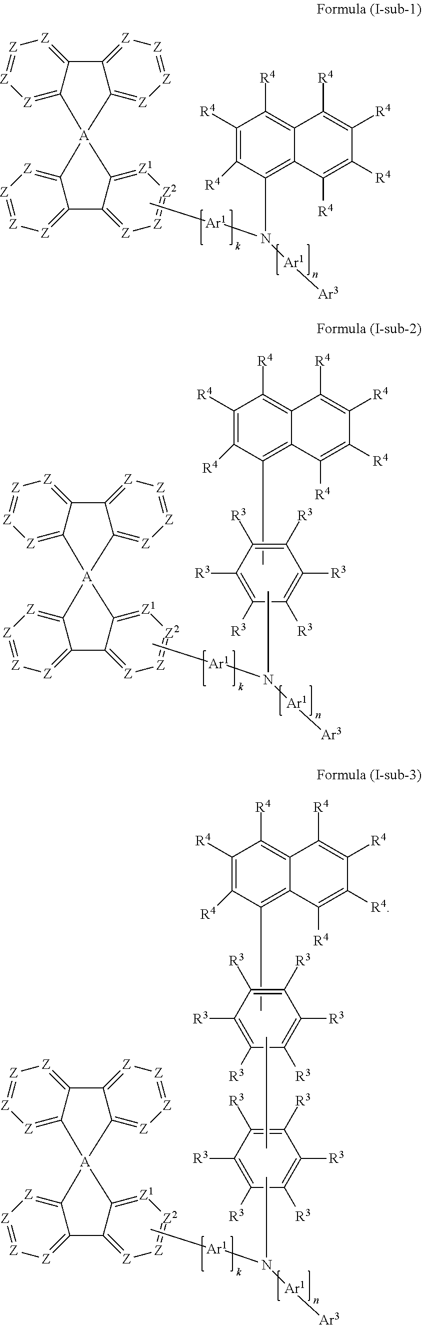

Preferred units

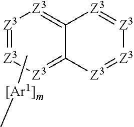

are selected from the following:

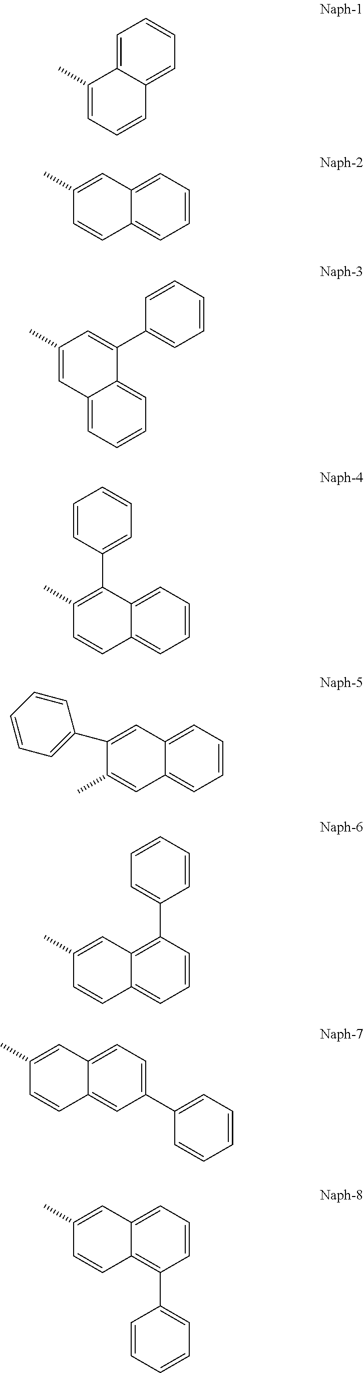

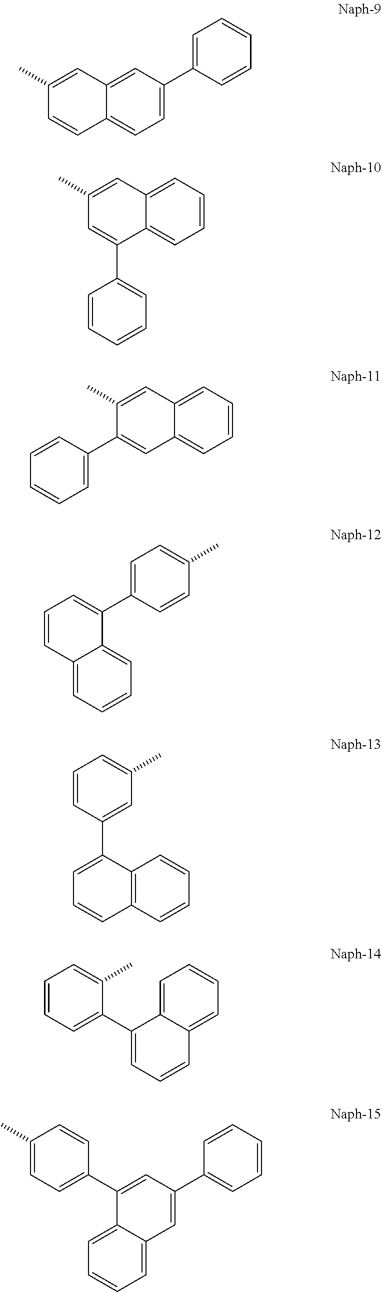

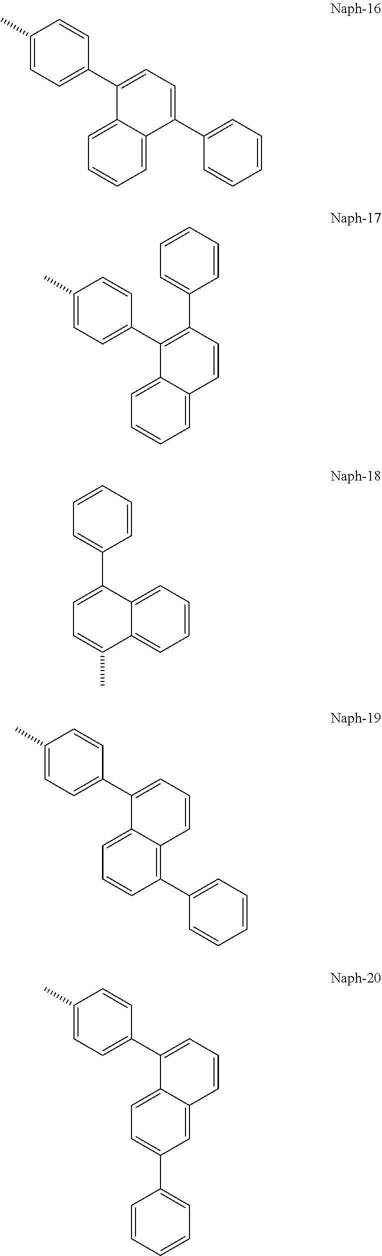

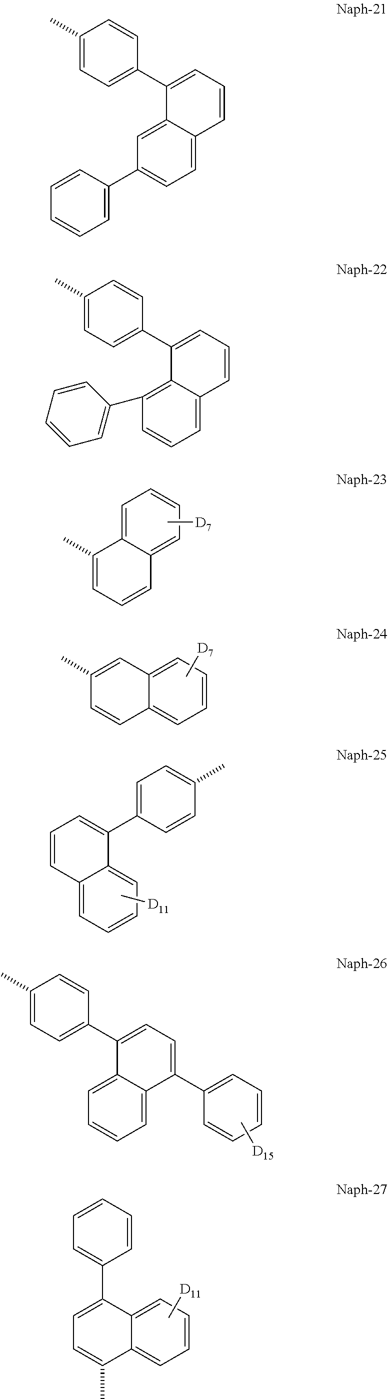

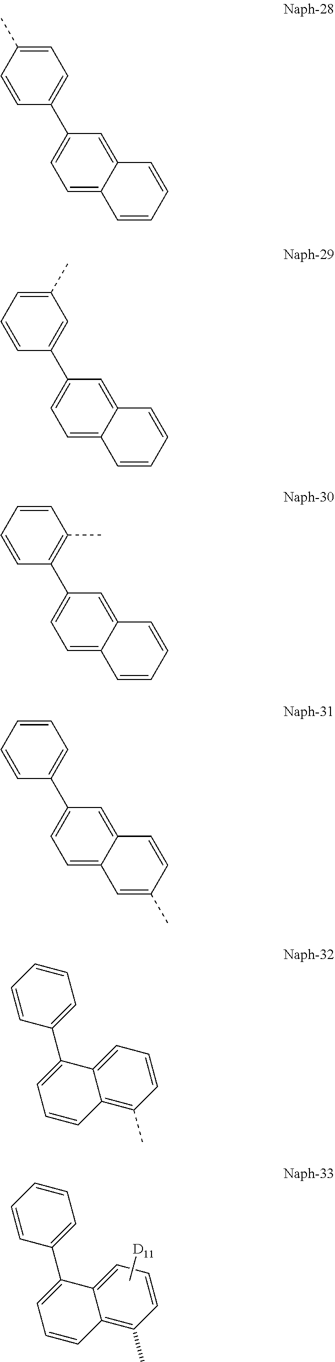

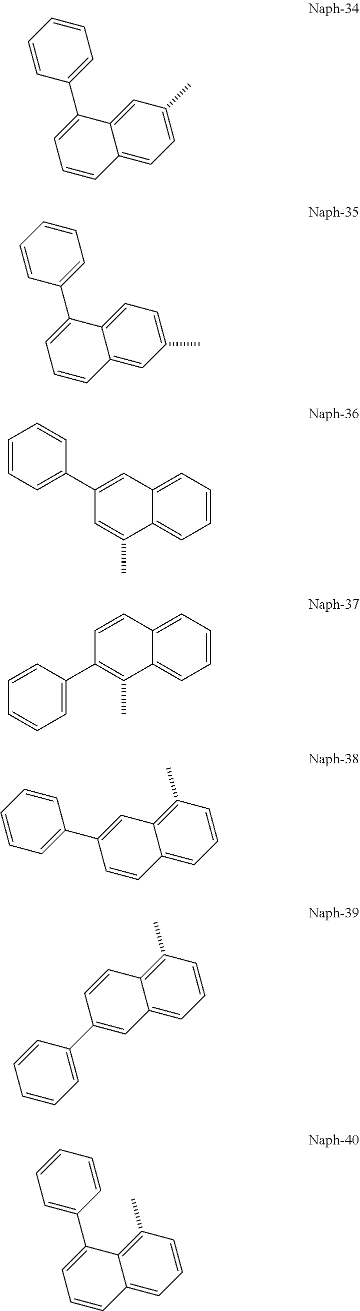

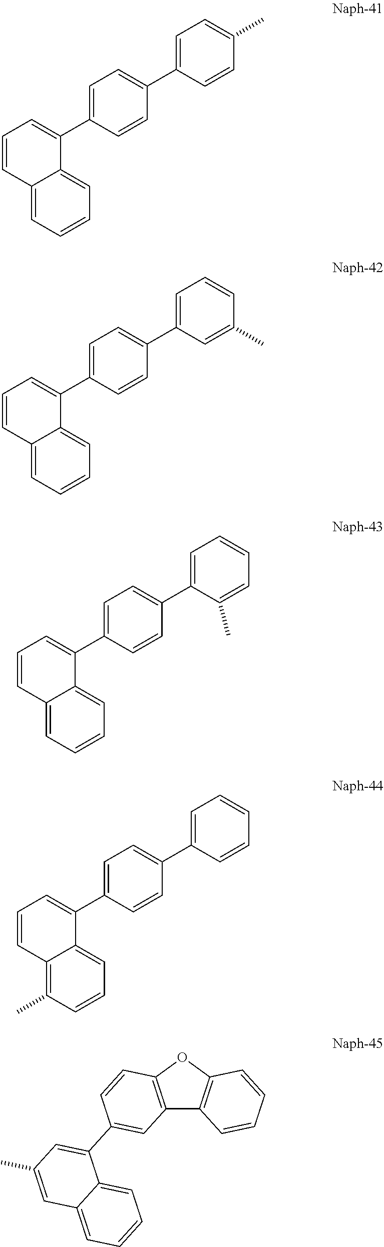

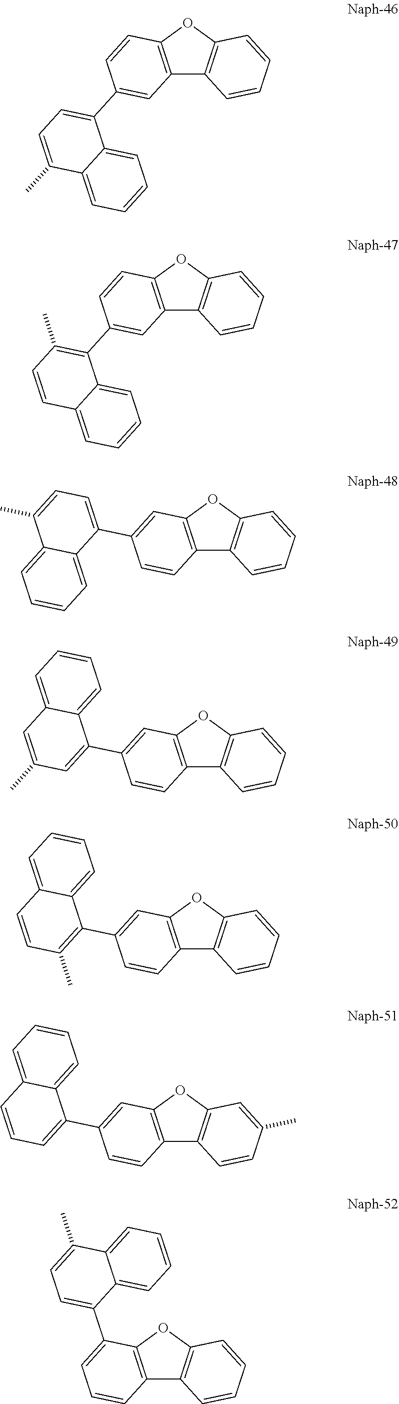

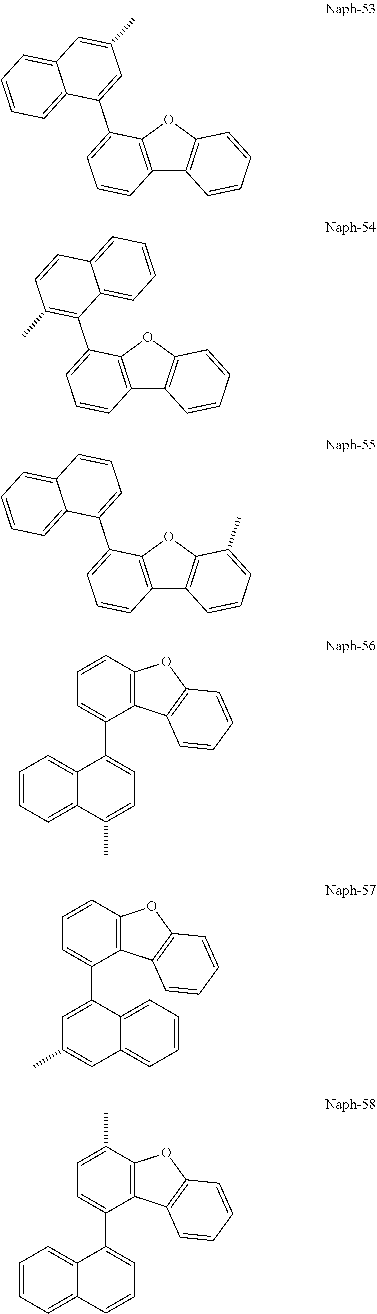

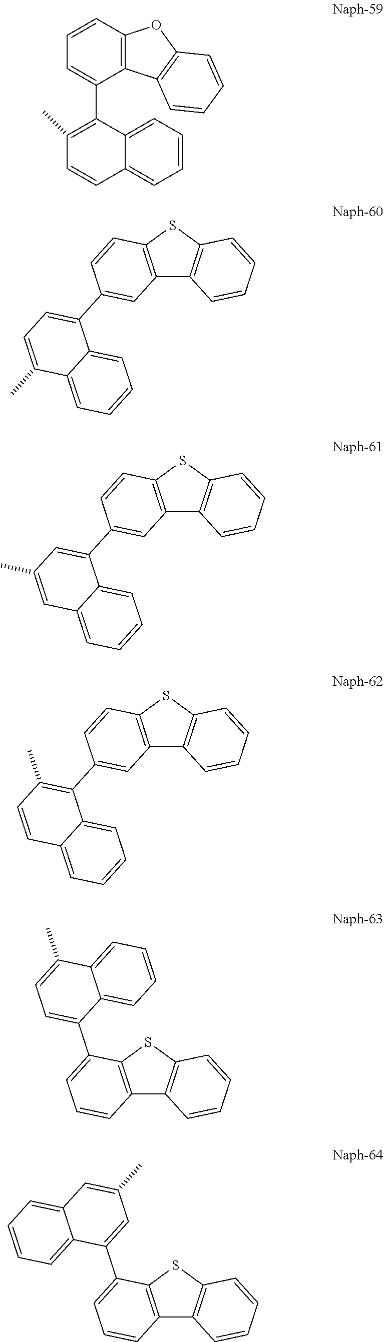

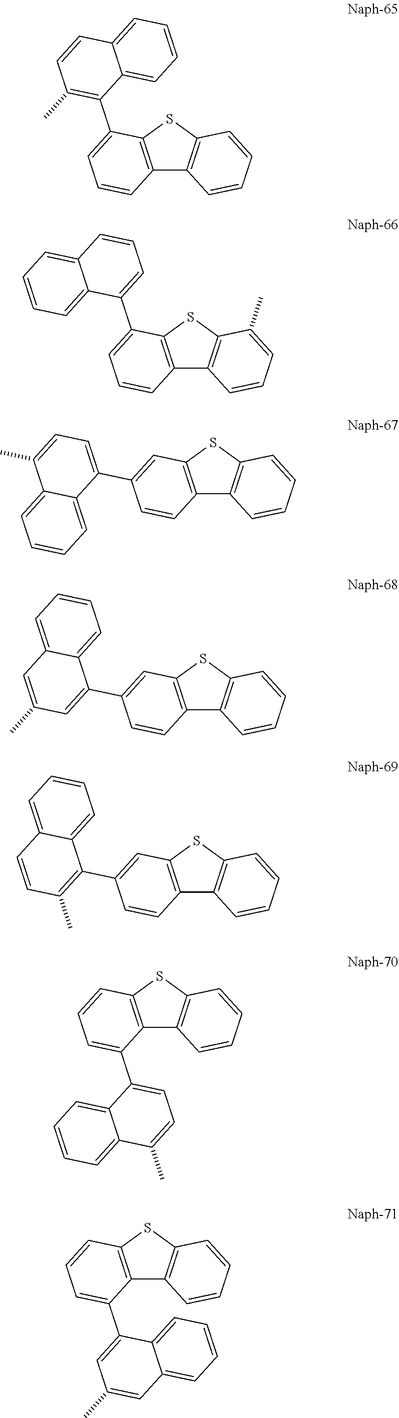

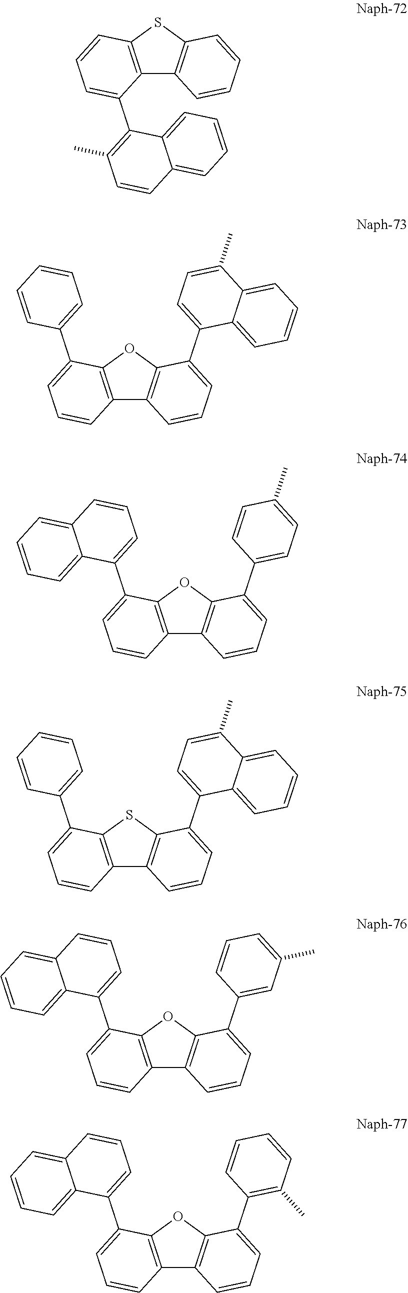

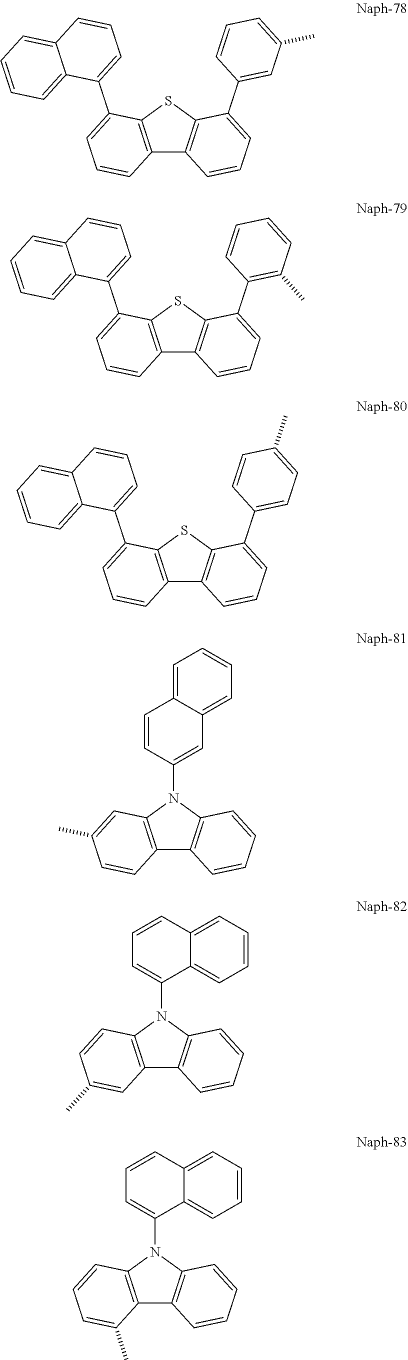

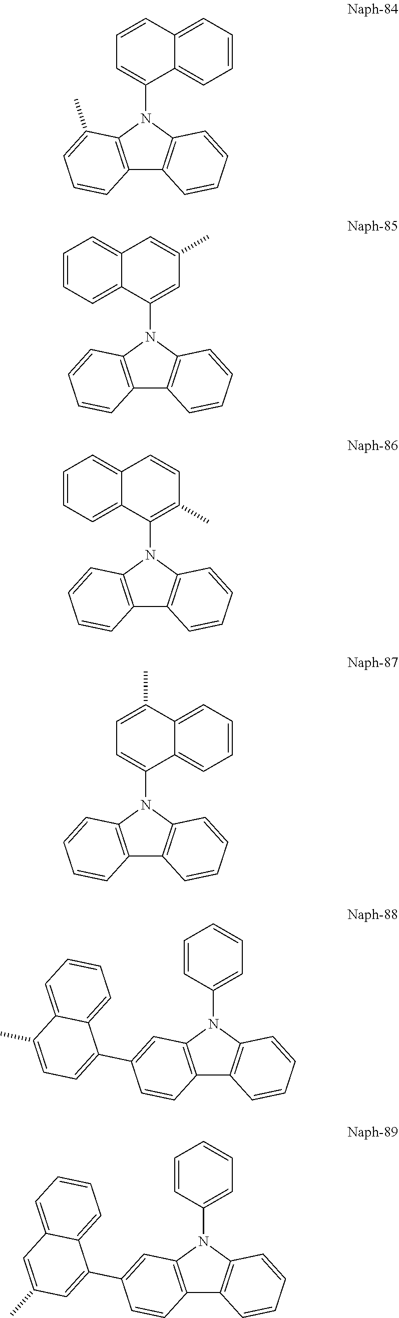

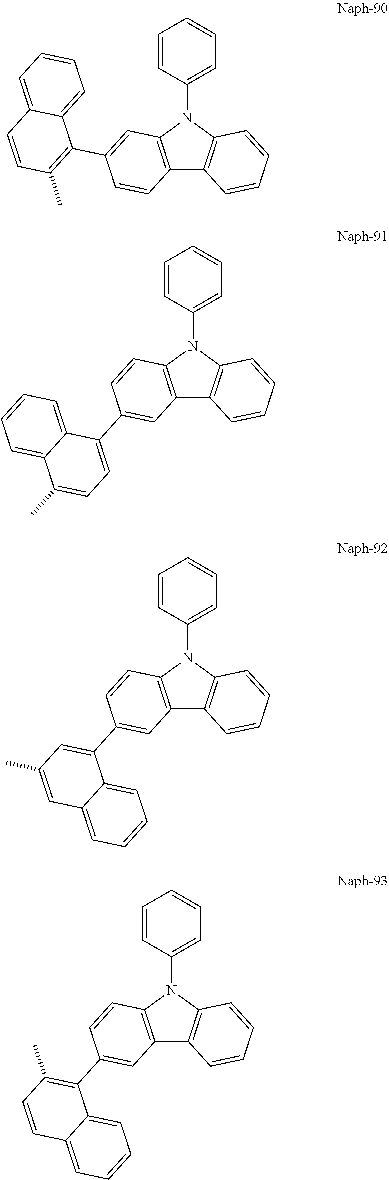

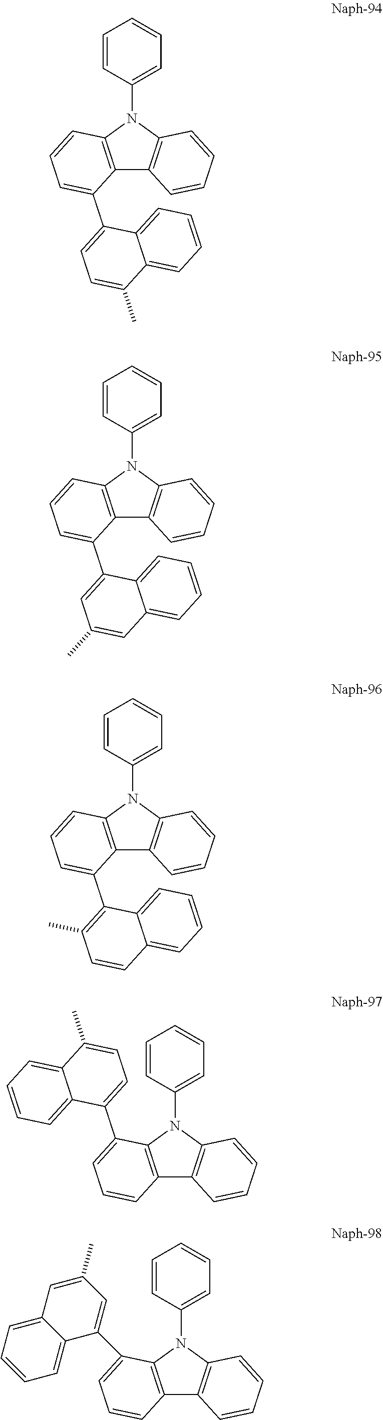

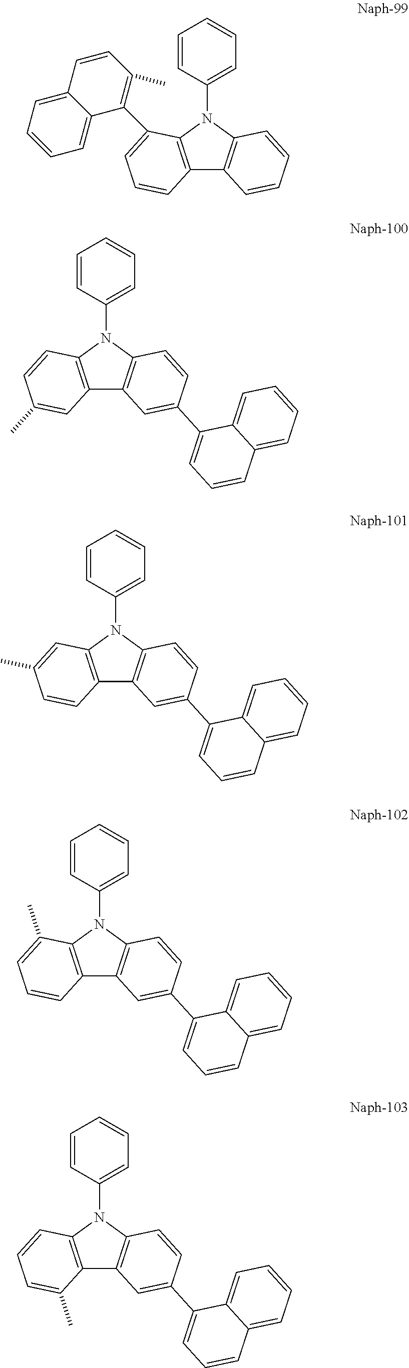

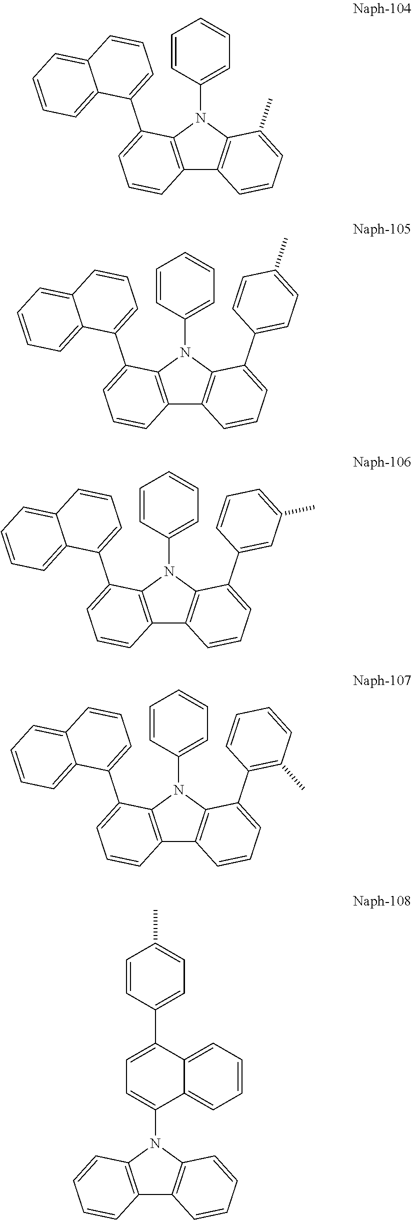

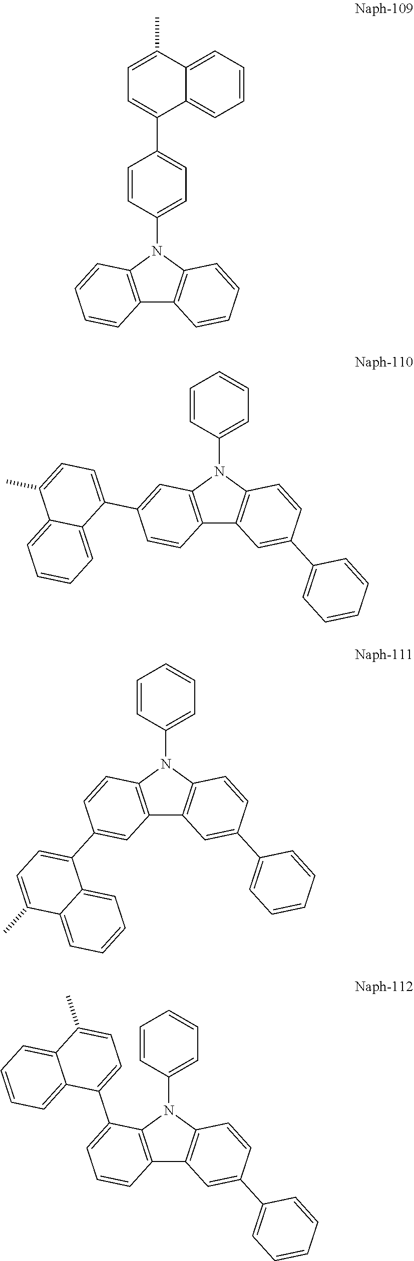

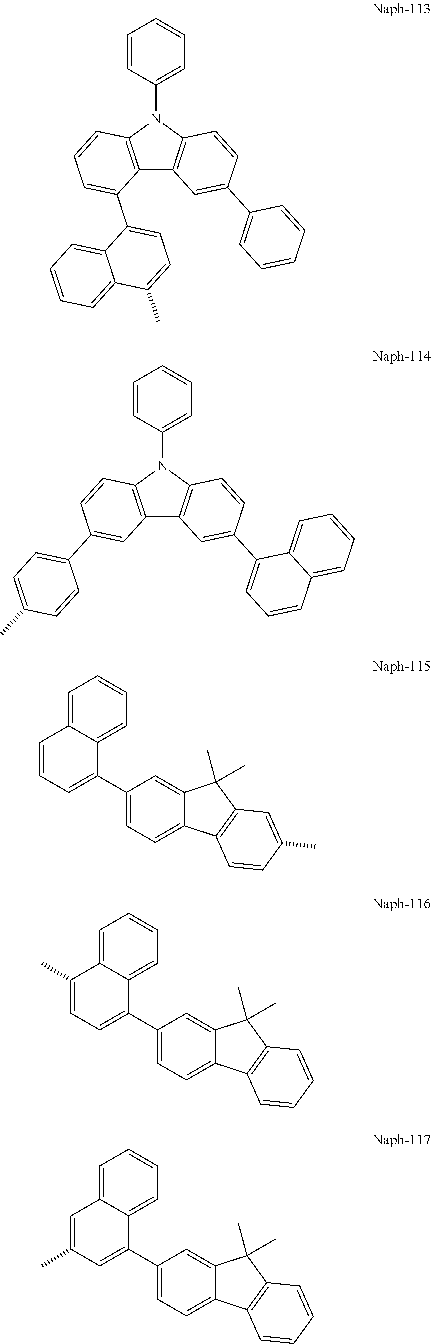

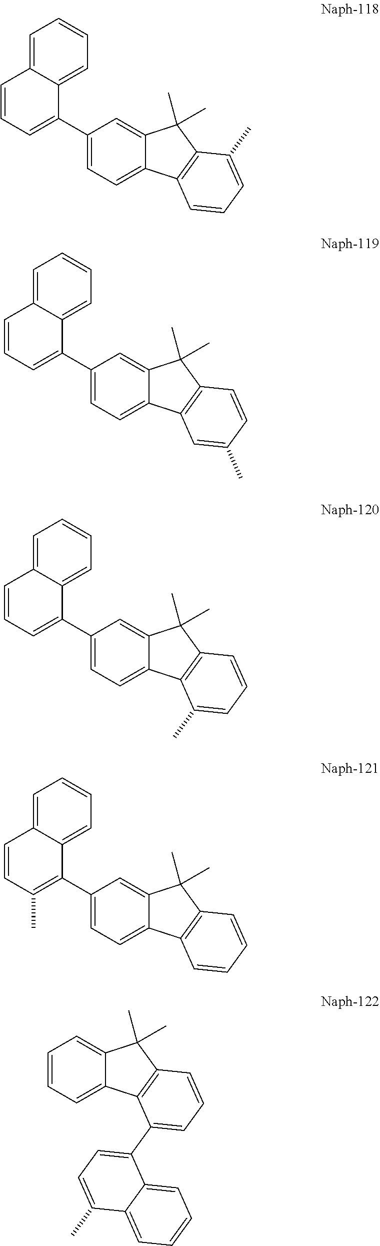

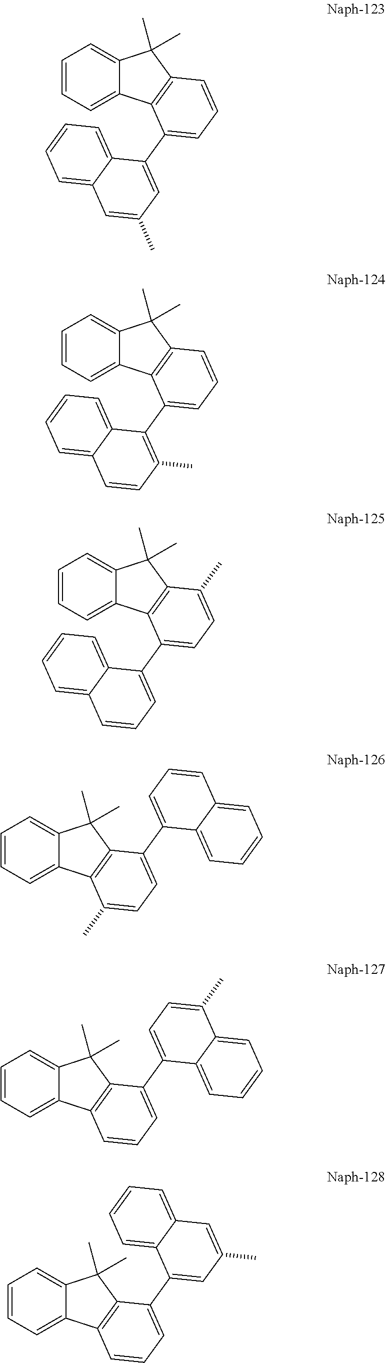

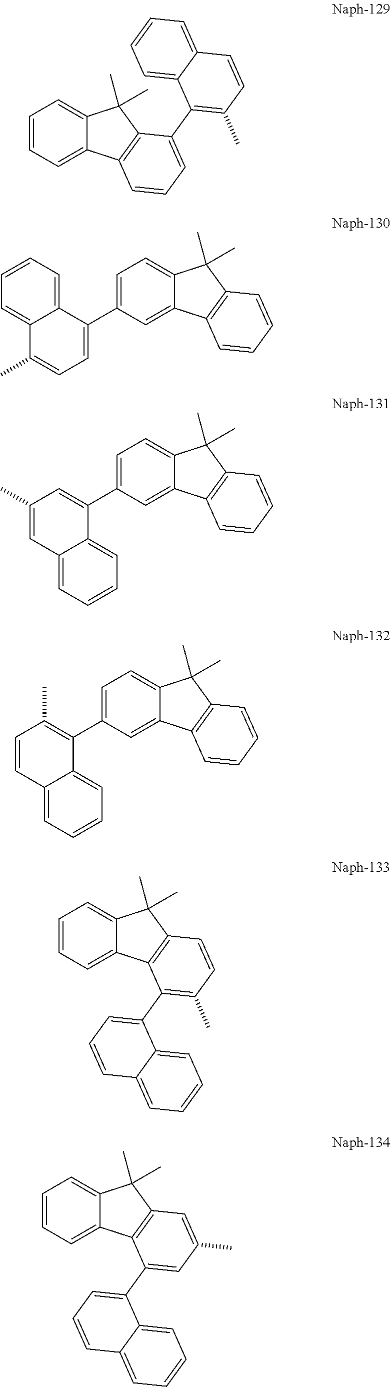

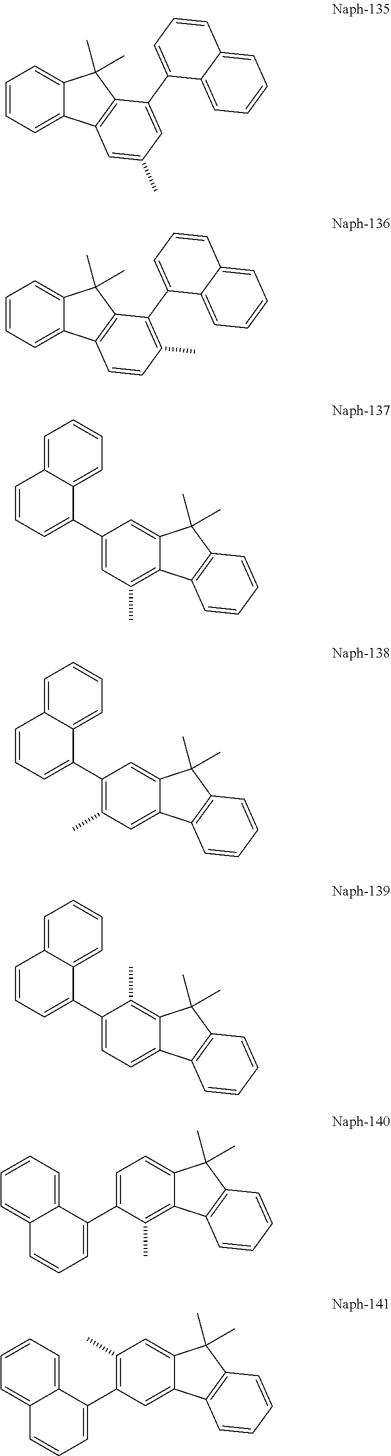

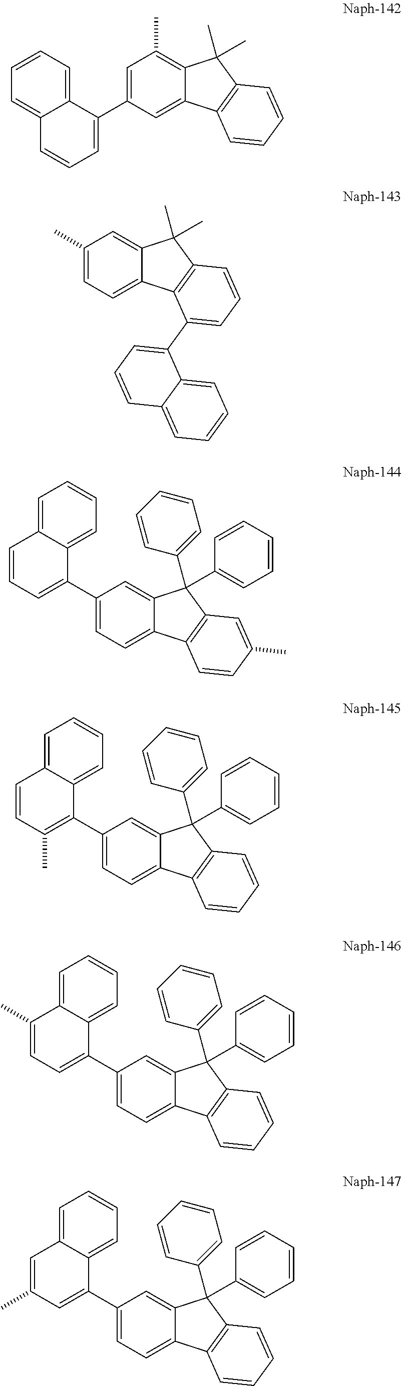

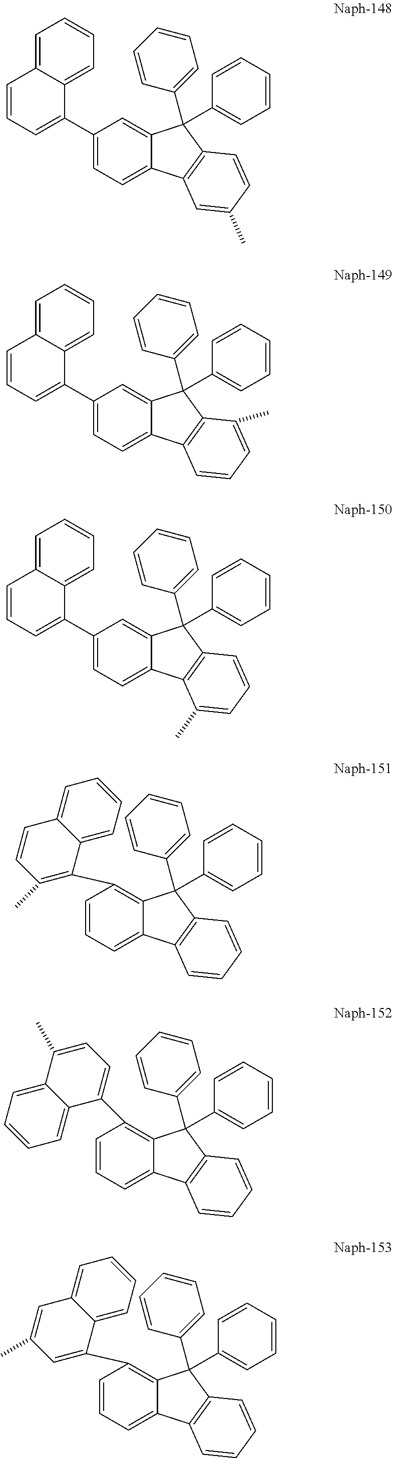

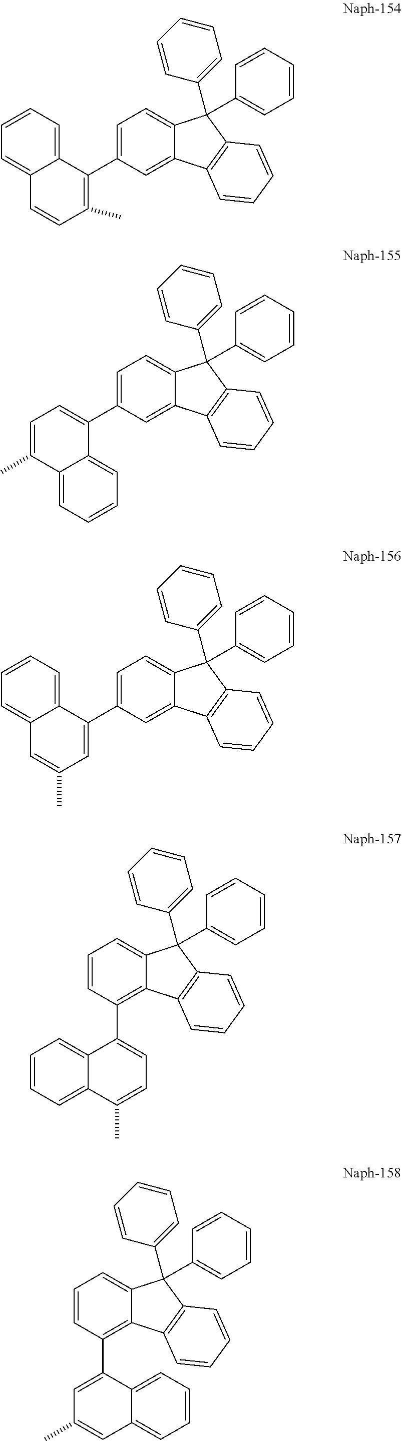

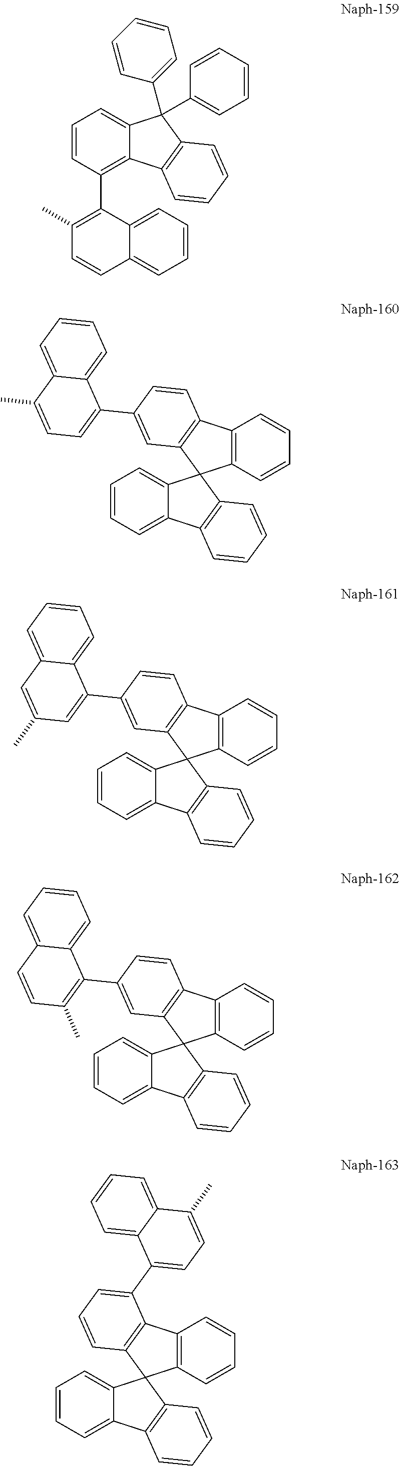

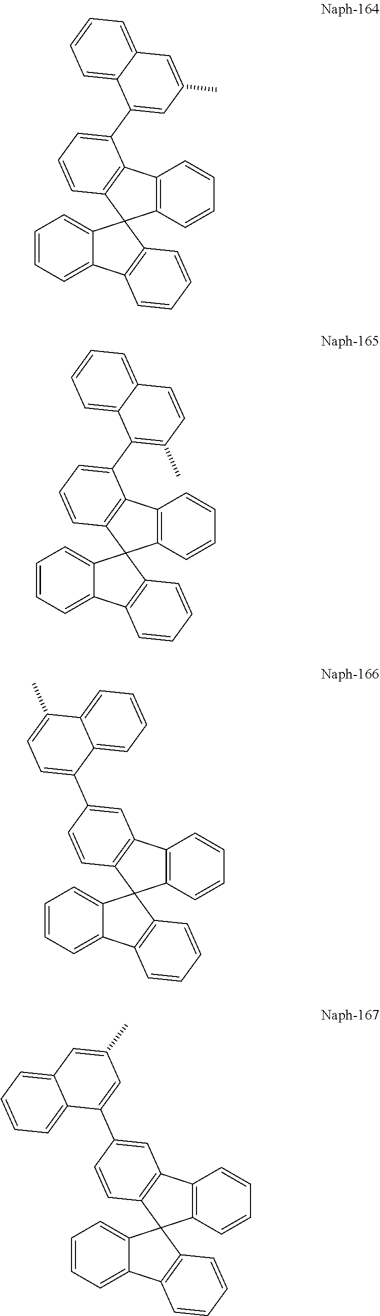

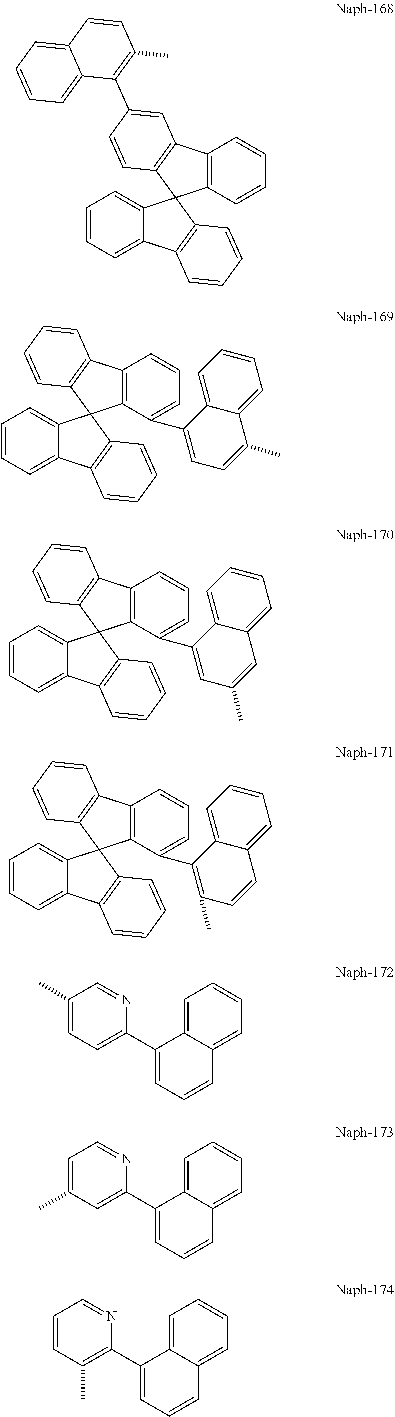

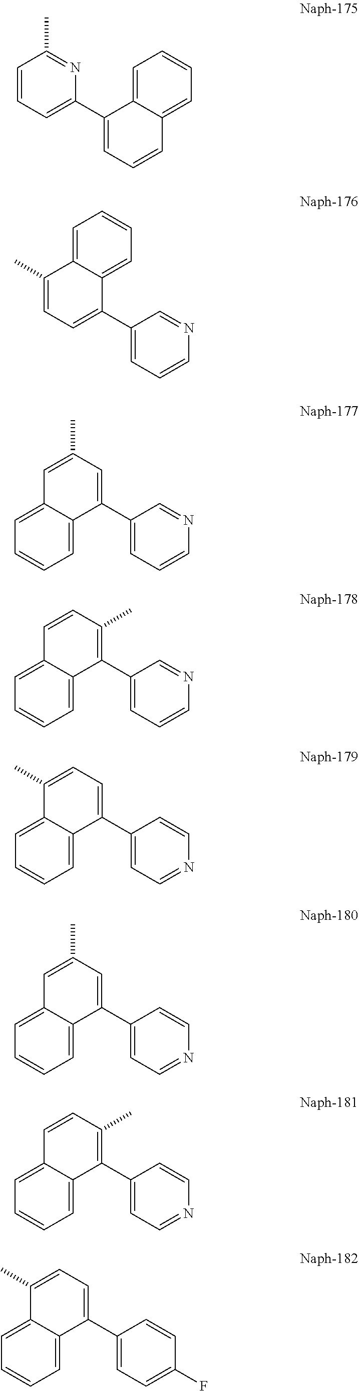

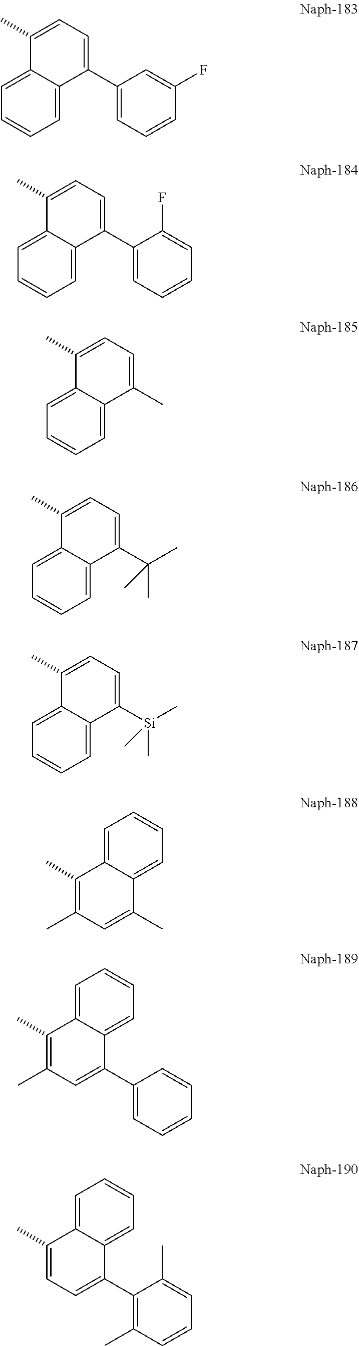

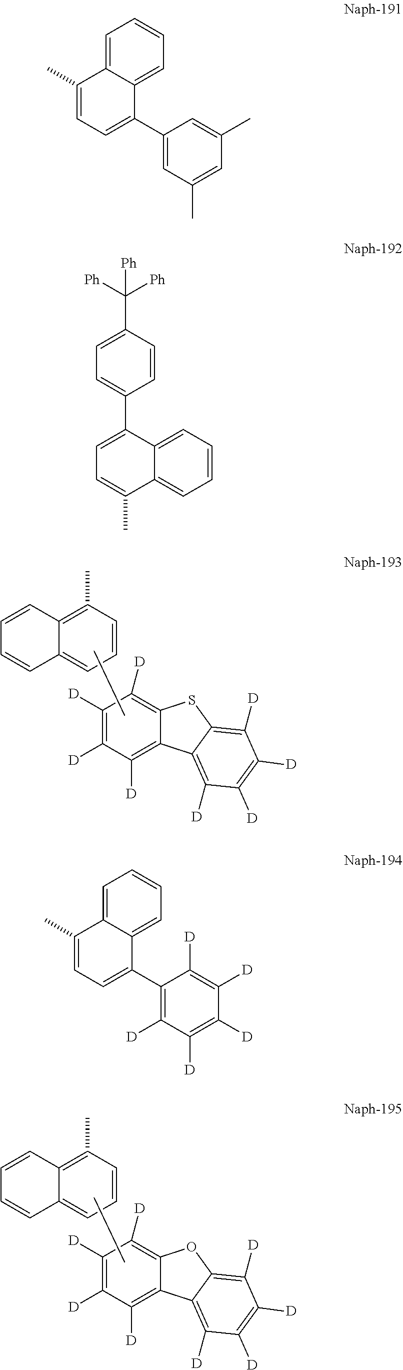

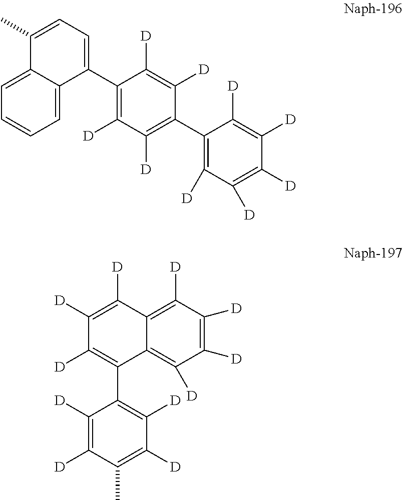

Among these groups particular preference is given to the groups Naph-1, Naph-12, Naph-18, Naph-23, Naph-25, Naph-26, Naph-27, Naph-41, Naph-52, Naph-56, Naph-82, Naph-87, Naph-108, Naph-109, Naph-116, Naph-122, Naph-194, Naph-195, Naph-197.

Preferably, Ar² is the same or different at each instance and is selected from phenyl and naphthyl, each of which are substituted by R⁵ radicals, very particularly preferably phenyl substituted by R⁵ radicals. Phenyl may be ortho-phenylene, meta-phenylene or para-phenylene, wherein para-phenylene is preferred.

Preferably, n is 1 or 2.

It is very particularly preferable when the unit —[Ar²]ₙ— is phenylene substituted by R⁵ radicals or biphenylene substituted by R⁵ radicals. Para-phenylene units are preferred over meta- or ortho-phenylene units.

Preferably, Ar³ is selected from groups of the formulae (Ar³-2), (Ar³-3) and (Ar³-4), particularly preferably groups of the formulae (Ar³-2) and (Ar³-3). In these cases it is preferable when Z⁴ is CR⁶ or C when the bond to the rest of the formula proceeds via this Z⁴.

Preferred Ar³ groups are selected from the following groups:

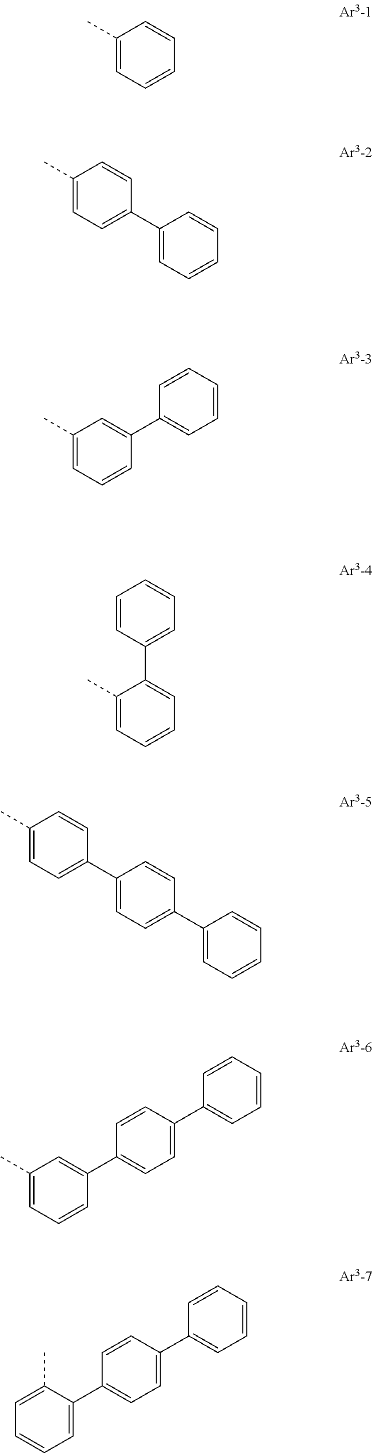

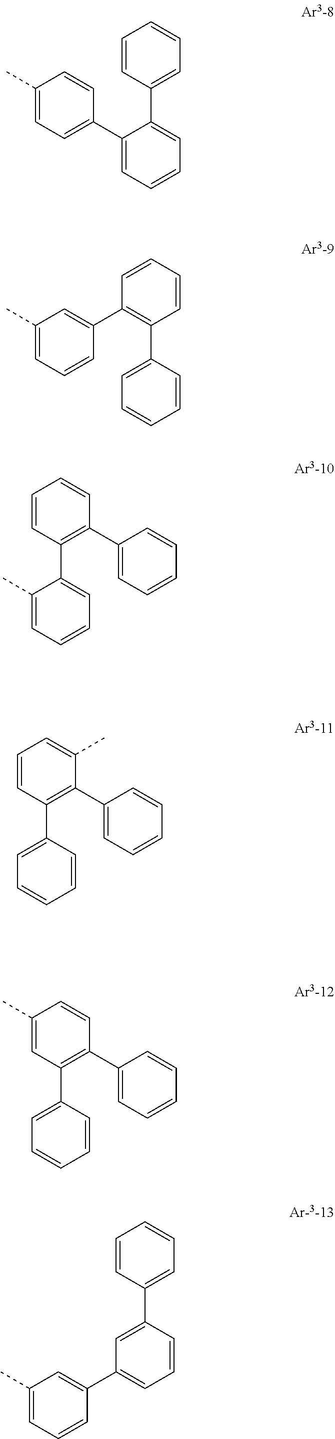

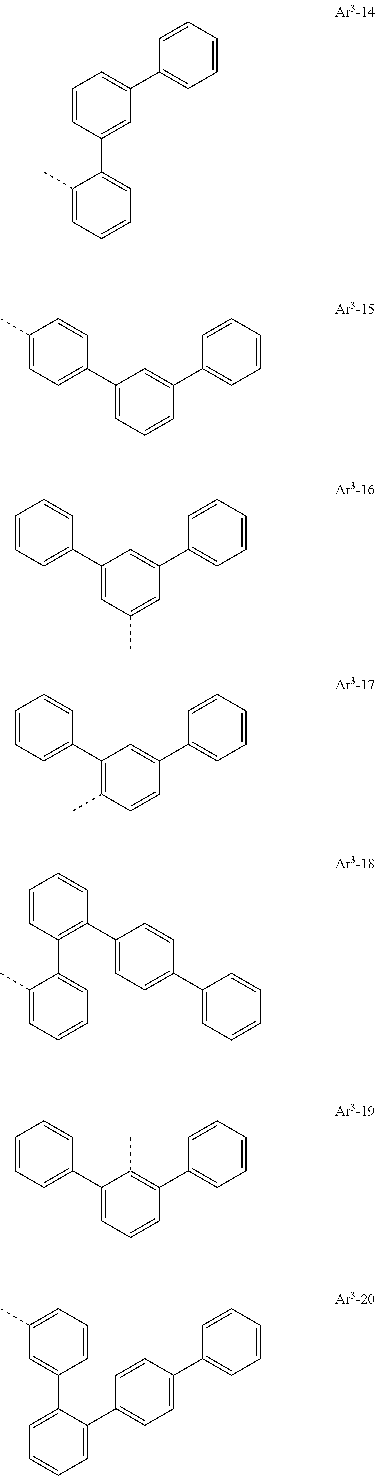

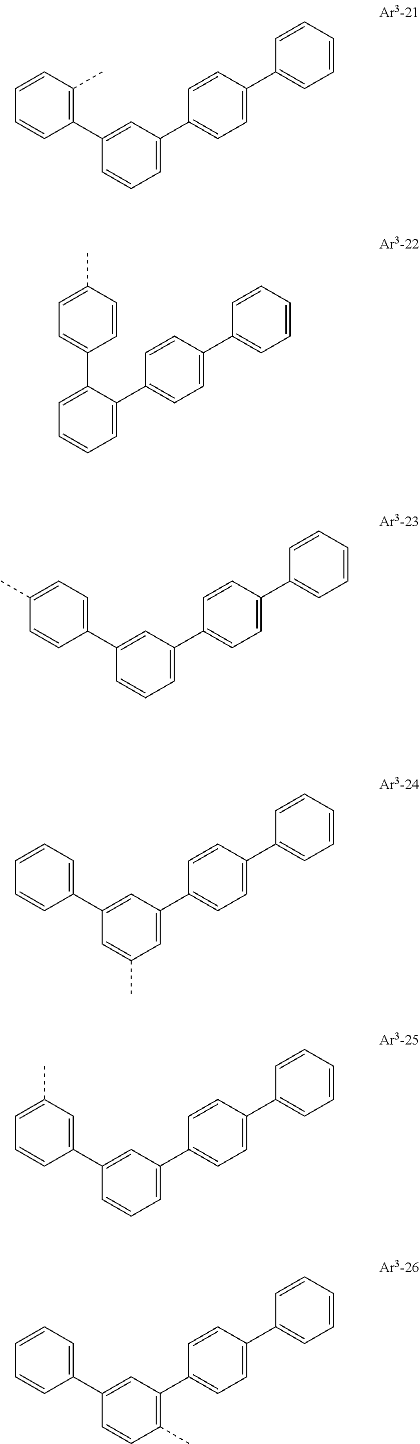

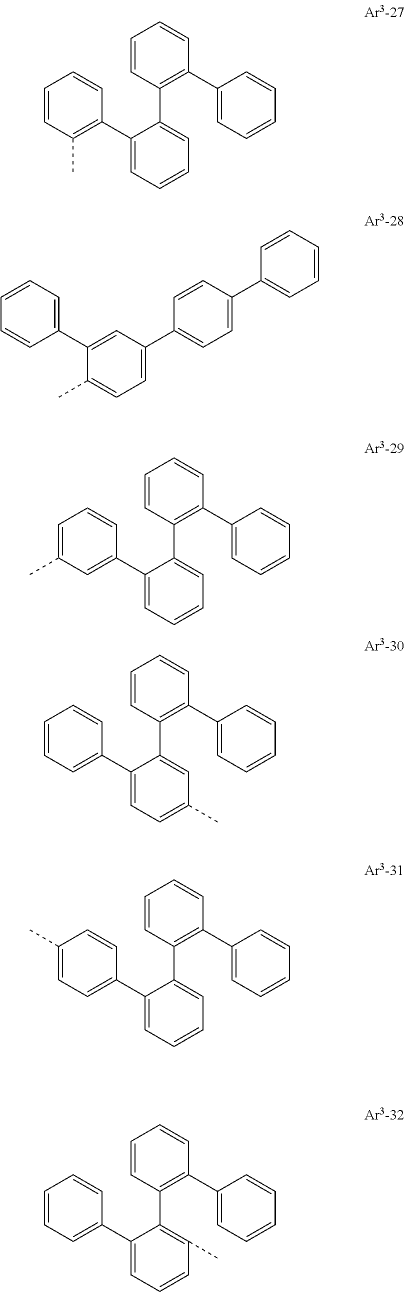

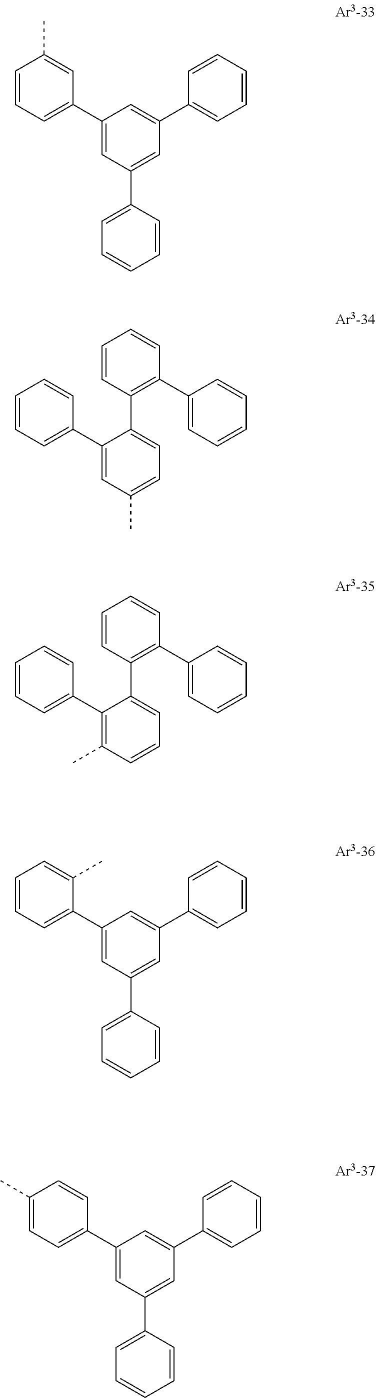

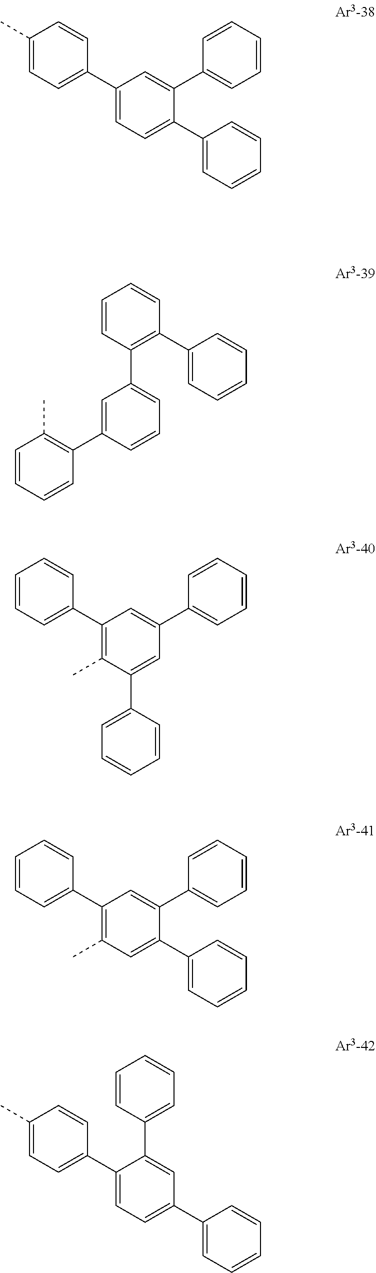

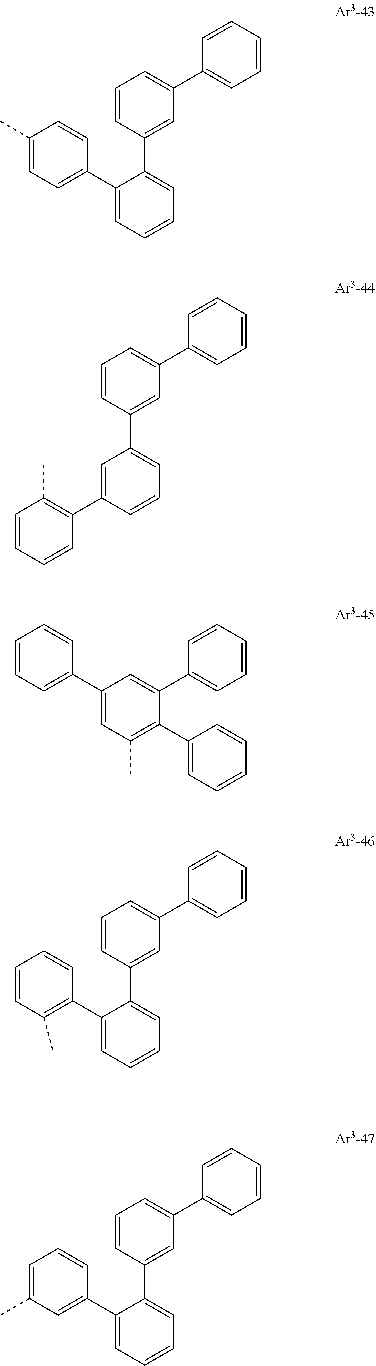

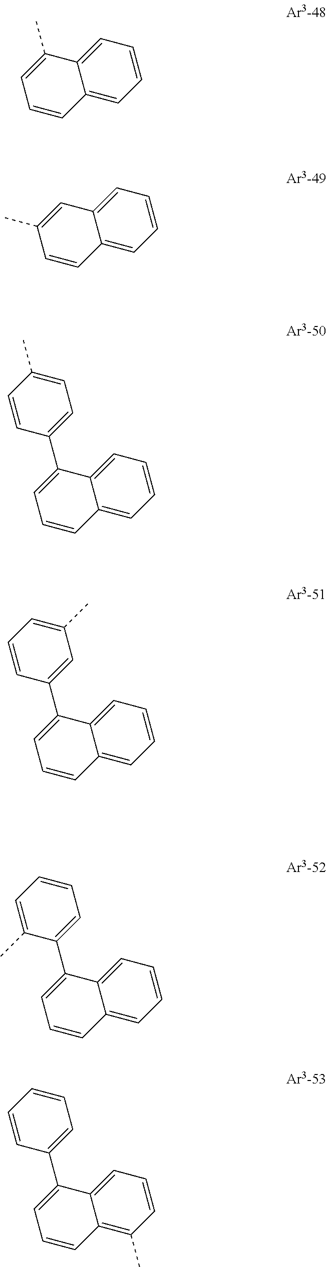

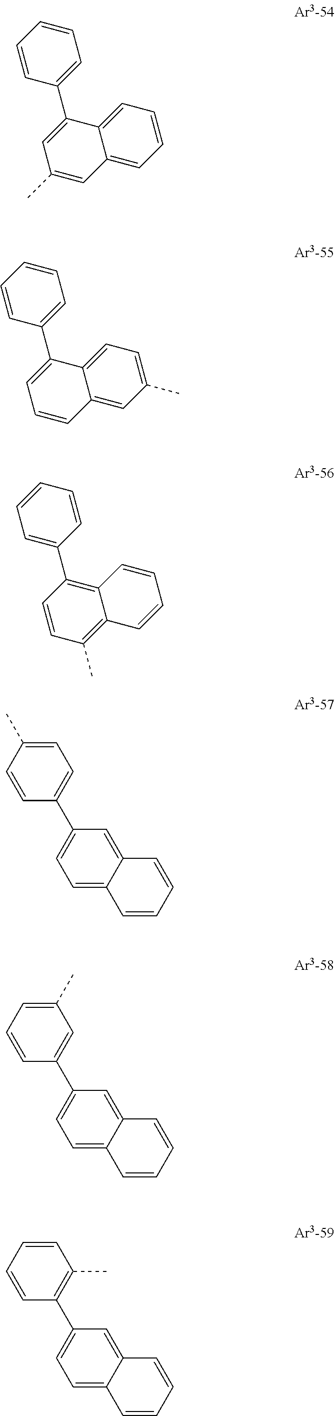

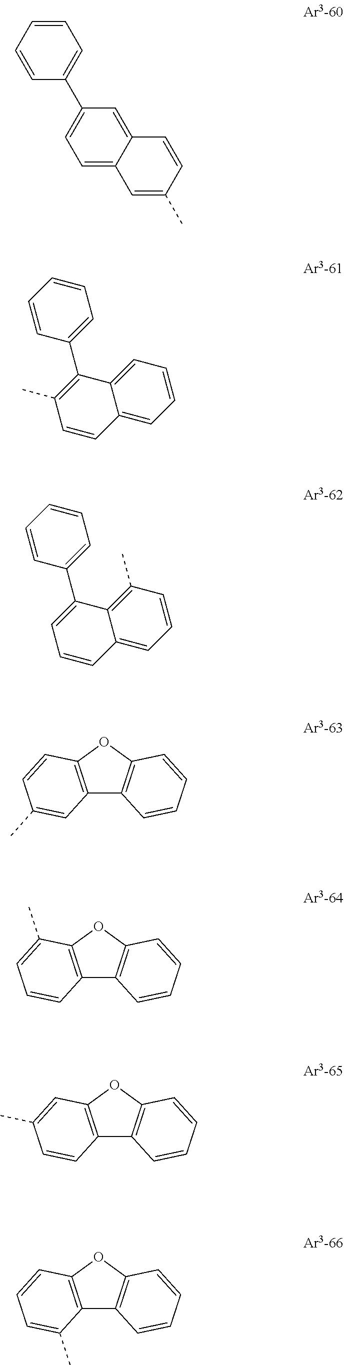

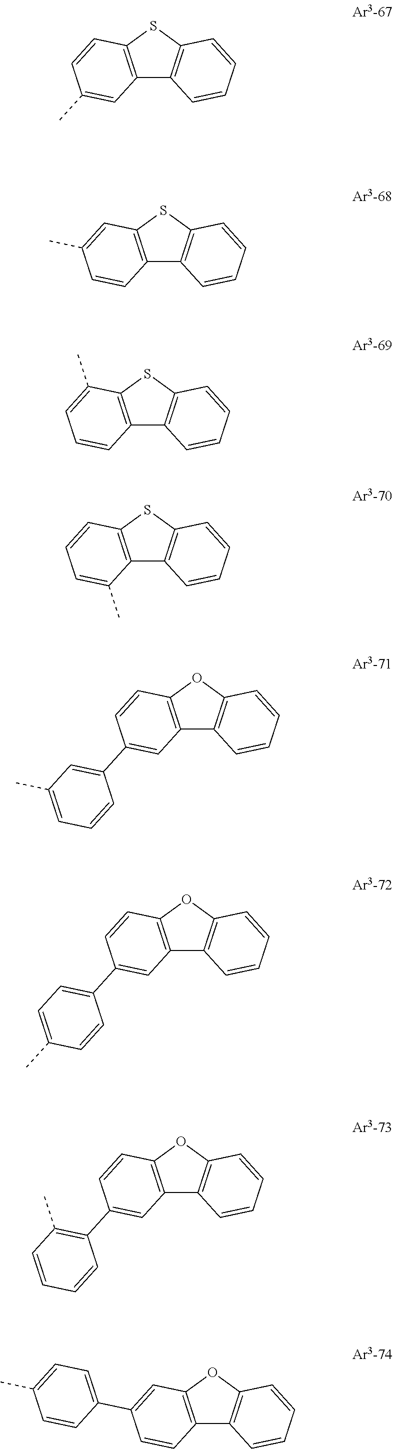

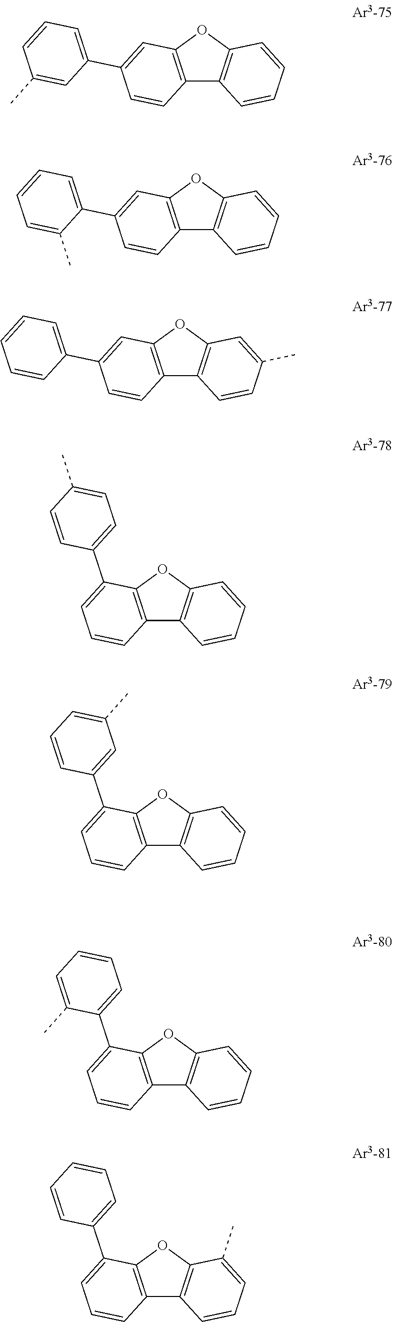

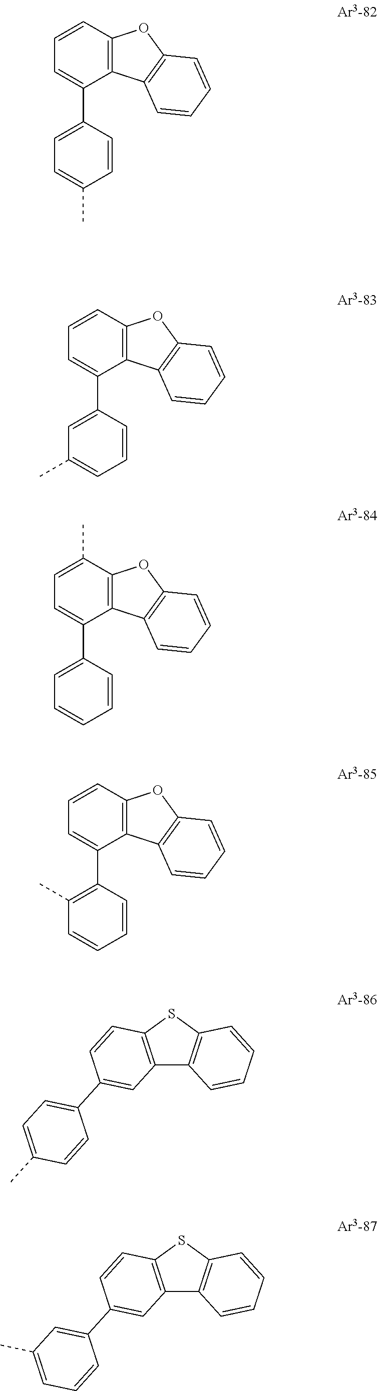

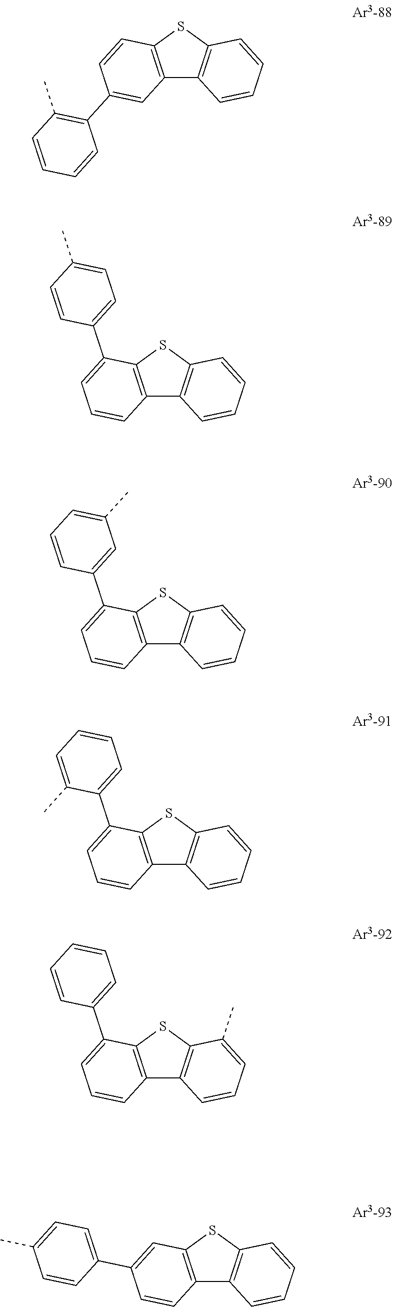

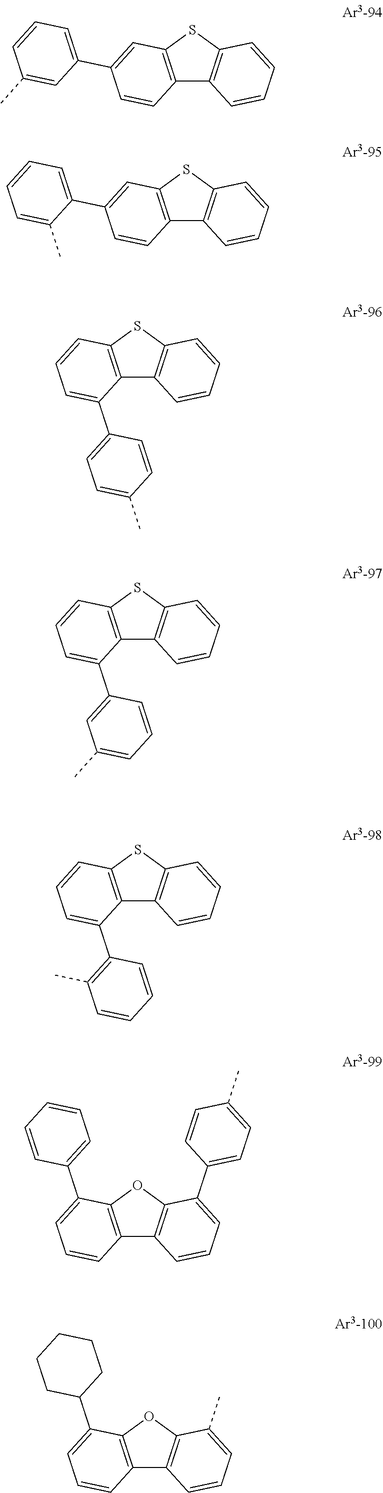

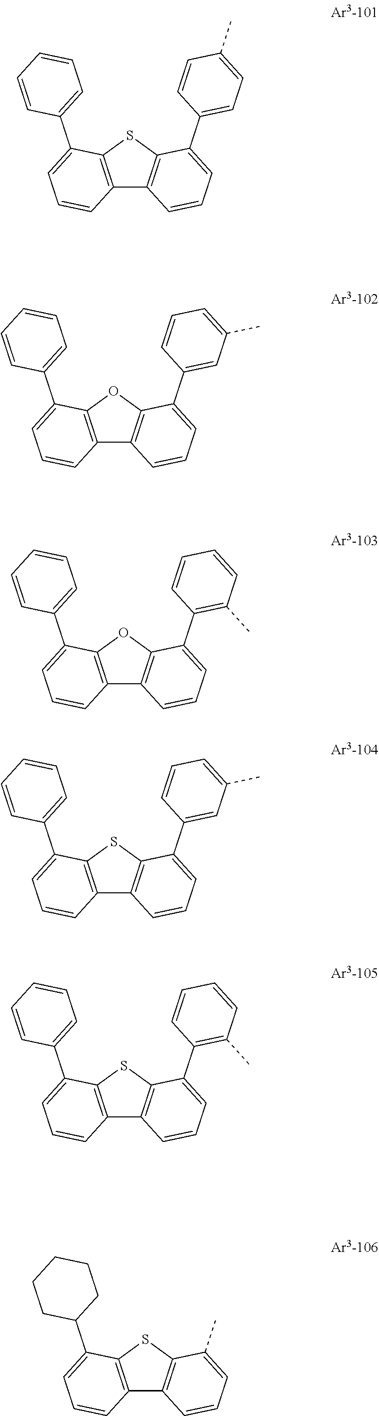

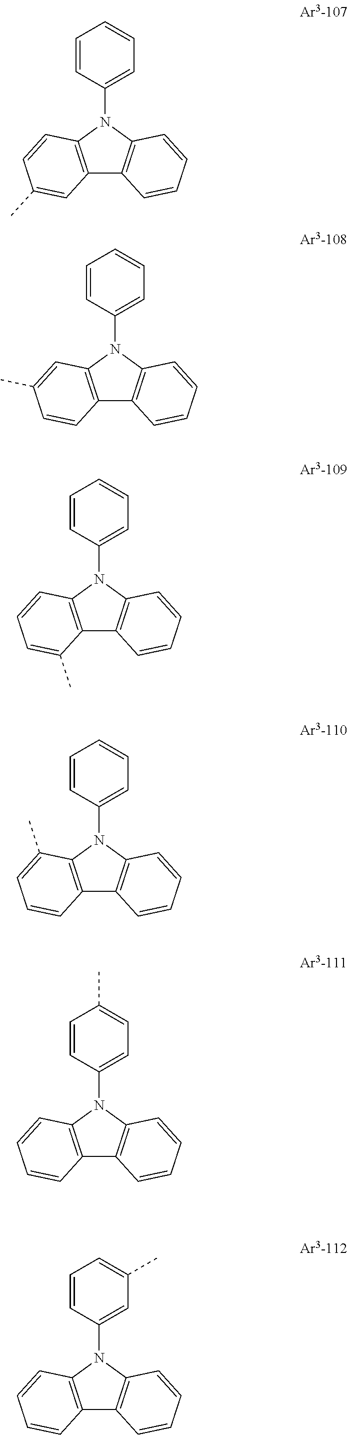

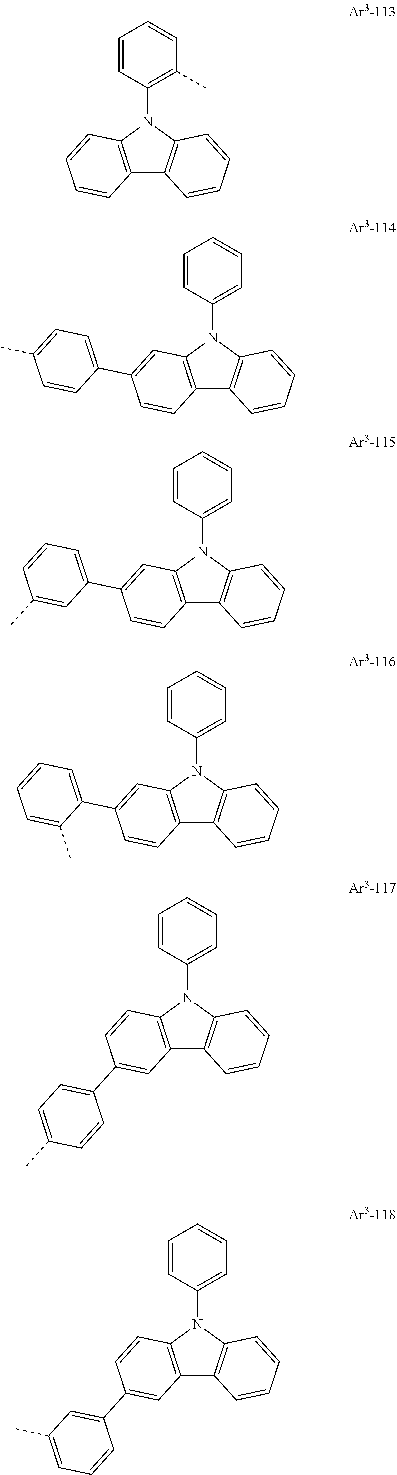

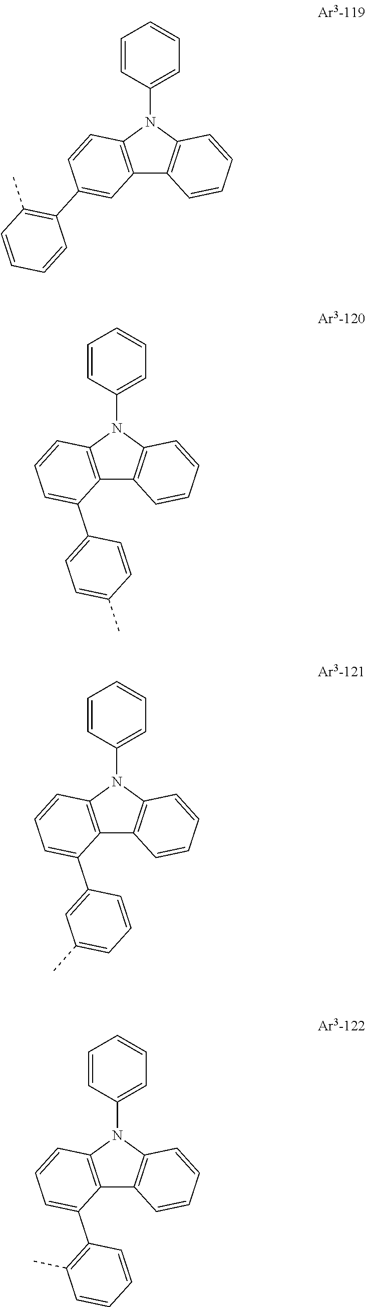

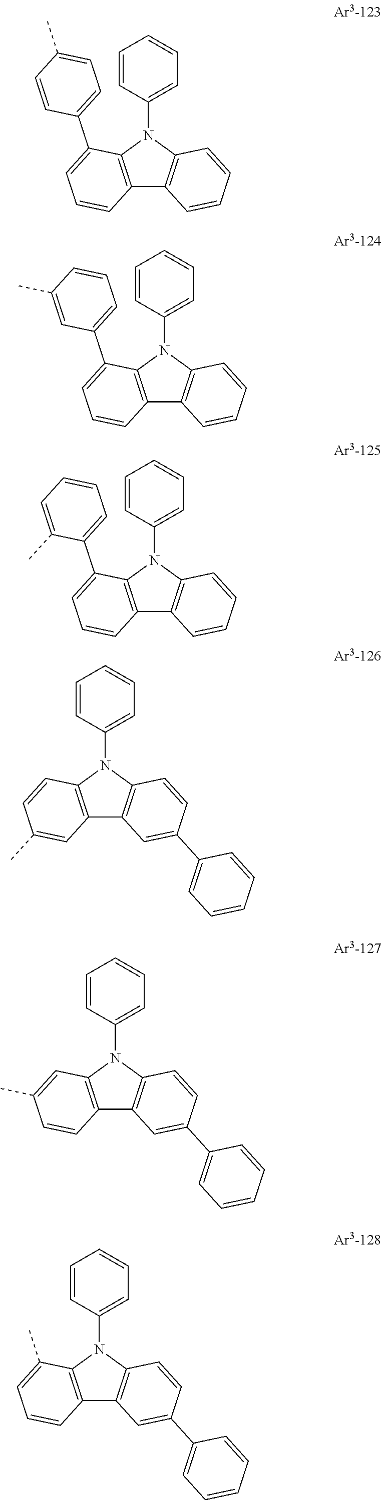

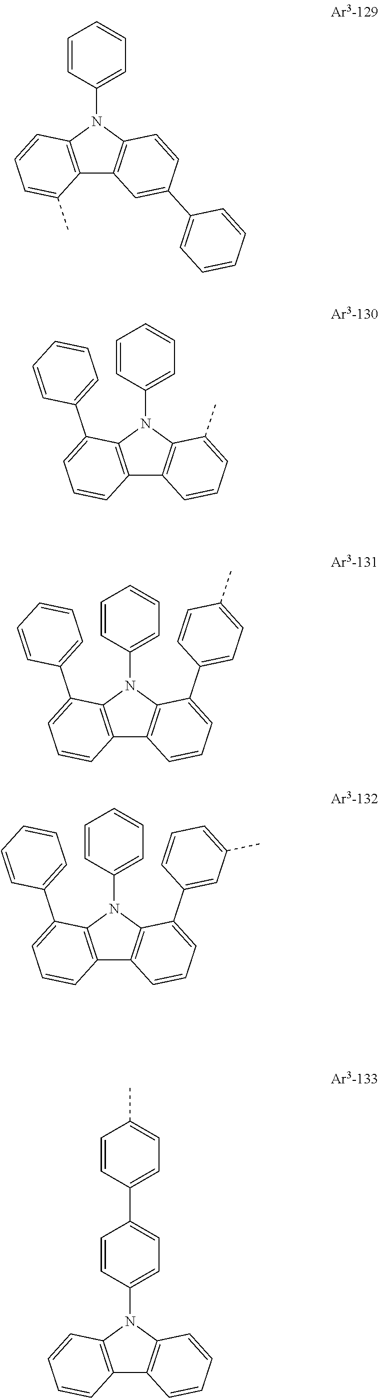

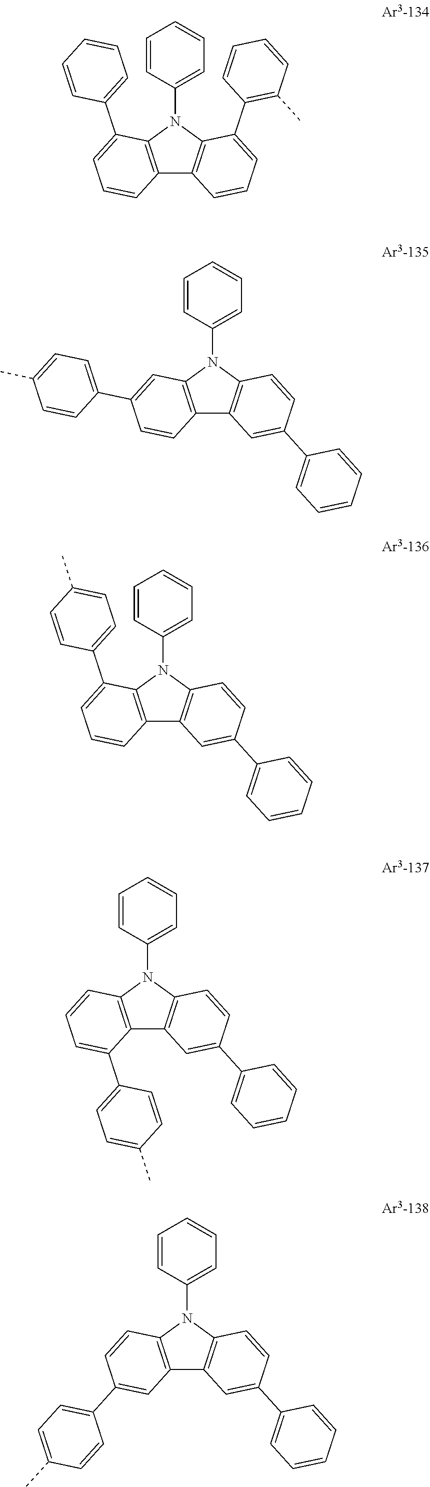

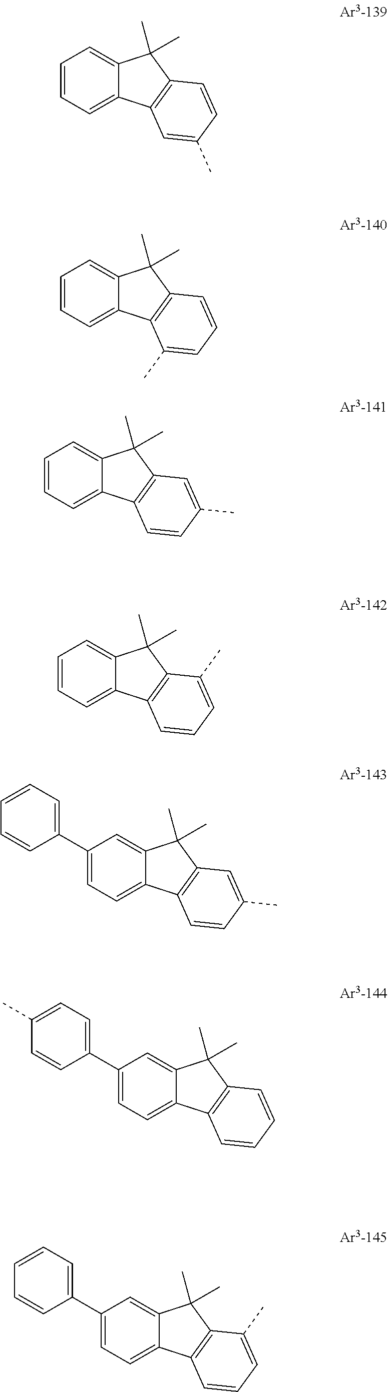

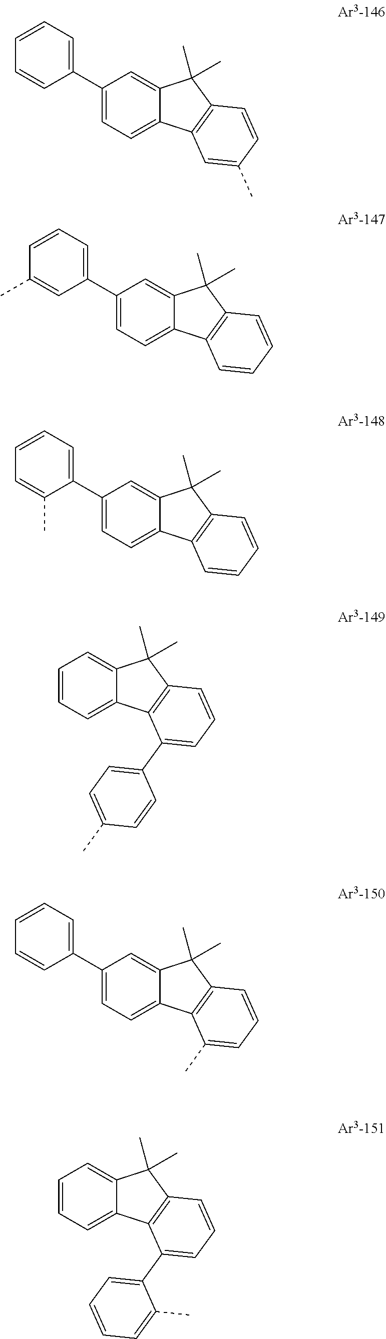

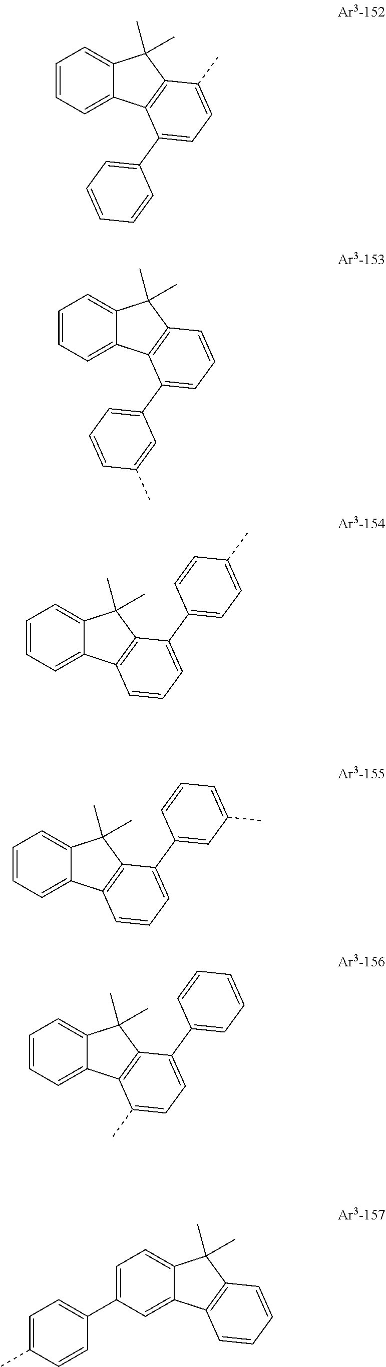

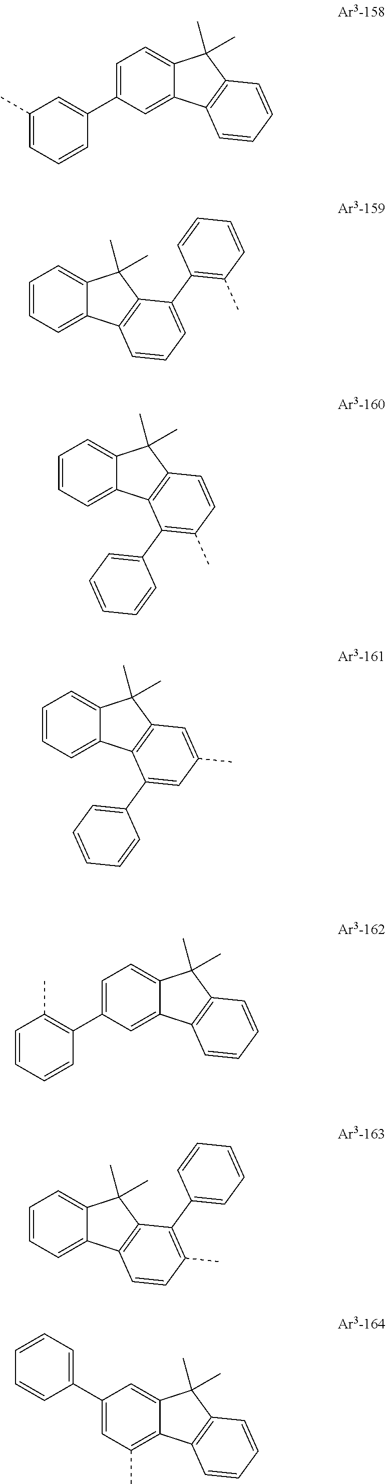

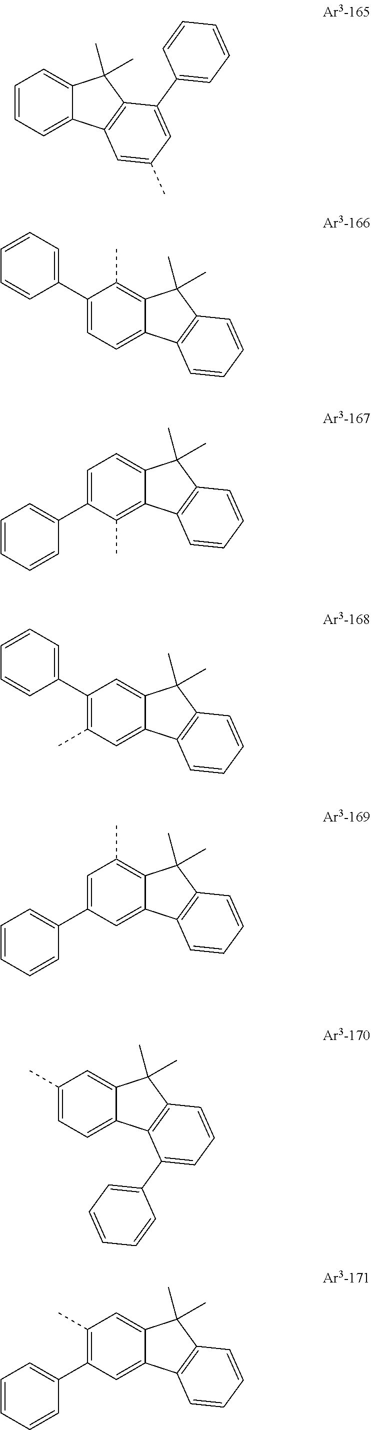

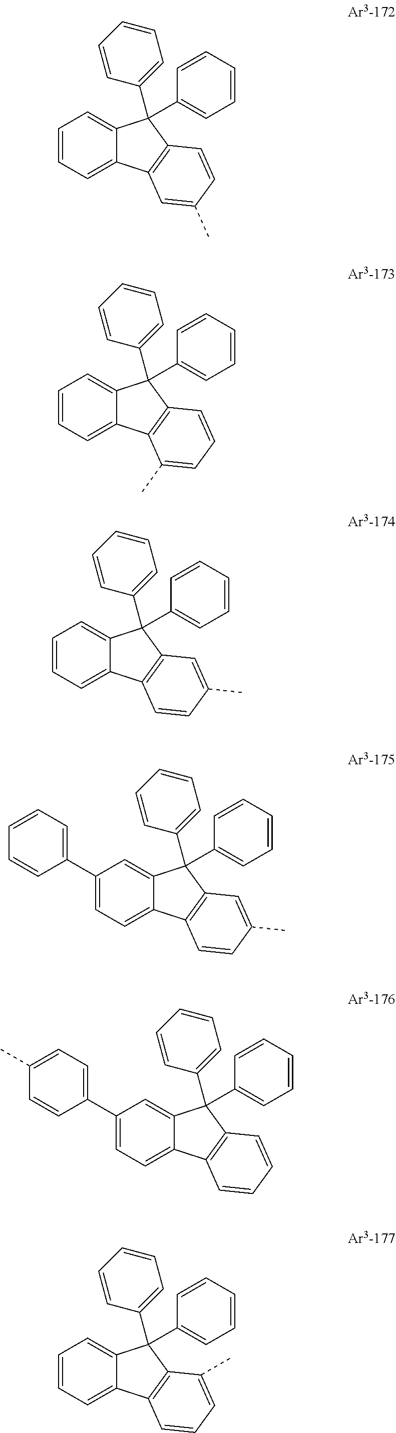

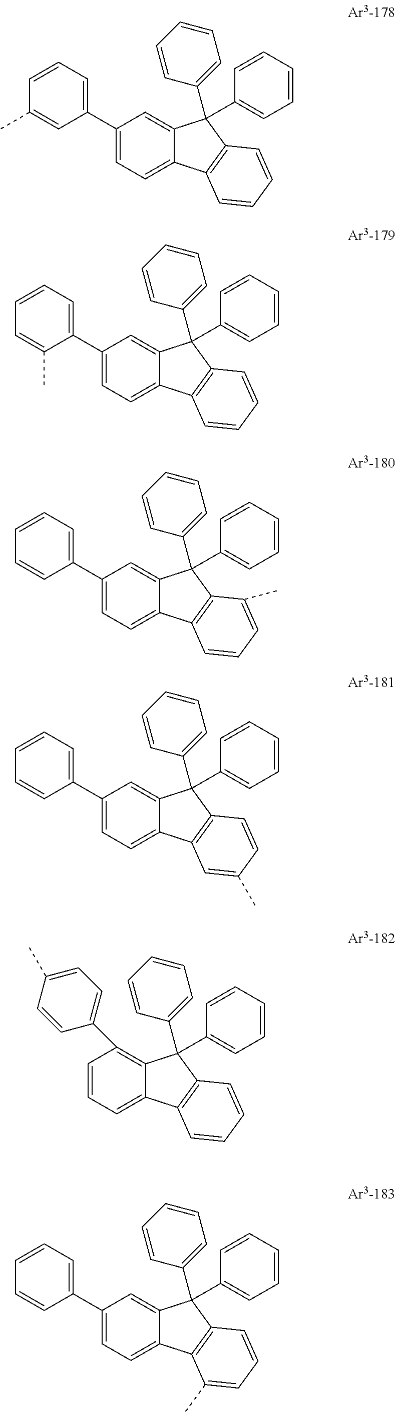

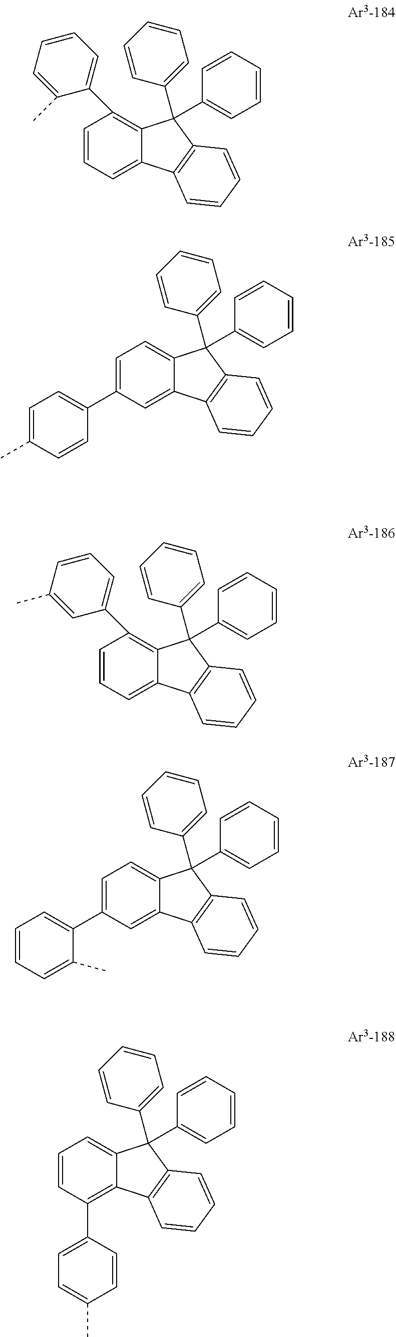

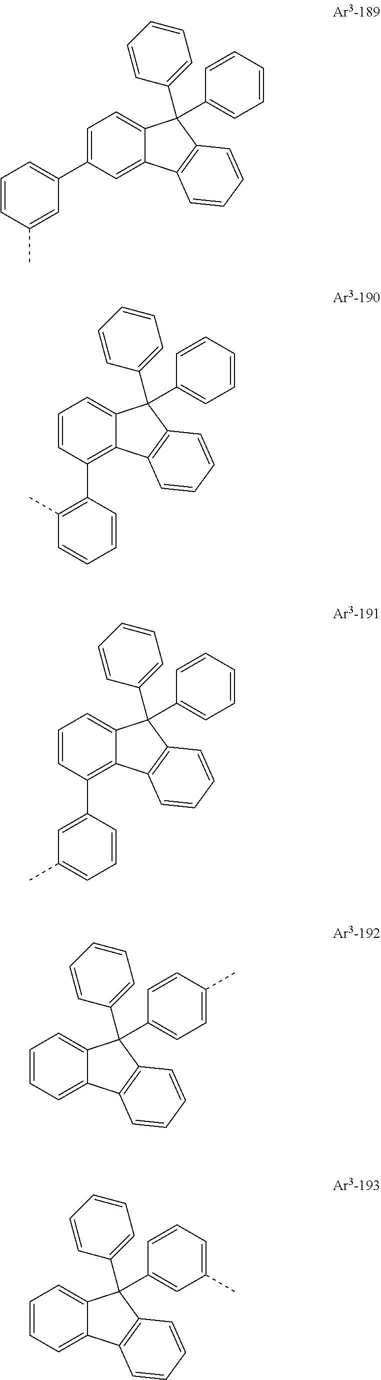

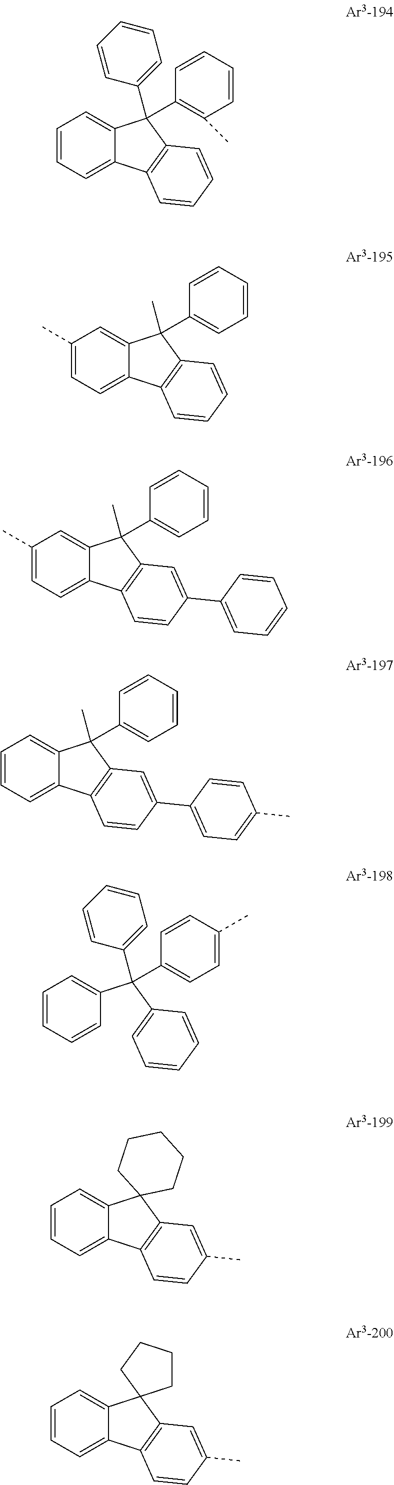

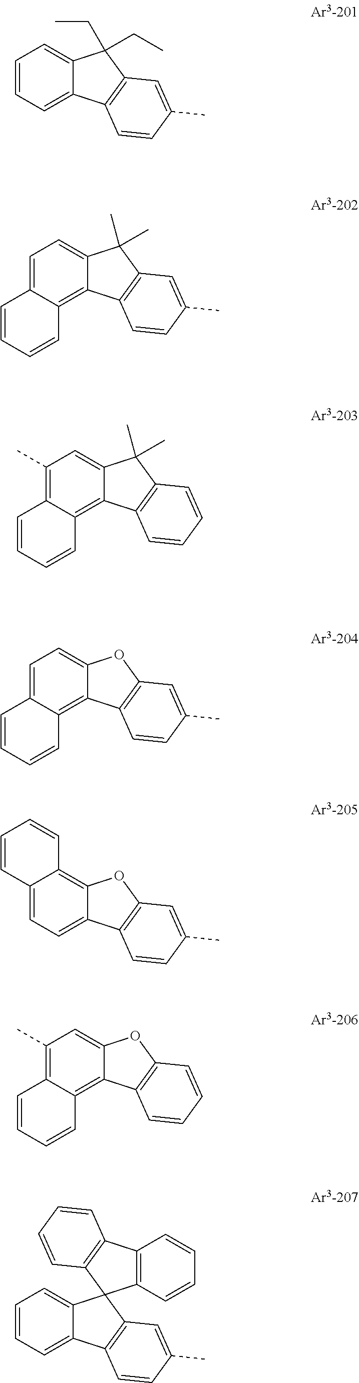

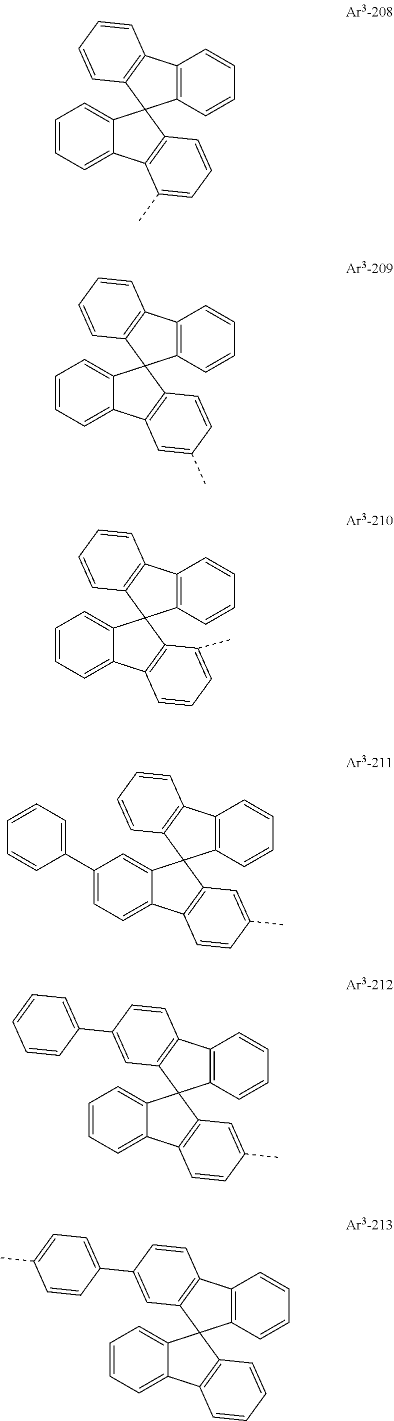

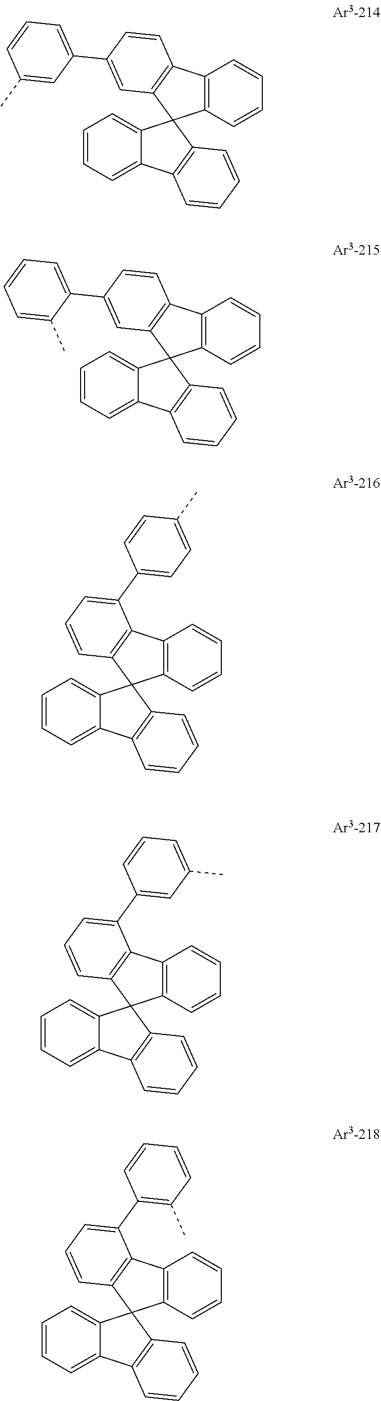

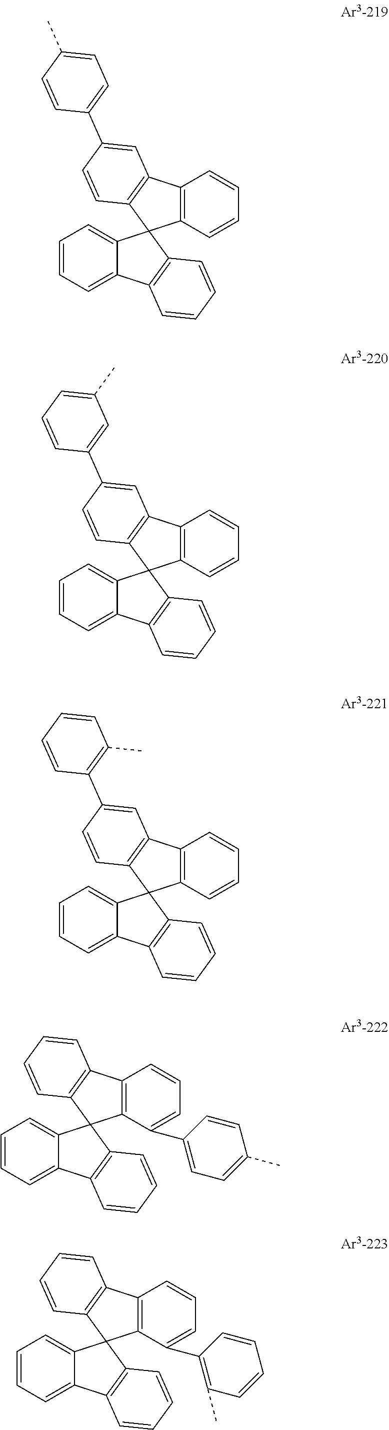

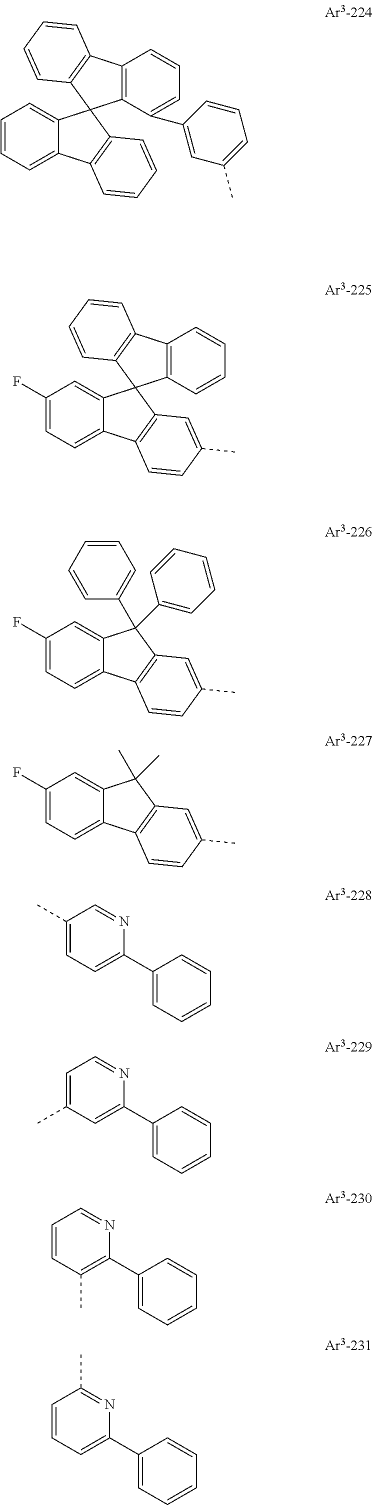

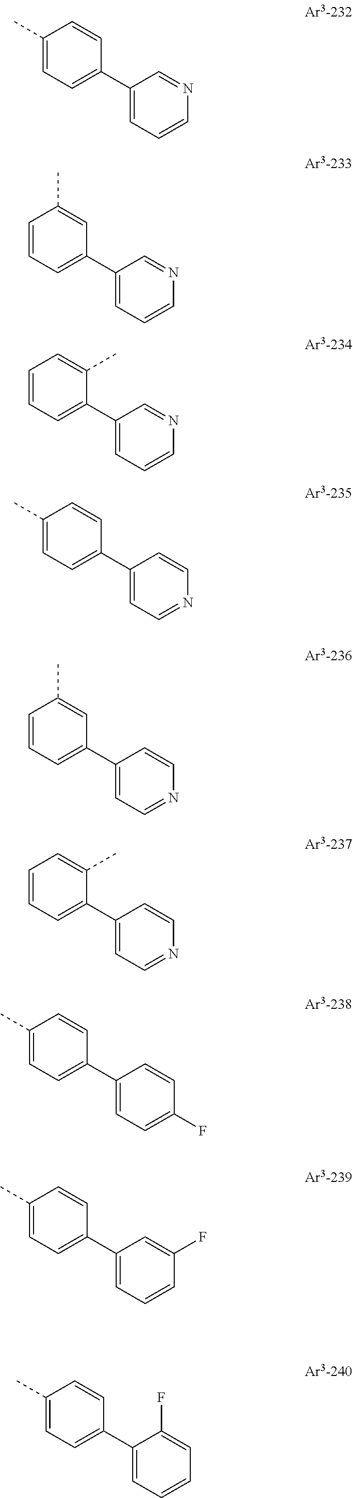

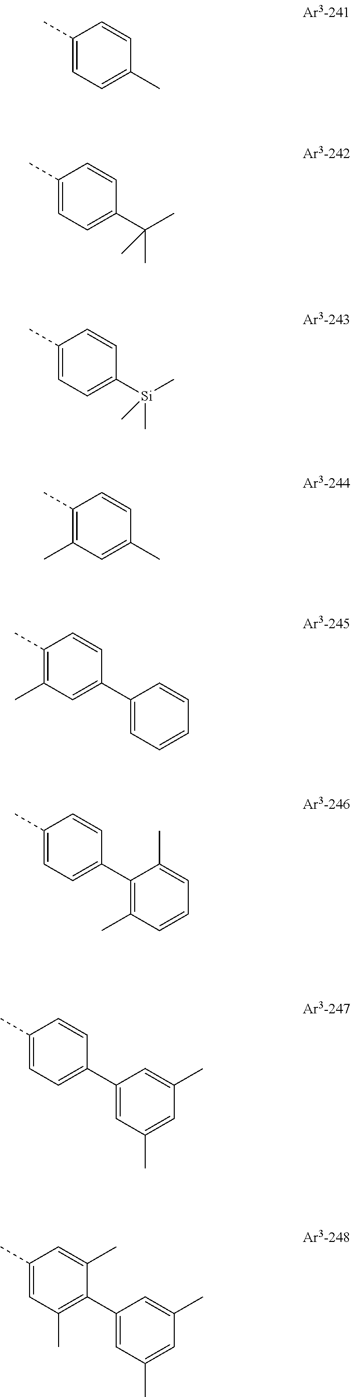

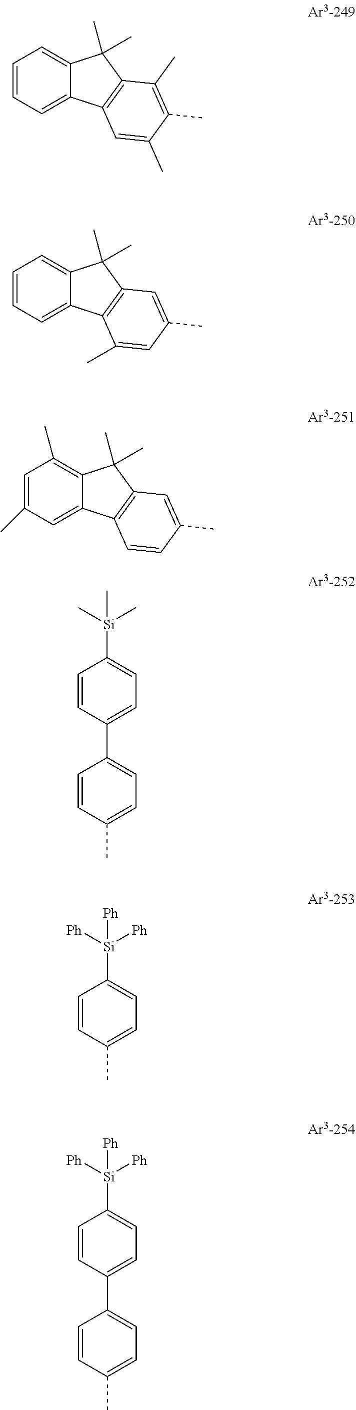

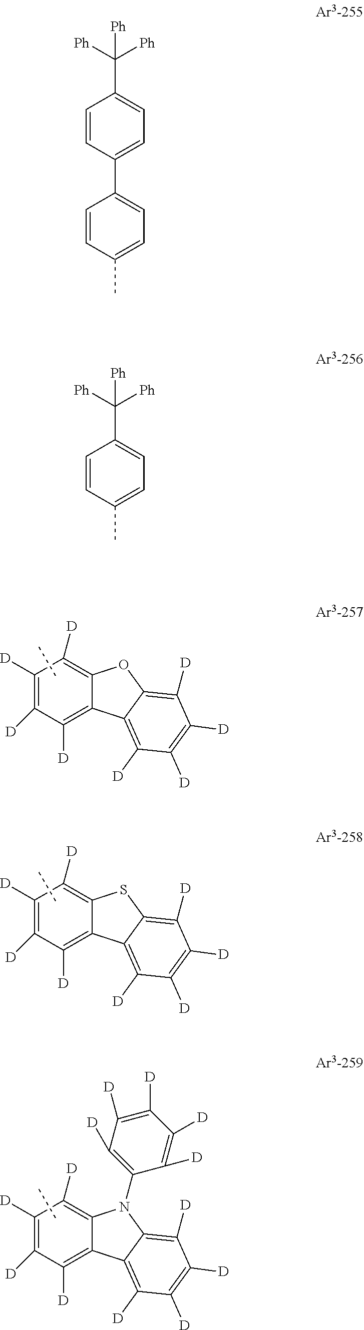

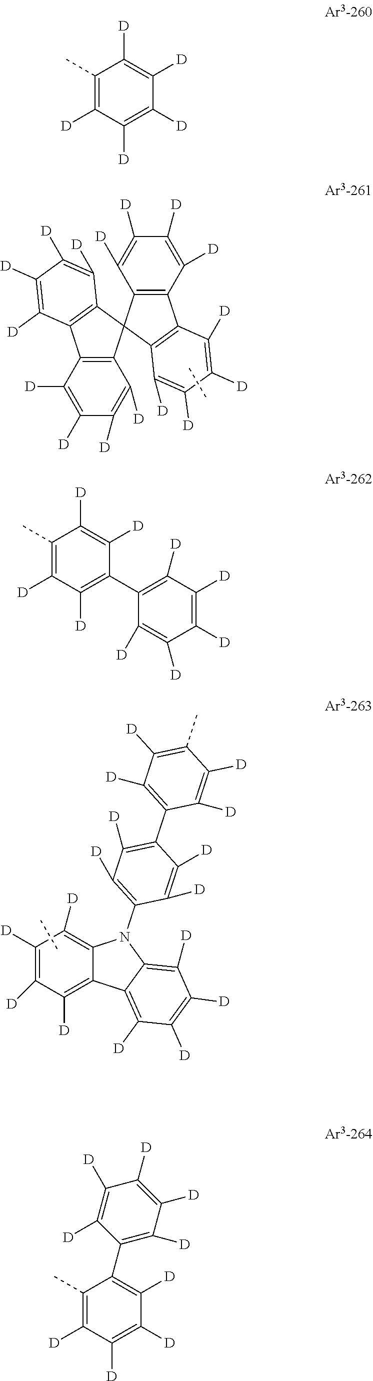

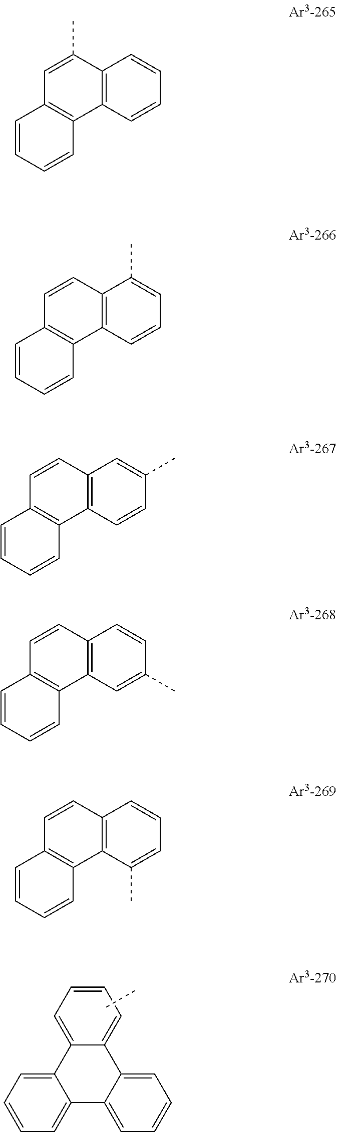

Among these particular preference is given to the groups Ar³-1, Ar³-2, Ar³-3, Ar³-5, Ar³-50, Ar³-56, Ar³-78, Ar³-82, Ar³-111, Ar³-114, Ar³-140, Ar³-141, Ar³-257, Ar³-262, Ar³-263.

Units —[Ar²]ₙ—Ar³ are preferably selected from the following units

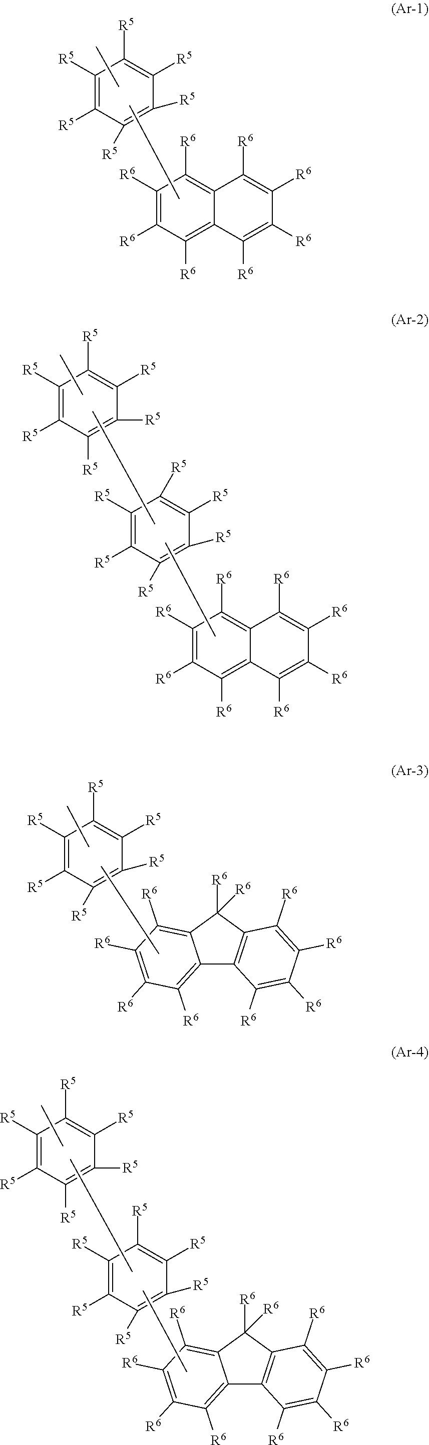

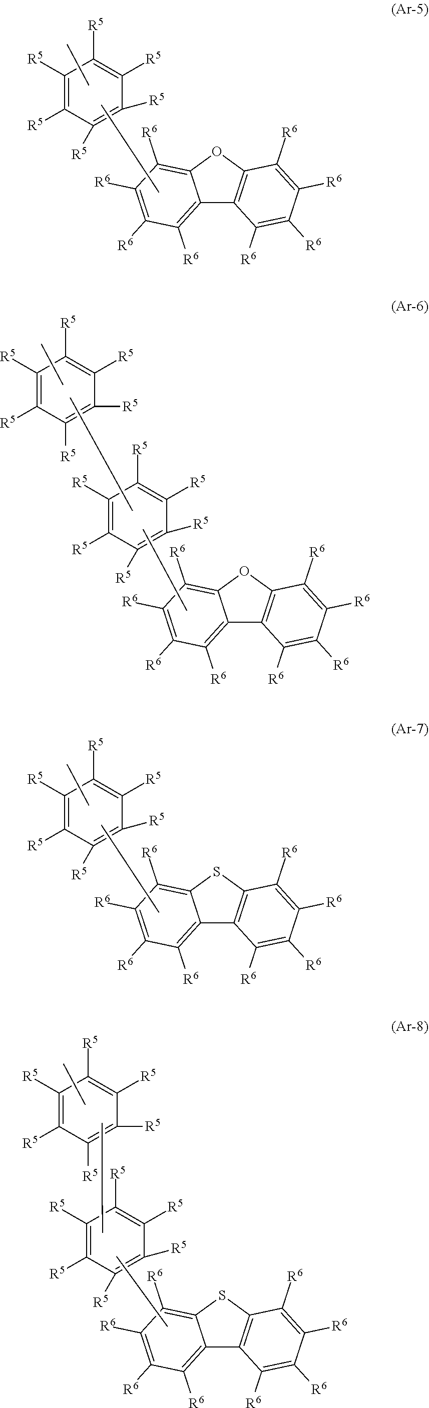

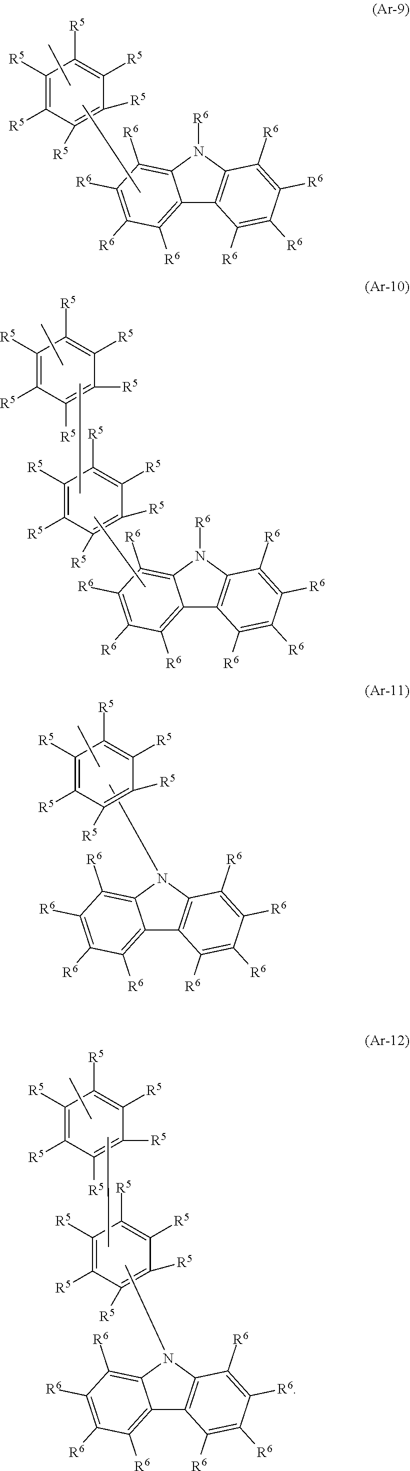

Preferred embodiments of the formula (I) are selected from the following formulae:

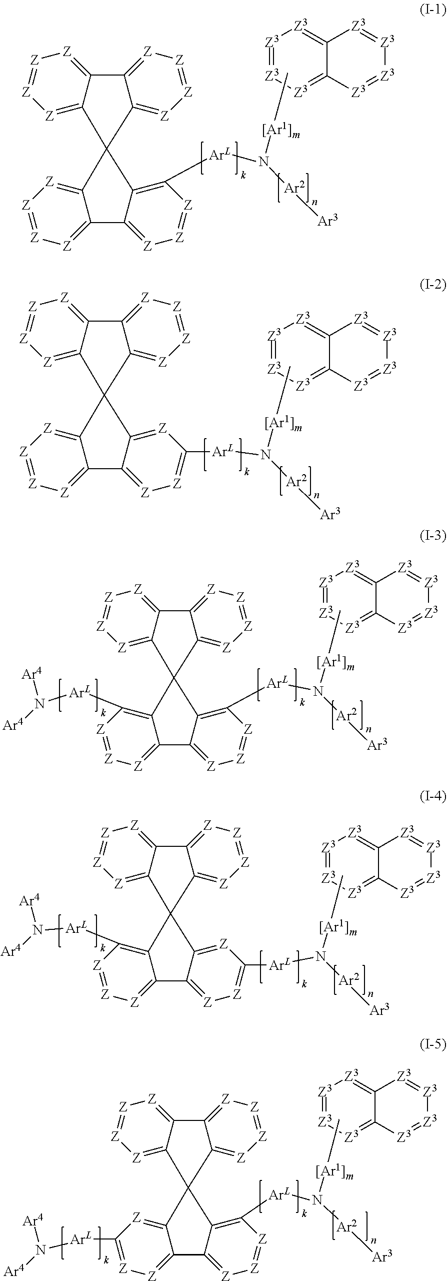

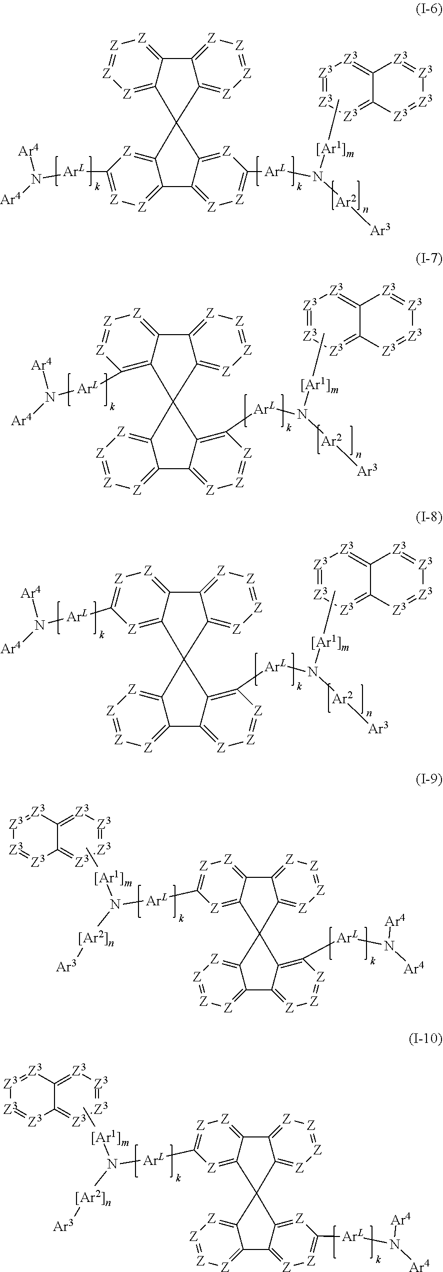

wherein the variables that occur are as defined above and wherein formulae (I-1) and (I-2) are preferred, particularly preferably formula (I-1). Preferably applying in the formulae are the preferred embodiments of the variable groups and indices, in particular the groups Z, Z³, Ar^{L}, Ar¹, Ar², Ar³, k, m and n. Z is especially preferably CR¹.

It is preferable when in the formulae (I-3) to (I-10) the group —[Ar^{L}]ₖ—N(Ar⁴)₂ is defined as a group of the formulae

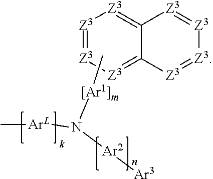

Corresponding preferred formulae for the compounds according to the present application are:

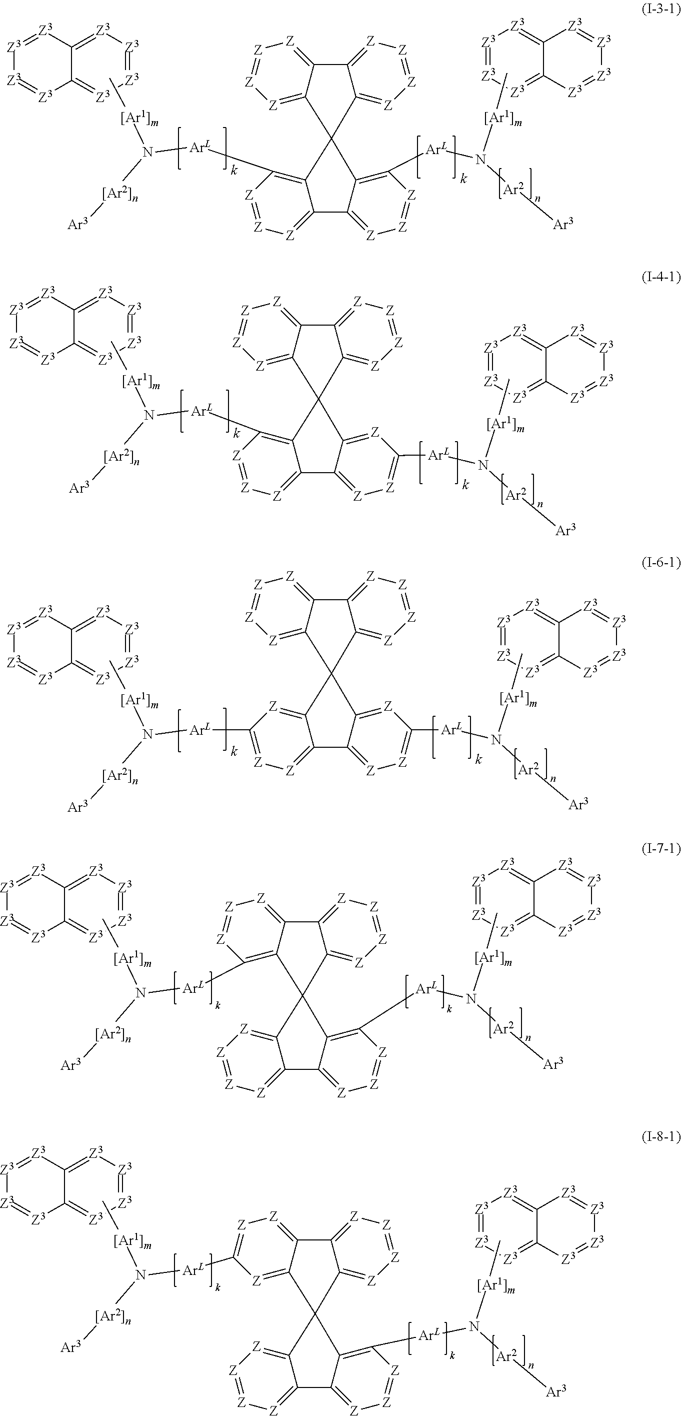

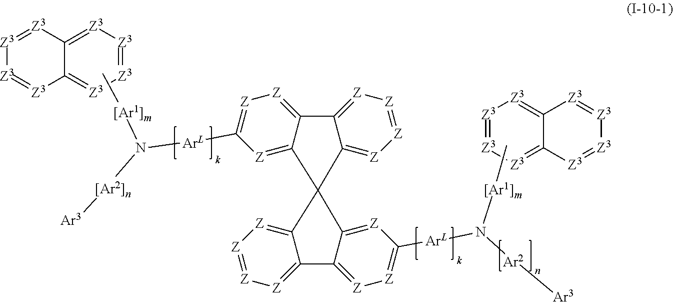

wherein the groups that occur are as defined above. Preferably applying in the formulae are the preferred embodiments of the variable groups and indices, in particular the groups Z, Z³, Ar^{L}, Ar¹, Ar², Ar³, k, m and n. Especially preferably, Z is CR¹ and Z³ is CR⁴.

Particularly preferred embodiments of the formula (I) are selected from the following formulae:

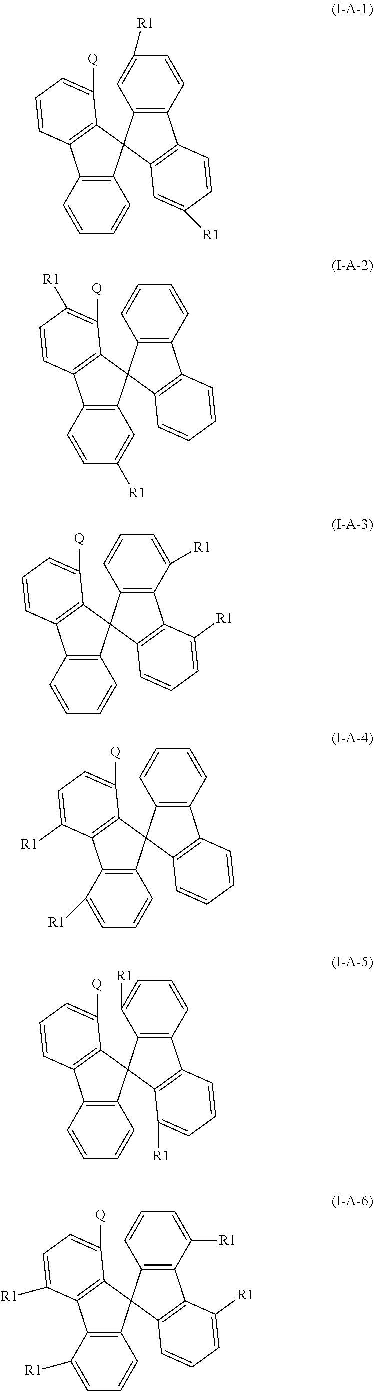

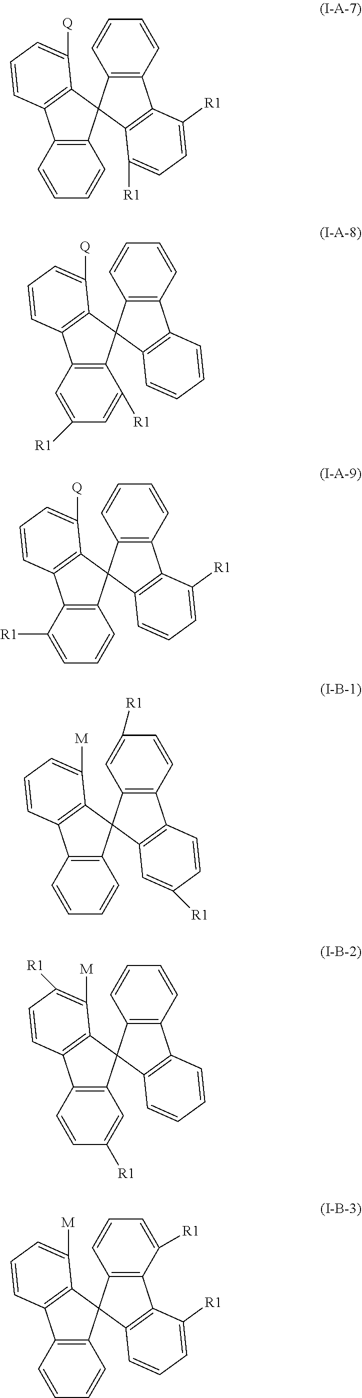

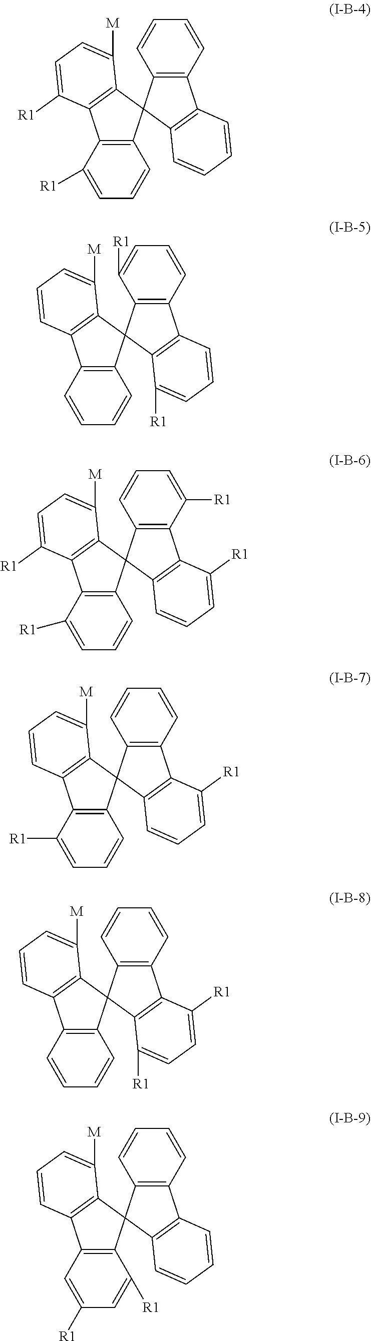

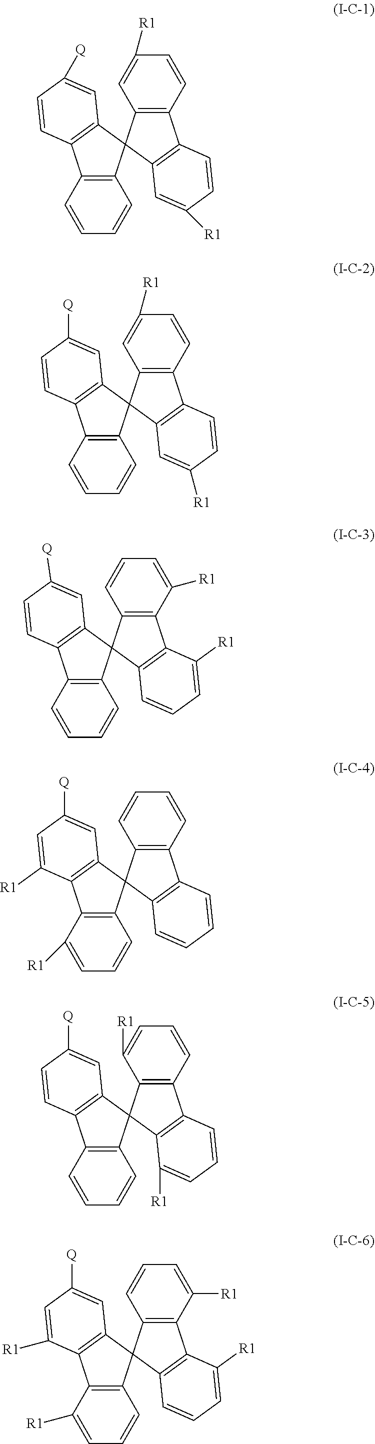

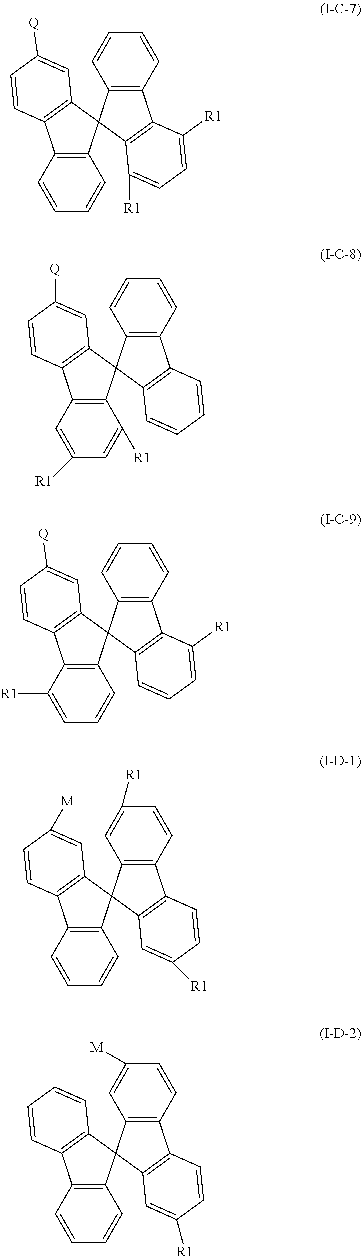

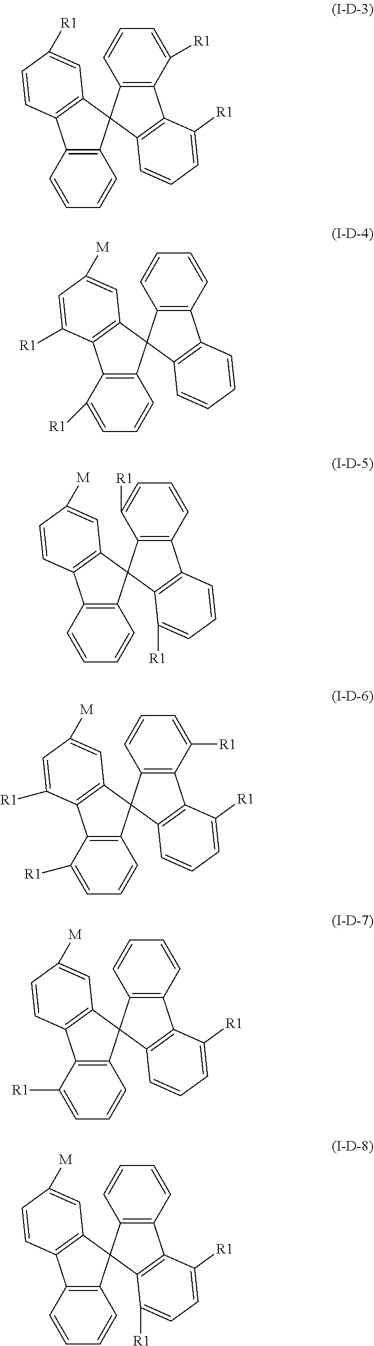

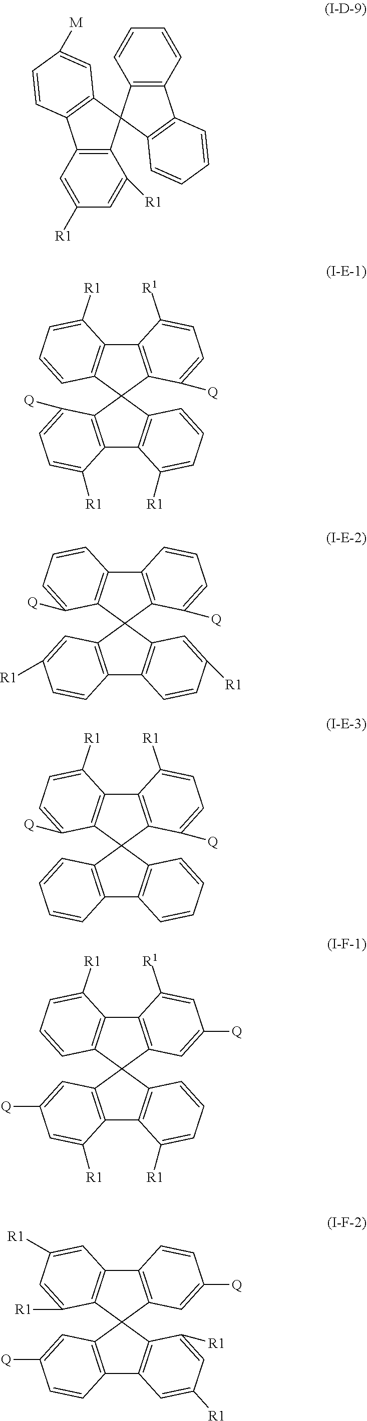

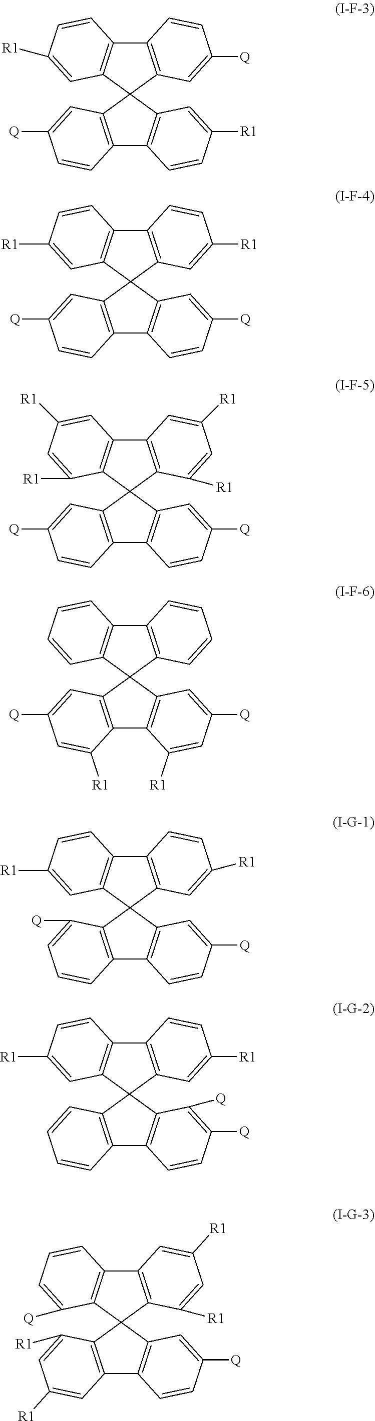

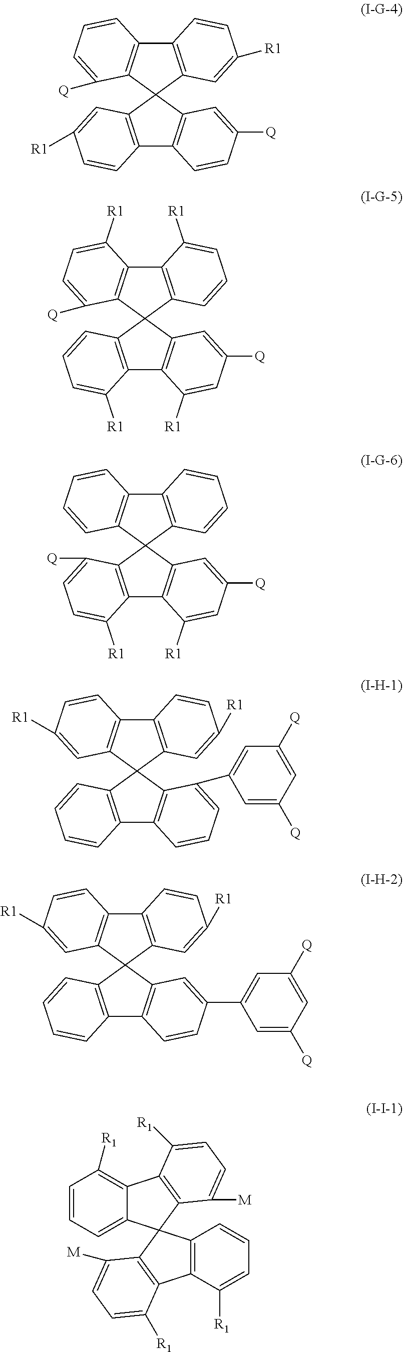

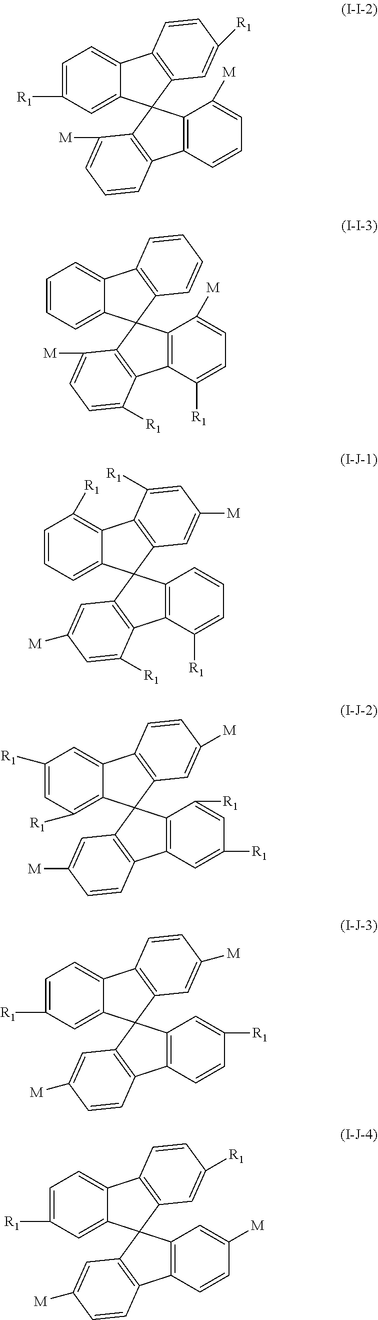

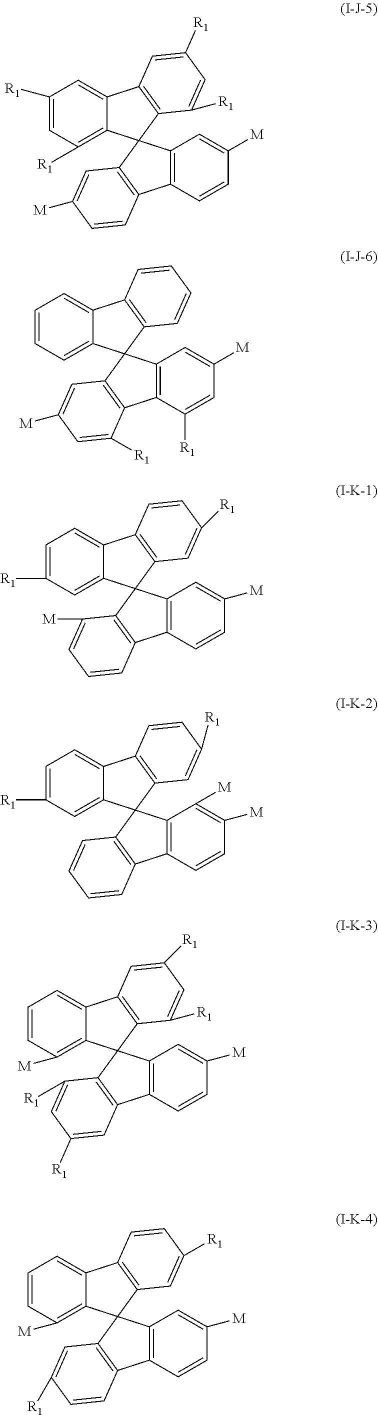

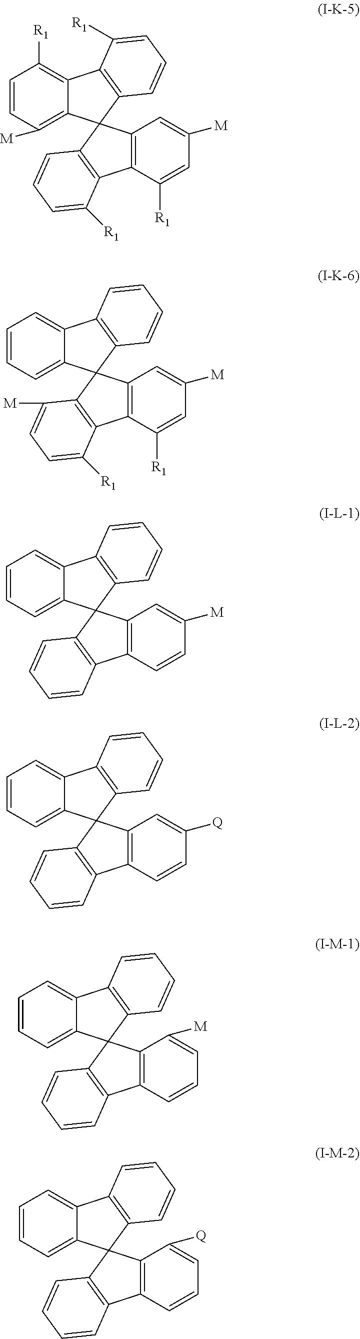

wherein groups M and Q are defined as follows:

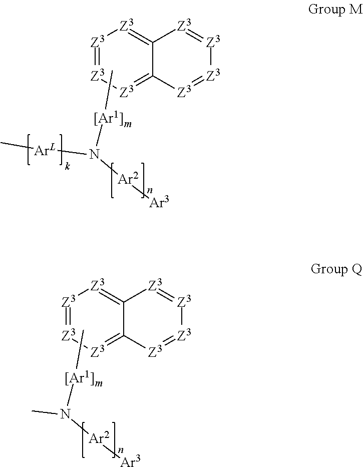

and wherein the other variables are as defined above and wherein R¹ has the same definition as R¹.

Preferred compounds are shown in the following table:

| | |
|---|---|
| 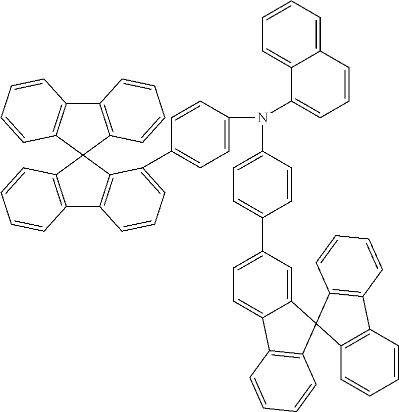 [SMILES: c1ccc2c(c1)-c1ccccc1C21c2ccccc2-c2ccc(-c3ccc(N(c4ccc(-c5cccc6c5C5(c7ccccc7-c7ccccc75)c5ccccc5-6)cc4)c4cccc5ccccc45)cc3)cc21] | 1 |
| 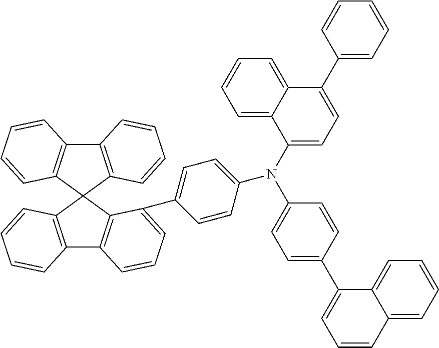 [SMILES: c1ccc(-c2ccc(N(c3ccc(-c4cccc5c4C4(c6ccccc6-c6ccccc64)c4ccccc4-5)cc3)c3ccc(-c4cccc5ccccc45)cc3)c3ccccc23)cc1] | 2 |
| 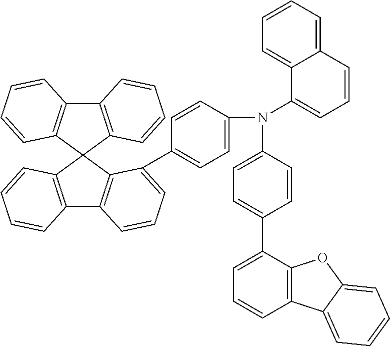 [SMILES: c1ccc2c(c1)-c1ccccc1C21c2ccccc2-c2cccc(-c3ccc(N(c4ccc(-c5cccc6c5oc5ccccc56)cc4)c4cccc5ccccc45)cc3)c21] | 3 |
| 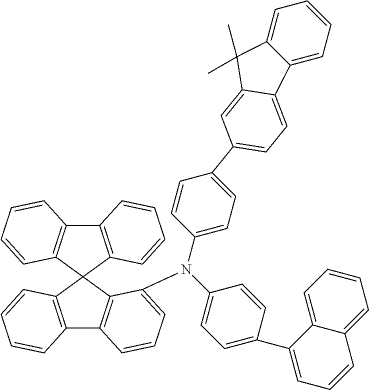 [SMILES: CC1(C)c2ccccc2-c2ccc(-c3ccc(N(c4ccc(-c5cccc6ccccc56)cc4)c4cccc5c4C4(c6ccccc6-c6ccccc64)c4ccccc4-5)cc3)cc21] | 4 |
| 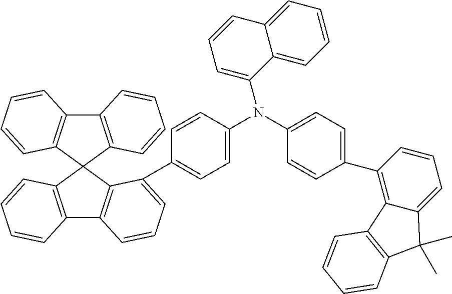 [SMILES: CC1(C)c2ccccc2-c2c(-c3ccc(N(c4ccc(-c5cccc6c5C5(c7ccccc7-c7ccccc75)c5ccccc5-6)cc4)c4cccc5ccccc45)cc3)cccc21] | 5 |
| 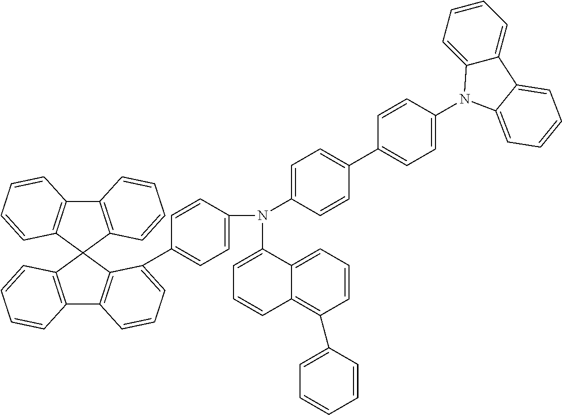 [SMILES: c1ccc(-c2cccc3c(N(c4ccc(-c5ccc(-n6c7ccccc7c7ccccc76)cc5)cc4)c4ccc(-c5cccc6c5C5(c7ccccc7-c7ccccc75)c5ccccc5-6)cc4)cccc23)cc1] | 6 |
| 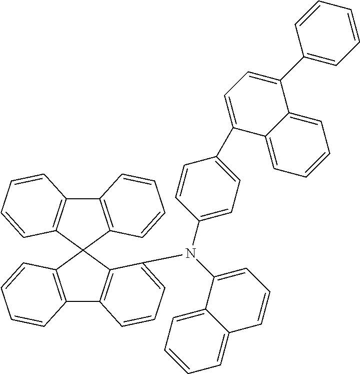 [SMILES: c1ccc(-c2ccc(-c3ccc(N(c4cccc5c4C4(c6ccccc6-c6ccccc64)c4ccccc4-5)c4cccc5ccccc45)cc3)c3ccccc23)cc1] | 7 |
| 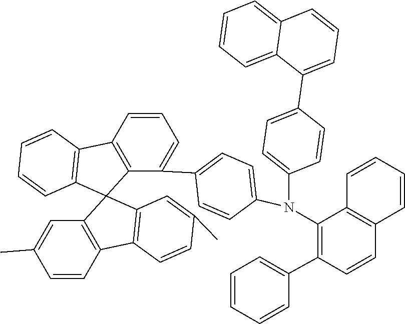 [SMILES: Cc1ccc2c(c1)C1(c3cc(C)ccc3-2)c2ccccc2-c2cccc(-c3ccc(N(c4ccc(-c5cccc6ccccc56)cc4)c4c(-c5ccccc5)ccc5ccccc45)cc3)c21] | 8 |
| 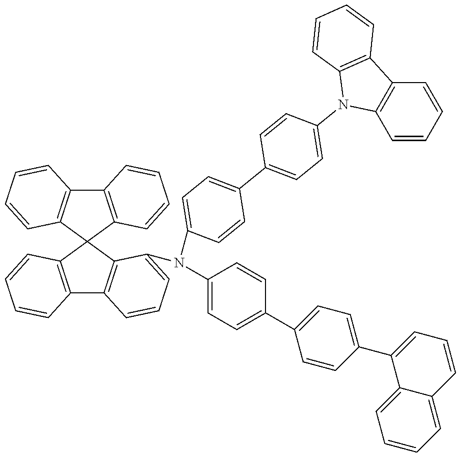 [SMILES: c1ccc2c(c1)-c1ccccc1C21c2ccccc2-c2cccc(N(c3ccc(-c4ccc(-c5cccc6ccccc56)cc4)cc3)c3ccc(-c4ccc(-n5c6ccccc6c6ccccc65)cc4)cc3)c21] | 9 |
| 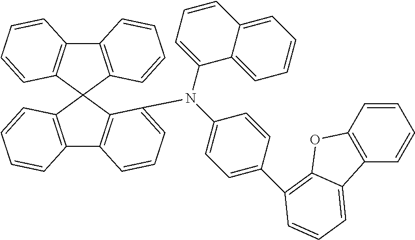 [SMILES: c1ccc2c(c1)-c1ccccc1C21c2ccccc2-c2cccc(N(c3ccc(-c4cccc5c4oc4ccccc45)cc3)c3cccc4ccccc34)c21] | 10 |
| 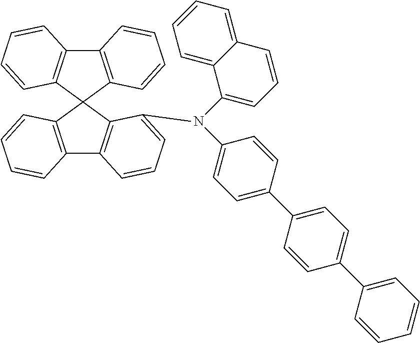 [SMILES: c1ccc(-c2ccc(-c3ccc(N(c4cccc5c4C4(c6ccccc6-c6ccccc64)c4ccccc4-5)c4cccc5ccccc45)cc3)cc2)cc1] | 11 |
| 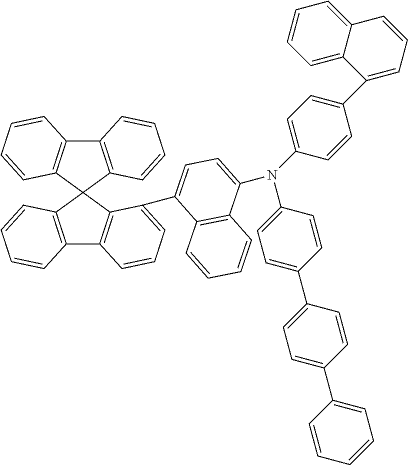 [SMILES: c1ccc(-c2ccc(-c3ccc(N(c4ccc(-c5cccc6ccccc56)cc4)c4ccc(-c5cccc6c5C5(c7ccccc7-c7ccccc75)c5ccccc5-6)c5ccccc45)cc3)cc2)cc1] | 12 |
| 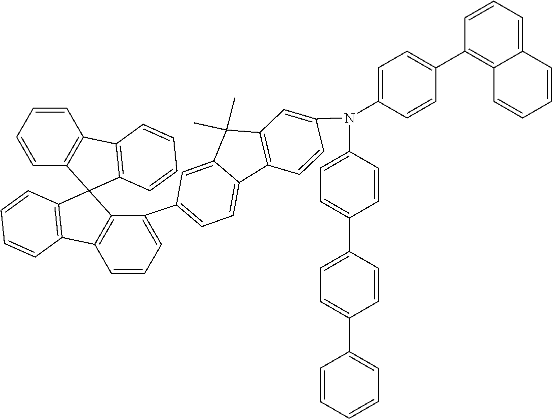 [SMILES: CC1(C)c2cc(-c3cccc4c3C3(c5ccccc5-c5ccccc53)c3ccccc3-4)ccc2-c2ccc(N(c3ccc(-c4ccc(-c5ccccc5)cc4)cc3)c3ccc(-c4cccc5ccccc45)cc3)cc21] | 13 |
| 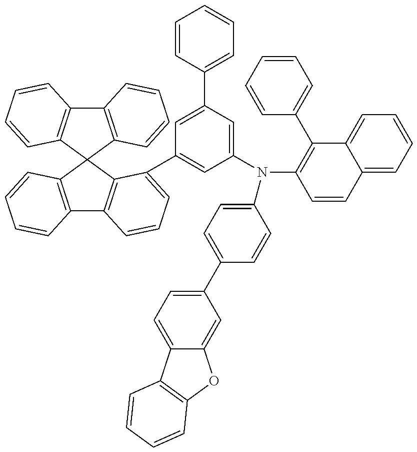 [SMILES: c1ccc(-c2cc(-c3cccc4c3C3(c5ccccc5-c5ccccc53)c3ccccc3-4)cc(N(c3ccc(-c4ccc5c(c4)oc4ccccc45)cc3)c3ccc4ccccc4c3-c3ccccc3)c2)cc1] | 14 |
| 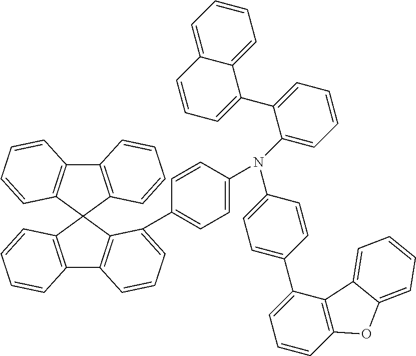 [SMILES: c1ccc(N(c2ccc(-c3cccc4c3C3(c5ccccc5-c5ccccc53)c3ccccc3-4)cc2)c2ccc(-c3cccc4oc5ccccc5c34)cc2)c(-c2cccc3ccccc23)c1] | 15 |
| 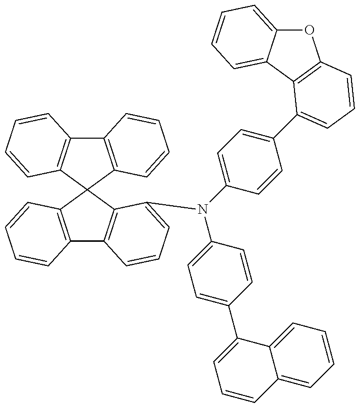 [SMILES: c1ccc2c(c1)-c1ccccc1C21c2ccccc2-c2cccc(N(c3ccc(-c4cccc5ccccc45)cc3)c3ccc(-c4cccc5oc6ccccc6c45)cc3)c21] | 16 |
| 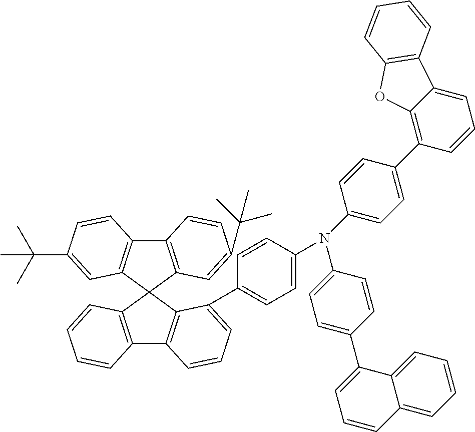 [SMILES: CC(C)(C)c1ccc2c(c1)C1(c3cc(C(C)(C)C)ccc3-2)c2ccccc2-c2cccc(-c3ccc(N(c4ccc(-c5cccc6ccccc56)cc4)c4ccc(-c5cccc6c5oc5ccccc56)cc4)cc3)c21] | 17 |
| 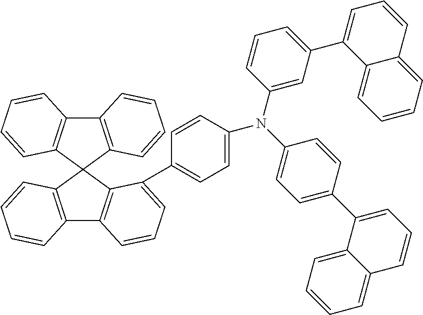 [SMILES: c1cc(-c2cccc3ccccc23)cc(N(c2ccc(-c3cccc4c3C3(c5ccccc5-c5ccccc53)c3ccccc3-4)cc2)c2ccc(-c3cccc4ccccc34)cc2)c1] | 18 |
| 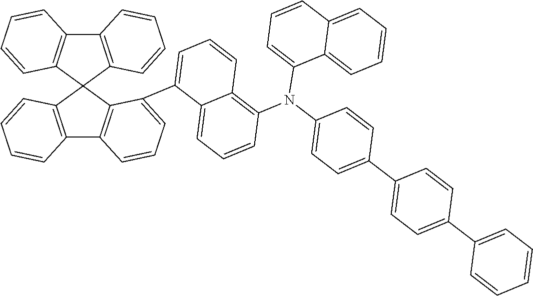 [SMILES: c1ccc(-c2ccc(-c3ccc(N(c4cccc5ccccc45)c4cccc5c(-c6cccc7c6C6(c8ccccc8-c8ccccc86)c6ccccc6-7)cccc45)cc3)cc2)cc1] | 19 |
| 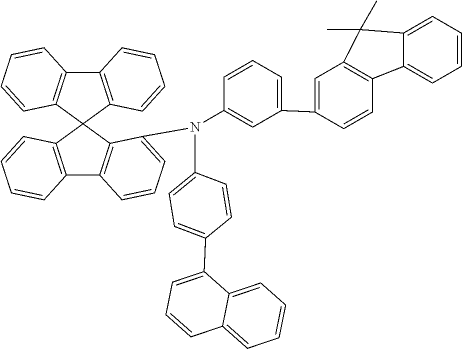 [SMILES: CC1(C)c2ccccc2-c2ccc(-c3cccc(N(c4ccc(-c5cccc6ccccc56)cc4)c4cccc5c4C4(c6ccccc6-c6ccccc64)c4ccccc4-5)c3)cc21] | 20 |
| 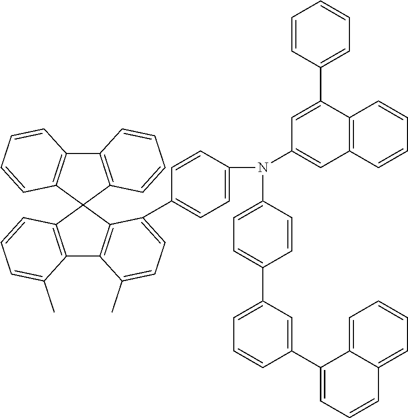 [SMILES: Cc1cccc2c1-c1c(C)ccc(-c3ccc(N(c4ccc(-c5cccc(-c6cccc7ccccc67)c5)cc4)c4cc(-c5ccccc5)c5ccccc5c4)cc3)c1C21c2ccccc2-c2ccccc21] | 21 |
| 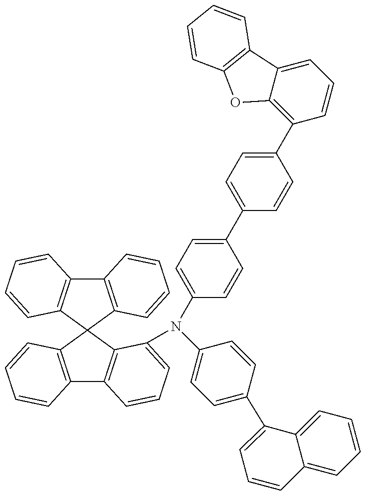 [SMILES: c1ccc2c(c1)-c1ccccc1C21c2ccccc2-c2cccc(N(c3ccc(-c4ccc(-c5cccc6c5oc5ccccc56)cc4)cc3)c3ccc(-c4cccc5ccccc45)cc3)c21] | 22 |
| 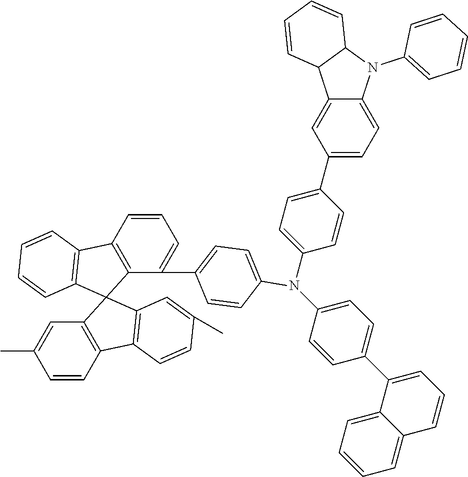 [SMILES: Cc1ccc2c(c1)C1(c3cc(C)ccc3-2)c2ccccc2-c2cccc(-c3ccc(N(c4ccc(-c5ccc6c(c5)C5C=CC=CC5N6c5ccccc5)cc4)c4ccc(-c5cccc6ccccc56)cc4)cc3)c21] | 23 |
| 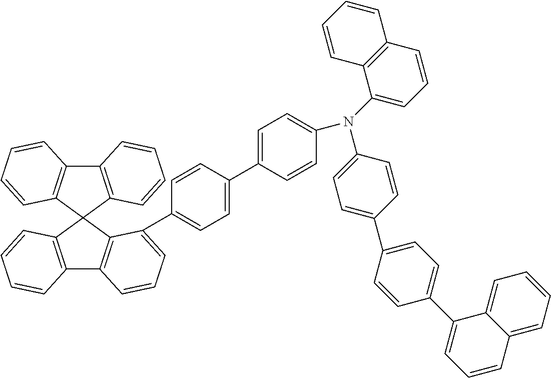 [SMILES: c1ccc2c(c1)-c1ccccc1C21c2ccccc2-c2cccc(-c3ccc(-c4ccc(N(c5ccc(-c6ccc(-c7cccc8ccccc78)cc6)cc5)c5cccc6ccccc56)cc4)cc3)c21] | 24 |
| 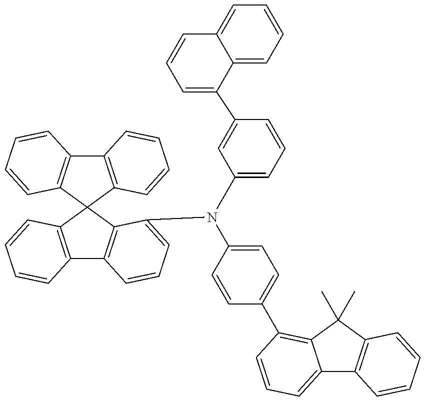 [SMILES: CC1(C)c2ccccc2-c2cccc(-c3ccc(N(c4cccc(-c5cccc6ccccc56)c4)c4cccc5c4C4(c6ccccc6-c6ccccc64)c4ccccc4-5)cc3)c21] | 25 |
| 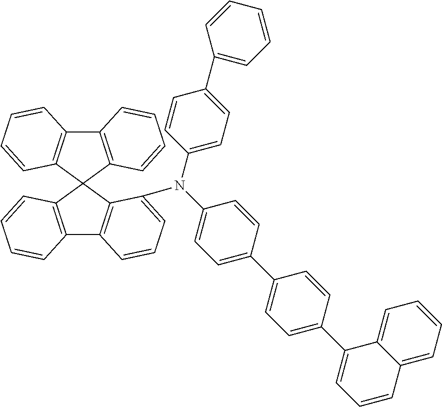 [SMILES: c1ccc(-c2ccc(N(c3ccc(-c4ccc(-c5cccc6ccccc56)cc4)cc3)c3cccc4c3C3(c5ccccc5-c5ccccc53)c3ccccc3-4)cc2)cc1] | 26 |
| 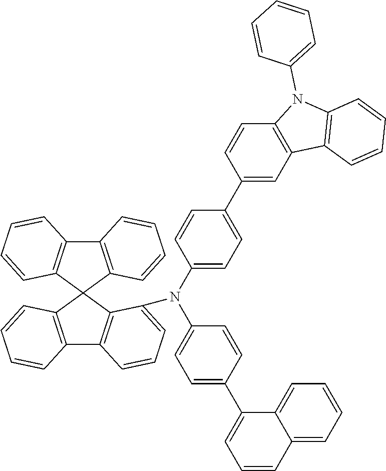 [SMILES: c1ccc(-n2c3ccccc3c3cc(-c4ccc(N(c5ccc(-c6cccc7ccccc67)cc5)c5cccc6c5C5(c7ccccc7-c7ccccc75)c5ccccc5-6)cc4)ccc32)cc1] | 27 |
| 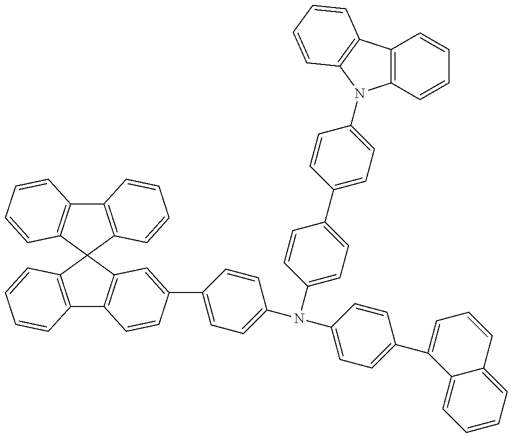 [SMILES: c1ccc2c(c1)-c1ccccc1C21c2ccccc2-c2ccc(-c3ccc(N(c4ccc(-c5ccc(-n6c7ccccc7c7ccccc76)cc5)cc4)c4ccc(-c5cccc6ccccc56)cc4)cc3)cc21] | 28 |
| 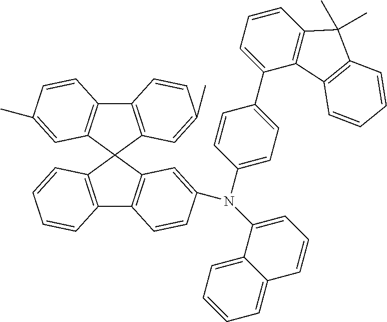 [SMILES: Cc1ccc2c(c1)C1(c3ccccc3-c3ccc(N(c4ccc(-c5cccc6c5-c5ccccc5C6(C)C)cc4)c4cccc5ccccc45)cc31)c1cc(C)ccc1-2] | 29 |
| 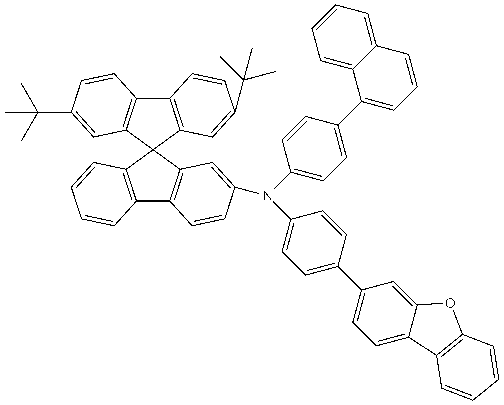 [SMILES: CC(C)(C)c1ccc2c(c1)C1(c3ccccc3-c3ccc(N(c4ccc(-c5ccc6c(c5)oc5ccccc56)cc4)c4ccc(-c5cccc6ccccc56)cc4)cc31)c1cc(C(C)(C)C)ccc1-2] | 30 |
| 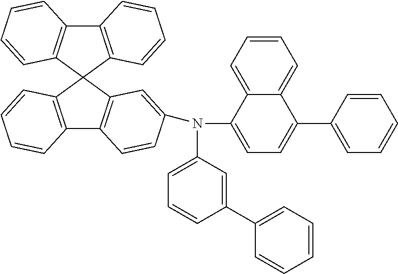 [SMILES: c1ccc(-c2cccc(N(c3ccc4c(c3)C3(c5ccccc5-c5ccccc53)c3ccccc3-4)c3ccc(-c4ccccc4)c4ccccc34)c2)cc1] | 31 |
| 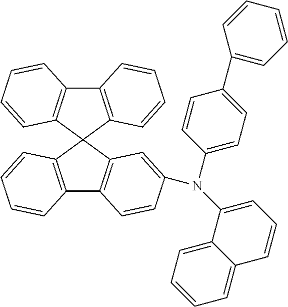 [SMILES: c1ccc(-c2ccc(N(c3ccc4c(c3)C3(c5ccccc5-c5ccccc53)c3ccccc3-4)c3cccc4ccccc34)cc2)cc1] | 32 |
| 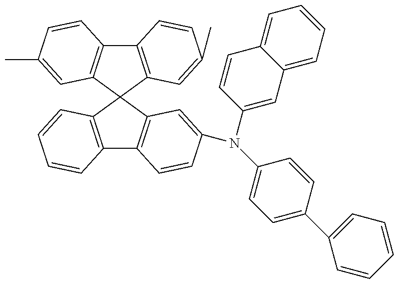 [SMILES: Cc1ccc2c(c1)C1(c3ccccc3-c3ccc(N(c4ccc(-c5ccccc5)cc4)c4ccc5ccccc5c4)cc31)c1cc(C)ccc1-2] | 33 |
| 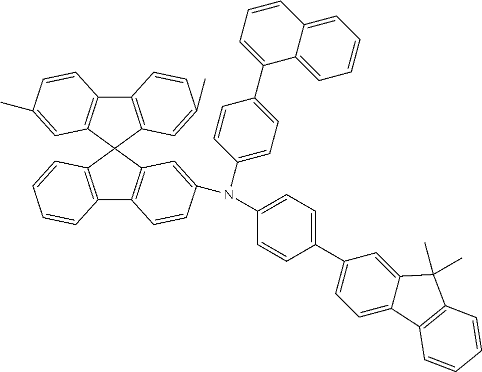 [SMILES: Cc1ccc2c(c1)C1(c3ccccc3-c3ccc(N(c4ccc(-c5ccc6c(c5)C(C)(C)c5ccccc5-6)cc4)c4ccc(-c5cccc6ccccc56)cc4)cc31)c1cc(C)ccc1-2] | 34 |
| 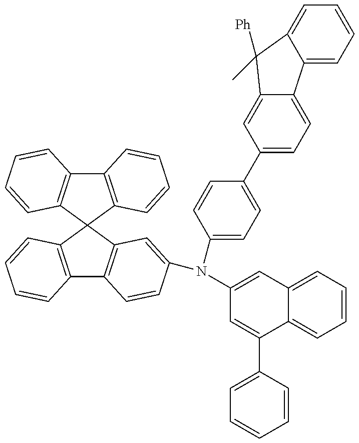 [SMILES: CC1(c2ccccc2)c2ccccc2-c2ccc(-c3ccc(N(c4ccc5c(c4)C4(c6ccccc6-c6ccccc64)c4ccccc4-5)c4cc(-c5ccccc5)c5ccccc5c4)cc3)cc21] | 35 |
| 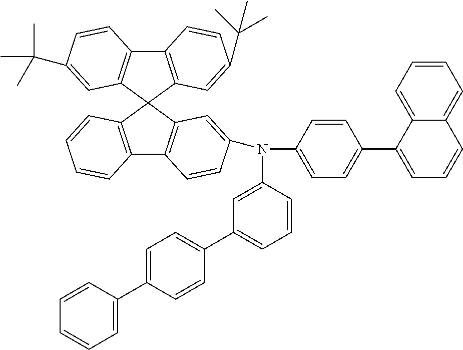 [SMILES: CC(C)(C)c1ccc2c(c1)C1(c3ccccc3-c3ccc(N(c4ccc(-c5cccc6ccccc56)cc4)c4cccc(-c5ccc(-c6ccccc6)cc5)c4)cc31)c1cc(C(C)(C)C)ccc1-2] | 36 |
| 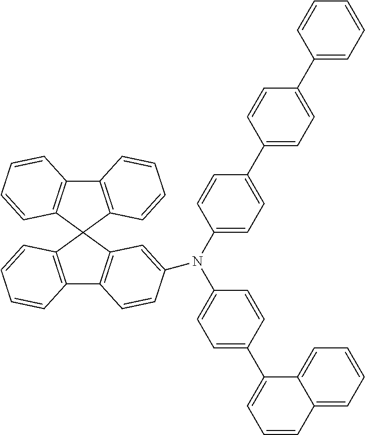 [SMILES: c1ccc(-c2ccc(-c3ccc(N(c4ccc(-c5cccc6ccccc56)cc4)c4ccc5c(c4)C4(c6ccccc6-c6ccccc64)c4ccccc4-5)cc3)cc2)cc1] | 37 |
| 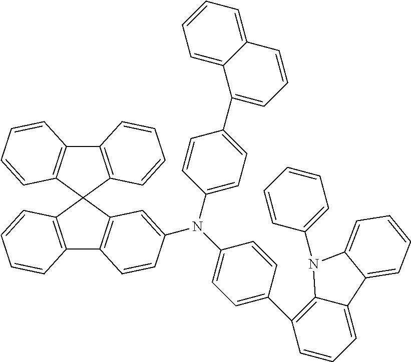 [SMILES: c1ccc(-n2c3ccccc3c3cccc(-c4ccc(N(c5ccc(-c6cccc7ccccc67)cc5)c5ccc6c(c5)C5(c7ccccc7-c7ccccc75)c5ccccc5-6)cc4)c32)cc1] | 38 |
| 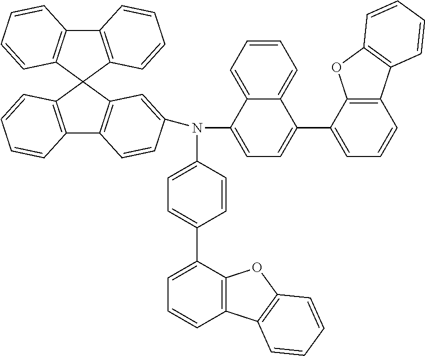 [SMILES: c1ccc2c(c1)-c1ccccc1C21c2ccccc2-c2ccc(N(c3ccc(-c4cccc5c4oc4ccccc45)cc3)c3ccc(-c4cccc5c4oc4ccccc45)c4ccccc34)cc21] | 39 |
| 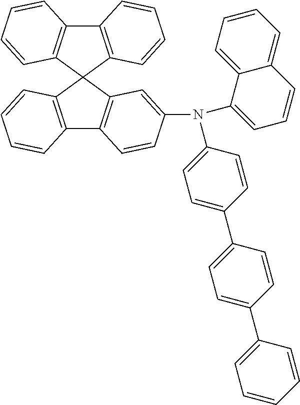 [SMILES: c1ccc(-c2ccc(-c3ccc(N(c4ccc5c(c4)C4(c6ccccc6-c6ccccc64)c4ccccc4-5)c4cccc5ccccc45)cc3)cc2)cc1] | 40 |
| 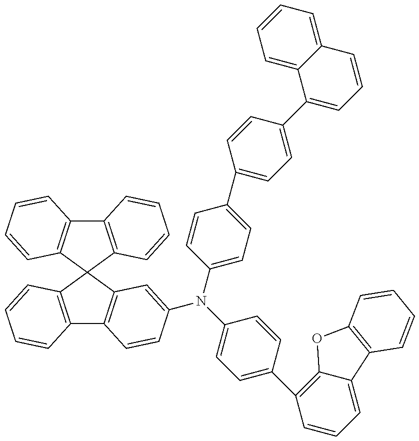 [SMILES: c1ccc2c(c1)-c1ccccc1C21c2ccccc2-c2ccc(N(c3ccc(-c4ccc(-c5cccc6ccccc56)cc4)cc3)c3ccc(-c4cccc5c4oc4ccccc45)cc3)cc21] | 41 |
| 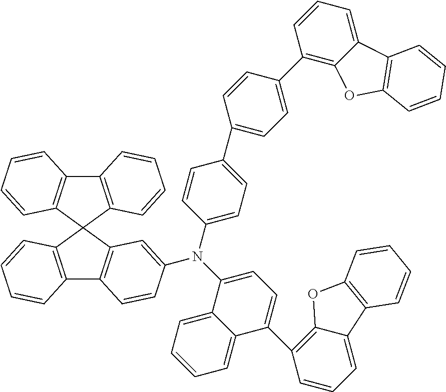 [SMILES: c1ccc2c(c1)-c1ccccc1C21c2ccccc2-c2ccc(N(c3ccc(-c4ccc(-c5cccc6c5oc5ccccc56)cc4)cc3)c3ccc(-c4cccc5c4oc4ccccc45)c4ccccc34)cc21] | 42 |
| 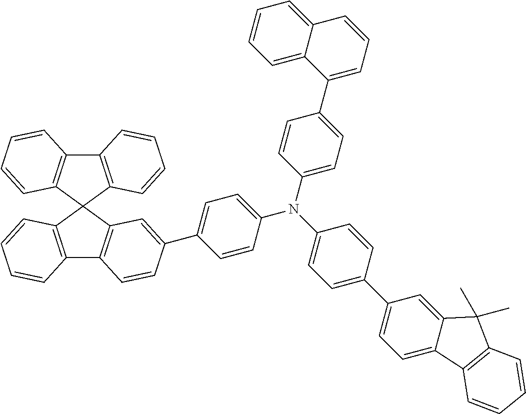 [SMILES: CC1(C)c2ccccc2-c2ccc(-c3ccc(N(c4ccc(-c5ccc6c(c5)C5(c7ccccc7-c7ccccc75)c5ccccc5-6)cc4)c4ccc(-c5cccc6ccccc56)cc4)cc3)cc21] | 43 |
| 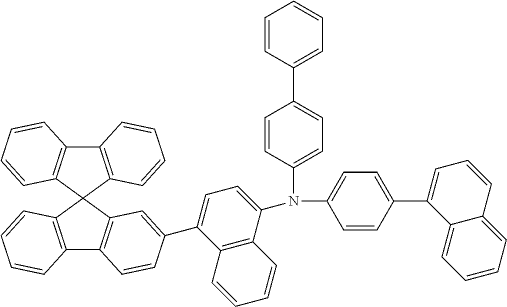 [SMILES: c1ccc(-c2ccc(N(c3ccc(-c4cccc5ccccc45)cc3)c3ccc(-c4ccc5c(c4)C4(c6ccccc6-c6ccccc64)c4ccccc4-5)c4ccccc34)cc2)cc1] | 44 |
| 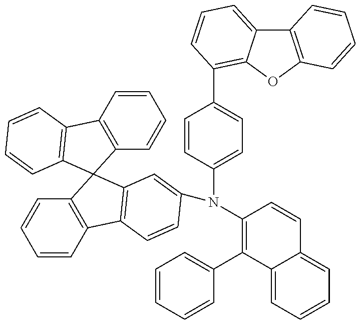 [SMILES: c1ccc(-c2c(N(c3ccc(-c4cccc5c4oc4ccccc45)cc3)c3ccc4c(c3)C3(c5ccccc5-c5ccccc53)c3ccccc3-4)ccc3ccccc23)cc1] | 45 |
| 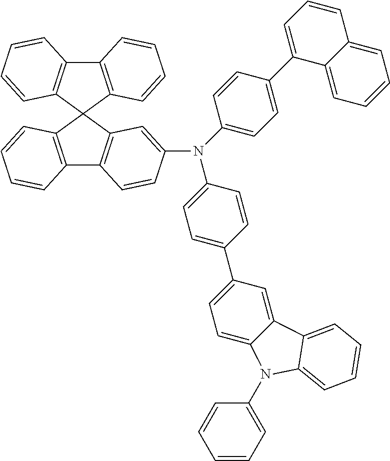 [SMILES: c1ccc(-n2c3ccccc3c3cc(-c4ccc(N(c5ccc(-c6cccc7ccccc67)cc5)c5ccc6c(c5)C5(c7ccccc7-c7ccccc75)c5ccccc5-6)cc4)ccc32)cc1] | 46 |
| 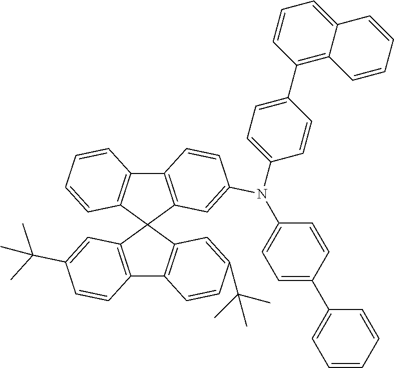 [SMILES: CC(C)(C)c1ccc2c(c1)C1(c3ccccc3-c3ccc(N(c4ccc(-c5ccccc5)cc4)c4ccc(-c5cccc6ccccc56)cc4)cc31)c1cc(C(C)(C)C)ccc1-2] | 47 |
| 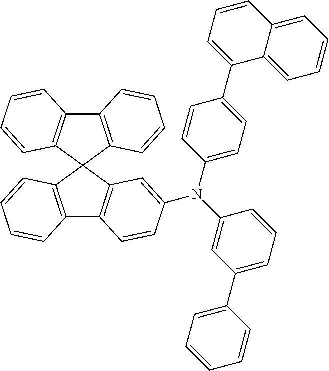 [SMILES: c1ccc(-c2cccc(N(c3ccc(-c4cccc5ccccc45)cc3)c3ccc4c(c3)C3(c5ccccc5-c5ccccc53)c3ccccc3-4)c2)cc1] | 48 |
| 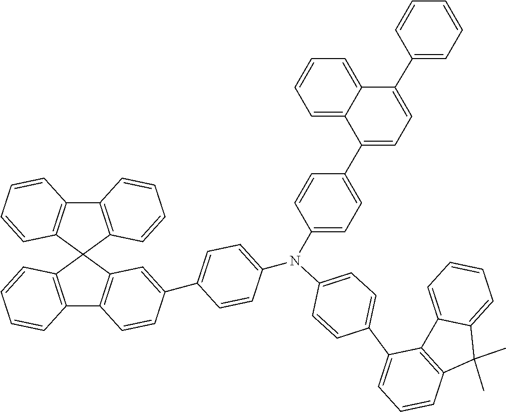 [SMILES: CC1(C)c2ccccc2-c2c(-c3ccc(N(c4ccc(-c5ccc6c(c5)C5(c7ccccc7-c7ccccc75)c5ccccc5-6)cc4)c4ccc(-c5ccc(-c6ccccc6)c6ccccc56)cc4)cc3)cccc21] | 49 |
| 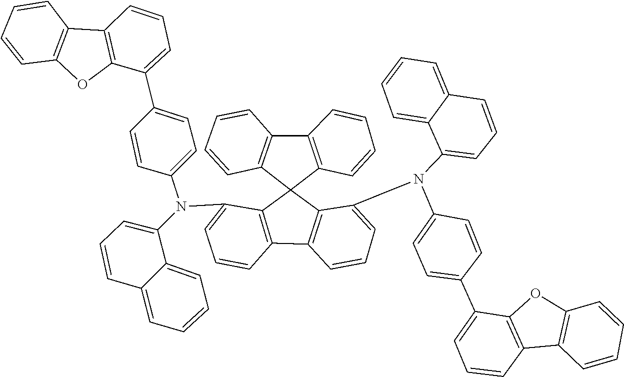 [SMILES: c1ccc2c(c1)-c1ccccc1C21c2c(cccc2N(c2ccc(-c3cccc4c3oc3ccccc34)cc2)c2cccc3ccccc23)-c2cccc(N(c3ccc(-c4cccc5c4oc4ccccc45)cc3)c3cccc4ccccc34)c21] | 50 |
| 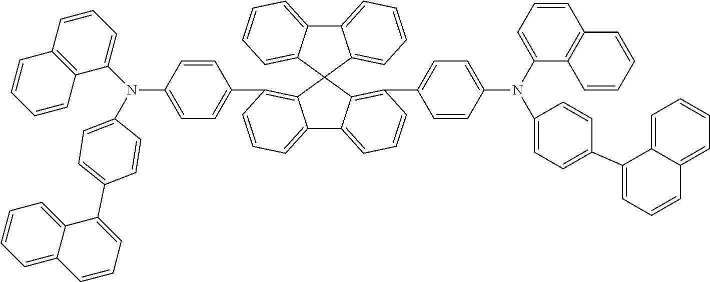 [SMILES: c1ccc2c(c1)-c1ccccc1C21c2c(-c3ccc(N(c4ccc(-c5cccc6ccccc56)cc4)c4cccc5ccccc45)cc3)cccc2-c2cccc(-c3ccc(N(c4ccc(-c5cccc6ccccc56)cc4)c4cccc5ccccc45)cc3)c21] | 51 |
| 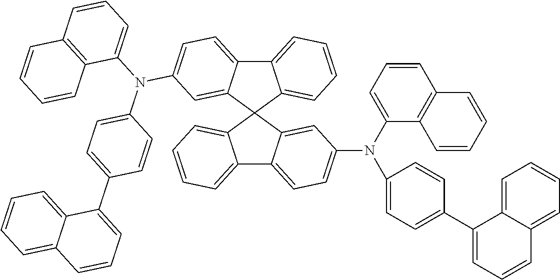 [SMILES: c1ccc2c(c1)-c1ccc(N(c3ccc(-c4cccc5ccccc45)cc3)c3cccc4ccccc34)cc1C21c2ccccc2-c2ccc(N(c3ccc(-c4cccc5ccccc45)cc3)c3cccc4ccccc34)cc21] | 52 |
| 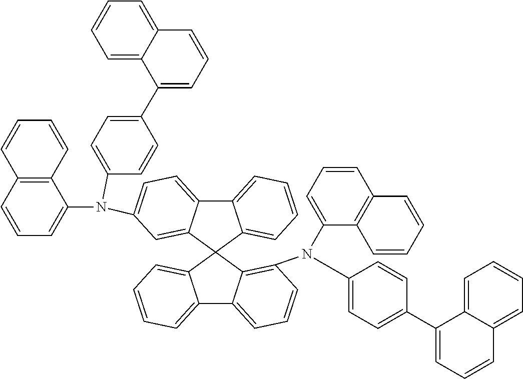 [SMILES: c1ccc2c(c1)-c1ccc(N(c3ccc(-c4cccc5ccccc45)cc3)c3cccc4ccccc34)cc1C21c2ccccc2-c2cccc(N(c3ccc(-c4cccc5ccccc45)cc3)c3cccc4ccccc34)c21] | 53 |
| 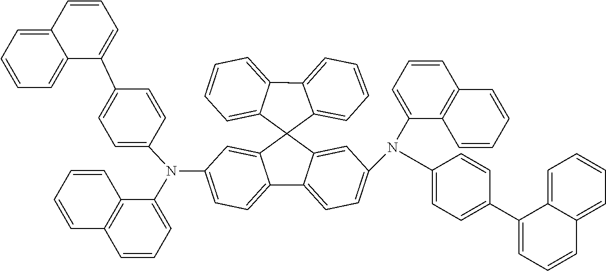 [SMILES: c1ccc2c(c1)-c1ccccc1C21c2cc(N(c3ccc(-c4cccc5ccccc45)cc3)c3cccc4ccccc34)ccc2-c2ccc(N(c3ccc(-c4cccc5ccccc45)cc3)c3cccc4ccccc34)cc21] | 54 |
| 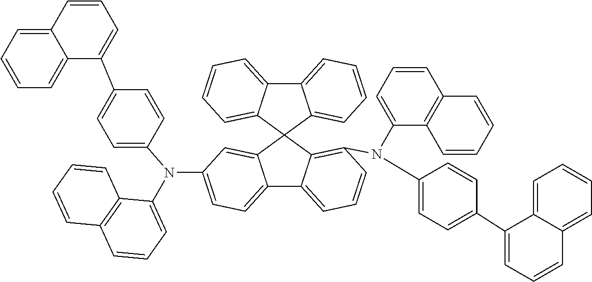 [SMILES: c1ccc2c(c1)-c1ccccc1C21c2cc(N(c3ccc(-c4cccc5ccccc45)cc3)c3cccc4ccccc34)ccc2-c2cccc(N(c3ccc(-c4cccc5ccccc45)cc3)c3cccc4ccccc34)c21] | 55 |
| 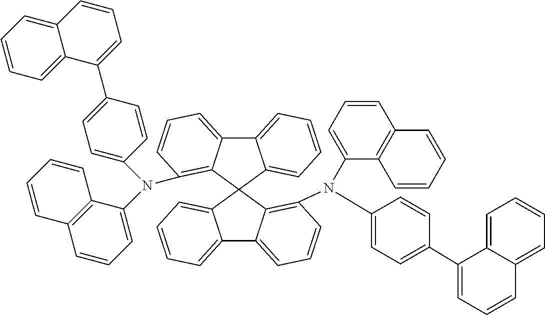 [SMILES: c1ccc2c(c1)-c1cccc(N(c3ccc(-c4cccc5ccccc45)cc3)c3cccc4ccccc34)c1C21c2ccccc2-c2cccc(N(c3ccc(-c4cccc5ccccc45)cc3)c3cccc4ccccc34)c21] | 56 |
| 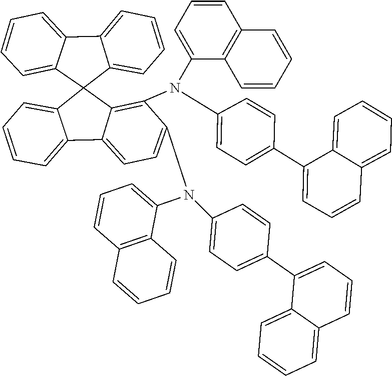 [SMILES: c1ccc2c(c1)-c1ccccc1C21c2ccccc2-c2ccc(N(c3ccc(-c4cccc5ccccc45)cc3)c3cccc4ccccc34)c(N(c3ccc(-c4cccc5ccccc45)cc3)c3cccc4ccccc34)c21] | 57 |
| 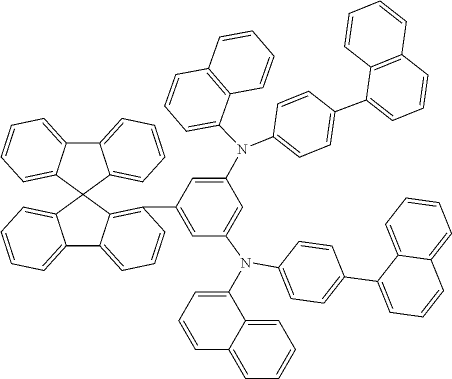 [SMILES: c1ccc2c(c1)-c1ccccc1C21c2ccccc2-c2cccc(-c3cc(N(c4ccc(-c5cccc6ccccc56)cc4)c4cccc5ccccc45)cc(N(c4ccc(-c5cccc6ccccc56)cc4)c4cccc5ccccc45)c3)c21] | 58 |
| 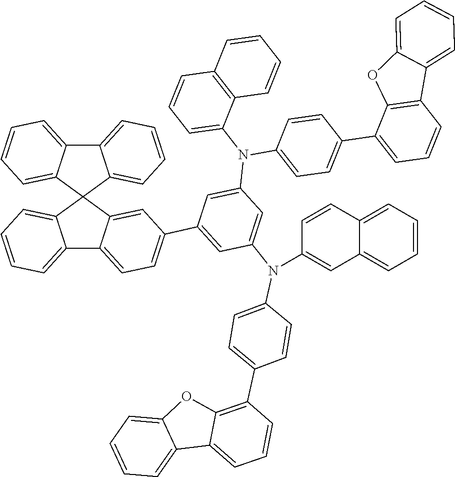 [SMILES: c1ccc2c(c1)-c1ccccc1C21c2ccccc2-c2ccc(-c3cc(N(c4ccc(-c5cccc6c5oc5ccccc56)cc4)c4ccc5ccccc5c4)cc(N(c4ccc(-c5cccc6c5oc5ccccc56)cc4)c4cccc5ccccc45)c3)cc21] | 59 |
| 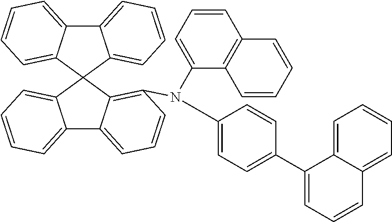 [SMILES: c1ccc2c(c1)-c1ccccc1C21c2ccccc2-c2cccc(N(c3ccc(-c4cccc5ccccc45)cc3)c3cccc4ccccc34)c21] | 60 |
| 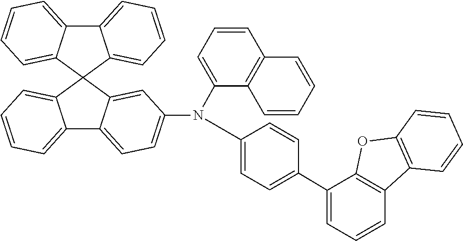 [SMILES: c1ccc2c(c1)-c1ccccc1C21c2ccccc2-c2ccc(N(c3ccc(-c4cccc5c4oc4ccccc45)cc3)c3cccc4ccccc34)cc21] | 61 |
| 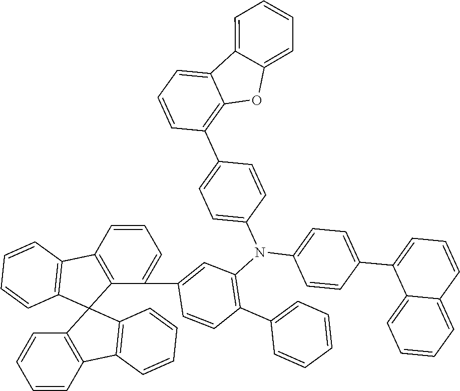 [SMILES: c1ccc(-c2ccc(-c3cccc4c3C3(c5ccccc5-c5ccccc53)c3ccccc3-4)cc2N(c2ccc(-c3cccc4ccccc34)cc2)c2ccc(-c3cccc4c3oc3ccccc34)cc2)cc1] | 62 |
| 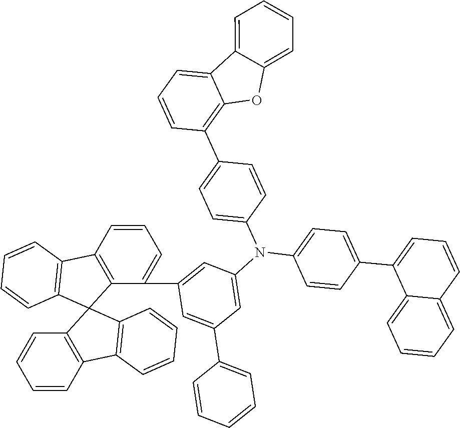 [SMILES: c1ccc(-c2cc(-c3cccc4c3C3(c5ccccc5-c5ccccc53)c3ccccc3-4)cc(N(c3ccc(-c4cccc5ccccc45)cc3)c3ccc(-c4cccc5c4oc4ccccc45)cc3)c2)cc1] | 63 |

Preferred processes for preparing compounds of the formula (I) are shown below.

According to the process shown in scheme 1 a halogen-substituted spirobifluorene is subjected to a Buchwald coupling reaction with a diarylamine to prepare a compound of the formula (I).

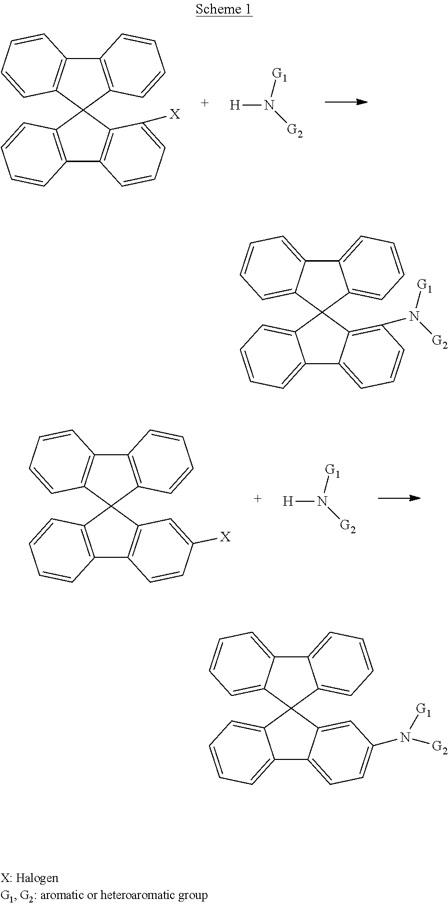

It is alternatively possible according to the process shown in scheme 2 to initially perform a Buchwald coupling between a halogen-substituted fluorenone and a diarylamine and subsequently prepare the spirobifluorene scaffold by addition of ortho-halogenylbiphenyl with an organometallic and ring-closing.

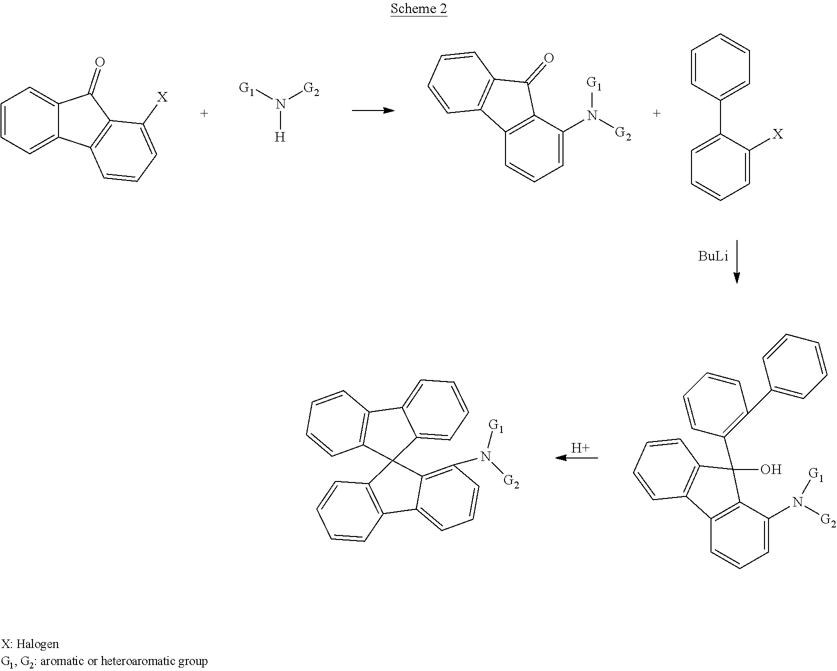

It is alternatively possible via the synthesis shown in scheme 3 to prepare compounds of the formula (I) which comprise a spacer group between the spirobifluorenyl group and the amino group. To this end a halogenyl-substituted fluorenone is in a Suzuki coupling reacted with a boronic acid-substituted aryl group bearing a halogenyl group. The coupling product is subsequently reacted with diaryl amine in a Buchwald reaction. This is followed by preparation of the spirobifluorene scaffold by addition of ortho-halogenylbiphenyl with an organometallic and ring-closing analogously to the synthesis shown in scheme 2.

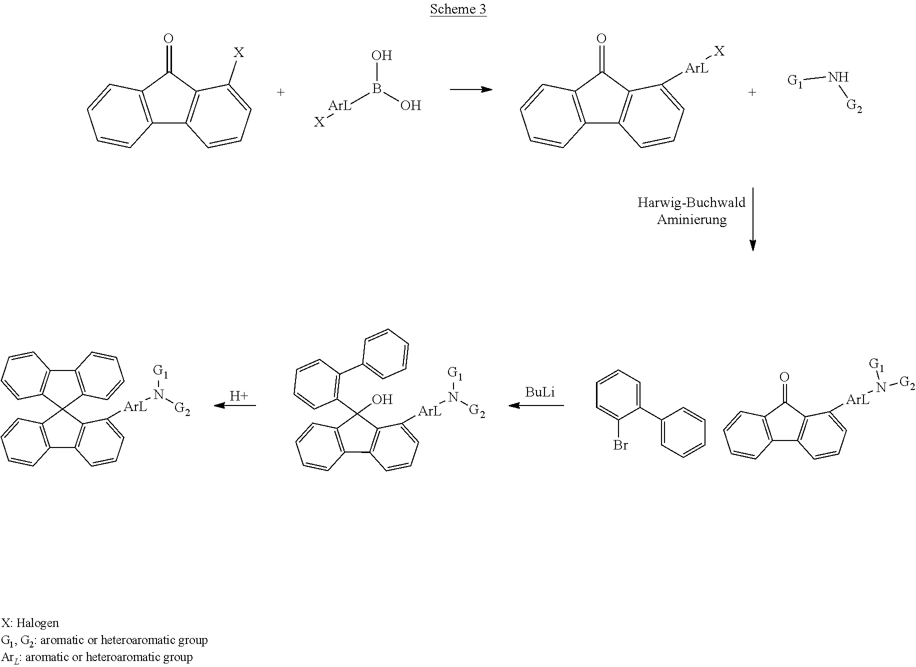

A further preferred process for preparing compounds of the formula (I) which comprise a spacer group between the spirobifluorenyl group and the amino group is shown in scheme 4. To this end a halogen-substituted spirobifluorene is in a Suzuki coupling reacted with an amine comprising three aromatic/heteroaromatic radicals, wherein one of these radicals has a boronic acid group.

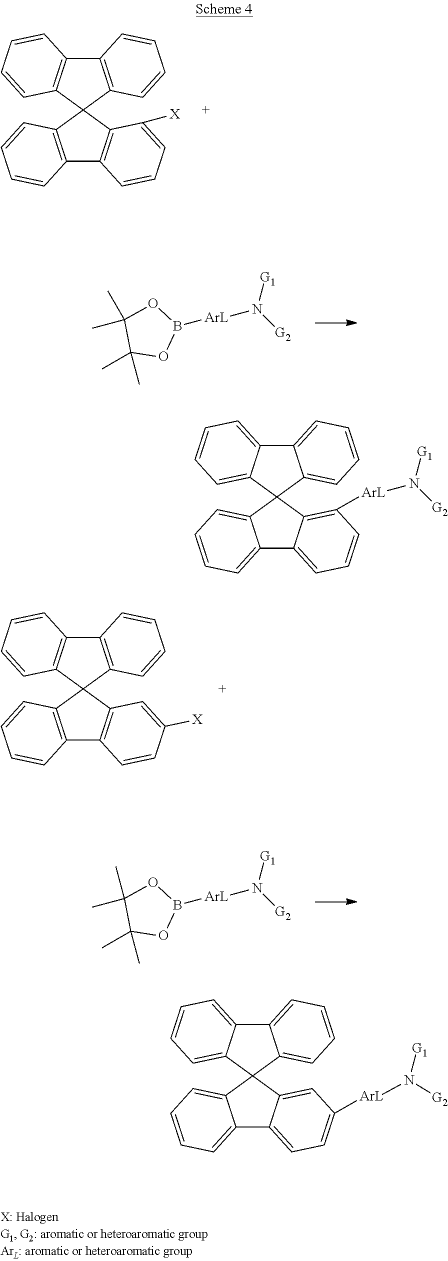

A further preferred process for preparing compounds of the formula (I) which comprise a spacer group between the spirobifluorenyl group and the amino group is shown in scheme 5.

To this end a spirobifluorene bearing a halogen group is in a Suzuki coupling reacted with a boronic acid-substituted aryl group bearing a halogen group. A diarylamino group is subsequently introduced in a Buchwald coupling.

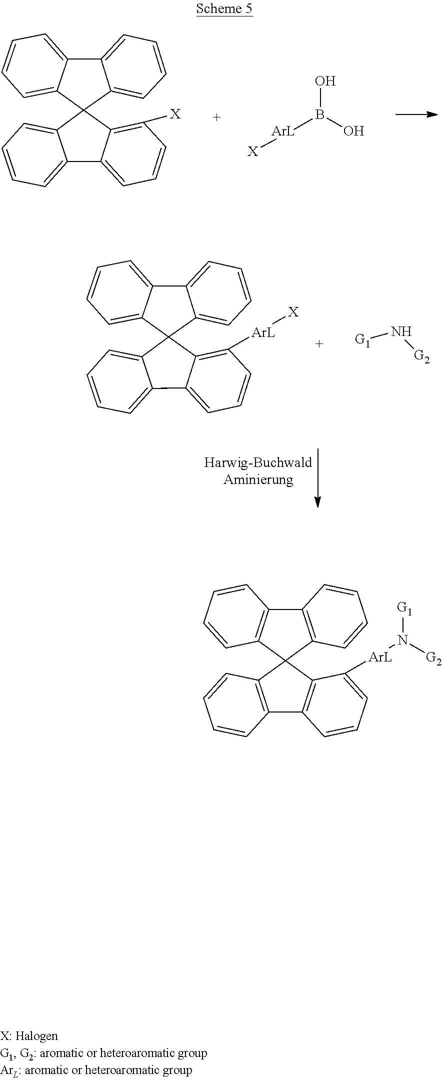

The present application thus provides a process for preparing a compound of the formula (I), characterized in that an amino group is introduced using an organometallic coupling reaction. The organometallic coupling reaction is preferably selected from a Suzuki coupling and a Buchwald coupling. The organometallic coupling reaction is furthermore preferably carried out between a spirobifluorenyl derivative having a halogen substituent, or a fluorenone derivative having a halogen substituent, and the amino group. The above-described compounds of the invention, especially compounds substituted by reactive leaving groups, such as bromine, iodine, chlorine, boronic acid or boronic ester, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C═C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more compounds of formula (I), wherein the bond(s) to the polymer, oligomer or dendrimer may be localized at any desired positions substituted by R¹, R², R³, R⁴, R⁵ or R⁶ in formula (I). According to the linkage of the compound of formula (I), the compound is part of a side chain of the oligomer or polymer or part of the main chain. An oligomer in the context of this invention is understood to mean a compound formed from at least three monomer units. A polymer in the context of the invention is understood to mean a compound formed from at least ten monomer units.

The polymers, oligomers or dendrimers of the invention may be conjugated, partly conjugated or nonconjugated. The oligomers or polymers of the invention may be linear, branched or dendritic. In the structures having linear linkage, the units of formula (I) may be joined directly to one another, or they may be joined to one another via a bivalent group, for example via a substituted or unsubstituted alkylene group, via a heteroatom or via a bivalent aromatic or heteroaromatic group. In branched and dendritic structures, it is possible, for example, for three or more units of formula (I) to be joined via a trivalent or higher-valency group, for example via a trivalent or higher-valency aromatic or heteroaromatic group, to give a branched or dendritic oligomer or polymer.

For the repeat units of formula (I) in oligomers, dendrimers and polymers, the same preferences apply as described above for compounds of formula (I).

For preparation of the oligomers or polymers, the monomers of the invention are homopolymerized or copolymerized with further monomers. Suitable and preferred comonomers are selected from fluorenes, spirobifluorenes, paraphenylenes, carbazoles, thiophenes, dihydrophenanthrenes, cis- and trans-indenofluorenes, ketones, phenanthrenes or else two or more of these units. The polymers, oligomers and dendrimers typically contain still further units, for example emitting (fluorescent or phosphorescent) units, for example vinyltriarylamines or phosphorescent metal complexes, and/or charge transport units, especially those based on triarylamines.

The polymers, oligomers and dendrimers of the invention have advantageous properties, especially high lifetimes, high efficiencies and good colour coordinates.

The polymers and oligomers of the invention are generally prepared by polymerization of one or more monomer types, of which at least one monomer leads to repeat units of the formula (I) in the polymer. Suitable polymerization reactions are known to those skilled in the art and are described in the literature. Particularly suitable and preferred polymerization reactions which lead to C—C and C—N couplings are as follows:
(A) SUZUKI polymerization;
(B) YAMAMOTO polymerization;
(C) STILLE polymerization; and
(D) HARTWIG-BUCHWALD polymerization.

How the polymerization can be conducted by these methods and how the polymers can then be separated from the reaction medium and purified is known to those skilled in the art and is described in detail in the literature.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (-)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, alpha-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, or mixtures of these solvents.

The invention therefore further provides a formulation, especially a solution, dispersion or emulsion, comprising at least one compound of formula (I) or at least one polymer, oligomer or dendrimer containing at least one unit of formula (I) and at least one solvent, preferably an organic solvent. The way in which such solutions can be prepared is known to those skilled in the art. The compound of formula (I) is suitable for use in an electronic device, especially an organic electroluminescent device (OLED). Depending on the substitution, the compound of the formula (I) can be used in different functions and layers. Preference is given to use as a hole-transporting material in a hole-transporting layer and/or as matrix material in an emitting layer, more preferably in combination with a phosphorescent emitter.

The invention therefore further provides for the use of a compound of formula (I) in an electronic device. This electronic device is preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and more preferably organic electroluminescent devices (OLEDs).

The invention further provides an electronic device comprising at least one compound of formula (I). This electronic device is preferably selected from the abovementioned devices.

Particular preference is given to an organic electroluminescent device comprising an anode, cathode and at least one emitting layer, characterized in that at least one organic layer comprising at least one compound of formula (I) is present in the device. Preference is given to an organic electroluminescent device comprising an anode, cathode and at least one emitting layer, characterized in that at least one organic layer in the device, selected from hole-transporting and emitting layers, comprises at least one compound of formula (I).

A hole-transporting layer is understood here to mean all layers disposed between anode and emitting layer, preferably hole injection layer, hole transport layer and electron blocker layer. A hole injection layer is understood here to mean a layer that directly adjoins the anode. A hole transport layer is understood here to mean a layer which is between the anode and emitting layer but does not directly adjoin the anode, and preferably does not directly adjoin the emitting layer either. An electron blocker layer is understood here to mean a layer which is between the anode and emitting layer and directly adjoins the emitting layer. An electron blocker layer preferably has a high-energy LUMO and hence prevents electrons from exiting from the emitting layer.

Apart from the cathode, anode and emitting layer, the electronic device may comprise further layers. These are selected, for example, from in each case one or more hole injection layers, hole transport layers, hole blocker layers, electron transport layers, electron injection layers, electron blocker layers, exciton blocker layers, interlayers, charge generation layers and/or organic or inorganic p/n junctions. However, it should be pointed out that not every one of these layers need necessarily be present and the choice of layers always depends on the compounds used and especially also on whether the device is a fluorescent or phosphorescent electroluminescent device.

The sequence of layers in the electronic device is preferably as follows:
anode-
hole injection layer-
hole transport layer-
optionally further hole transport layers-
emitting layer-
optionally hole blocker layer-
electron transport layer-
electron injection layer-
cathode-.

At the same time, it should be pointed out again that not all the layers mentioned need be present and/or that further layers may additionally be present.

The organic electroluminescent device of the invention may contain two or more emitting layers. More preferably, these emission layers have several emission maxima between 380 nm and 750 nm overall, such that the overall result is white emission; in other words, various emitting compounds which may fluoresce or phosphoresce and which emit blue, green, yellow, orange or red light are used in the emitting layers. Especially preferred are three-layer systems, i.e. systems having three emitting layers, wherein one of the three layers in each case shows blue emission, one of the three layers in each case shows green emission, and one of the three layers in each case shows orange or red emission. The compounds of the invention here are preferably present in a hole-transporting layer or in the emitting layer. It should be noted that, for the production of white light, rather than a plurality of colour-emitting emitter compounds, an emitter compound used individually which emits over a broad wavelength range may also be suitable.

It is preferable that the compound of the formula (I) is used as hole transport material. The emitting layer here may be a fluorescent emitting layer, or it may be a phosphorescent emitting layer. The emitting layer is preferably a blue-fluorescing layer or a green-phosphorescing layer.

When the device containing the compound of the formula (I) contains a phosphorescent emitting layer, it is preferable that this layer contains two or more, preferably exactly two, different matrix materials (mixed matrix system). Preferred embodiments of mixed matrix systems are described in detail further down.

If the compound of formula (I) is used as hole transport material in a hole transport layer, a hole injection layer or an electron blocker layer, the compound can be used as pure material, i.e. in a proportion of 100%, in the hole transport layer, or it can be used in combination with one or more further compounds.

In a preferred embodiment, a hole-transporting layer comprising the compound of the formula (I) additionally comprises one or more further hole-transporting compounds. These further hole-transporting compounds are preferably selected from triarylamine compounds, more preferably from monotriarylamine compounds. They are most preferably selected from the preferred embodiments of hole transport materials that are specified further down. In the preferred embodiment described, the compound of the formula (I) and the one or more further hole-transporting compounds are preferably each present in a proportion of at least 10%, more preferably each in a proportion of at least 20%.

In a preferred embodiment, a hole-transporting layer comprising the compound of the formula (I) additionally contains one or more p-dopants. p-Dopants used according to the present invention are preferably those organic electron acceptor compounds capable of oxidizing one or more of the other compounds in the mixture.

Particularly preferred as p-dopants are quinodimethane compounds, azaindenofluorenediones, azaphenalenes, azatriphenylenes, I₂, metal halides, preferably transition metal halides, metal oxides, preferably metal oxides comprising at least one transition metal or a metal from main group 3, and transition metal complexes, preferably complexes of Cu, Co, Ni, Pd and Pt with ligands containing at least one oxygen atom as binding site.

Preference is further given to transition metal oxides as dopants, preferably oxides of rhenium, molybdenum and tungsten, more preferably Re₂O₇, MoO₃, WO₃ and ReO₃. Still further preference is given to complexes of bismuth in the (III) oxidation state, more particularly bismuth(III) complexes with electron-deficient ligands, more particularly carboxylate ligands.

The p-dopants are preferably in substantially homogeneous distribution in the p-doped layers. This can be achieved, for example, by co-evaporation of the p-dopant and the hole transport material matrix. The p-dopant is preferably present in a proportion of 1% to 10% in the p-doped layer.

Preferred p-dopants are especially the following compounds:

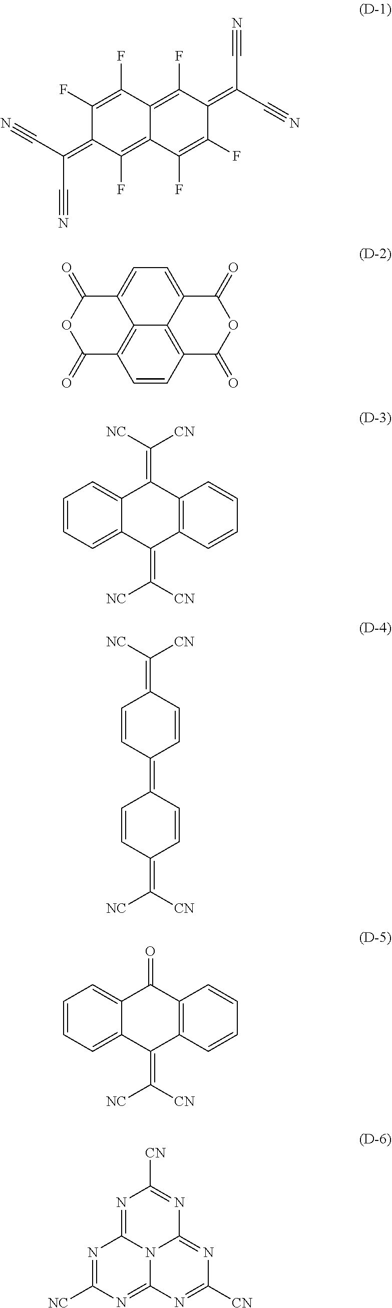

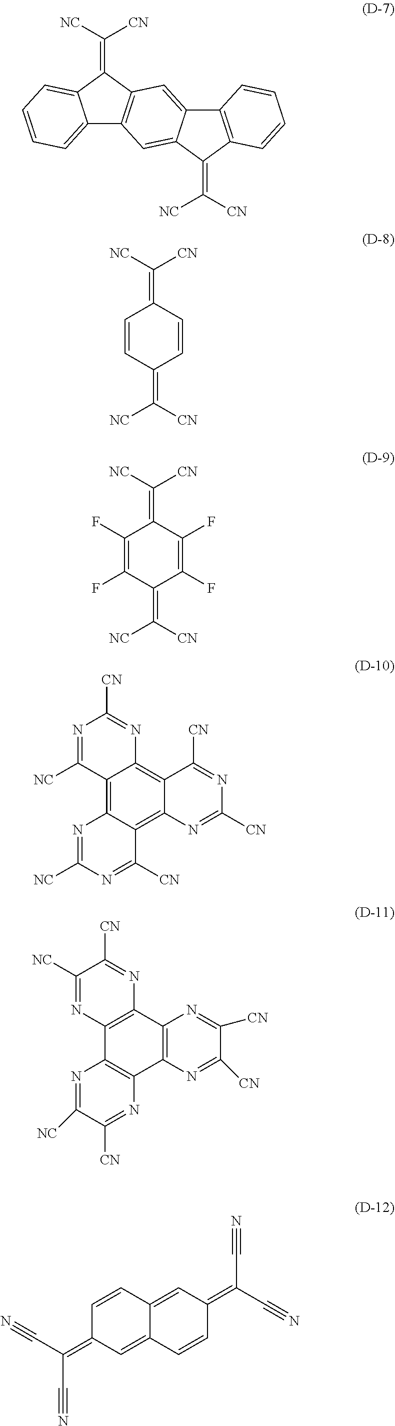

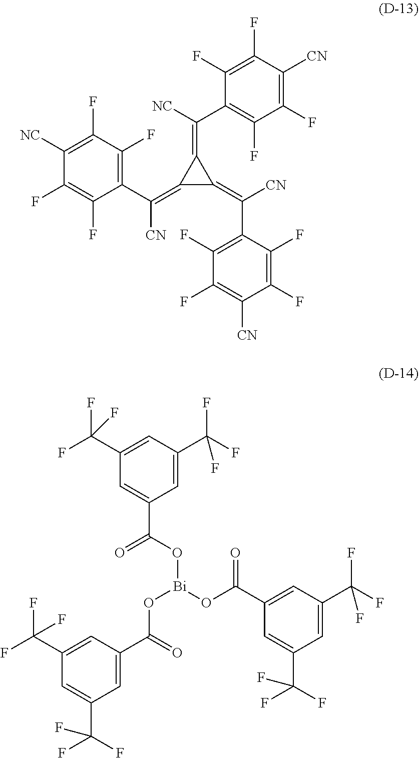

In a preferred embodiment, a hole injection layer that conforms to one of the following embodiments is present in the device: a) it contains a triarylamine and a p-dopant; or b) it contains a single electron-deficient material (electron acceptor). In a preferred embodiment of embodiment a), the triarylamine is a monotriarylamine, especially one of the preferred triarylamine derivatives mentioned further down. In a preferred embodiment of embodiment b), the electron-deficient material is a hexaazatriphenylene derivative as described in US 2007/0092755.

The compound of the formula (I) may be present in a hole injection layer, in a hole transport layer and/or in an electron blocker layer of the device. When the compound is present in a hole injection layer or in a hole transport layer, it has preferably been p-doped, meaning that it is in mixed form with a p-dopant, as described above, in the layer.

The compound of the formula (I) is preferably present in an electron blocker layer. In this case, it is preferably not p-doped. Further preferably, in this case, it is preferably in the form of a single compound in the layer without addition of a further compound.

In an alternative preferred embodiment, the compound of the formula (I) is used in an emitting layer as matrix material in combination with one or more emitting compounds, preferably phosphorescent emitting compounds. The phosphorescent emitting compounds here are preferably selected from red-phosphorescing and green-phosphorescing compounds.

The proportion of the matrix material in the emitting layer in this case is between 50.0% and 99.9% by volume, preferably between 80.0% and 99.5% by volume, and more preferably between 85.0% and 97.0% by volume.

Correspondingly, the proportion of the emitting compound is between 0.1% and 50.0% by volume, preferably between 0.5% and 20.0% by volume, and more preferably between 3.0% and 15.0% by volume.

An emitting layer of an organic electroluminescent device may also contain systems comprising a plurality of matrix materials (mixed matrix systems) and/or a plurality of emitting compounds. In this case too, the emitting compounds are generally those compounds having the smaller proportion in the system and the matrix materials are those compounds having the greater proportion in the system. In individual cases, however, the proportion of a single matrix material in the system may be less than the proportion of a single emitting compound.

It is preferable that the compounds of formula (I) are used as a component of mixed matrix systems, preferably for phosphorescent emitters. The mixed matrix systems preferably comprise two or three different matrix materials, more preferably two different matrix materials. Preferably, in this case, one of the two materials is a material having hole-transporting properties and the other material is a material having electron-transporting properties. It is further preferable when one of the materials is selected from compounds having a large energy differential between HOMO and LUMO (wide-bandgap materials). The compound of the formula (I) in a mixed matrix system is preferably the matrix material having hole-transporting properties. Correspondingly, when the compound of the formula (I) is used as matrix material for a phosphorescent emitter in the emitting layer of an OLED, a second matrix compound having electron-transporting properties is present in the emitting layer. The two different matrix materials may be present here in a ratio of 1:50 to 1:1, preferably 1:20 to 1:1, more preferably 1:10 to 1:1 and most preferably 1:4 to 1:1.

The desired electron-transporting and hole-transporting properties of the mixed matrix components may, however, also be combined mainly or entirely in a single mixed matrix component, in which case the further mixed matrix component(s) fulfil(s) other functions.

Preference is given to using the following material classes in the abovementioned layers of the device:

Phosphorescent Emitters:

The term “phosphorescent emitters” typically encompasses compounds where the emission of light is effected through a spin-forbidden transition, for example a transition from an excited triplet state or a state having a higher spin quantum number, for example a quintet state.

Suitable phosphorescent emitters are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitters, compounds containing copper, molybdenum, tungsten, rhenium, ruthenium, osmium, rhodium, iridium, palladium, platinum, silver, gold or europium, especially compounds containing iridium, platinum or copper.

In the context of the present invention, all luminescent iridium, platinum or copper complexes are considered to be phosphorescent compounds.

In general, all phosphorescent complexes as used for phosphorescent OLEDs according to the prior art and as known to those skilled in the art in the field of organic electroluminescent devices are suitable for use in the devices of the invention. Further examples of suitable phosphorescent emitters are shown in the following table:

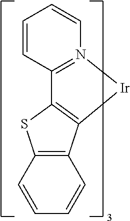

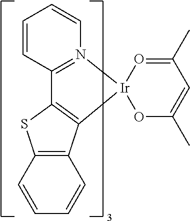

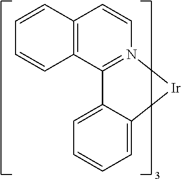

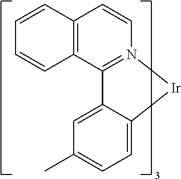

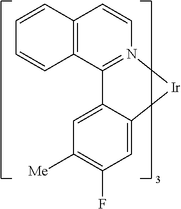

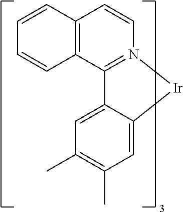

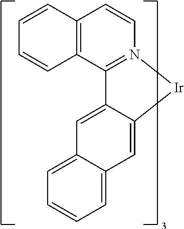

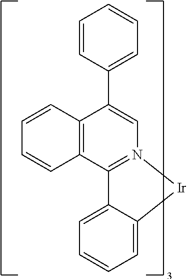

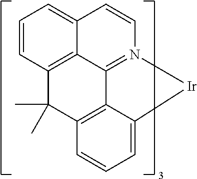

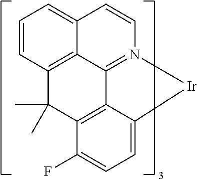

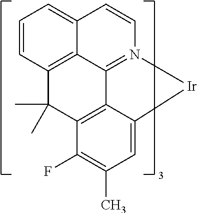

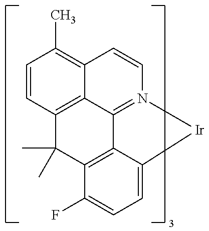

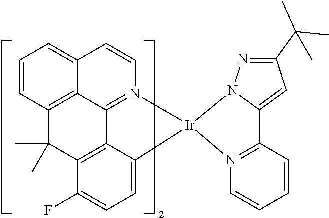

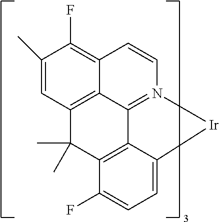

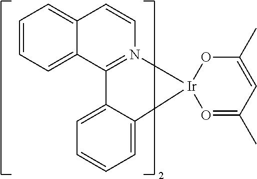

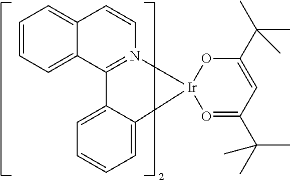

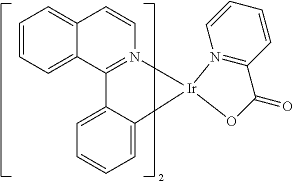

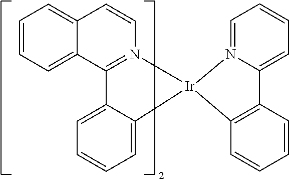

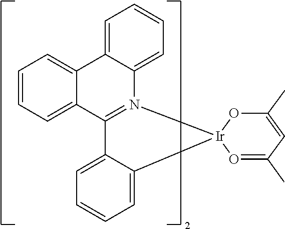

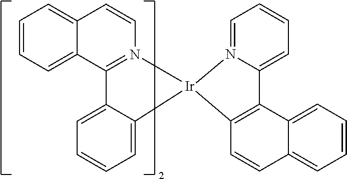

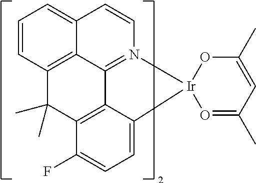

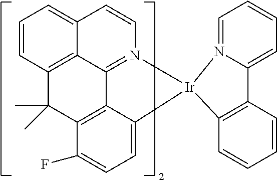

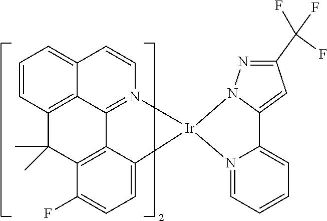

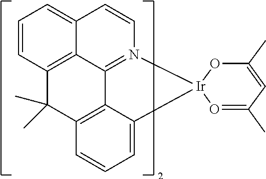

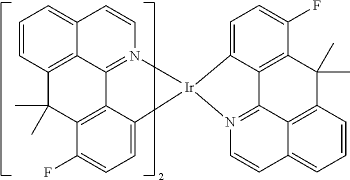

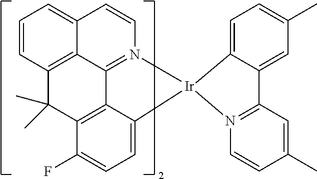

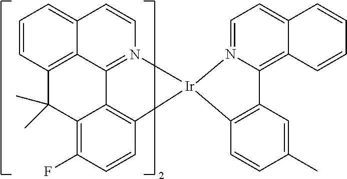

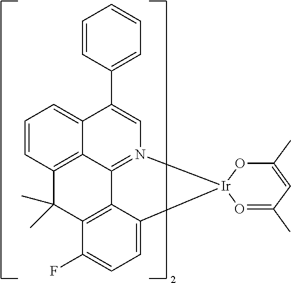

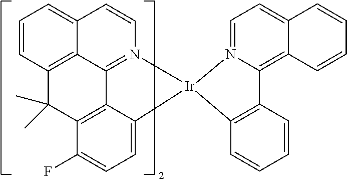

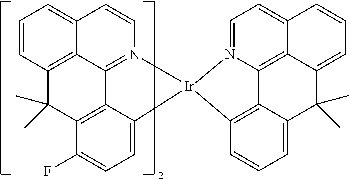

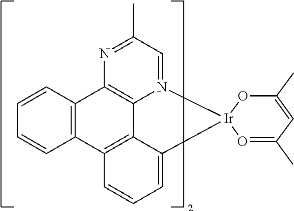

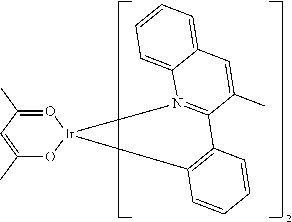

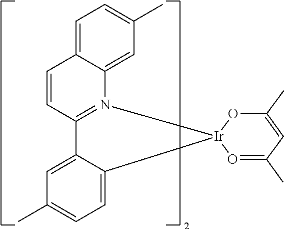

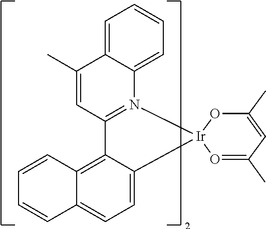

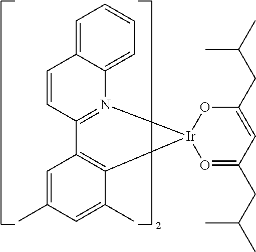

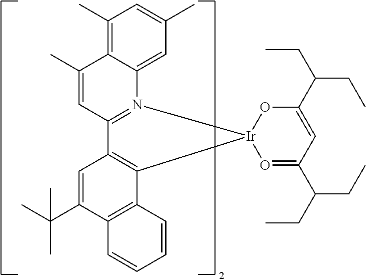

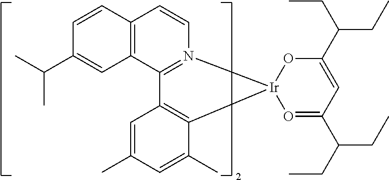

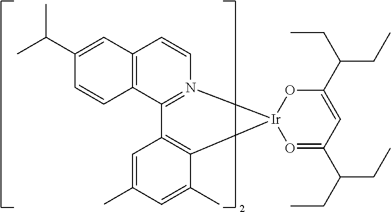

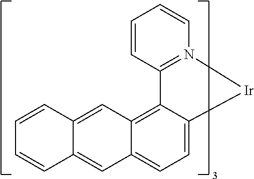

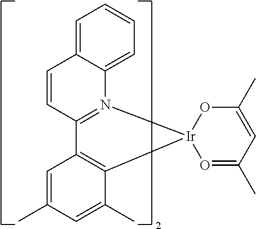

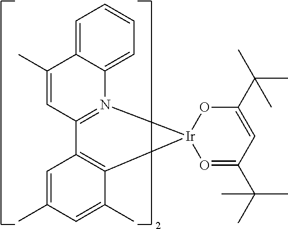

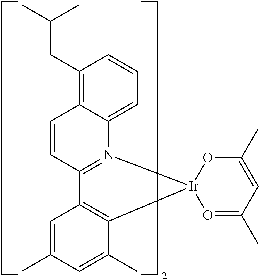

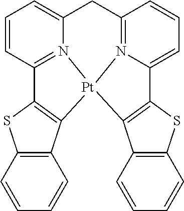

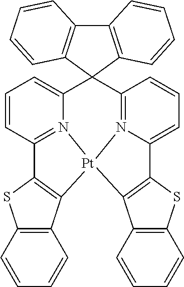

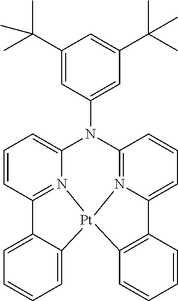

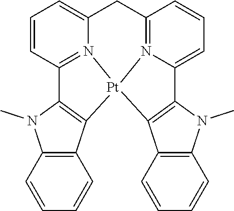

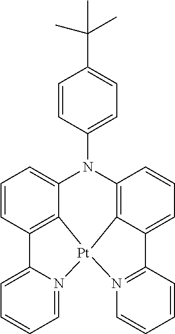

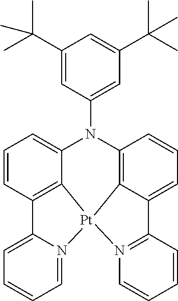

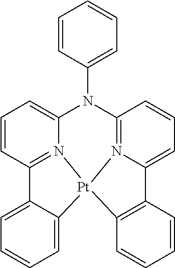

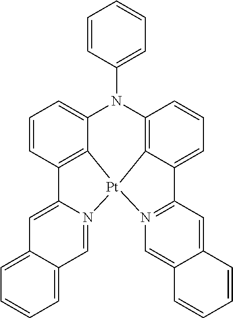

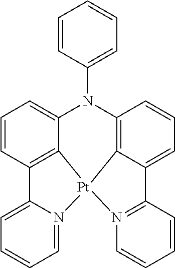

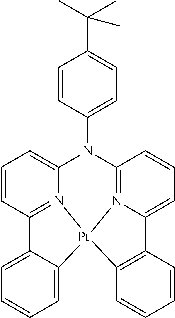

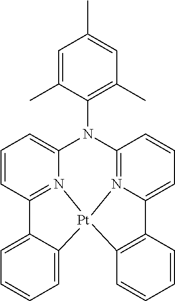

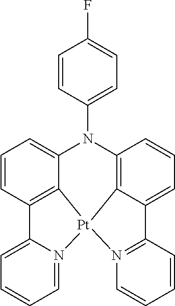

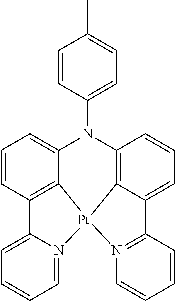

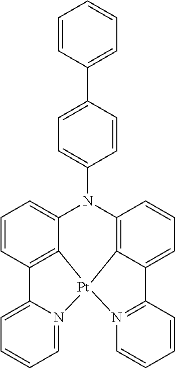

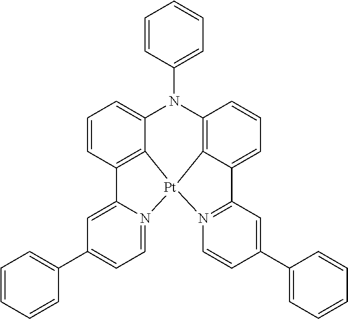

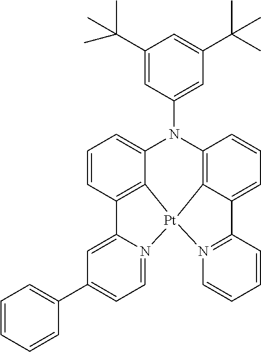

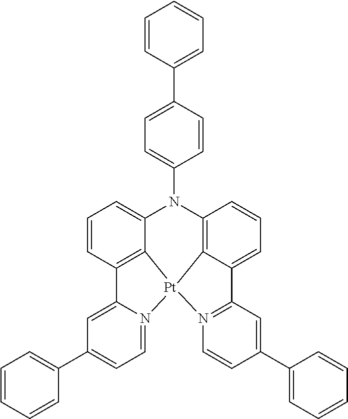

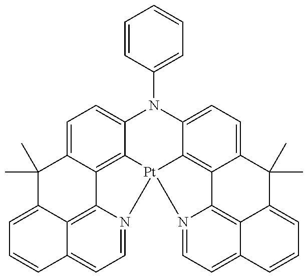

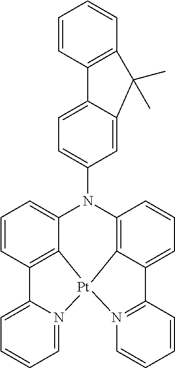

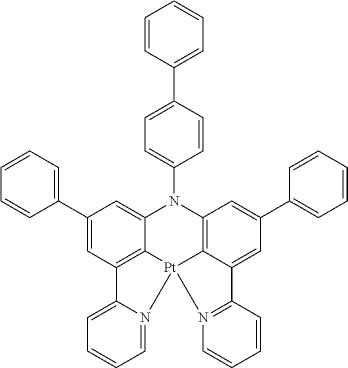

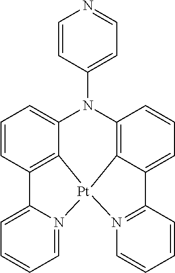

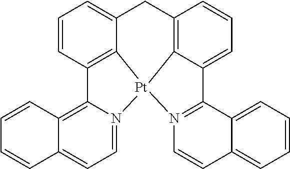

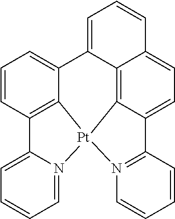

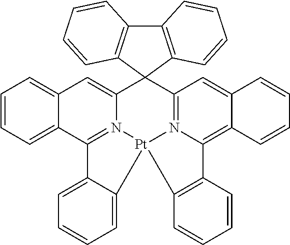

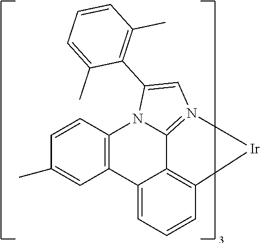

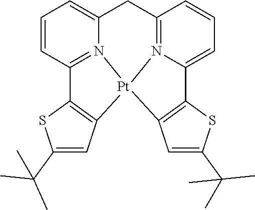

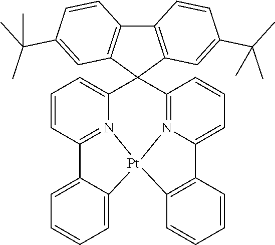

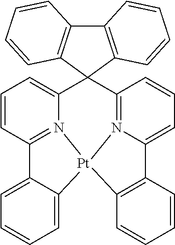

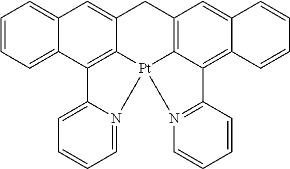

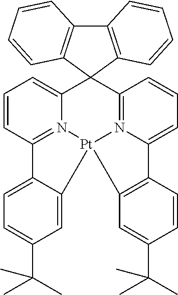

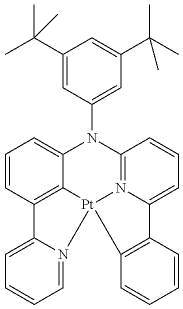

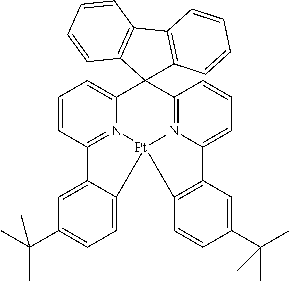

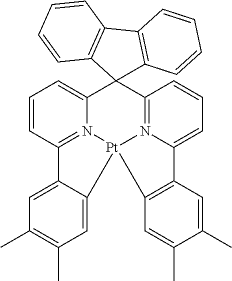

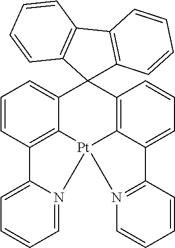

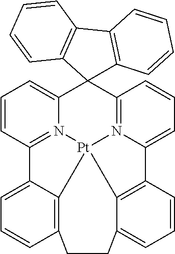

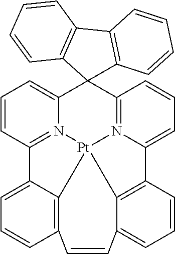

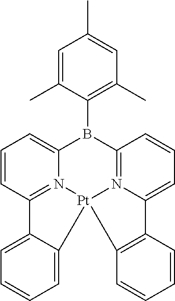

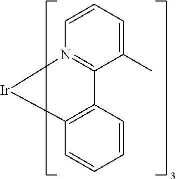

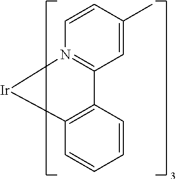

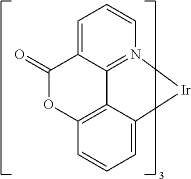

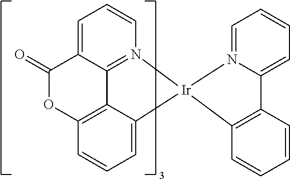

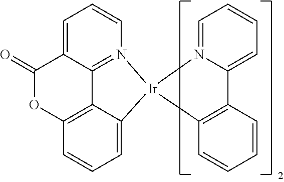

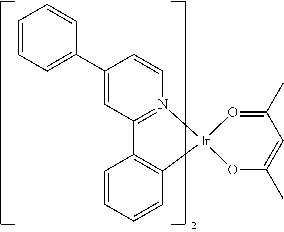

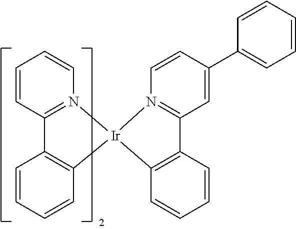

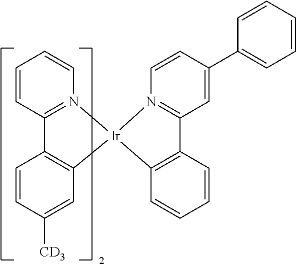

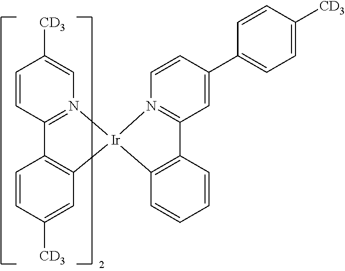

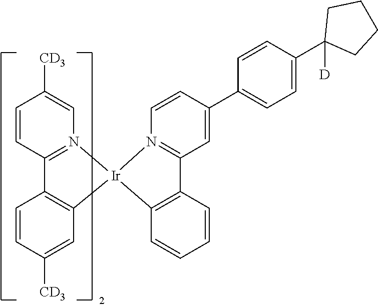

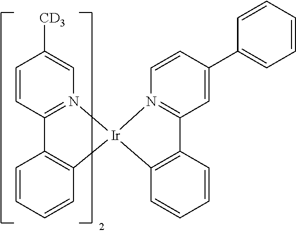

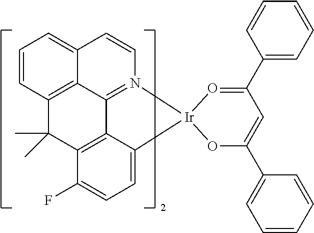

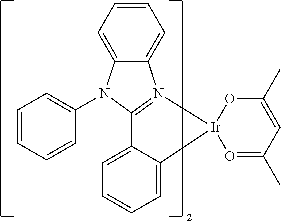

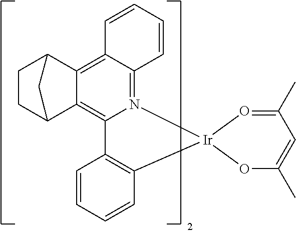

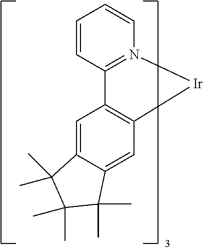

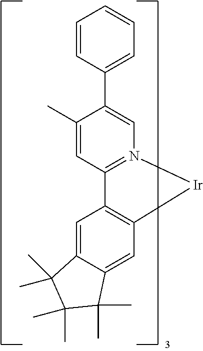

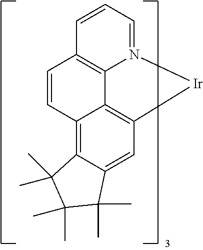

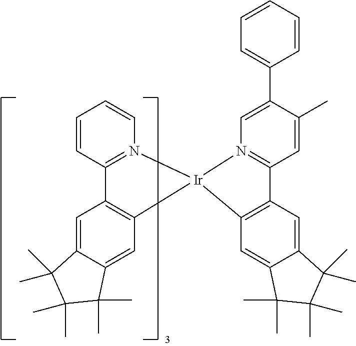

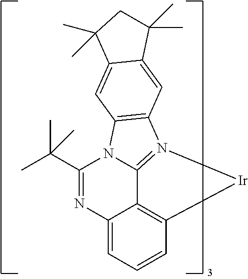

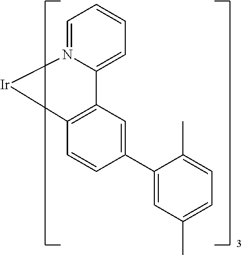

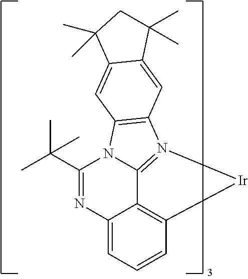

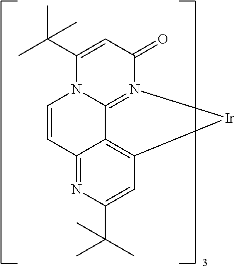

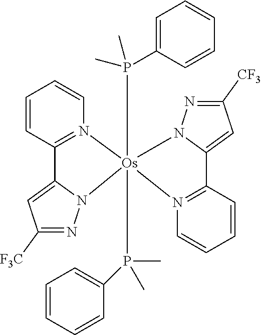

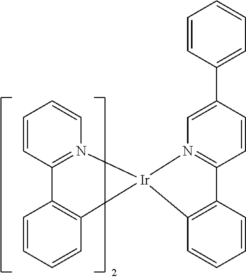

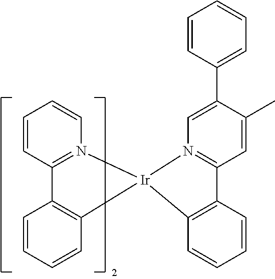

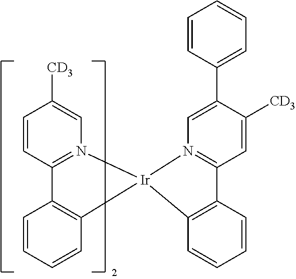

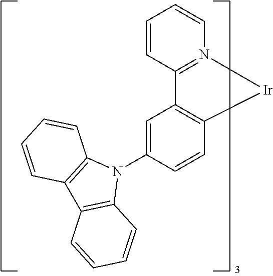

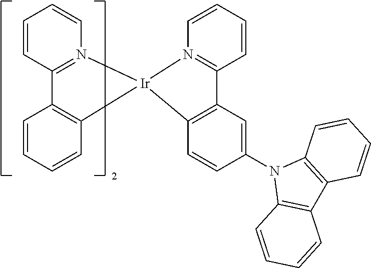

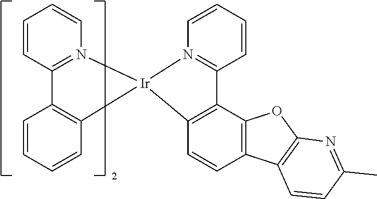

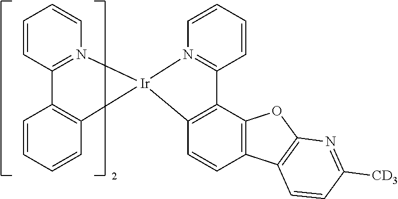

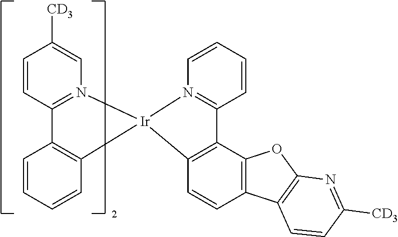

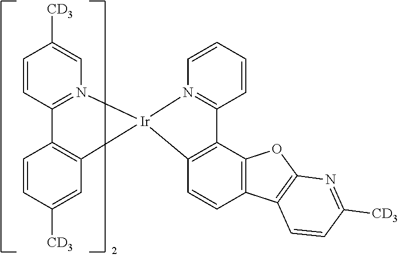

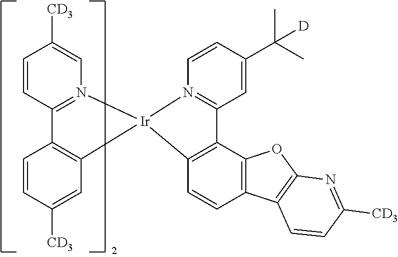

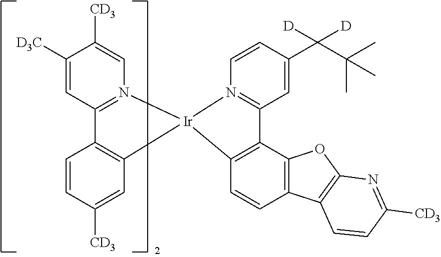

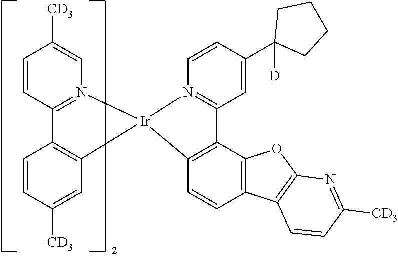

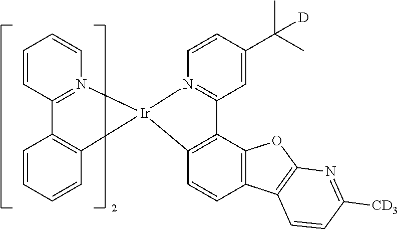

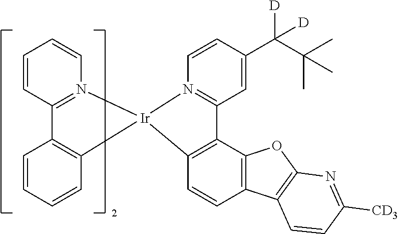

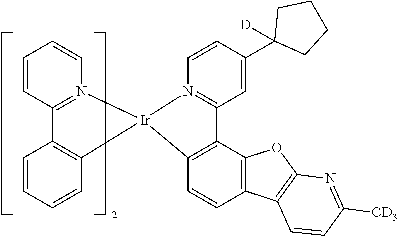

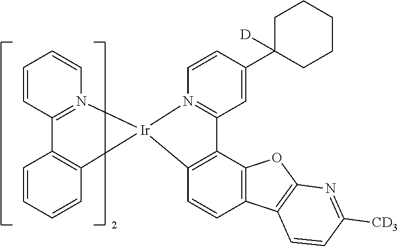

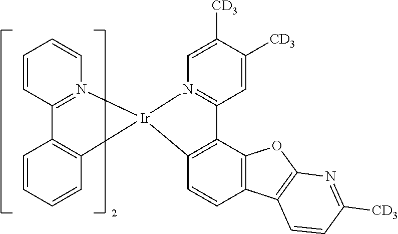

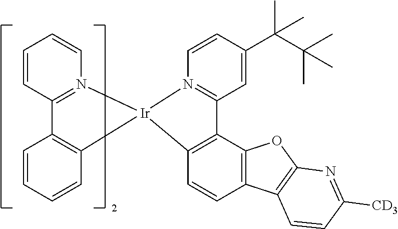

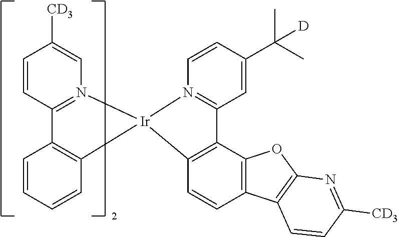

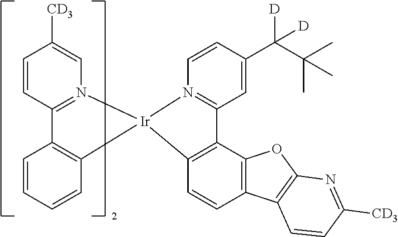

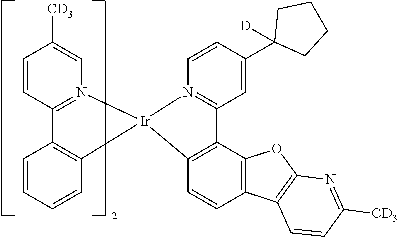

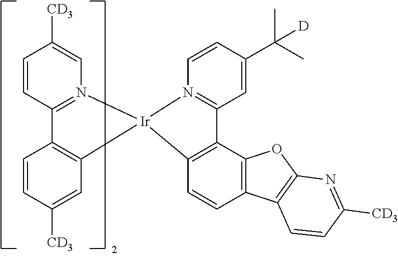

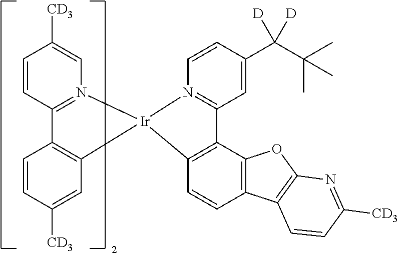

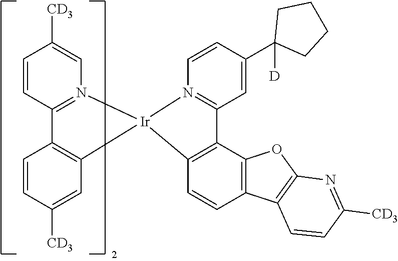

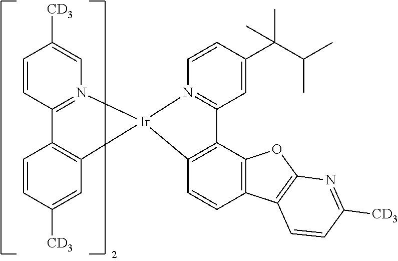

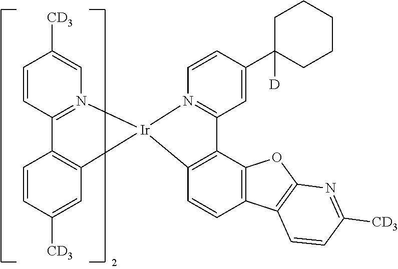

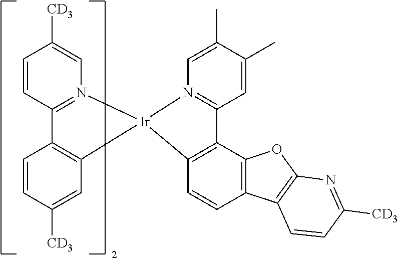

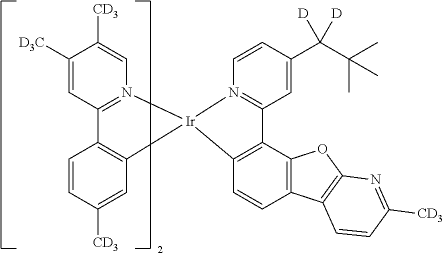

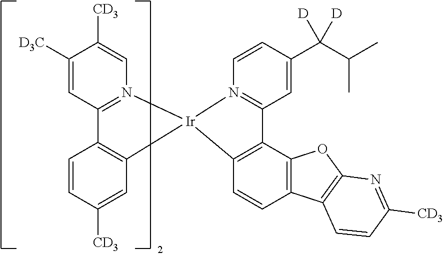

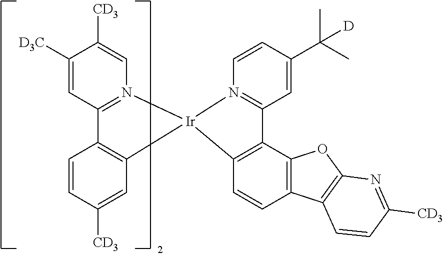

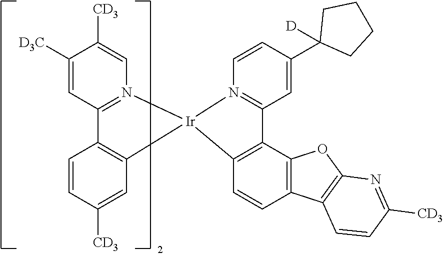

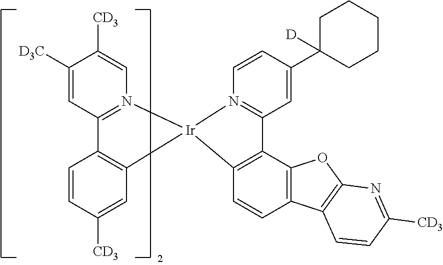

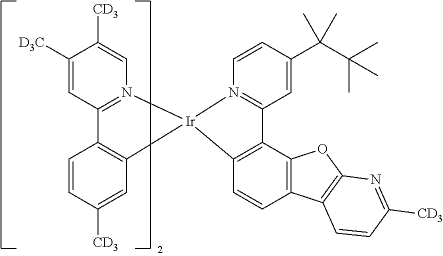

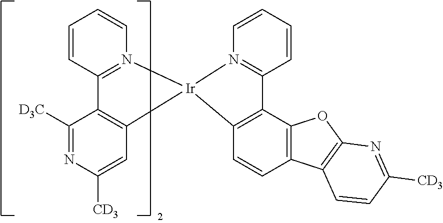

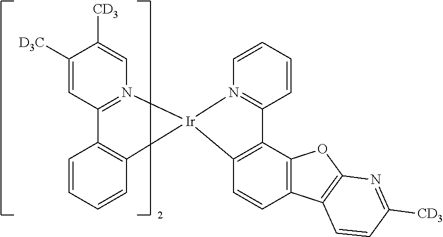

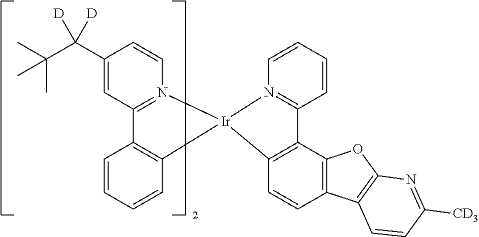

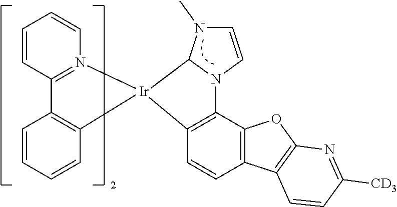

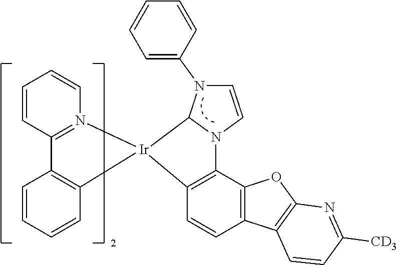

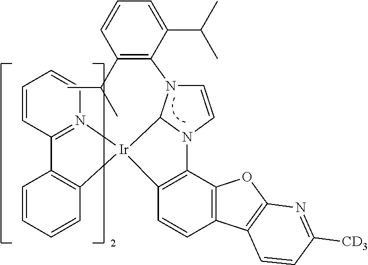

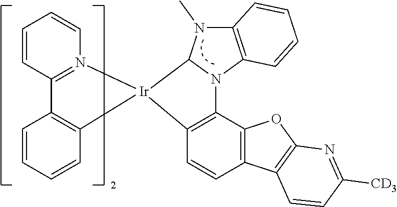

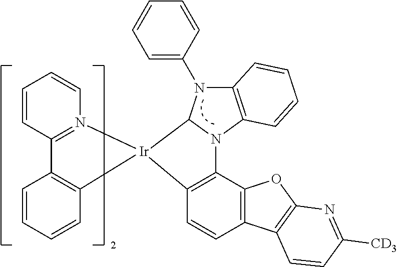

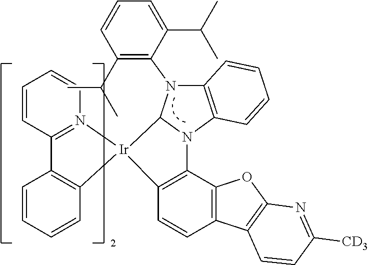

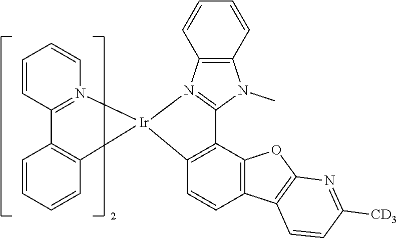

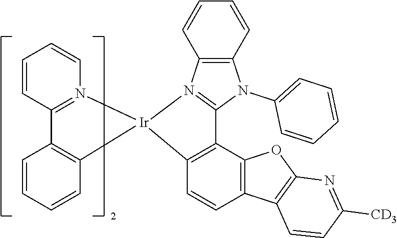

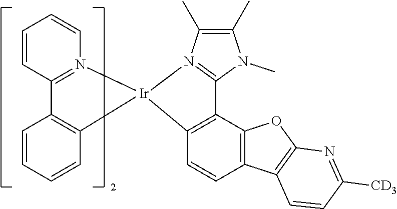

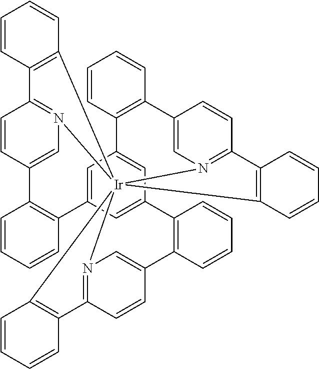

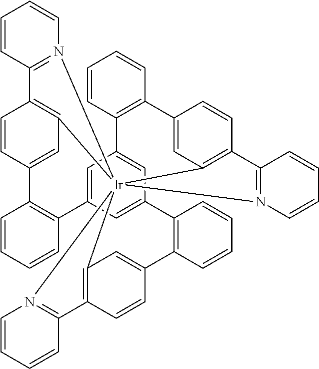

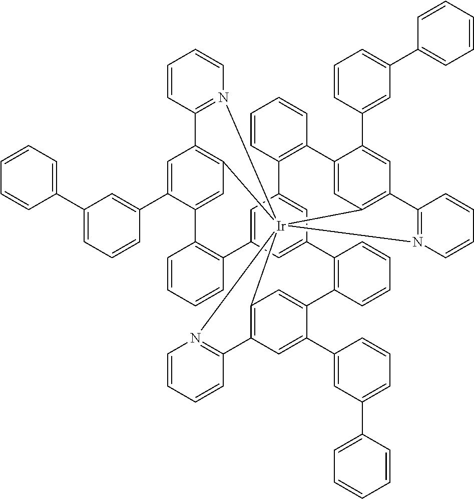

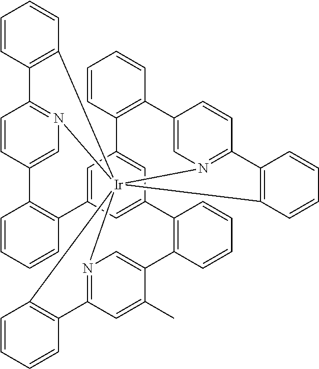

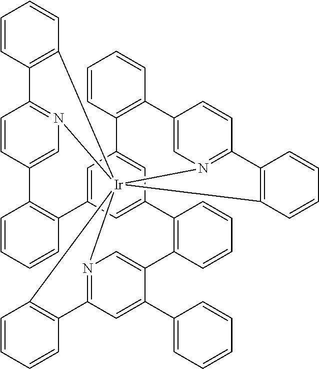

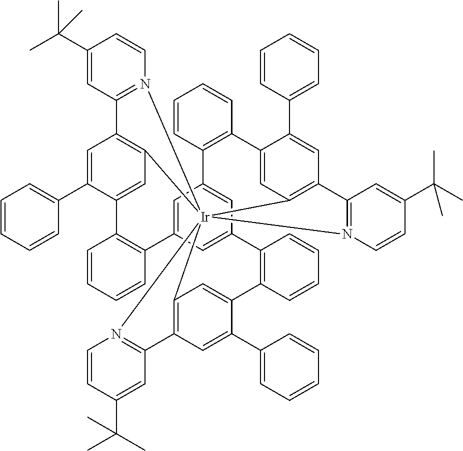

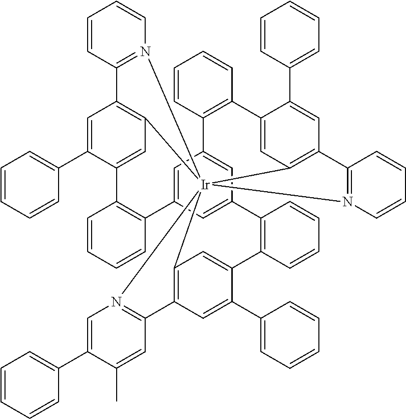

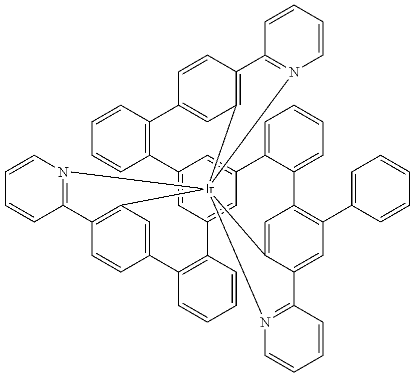

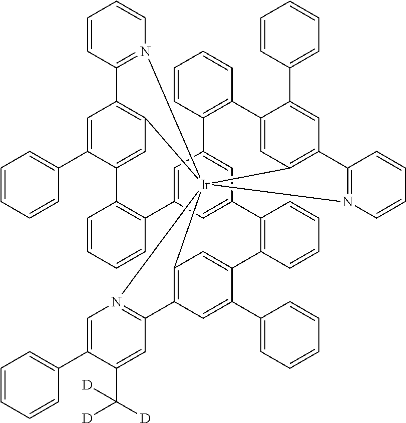

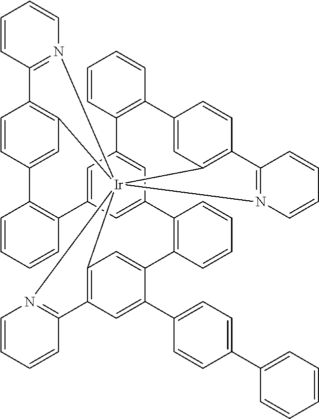

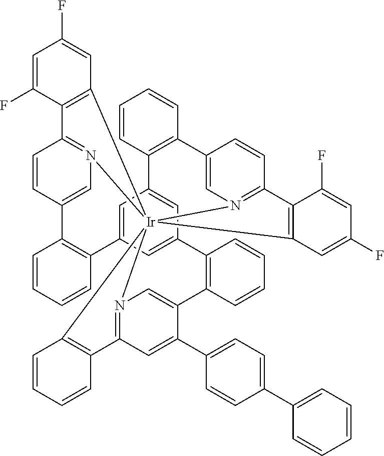

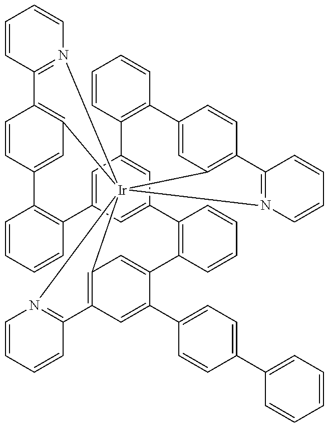

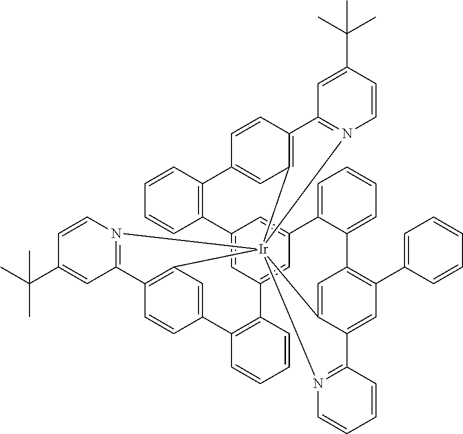

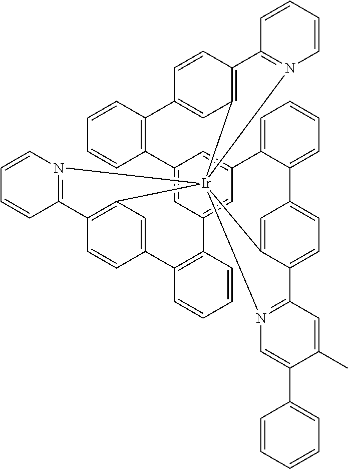

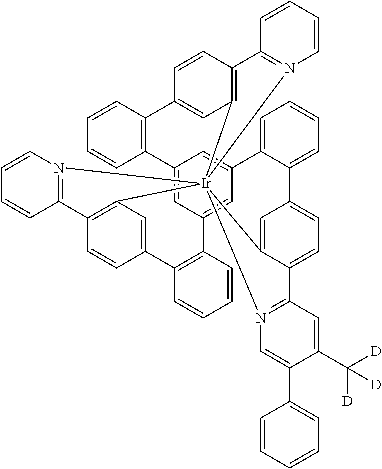

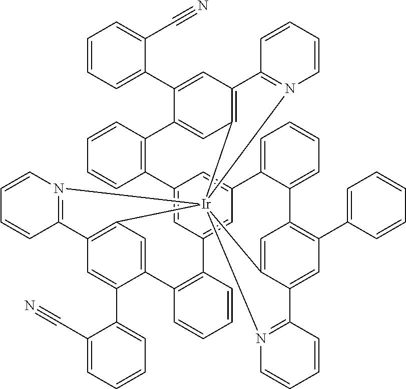

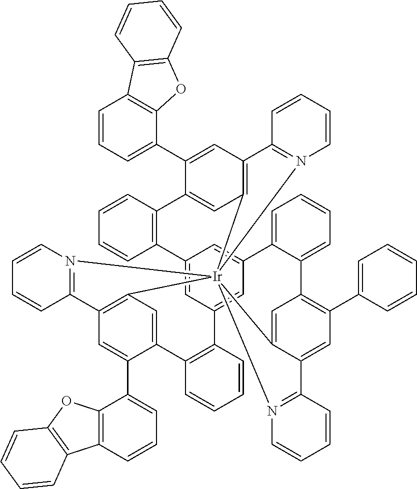

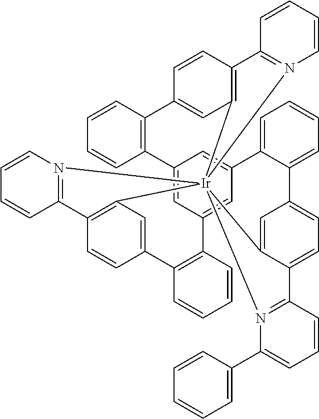

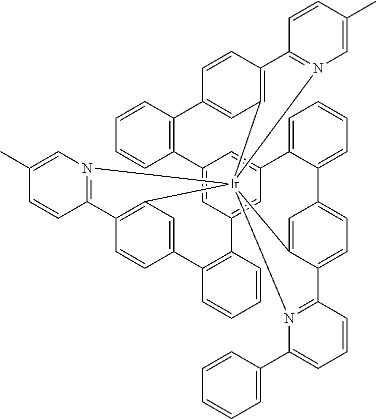

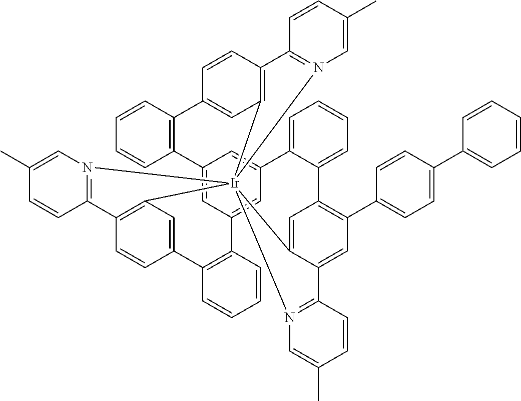

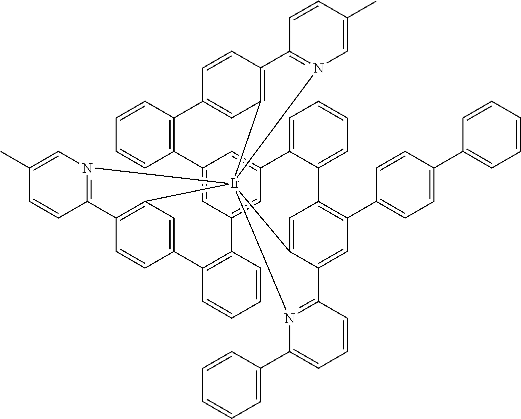

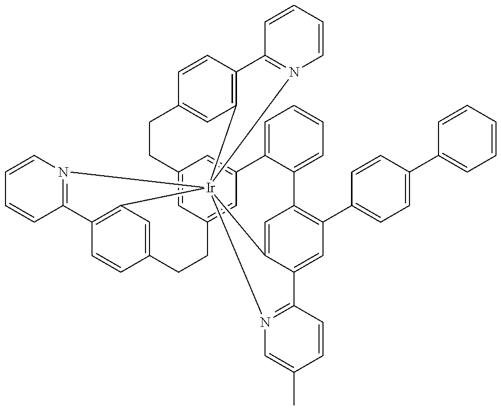

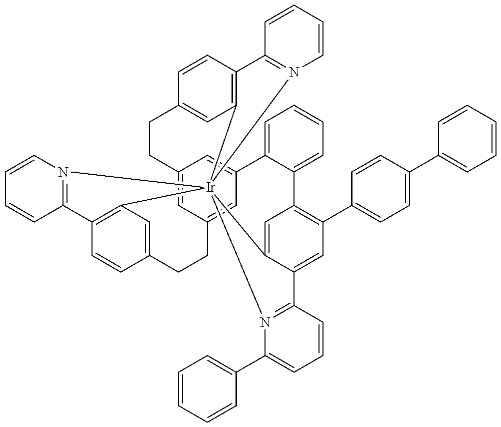

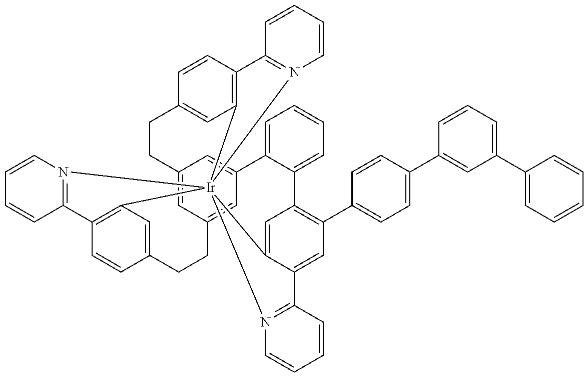

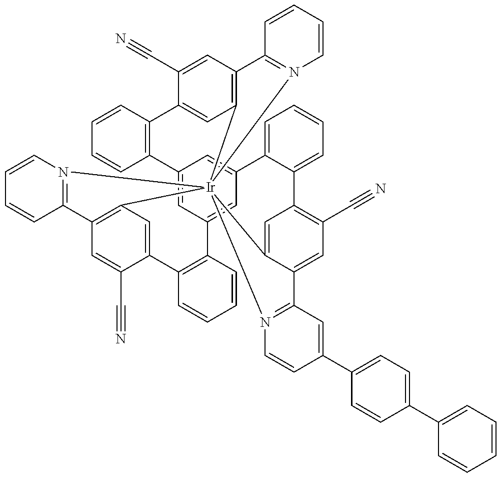

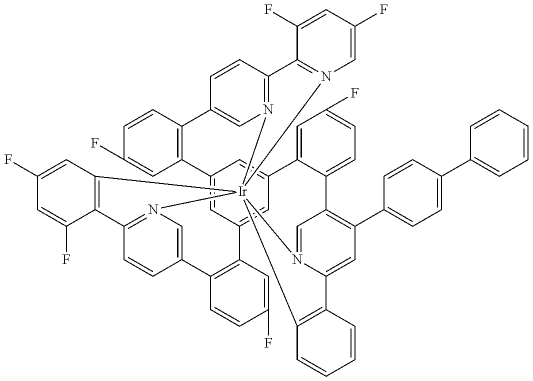

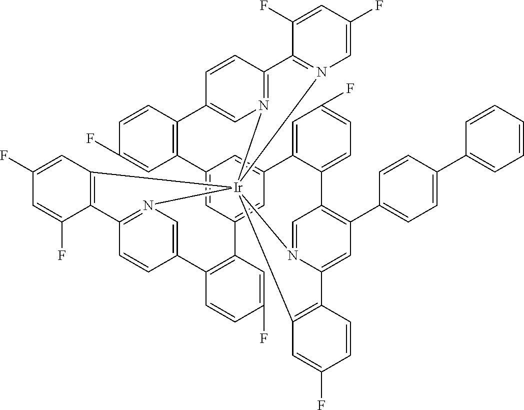

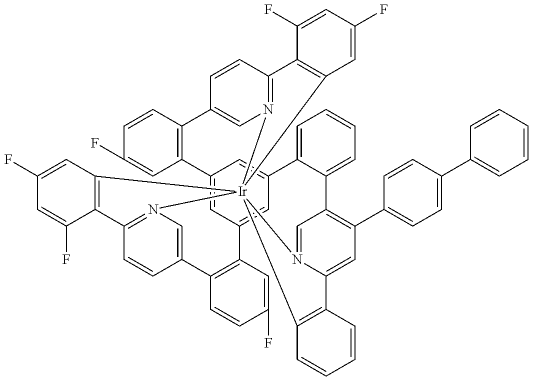

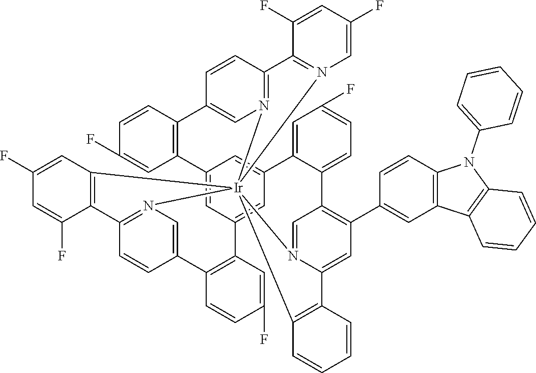

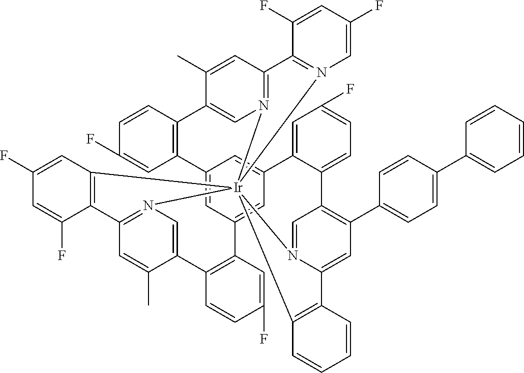

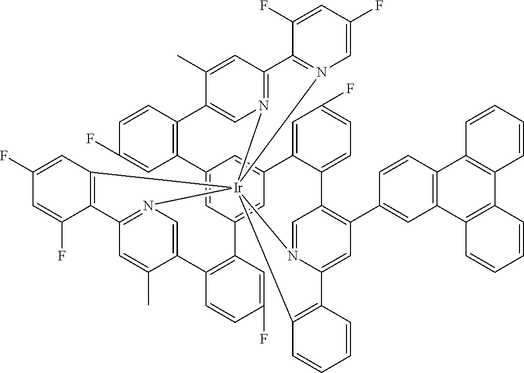

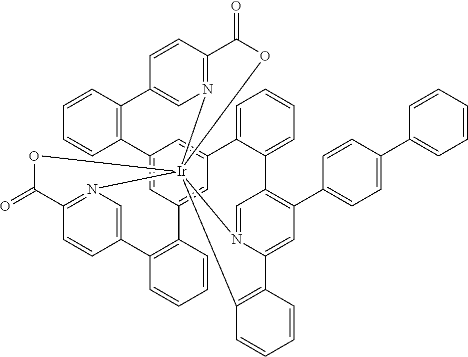

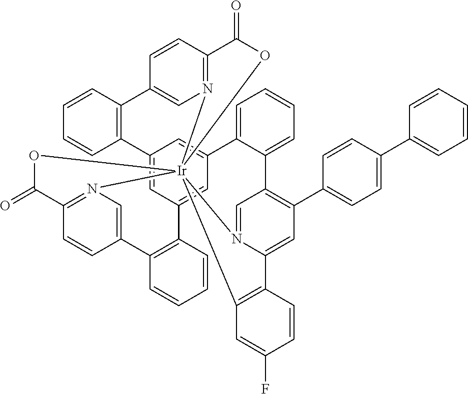

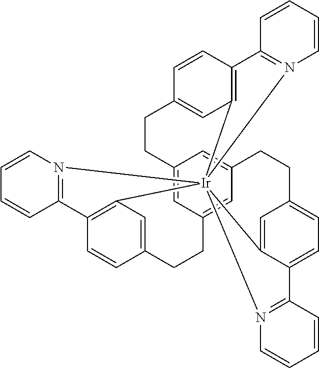

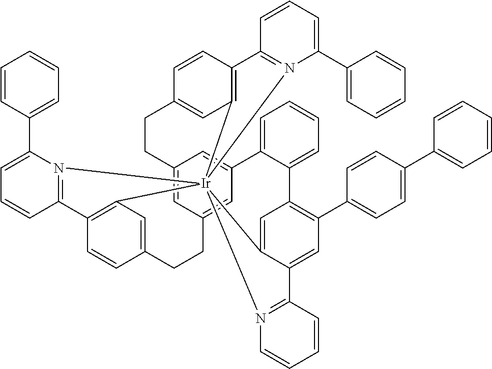

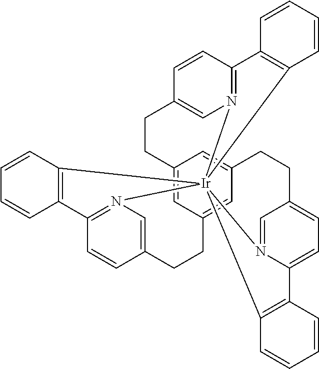

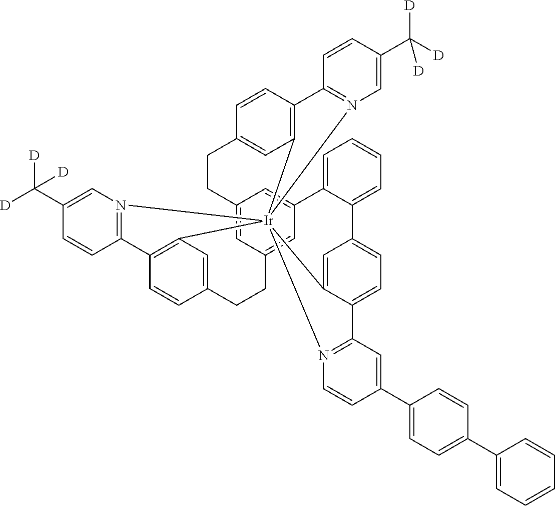

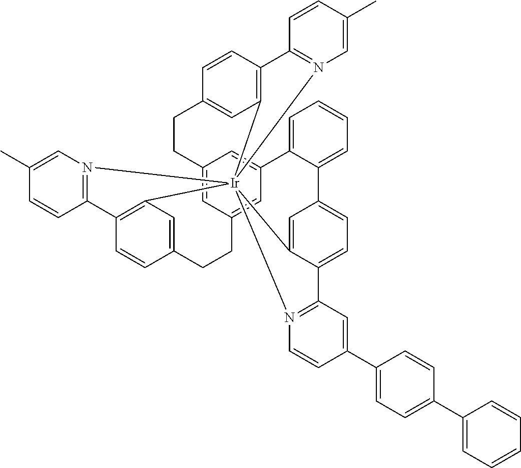

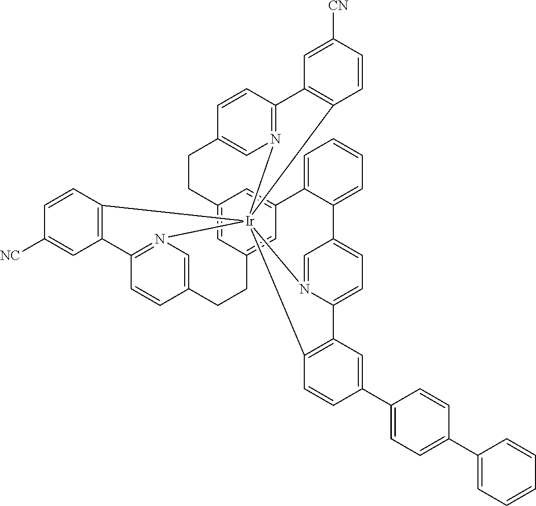

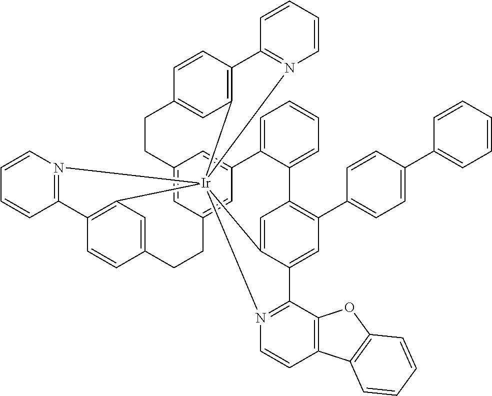

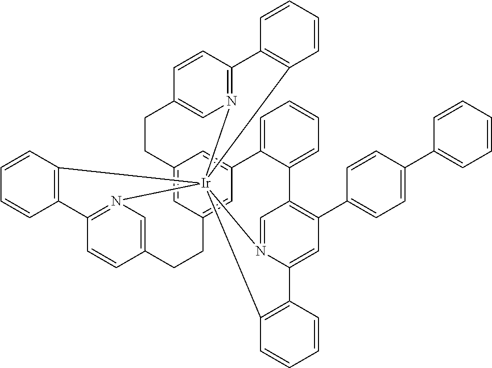

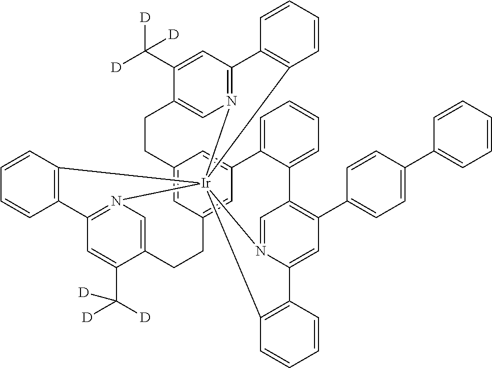

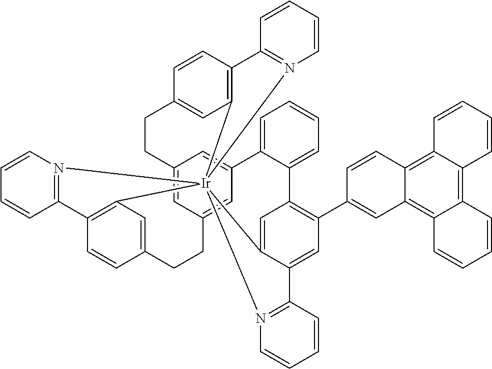

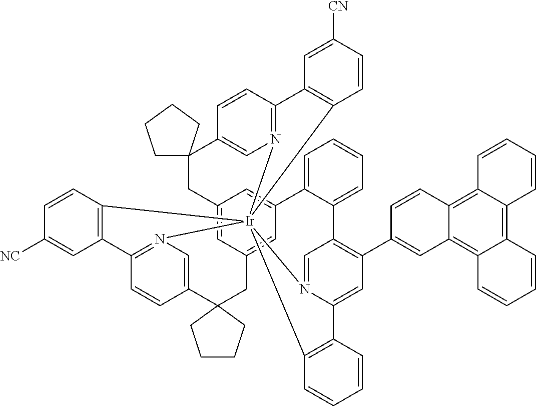

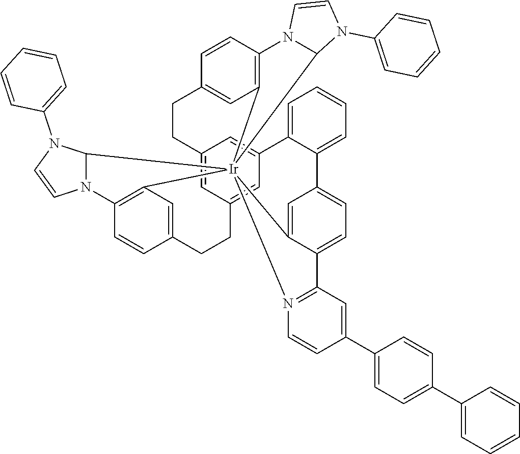

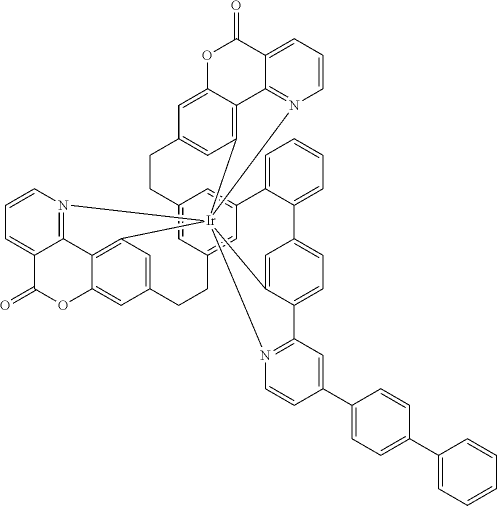

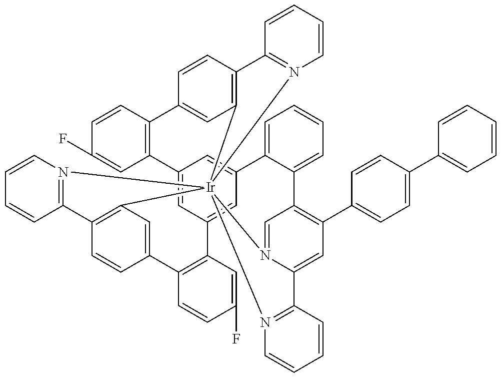

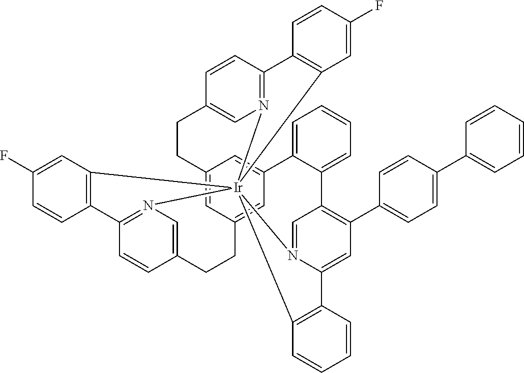

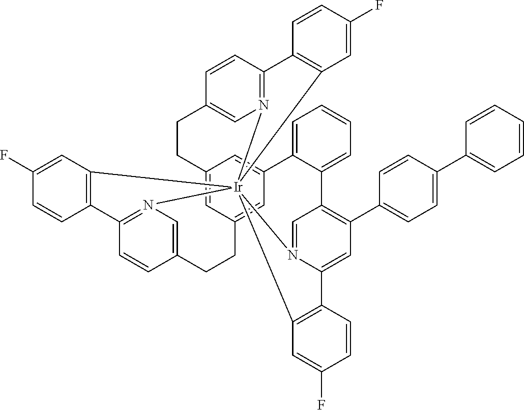

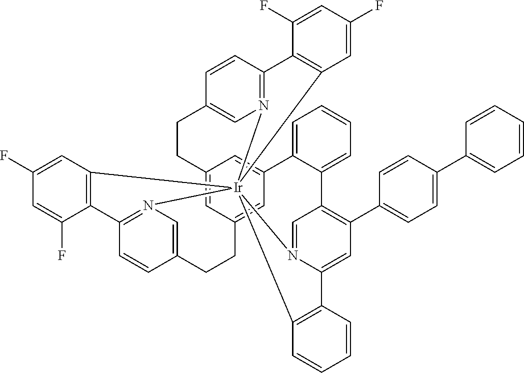

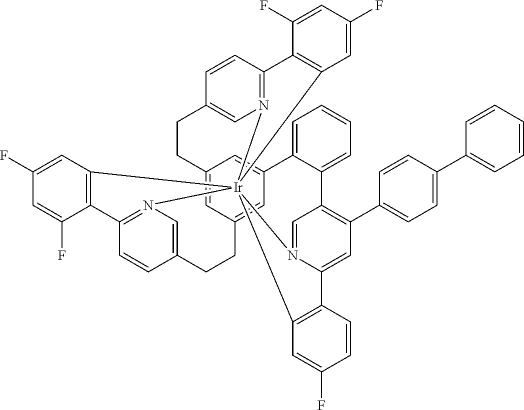

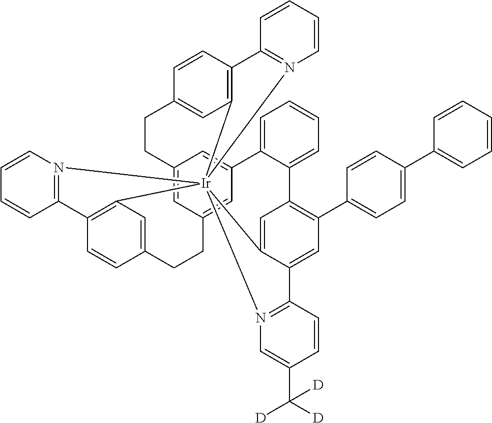

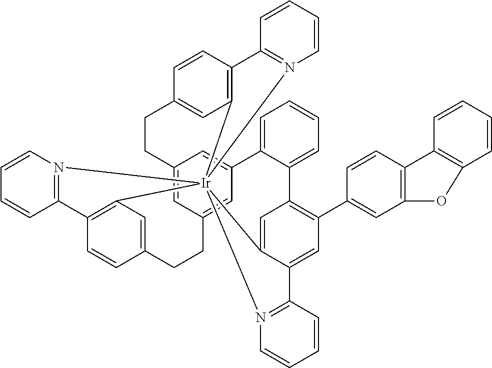

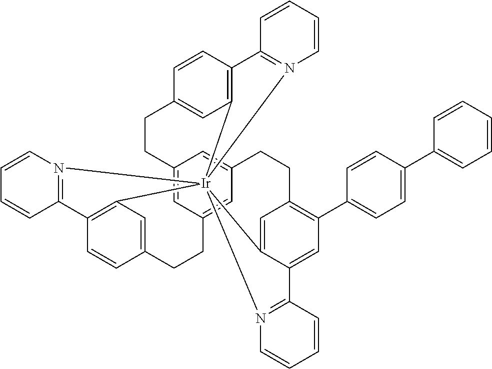

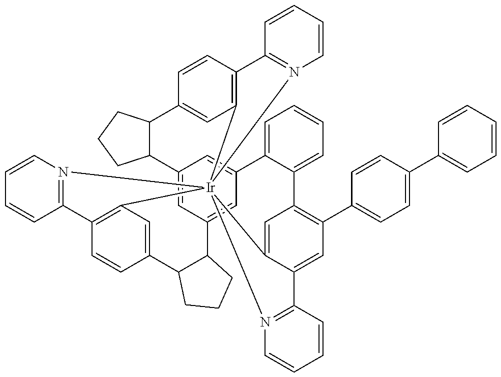

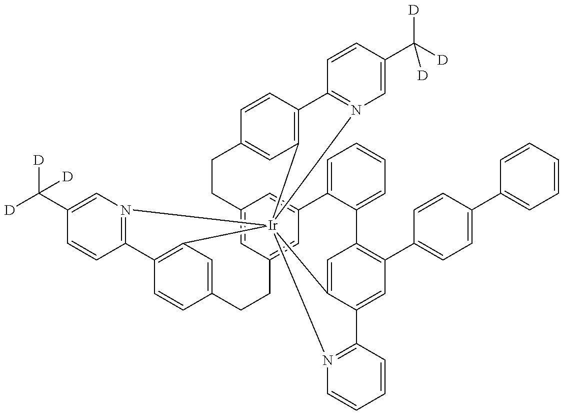

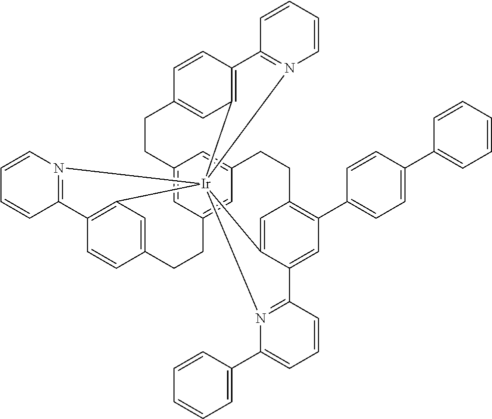

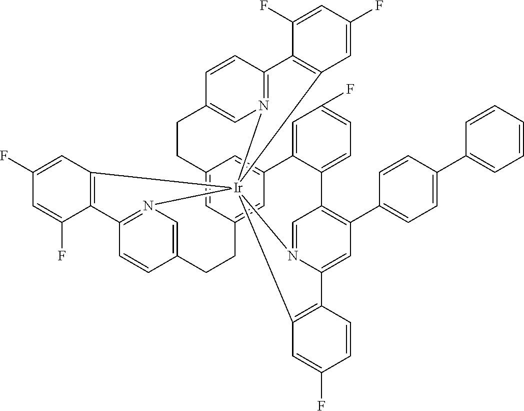

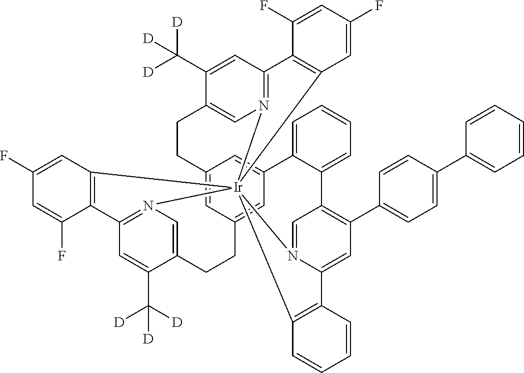

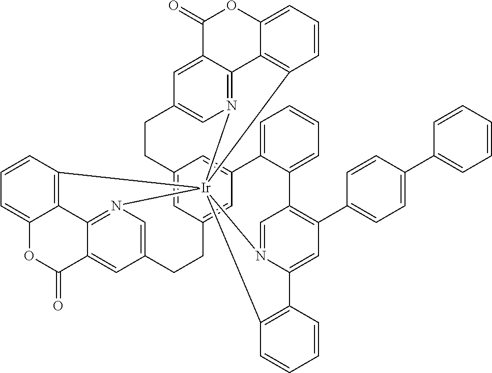

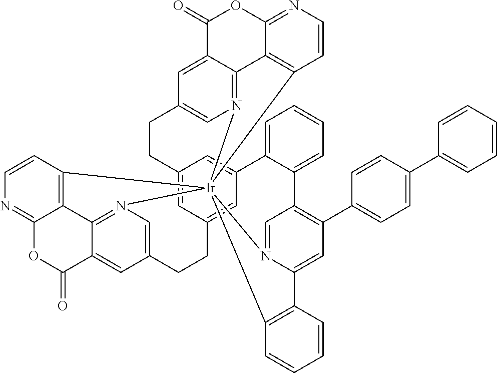

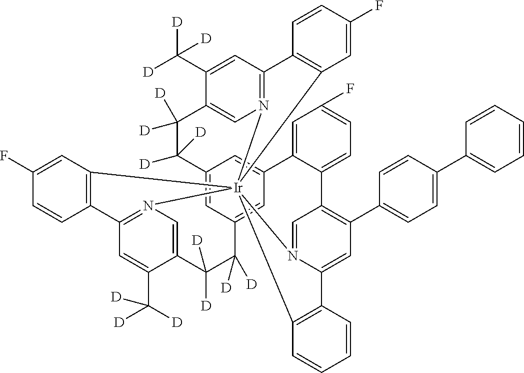

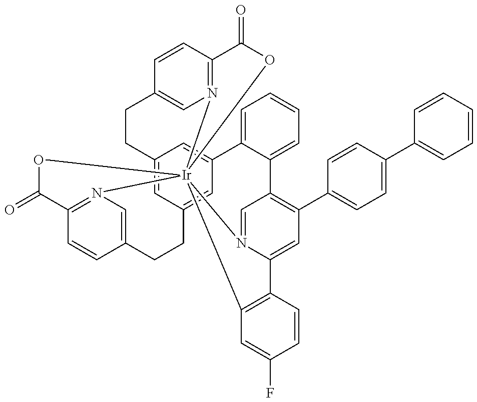

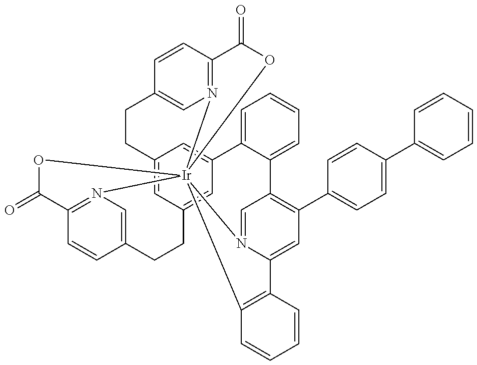

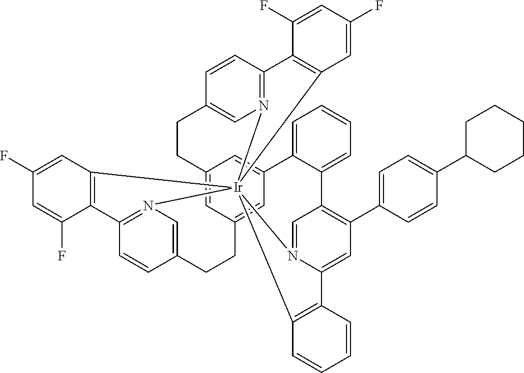

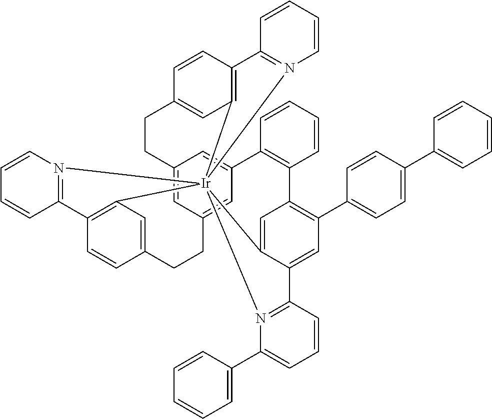

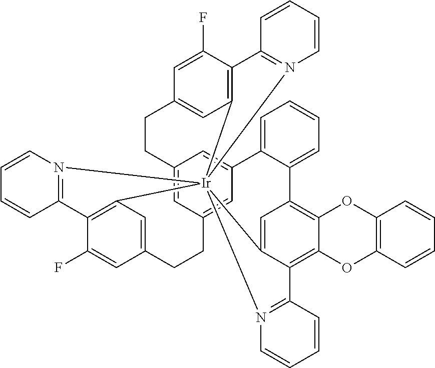

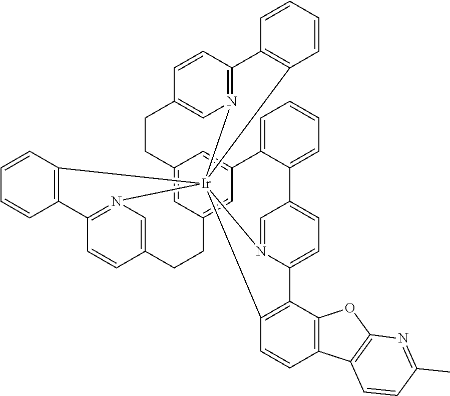

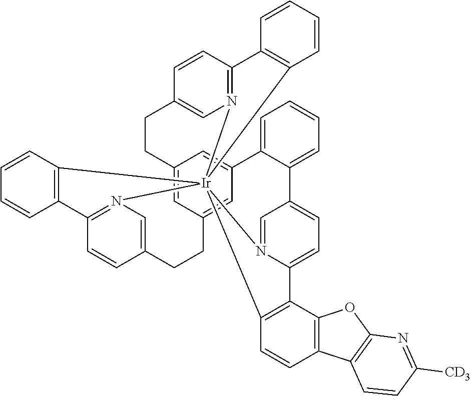

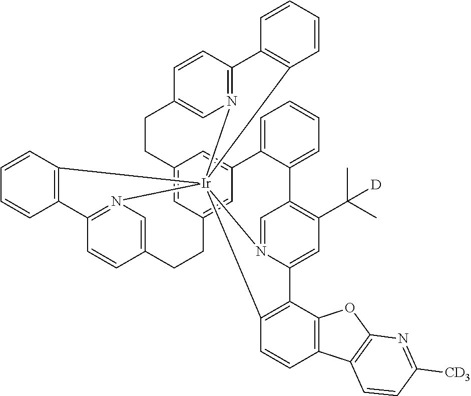

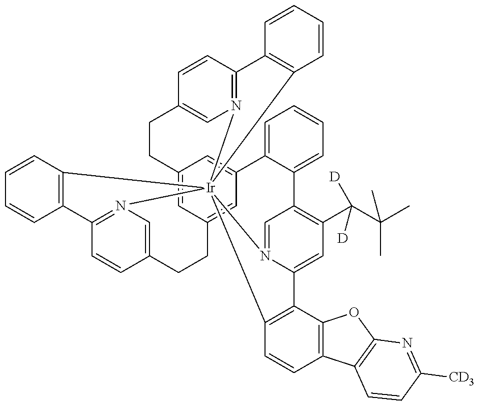

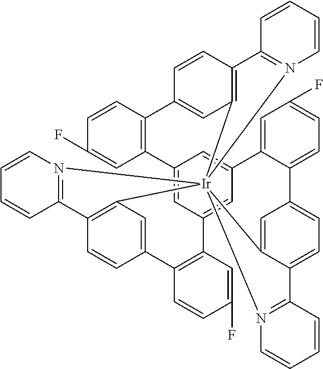

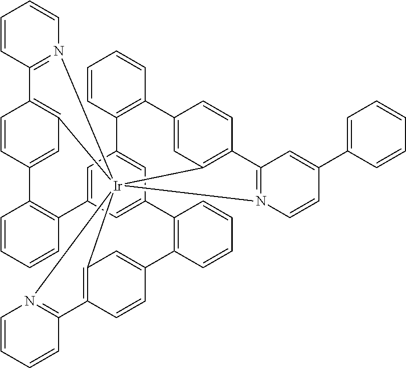

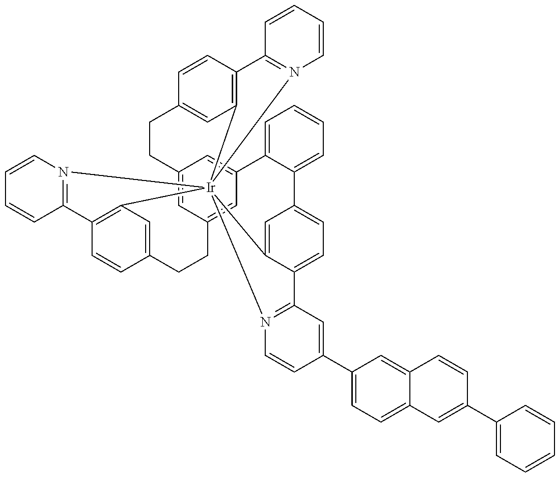

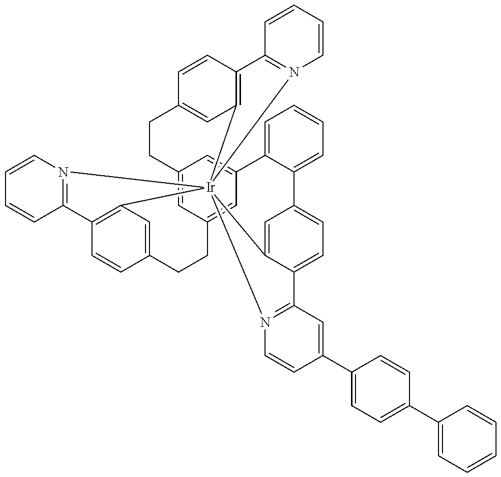

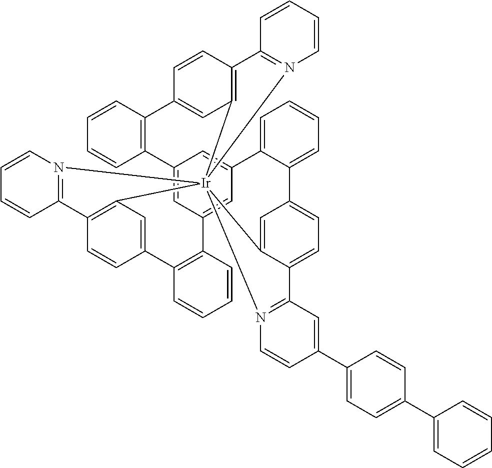

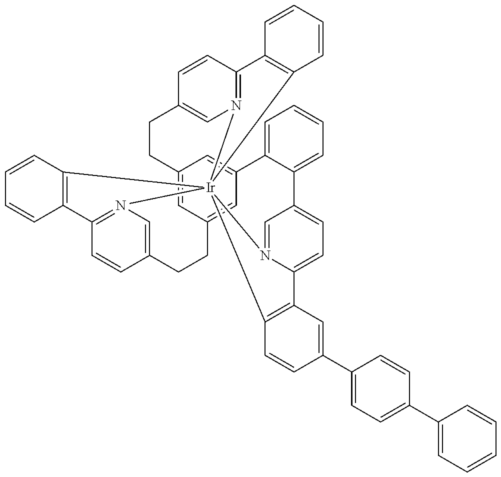

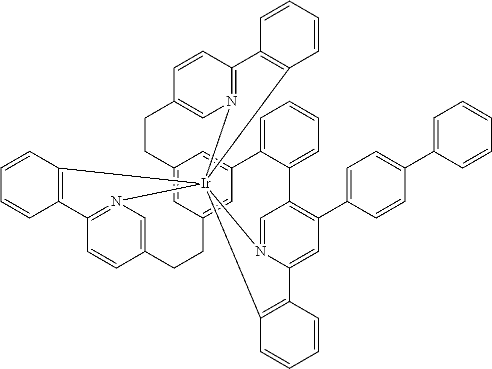

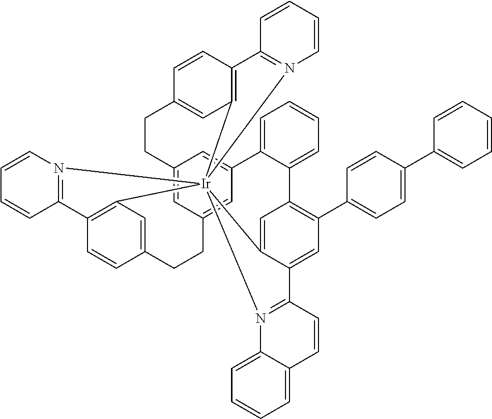

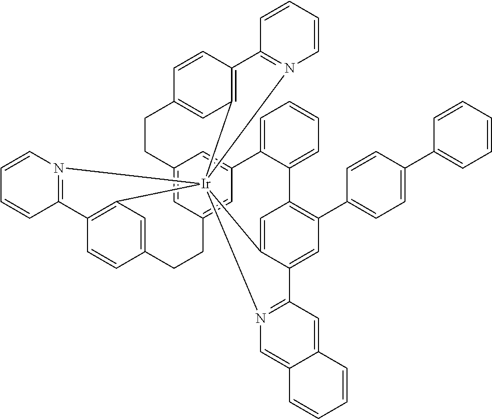

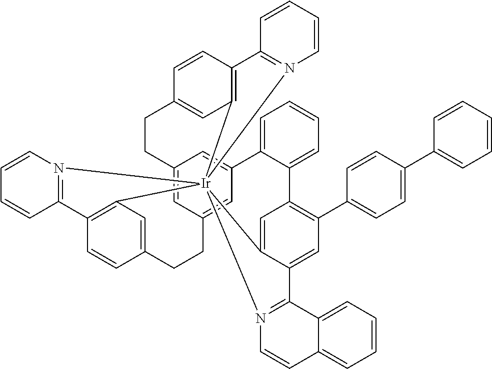

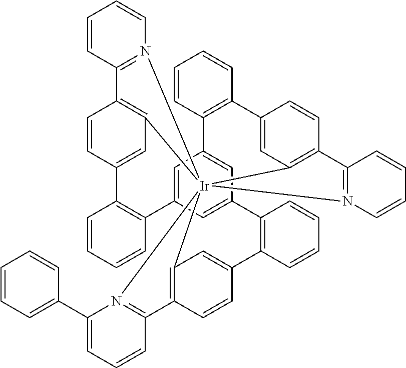

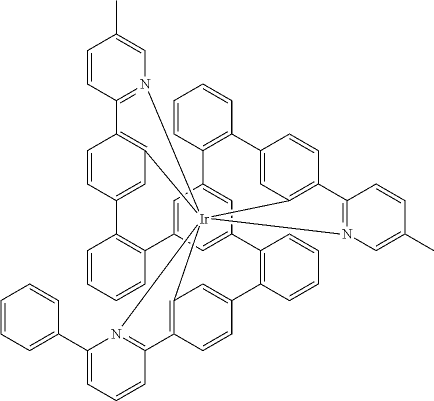

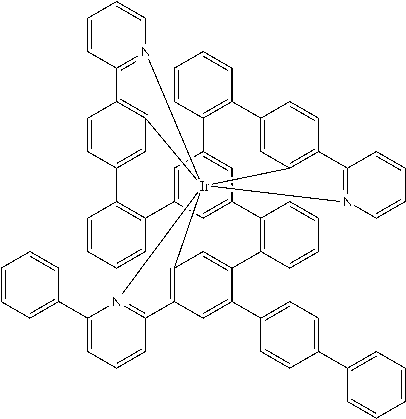

Fluorescent Emitters:

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 position. Aromatic pyreneamines, pyrenediamines, chryseneamines and chrysenediamines are defined analogously, where the diarylamino groups are bonded to the pyrene preferably in the 1 position or 1,6 position. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units.

Matrix Materials for Fluorescent Emitters:

Preferred matrix materials for fluorescent emitters are selected from the classes of the oligoarylenes (e.g. 2,2′,7,7′-tetraphenylspirobifluorene), especially the oligoarylenes containing fused aromatic groups, the oligoarylenevinylenes, the polypodal metal complexes, the hole-conducting compounds, the electron-conducting compounds, especially ketones, phosphine oxides and sulfoxides; the atropisomers, the boronic acid derivatives or the benzanthracenes. Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another.

Matrix Materials for Phosphorescent Emitters:

Preferred matrix materials for phosphorescent emitters are, as well as the compounds of the formula (I), aromatic ketones, aromatic phosphine oxides or aromatic sulfoxides or sulfones, triarylamines, carbazole derivatives, e.g. CBP (N,N-biscarbazolylbiphenyl), indolocarbazole derivatives, indenocarbazole derivatives, azacarbazole derivatives, bipolar matrix materials, silanes, azaboroles or boronic esters, triazine derivatives, zinc complexes, diazasilole or tetraazasilole derivatives, diazaphosphole derivatives, bridged carbazole derivatives, triphenylene derivatives, or lactams.

Electron-Transporting Materials:

Suitable electron-transporting materials are, for example, the compounds disclosed in Y. Shirota et al., Chem. Rev. 2007, 107(4), 953-1010, or other materials used in these layers according to the prior art.

Materials used for the electron transport layer may be any materials that are used as electron transport materials in the electron transport layer according to the prior art. Especially suitable are aluminium complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives.

Preferred electron transport and electron injection materials are shown in the following table:

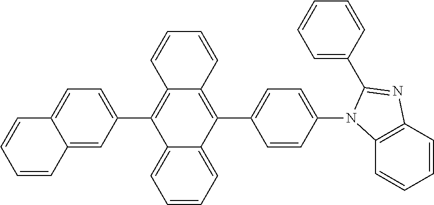 [SMILES: c1ccc(-c2nc3ccccc3n2-c2ccc(-c3c4ccccc4c(-c4ccc5ccccc5c4)c4ccccc34)cc2)cc1]

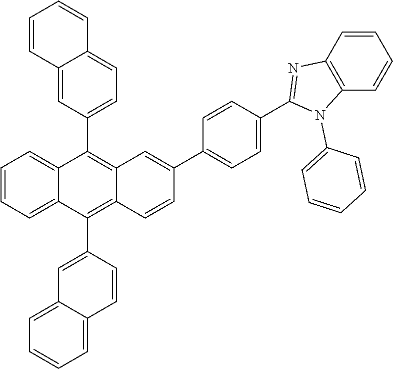 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

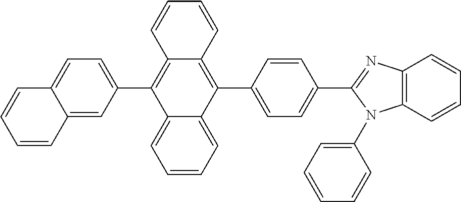 [SMILES: c1ccc(-n2c(-c3ccc(-c4c5ccccc5c(-c5ccc6ccccc6c5)c5ccccc45)cc3)nc3ccccc32)cc1]

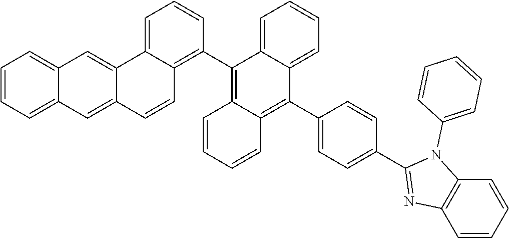 [SMILES: c1ccc(-n2c(-c3ccc(-c4c5ccccc5c(-c5cccc6c5ccc5cc7ccccc7cc56)c5ccccc45)cc3)nc3ccccc32)cc1]

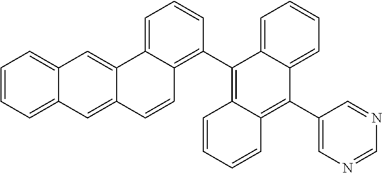 [SMILES: c1ccc2cc3c(ccc4c(-c5c6ccccc6c(-c6cncnc6)c6ccccc56)cccc43)cc2c1]

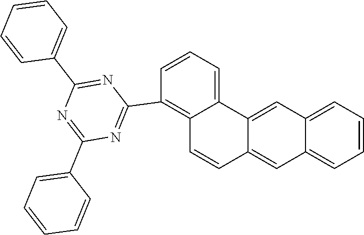 [SMILES: c1ccc(-c2nc(-c3ccccc3)nc(-c3cccc4c3ccc3cc5ccccc5cc34)n2)cc1]

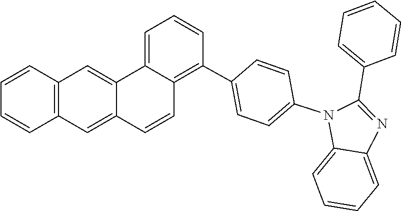 [SMILES: c1ccc(-c2nc3ccccc3n2-c2ccc(-c3cccc4c3ccc3cc5ccccc5cc34)cc2)cc1]

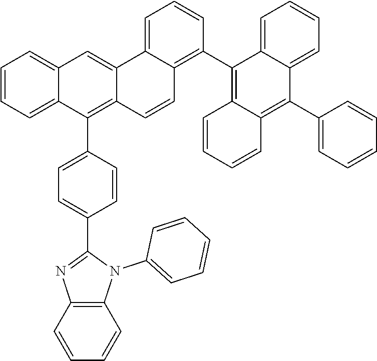 [SMILES: c1ccc(-c2c3ccccc3c(-c3cccc4c3ccc3c(-c5ccc(-c6nc7ccccc7n6-c6ccccc6)cc5)c5ccccc5cc34)c3ccccc23)cc1]

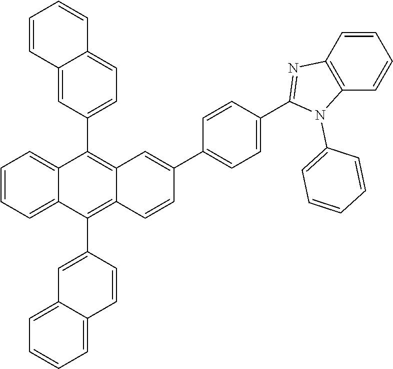 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

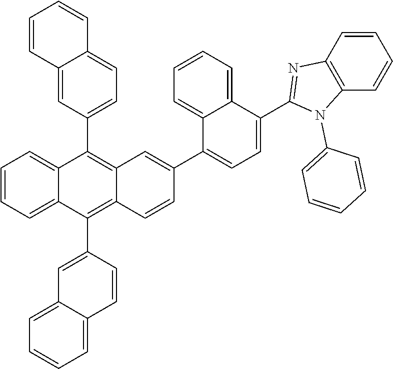 [SMILES: c1ccc(-n2c(-c3ccc(-c4ccc5c(-c6ccc7ccccc7c6)c6ccccc6c(-c6ccc7ccccc7c6)c5c4)c4ccccc34)nc3ccccc32)cc1]

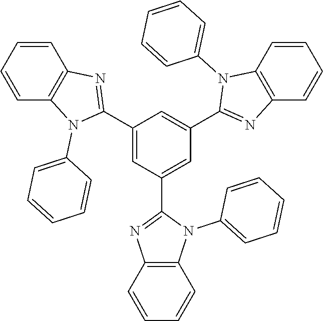 [SMILES: c1ccc(-n2c(-c3cc(-c4nc5ccccc5n4-c4ccccc4)cc(-c4nc5ccccc5n4-c4ccccc4)c3)nc3ccccc32)cc1]

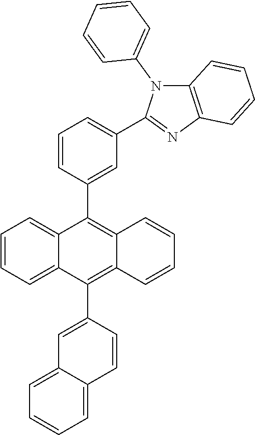 [SMILES: c1ccc(-n2c(-c3cccc(-c4c5ccccc5c(-c5ccc6ccccc6c5)c5ccccc45)c3)nc3ccccc32)cc1]

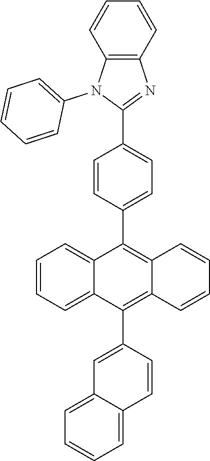 [SMILES: c1ccc(-n2c(-c3ccc(-c4c5ccccc5c(-c5ccc6ccccc6c5)c5ccccc45)cc3)nc3ccccc32)cc1]

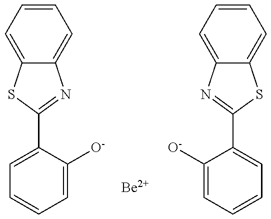

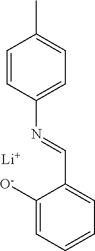 [SMILES: [Li+] ; Cc1ccc(/N=C/c2ccccc2[O-])cc1]

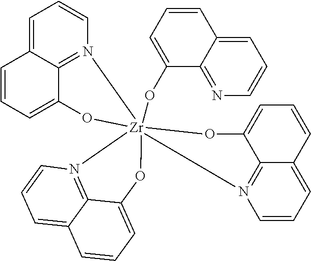

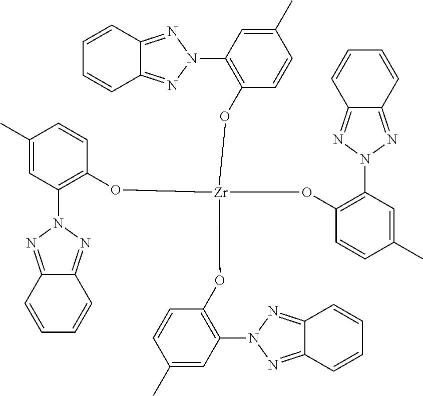

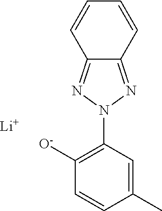 [SMILES: [Li+] ; Cc1ccc([O-])c(-n2nc3ccccc3n2)c1]

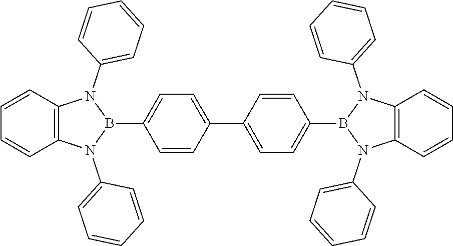 [SMILES: c1ccc(N2B(c3ccc(-c4ccc(B5N(c6ccccc6)c6ccccc6N5c5ccccc5)cc4)cc3)N(c3ccccc3)c3ccccc32)cc1]

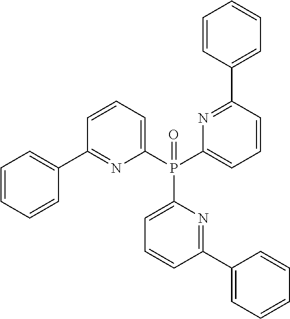 [SMILES: O=P(c1cccc(-c2ccccc2)n1)(c1cccc(-c2ccccc2)n1)c1cccc(-c2ccccc2)n1]

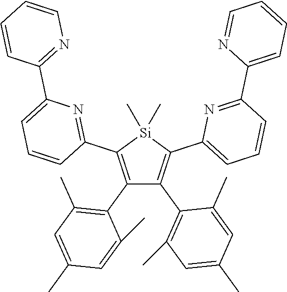 [SMILES: Cc1cc(C)c(C2=C(c3cccc(-c4ccccn4)n3)[Si](C)(C)C(c3cccc(-c4ccccn4)n3)=C2c2c(C)cc(C)cc2C)c(C)c1]

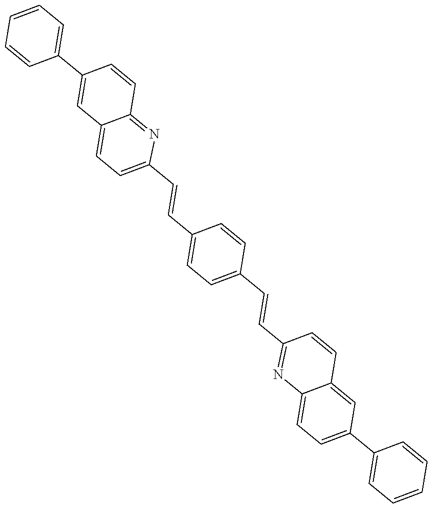 [SMILES: C(=C\c1ccc2cc(-c3ccccc3)ccc2n1)/c1ccc(/C=C/c2ccc3cc(-c4ccccc4)ccc3n2)cc1]

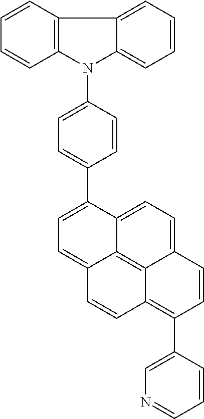 [SMILES: c1cncc(-c2ccc3ccc4c(-c5ccc(-n6c7ccccc7c7ccccc76)cc5)ccc5ccc2c3c54)c1]

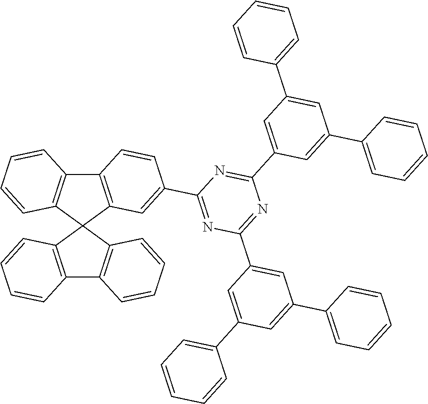 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

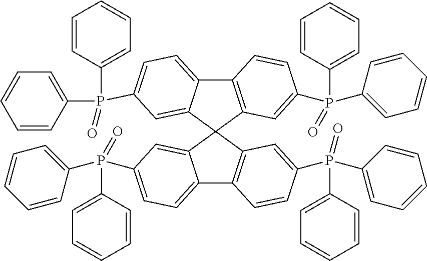 [SMILES: O=P(c1ccccc1)(c1ccccc1)c1ccc2c(c1)C1(c3cc(P(=O)(c4ccccc4)c4ccccc4)ccc3-2)c2cc(P(=O)(c3ccccc3)c3ccccc3)ccc2-c2ccc(P(=O)(c3ccccc3)c3ccccc3)cc21]

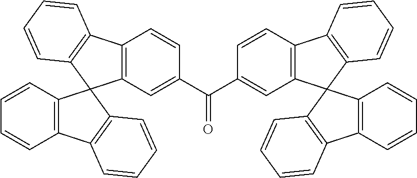 [SMILES: O=C(c1ccc2c(c1)C1(c3ccccc3-c3ccccc31)c1ccccc1-2)c1ccc2c(c1)C1(c3ccccc3-c3ccccc31)c1ccccc1-2]

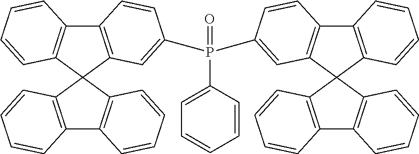 [SMILES: O=P(c1ccccc1)(c1ccc2c(c1)C1(c3ccccc3-c3ccccc31)c1ccccc1-2)c1ccc2c(c1)C1(c3ccccc3-c3ccccc31)c1ccccc1-2]

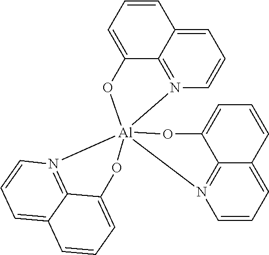

Hole-Transporting Materials:

Further compounds which, in addition to the compounds of the formula (I), are preferably used in hole-transporting layers of the OLEDs of the invention are indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups. Especially preferred therefor are the compounds disclosed in the table on pages 76-80 of WO 2020/109434 A1.

The abovementioned compounds selected from indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluorene amines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines and 9,10-dihydroanthracene spiro compounds having diarylamino groups and the compounds disclosed in the table on pages 76-80 of WO 2020/109434 A1 are generally suitable for use in layers having a hole-transporting function. The layers having a hole-transporting function include hole injection layers, hole transport layers, electron blocker layers and also emitting layers. When used in emitting layers the compounds are suitable as matrix materials, in particular as matrix materials having hole-transporting properties. The same applies to OLEDs of any construction, not only for OLEDs according to the definitions of the present application.

Compounds especially suitable for use in layers having a hole-transporting function in any OLEDs, not only the OLEDs according to the definitions of the present application, further include the following:

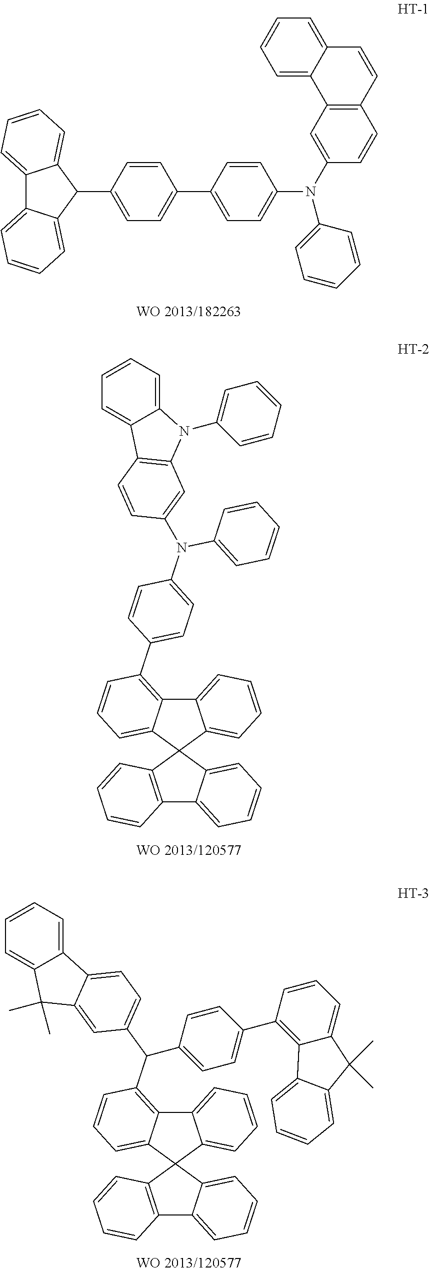

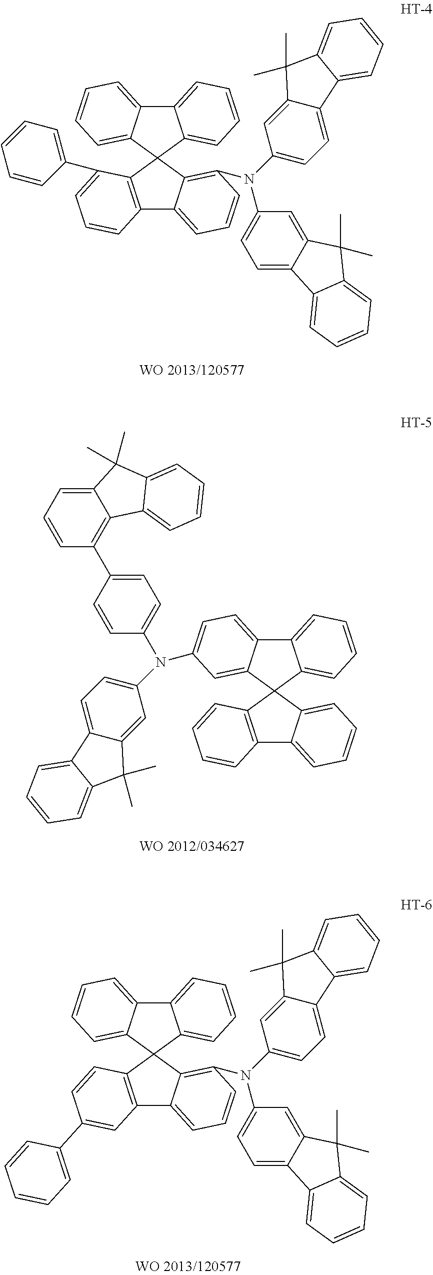

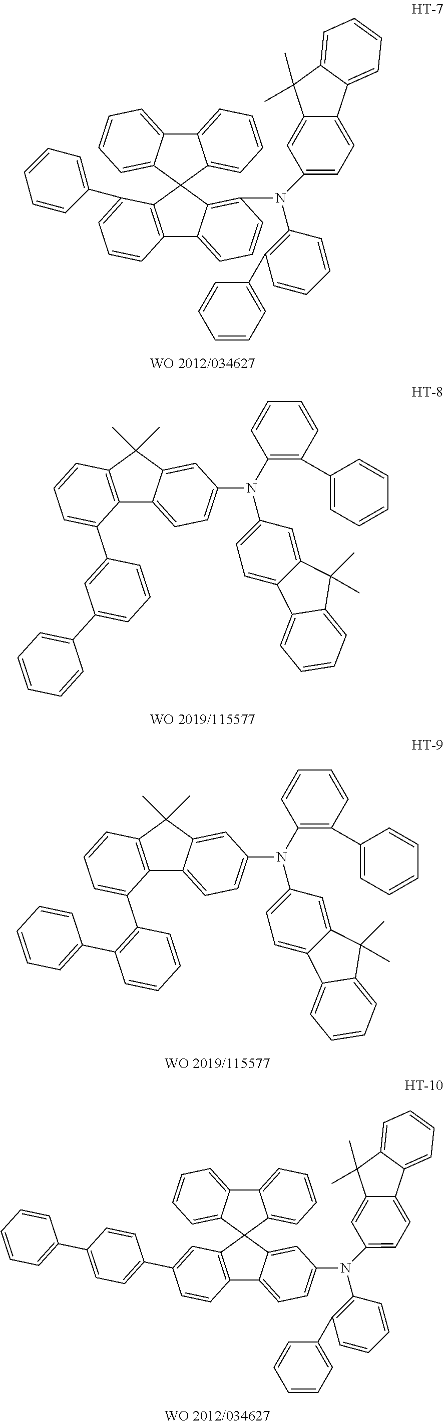 [SMILES: CC1(C)c2ccccc2-c2ccc(N(c3ccccc3-c3ccccc3)c3cccc4c3C3(c5ccccc5-c5ccccc53)c3c(-c5ccccc5)cccc3-4)cc21 ; CC1(C)c2ccccc2-c2ccc(N(c3ccc4c(c3)C(C)(C)c3cccc(-c5cccc(-c6ccccc6)c5)c3-4)c3ccccc3-c3ccccc3)cc21 ; CC1(C)c2ccccc2-c2ccc(N(c3ccc4c(c3)C(C)(C)c3cccc(-c5ccccc5-c5ccccc5)c3-4)c3ccccc3-c3ccccc3)cc21 ; CC1(C)c2ccccc2-c2ccc(N(c3ccc4c(c3)C3(c5ccccc5-c5ccccc53)c3cc(-c5ccc(-c6ccccc6)cc5)ccc3-4)c3ccccc3-c3ccccc3)cc21]

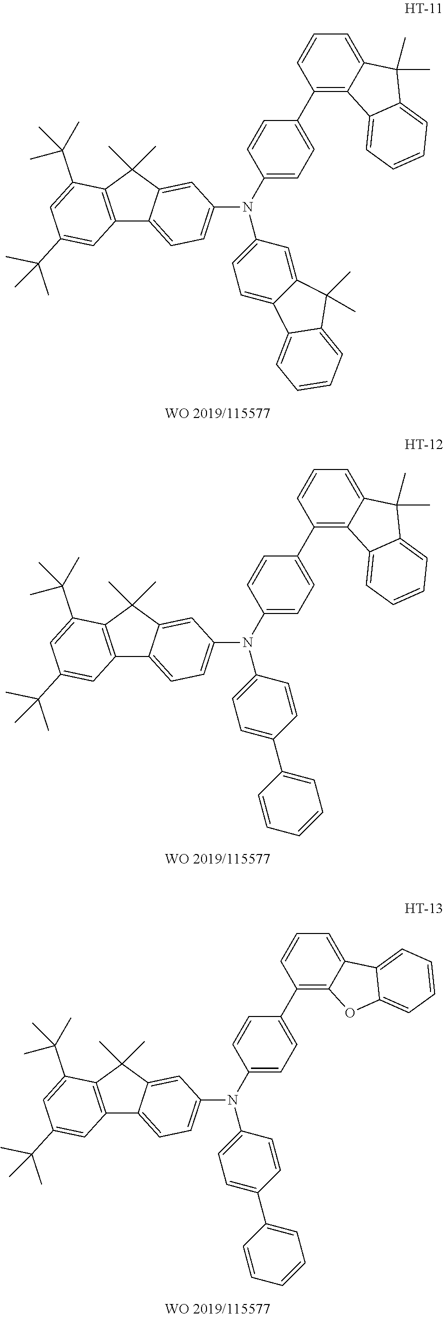

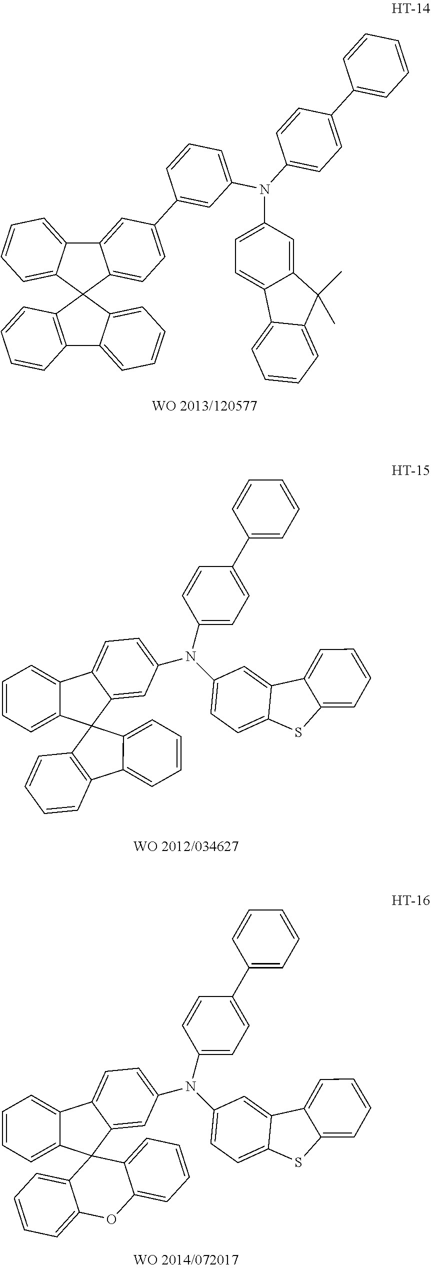 [SMILES: CC1(C)c2ccccc2-c2ccc(N(c3ccc(-c4ccccc4)cc3)c3cccc(-c4ccc5c(c4)-c4ccccc4C54c5ccccc5-c5ccccc54)c3)cc21 ; c1ccc(-c2ccc(N(c3ccc4c(c3)C3(c5ccccc5-c5ccccc53)c3ccccc3-4)c3ccc4sc5ccccc5c4c3)cc2)cc1 ; c1ccc(-c2ccc(N(c3ccc4c(c3)C3(c5ccccc5Oc5ccccc53)c3ccccc3-4)c3ccc4sc5ccccc5c4c3)cc2)cc1]

The compounds HT-1 to HT-16 are generally suitable for use in hole-transporting layers. Their use is not limited to particular OLEDs, such as for example the OLEDs described in the present application.

The compounds HT-1 to HT-16 may be prepared according to the procedures disclosed in the laid-open specifications recited in the table above. The further teaching relating to the use and preparation of the compounds disclosed in the laid-open specifications recited in the above table is hereby explicitly incorporated by reference and is preferably to be combined with the teaching relating to the use of the abovementioned compounds as hole-transporting materials recited above. The compounds HT-1 to HT-16 show exceptional properties when used in OLEDs, in particular exceptional lifetime and efficiency.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the emission of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

In a preferred embodiment, the electronic device is characterized in that one or more layers are coated by a sublimation process. In this case, the materials are applied by vapour deposition in vacuum sublimation systems at an initial pressure of less than 10-5 mbar, preferably less than 10⁻⁶ mbar. In this case, however, it is also possible that the initial pressure is even lower, for example less than 10-7 mbar.

Preference is likewise given to an electronic device, characterized in that one or more layers are coated by the OVPD (organic vapour phase deposition) method or with the aid of a carrier gas sublimation. In this case, the materials are applied at a pressure between 10-5 mbar and 1 bar. A special case of this method is the OVJP (organic vapour jet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds of formula (I) are needed. High solubility can be achieved by suitable substitution of the compounds.

It is further preferable that an electronic device of the invention is produced by applying one or more layers from solution and one or more layers by a sublimation method.

After application of the layers, according to the use, the device is structured, contact-connected and finally sealed, in order to rule out damaging effects of water and air.

According to the invention, the electronic devices comprising one or more compounds of formula (I) can be used in displays, as light sources in lighting applications and as light sources in medical and/or cosmetic applications.

### EXAMPLES

### A) Synthesis Examples

### 1) Synthesis of N-[4-(naphthalene-1-yl)phenyl]-N-(4-{8-oxatricyclo[7.4.0.0^{2,7}]trideca-1(13),2,4,6,9,11-hexaen-6-yl}phenyl)-9,9′-spirobi[fluoren]-1-amine 1a

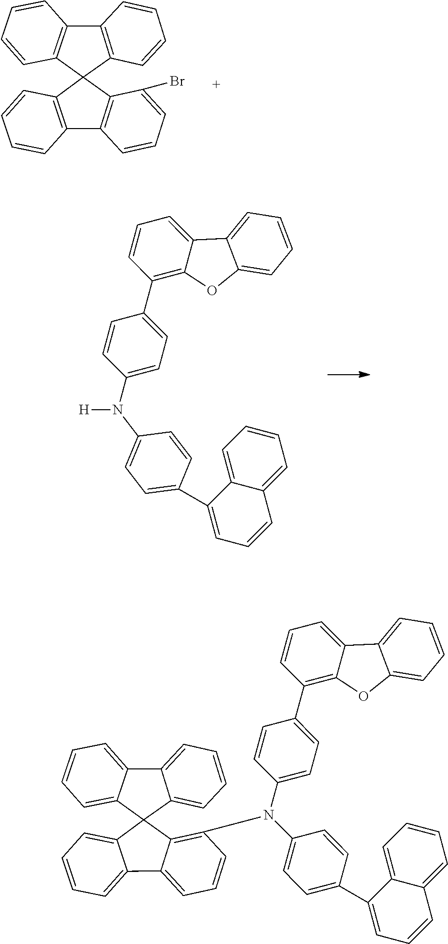 [SMILES: Brc1cccc2c1C1(c3ccccc3-c3ccccc31)c1ccccc1-2 ; c1ccc2c(-c3ccc(Nc4ccc(-c5cccc6c5oc5ccccc56)cc4)cc3)cccc2c1 ; c1ccc2c(c1)-c1ccccc1C21c2ccccc2-c2cccc(N(c3ccc(-c4cccc5ccccc45)cc3)c3ccc(-c4cccc5c4oc4ccccc45)cc3)c21]

16.8 g of 4-(naphthalen-1-yl)-N-(4-{8-oxatricyclo[7.4.0.0^{2,7}]trideca-1(13),2,4,6,9,11-hexaen-6yl}phenyl)aniline (36.4 mmol), and 1-bromo-9,9′-spirobi[fluorene] (13.8 g, 34.7 mol) are dissolved in 250 ml of toluene. The solution is degassed and saturated with N₂. It is subsequently admixed with 1 g (5.1 mmol) of S-Phos and 1.6 g (1.7 mmol) of Pd₂(dba)₃ and then 5 g of sodium tert-butoxide (52.05 mmol) are added. The reaction mixture is heated to boiling under a protective atmosphere overnight. The mixture is subsequently partitioned between toluene and water, and the organic phase is washed three times with water and dried over Na₂SO₄ and concentrated by rotary evaporation. After the crude product has been filtered through silica gel with toluene, the remaining residue is recrystallized from heptane/toluene. The substance is finally sublimed under high vacuum, purity is 99.9% determined by HPLC. The yield is 7.1 g (26% of theory).

The following compounds are prepared in an analogous manner:

| Ex. | Br-spiro | Amine | Product |
|---|---|---|---|
| 1b | 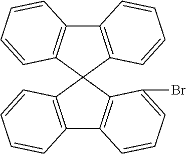 [SMILES: Brc1cccc2c1C1(c3ccccc3-c3ccccc31)c1ccccc1-2] | 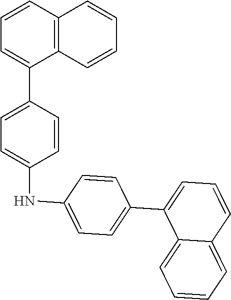 [SMILES: c1ccc2c(-c3ccc(Nc4ccc(-c5cccc6ccccc56)cc4)cc3)cccc2c1] | 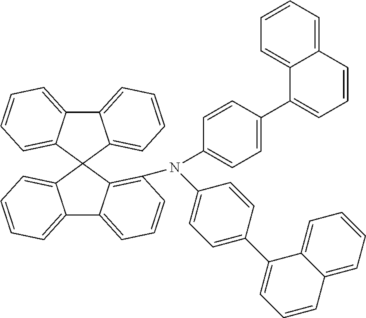 [SMILES: c1ccc2c(c1)-c1ccccc1C21c2ccccc2-c2cccc(N(c3ccc(-c4cccc5ccccc45)cc3)c3ccc(-c4cccc5ccccc45)cc3)c21] |
| 1c | 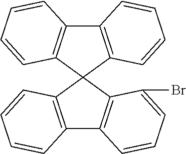 [SMILES: Brc1cccc2c1C1(c3ccccc3-c3ccccc31)c1ccccc1-2] | 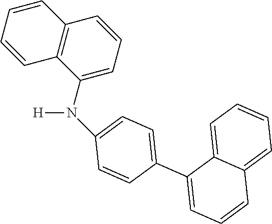 [SMILES: c1ccc2c(Nc3ccc(-c4cccc5ccccc45)cc3)cccc2c1] | 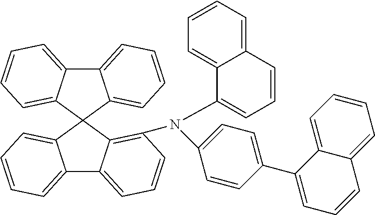 [SMILES: c1ccc2c(c1)-c1ccccc1C21c2ccccc2-c2cccc(N(c3ccc(-c4cccc5ccccc45)cc3)c3cccc4ccccc34)c21] |
| 1d | 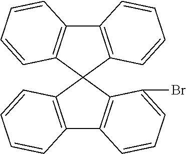 [SMILES: Brc1cccc2c1C1(c3ccccc3-c3ccccc31)c1ccccc1-2] | 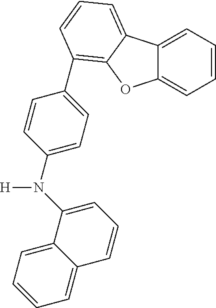 [SMILES: c1ccc2c(Nc3ccc(-c4cccc5c4oc4ccccc45)cc3)cccc2c1] | 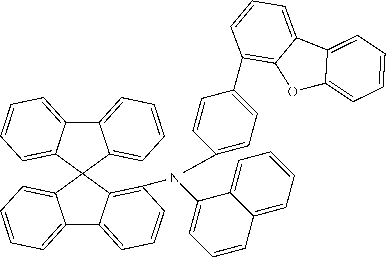 [SMILES: c1ccc2c(c1)-c1ccccc1C21c2ccccc2-c2cccc(N(c3ccc(-c4cccc5c4oc4ccccc45)cc3)c3cccc4ccccc34)c21] |
| 1e | 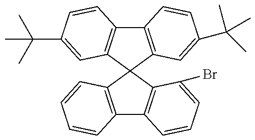 [SMILES: CC(C)(C)c1ccc2c(c1)C1(c3cc(C(C)(C)C)ccc3-2)c2ccccc2-c2cccc(Br)c21] | 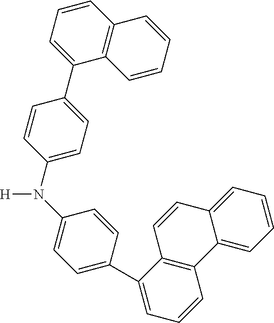 [SMILES: c1ccc2c(-c3ccc(Nc4ccc(-c5cccc6c5ccc5ccccc56)cc4)cc3)cccc2c1] | 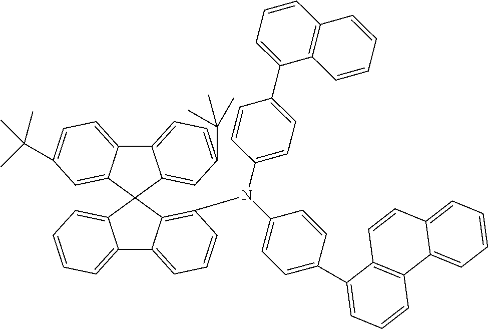 [SMILES: CC(C)(C)c1ccc2c(c1)C1(c3cc(C(C)(C)C)ccc3-2)c2ccccc2-c2cccc(N(c3ccc(-c4cccc5ccccc45)cc3)c3ccc(-c4cccc5c4ccc4ccccc45)cc3)c21] |
| 1f | 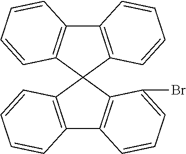 [SMILES: Brc1cccc2c1C1(c3ccccc3-c3ccccc31)c1ccccc1-2] | 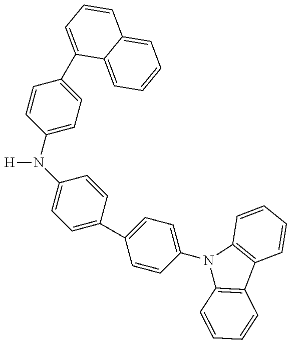 [SMILES: c1ccc2c(-c3ccc(Nc4ccc(-c5ccc(-n6c7ccccc7c7ccccc76)cc5)cc4)cc3)cccc2c1] | 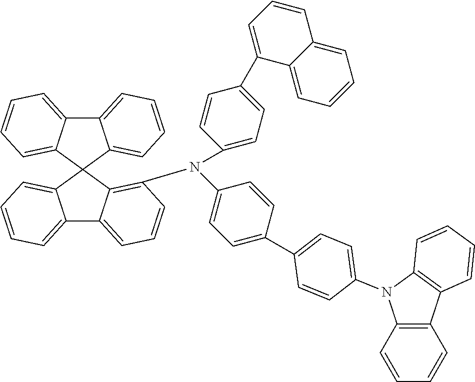 [SMILES: c1ccc2c(c1)-c1ccccc1C21c2ccccc2-c2cccc(N(c3ccc(-c4ccc(-n5c6ccccc6c6ccccc65)cc4)cc3)c3ccc(-c4cccc5ccccc45)cc3)c21] |
| 1g | 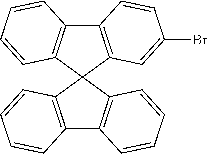 [SMILES: Brc1ccc2c(c1)C1(c3ccccc3-c3ccccc31)c1ccccc1-2] | 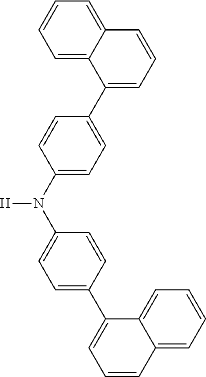 [SMILES: c1ccc2c(-c3ccc(Nc4ccc(-c5cccc6ccccc56)cc4)cc3)cccc2c1] | 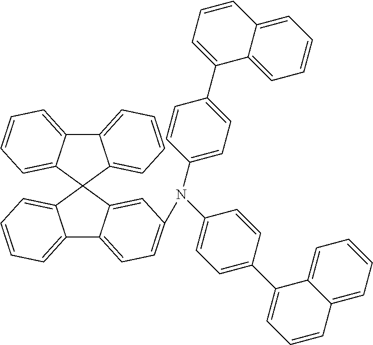 [SMILES: c1ccc2c(c1)-c1ccccc1C21c2ccccc2-c2ccc(N(c3ccc(-c4cccc5ccccc45)cc3)c3ccc(-c4cccc5ccccc45)cc3)cc21] |
| 1h | 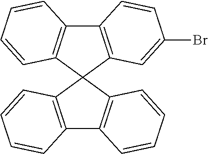 [SMILES: Brc1ccc2c(c1)C1(c3ccccc3-c3ccccc31)c1ccccc1-2] | 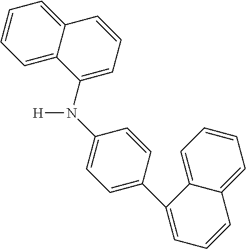 [SMILES: c1ccc2c(Nc3ccc(-c4cccc5ccccc45)cc3)cccc2c1] | 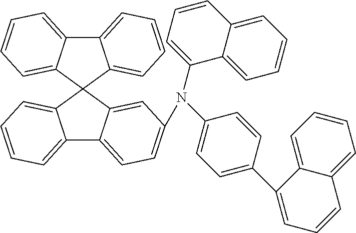 [SMILES: c1ccc2c(c1)-c1ccccc1C21c2ccccc2-c2ccc(N(c3ccc(-c4cccc5ccccc45)cc3)c3cccc4ccccc34)cc21] |
| 1i | 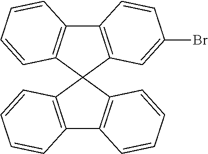 [SMILES: Brc1ccc2c(c1)C1(c3ccccc3-c3ccccc31)c1ccccc1-2] | 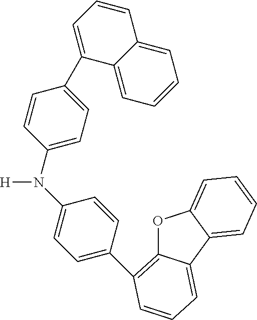 [SMILES: c1ccc2c(-c3ccc(Nc4ccc(-c5cccc6c5oc5ccccc56)cc4)cc3)cccc2c1] | 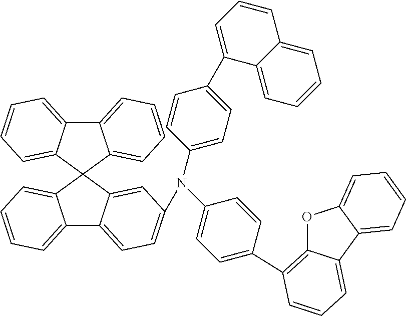 [SMILES: c1ccc2c(c1)-c1ccccc1C21c2ccccc2-c2ccc(N(c3ccc(-c4cccc5ccccc45)cc3)c3ccc(-c4cccc5c4oc4ccccc45)cc3)cc21] |
| 1j | 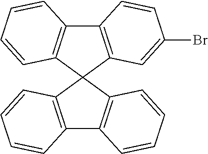 [SMILES: Brc1ccc2c(c1)C1(c3ccccc3-c3ccccc31)c1ccccc1-2] | 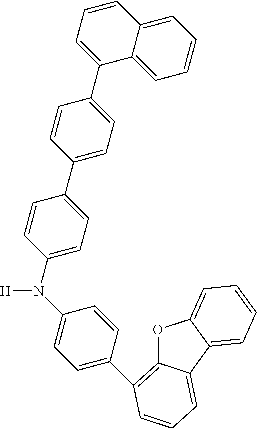 [SMILES: c1ccc2c(-c3ccc(-c4ccc(Nc5ccc(-c6cccc7c6oc6ccccc67)cc5)cc4)cc3)cccc2c1] | 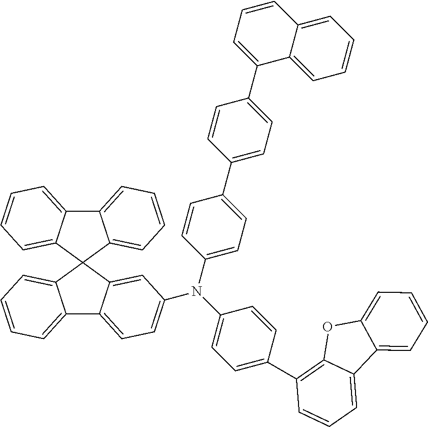 [SMILES: c1ccc2c(c1)-c1ccccc1C21c2ccccc2-c2ccc(N(c3ccc(-c4ccc(-c5cccc6ccccc56)cc4)cc3)c3ccc(-c4cccc5c4oc4ccccc45)cc3)cc21] |
| 1k | 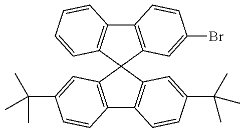 [SMILES: CC(C)(C)c1ccc2c(c1)C1(c3ccccc3-c3ccc(Br)cc31)c1cc(C(C)(C)C)ccc1-2] | 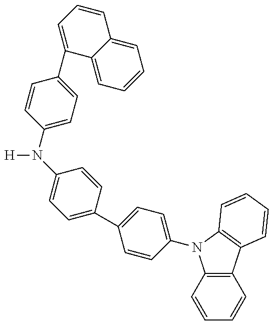 [SMILES: c1ccc2c(-c3ccc(Nc4ccc(-c5ccc(-n6c7ccccc7c7ccccc76)cc5)cc4)cc3)cccc2c1] | 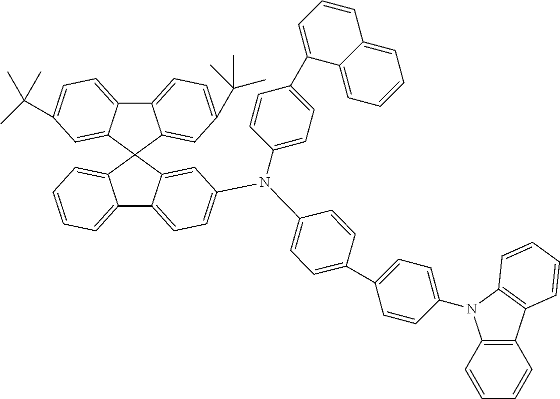 [SMILES: CC(C)(C)c1ccc2c(c1)C1(c3ccccc3-c3ccc(N(c4ccc(-c5ccc(-n6c7ccccc7c7ccccc76)cc5)cc4)c4ccc(-c5cccc6ccccc56)cc4)cc31)c1cc(C(C)(C)C)ccc1-2] |
| 1l | 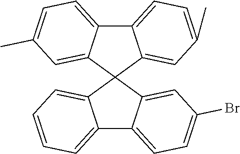 [SMILES: Cc1ccc2c(c1)C1(c3ccccc3-c3ccc(Br)cc31)c1cc(C)ccc1-2] | 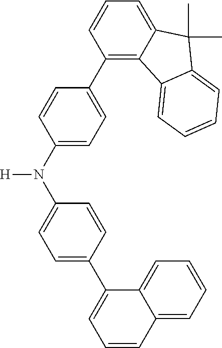 [SMILES: CC1(C)c2ccccc2-c2c(-c3ccc(Nc4ccc(-c5cccc6ccccc56)cc4)cc3)cccc21] | 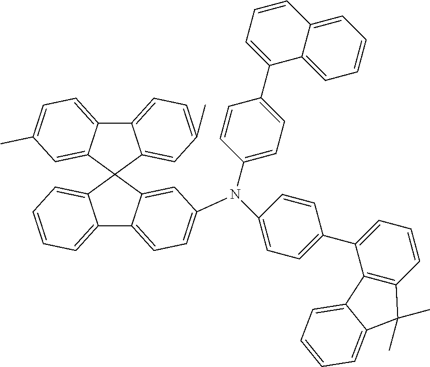 |
| 1m | 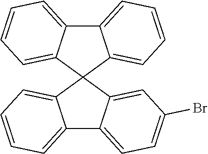 [SMILES: Brc1ccc2c(c1)C1(c3ccccc3-c3ccccc31)c1ccccc1-2] | 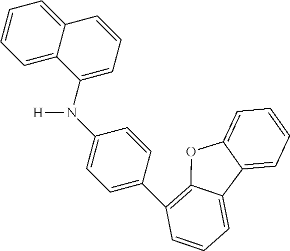 [SMILES: c1ccc2c(Nc3ccc(-c4cccc5c4oc4ccccc45)cc3)cccc2c1] | 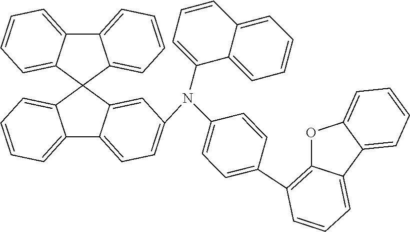 [SMILES: c1ccc2c(c1)-c1ccccc1C21c2ccccc2-c2ccc(N(c3ccc(-c4cccc5c4oc4ccccc45)cc3)c3cccc4ccccc34)cc21] |
| 1n | 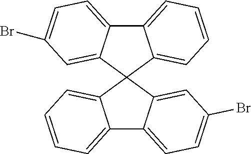 [SMILES: Brc1ccc2c(c1)C1(c3ccccc3-2)c2ccccc2-c2ccc(Br)cc21] | 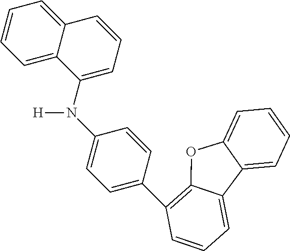 [SMILES: c1ccc2c(Nc3ccc(-c4cccc5c4oc4ccccc45)cc3)cccc2c1] | 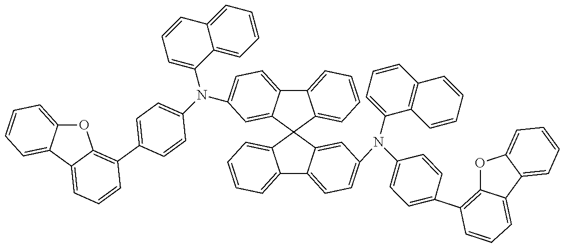 [SMILES: c1ccc2c(c1)-c1ccc(N(c3ccc(-c4cccc5c4oc4ccccc45)cc3)c3cccc4ccccc34)cc1C21c2ccccc2-c2ccc(N(c3ccc(-c4cccc5c4oc4ccccc45)cc3)c3cccc4ccccc34)cc21] |
| 1o | 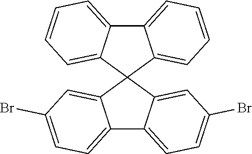 [SMILES: Brc1ccc2c(c1)C1(c3ccccc3-c3ccccc31)c1cc(Br)ccc1-2] | 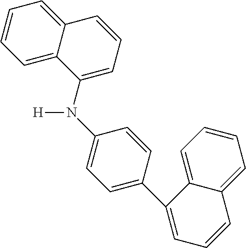 [SMILES: c1ccc2c(Nc3ccc(-c4cccc5ccccc45)cc3)cccc2c1] | 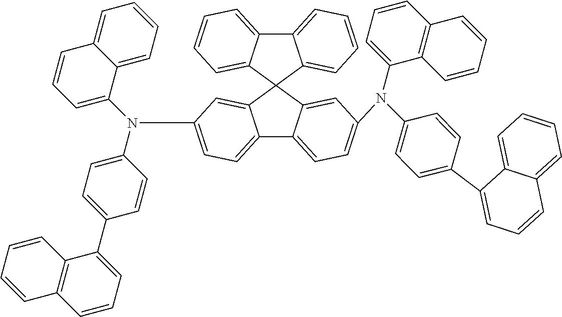 [SMILES: c1ccc2c(c1)-c1ccccc1C21c2cc(N(c3ccc(-c4cccc5ccccc45)cc3)c3cccc4ccccc34)ccc2-c2ccc(N(c3ccc(-c4cccc5ccccc45)cc3)c3cccc4ccccc34)cc21] |
| 1p | 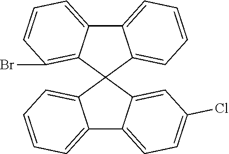 [SMILES: Clc1ccc2c(c1)C1(c3ccccc3-2)c2ccccc2-c2cccc(Br)c21] | 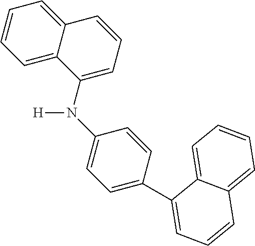 [SMILES: c1ccc2c(Nc3ccc(-c4cccc5ccccc45)cc3)cccc2c1] | 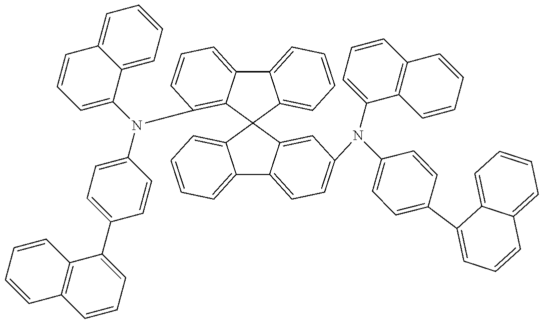 [SMILES: c1ccc2c(c1)-c1ccc(N(c3ccc(-c4cccc5ccccc45)cc3)c3cccc4ccccc34)cc1C21c2ccccc2-c2cccc(N(c3ccc(-c4cccc5ccccc45)cc3)c3cccc4ccccc34)c21] |
| 1p | 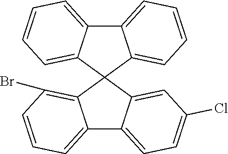 [SMILES: Clc1ccc2c(c1)C1(c3ccccc3-c3ccccc31)c1c(Br)cccc1-2] | 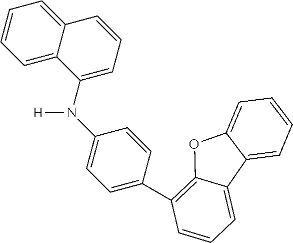 [SMILES: c1ccc2c(Nc3ccc(-c4cccc5c4oc4ccccc45)cc3)cccc2c1] | 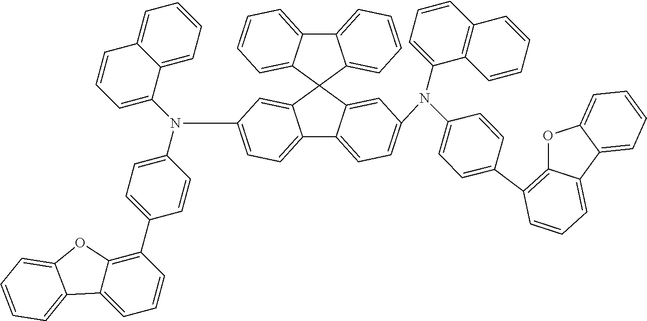 [SMILES: c1ccc2c(c1)-c1ccccc1C21c2cc(N(c3ccc(-c4cccc5c4oc4ccccc45)cc3)c3cccc4ccccc34)ccc2-c2ccc(N(c3ccc(-c4cccc5c4oc4ccccc45)cc3)c3cccc4ccccc34)cc21] |

### 2) Synthesis of 1-{[4-(naphthalen-1-yl)phenyl](4-{8-oxatricyclo[7.4.0.0^{2,7}]trideca-1(13),2,4,6,9,11-hexaen-6-yl}phenyl)amino}-9H-fluoren-9-one 2a

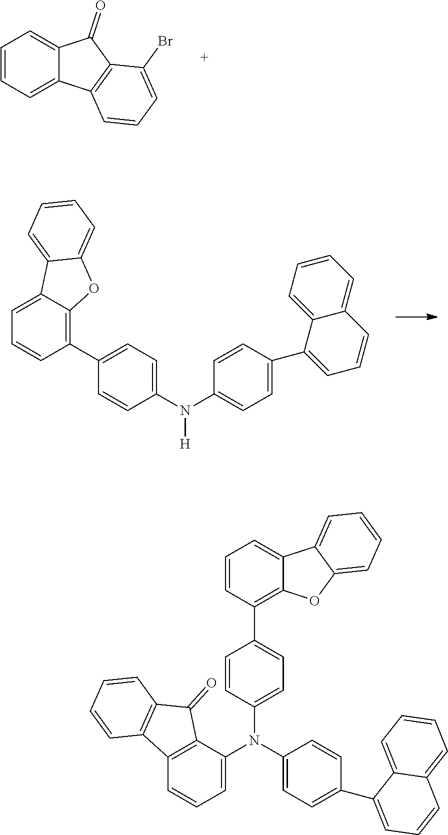 [SMILES: O=C1c2ccccc2-c2cccc(Br)c21 ; c1ccc2c(-c3ccc(Nc4ccc(-c5cccc6c5oc5ccccc56)cc4)cc3)cccc2c1 ; O=C1c2ccccc2-c2cccc(N(c3ccc(-c4cccc5ccccc45)cc3)c3ccc(-c4cccc5c4oc4ccccc45)cc3)c21]

N-[4-(naphthalen-1-yl)phenyl]-4-{8-oxatricyclo[7.4.0.0^{2,7}]trideca-1(9),2(7),3,5,10,12-hexaen-6-yl}aniline (40.3 g; 87.4 mmol), 1-bromofluoren-9-one (22.6 g, 87.4 mmol) and sodium tert-pentoxide (20.2 g, 174.7 mmol) are dissolved in 700 ml of toluene. The solution is degassed and saturated with N₂. It is subsequently admixed with tri-tert-butylphosphine (3.5 ml; 3.5 mmol, 1M in toluene) and 1.6 g (1.7 mmol) of Pd₂(dba)₃. The reaction mixture is heated to boiling under a protective atmosphere overnight. The mixture is cooled and partitioned between toluene and water and the organic phase is washed three times with water and dried over Na₂SO₄ and concentrated by rotary evaporation. After the crude product has been filtered through silica gel with toluene, the remaining residue is recrystallized from heptane/toluene. The yield is 49.2 g (80% of theory).

The following compounds are prepared in an analogous manner:

| | | | Product: |
|---|---|---|---|
| Ex. | Bromofluorenone | Amine | 1-Aminofluorenone |
| 2b | 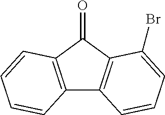 [SMILES: O=C1c2ccccc2-c2cccc(Br)c21] | 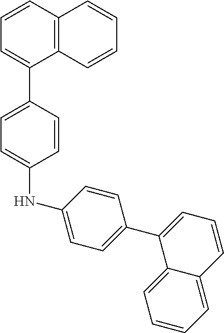 [SMILES: c1ccc2c(-c3ccc(Nc4ccc(-c5cccc6ccccc56)cc4)cc3)cccc2c1] | 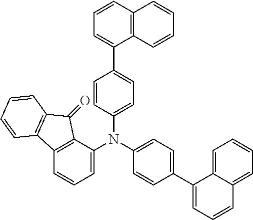 [SMILES: O=C1c2ccccc2-c2cccc(N(c3ccc(-c4cccc5ccccc45)cc3)c3ccc(-c4cccc5ccccc45)cc3)c21] |
| 2c | 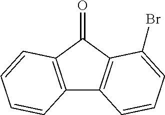 [SMILES: O=C1c2ccccc2-c2cccc(Br)c21] | 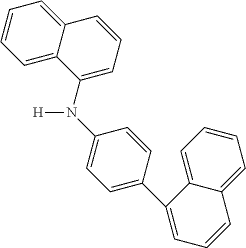 [SMILES: c1ccc2c(Nc3ccc(-c4cccc5ccccc45)cc3)cccc2c1] | 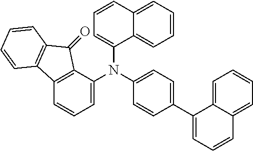 [SMILES: O=C1c2ccccc2-c2cccc(N(c3ccc(-c4cccc5ccccc45)cc3)c3cccc4ccccc34)c21] |
| 2d | 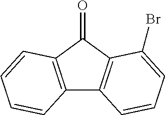 [SMILES: O=C1c2ccccc2-c2cccc(Br)c21] | 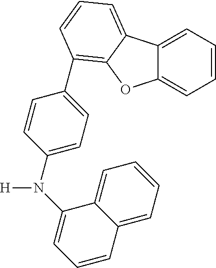 [SMILES: c1ccc2c(Nc3ccc(-c4cccc5c4oc4ccccc45)cc3)cccc2c1] | 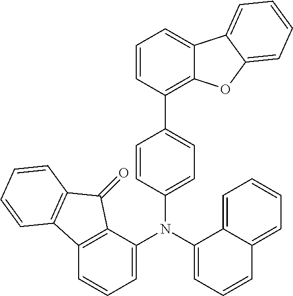 [SMILES: O=C1c2ccccc2-c2cccc(N(c3ccc(-c4cccc5c4oc4ccccc45)cc3)c3cccc4ccccc34)c21] |
| 2e | 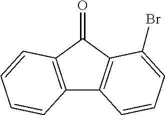 [SMILES: O=C1c2ccccc2-c2cccc(Br)c21] | 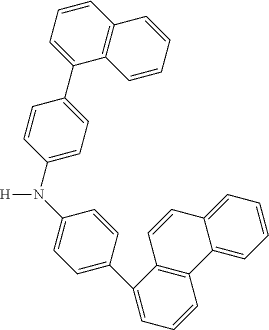 [SMILES: c1ccc2c(-c3ccc(Nc4ccc(-c5cccc6c5ccc5ccccc56)cc4)cc3)cccc2c1] | 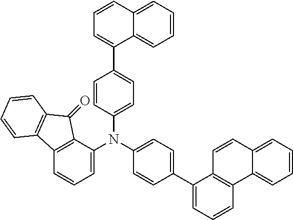 [SMILES: O=C1c2ccccc2-c2cccc(N(c3ccc(-c4cccc5ccccc45)cc3)c3ccc(-c4cccc5c4ccc4ccccc45)cc3)c21] |
| 2f | 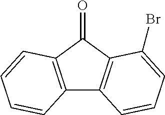 [SMILES: O=C1c2ccccc2-c2cccc(Br)c21] | 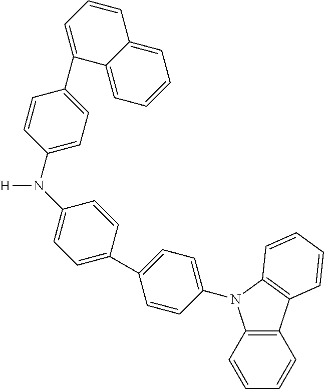 [SMILES: c1ccc2c(-c3ccc(Nc4ccc(-c5ccc(-n6c7ccccc7c7ccccc76)cc5)cc4)cc3)cccc2c1] | 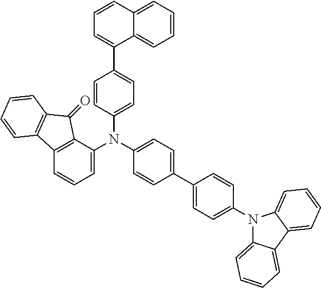 [SMILES: O=C1c2ccccc2-c2cccc(N(c3ccc(-c4ccc(-n5c6ccccc6c6ccccc65)cc4)cc3)c3ccc(-c4cccc5ccccc45)cc3)c21] |
| 2g | 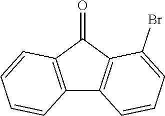 [SMILES: O=C1c2ccccc2-c2cccc(Br)c21] | 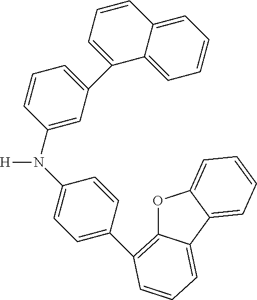 [SMILES: c1cc(Nc2ccc(-c3cccc4c3oc3ccccc34)cc2)cc(-c2cccc3ccccc23)c1] | 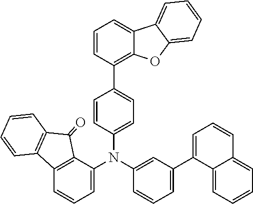 [SMILES: O=C1c2ccccc2-c2cccc(N(c3ccc(-c4cccc5c4oc4ccccc45)cc3)c3cccc(-c4cccc5ccccc45)c3)c21] |
| 2h | 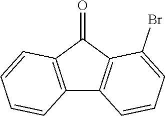 [SMILES: O=C1c2ccccc2-c2cccc(Br)c21] | 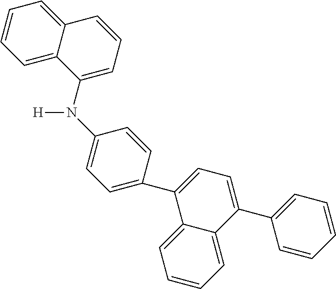 [SMILES: c1ccc(-c2ccc(-c3ccc(Nc4cccc5ccccc45)cc3)c3ccccc23)cc1] | 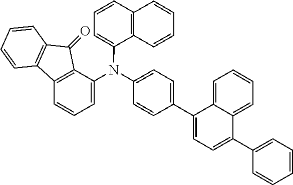 [SMILES: O=C1c2ccccc2-c2cccc(N(c3ccc(-c4ccc(-c5ccccc5)c5ccccc45)cc3)c3cccc4ccccc34)c21] |
| 2i | 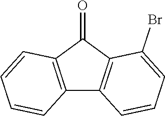 [SMILES: O=C1c2ccccc2-c2cccc(Br)c21] | 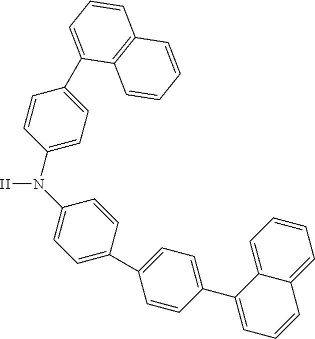 [SMILES: c1ccc2c(-c3ccc(Nc4ccc(-c5ccc(-c6cccc7ccccc67)cc5)cc4)cc3)cccc2c1] | 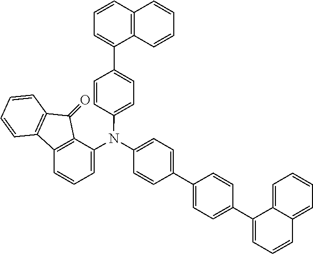 [SMILES: O=C1c2ccccc2-c2cccc(N(c3ccc(-c4ccc(-c5cccc6ccccc56)cc4)cc3)c3ccc(-c4cccc5ccccc45)cc3)c21] |
| 2j | 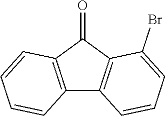 [SMILES: O=C1c2ccccc2-c2cccc(Br)c21] | 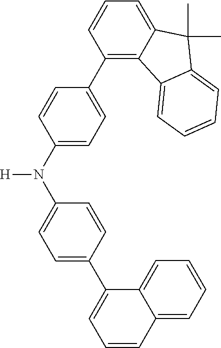 [SMILES: CC1(C)c2ccccc2-c2c(-c3ccc(Nc4ccc(-c5cccc6ccccc56)cc4)cc3)cccc21] | 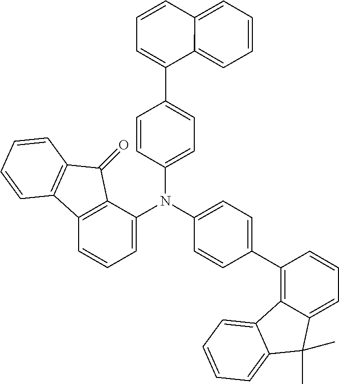 [SMILES: CC1(C)c2ccccc2-c2c(-c3ccc(N(c4ccc(-c5cccc6ccccc56)cc4)c4cccc5c4C(=O)c4ccccc4-5)cc3)cccc21] |

### 3) Synthesis of N-[4-(naphthalen-1-yl)phenyl]-N-(4-f8-oxatricyclo[7.4.0.0^{2,7}]trideca-1(13),2,4,6,9,11-hexaen-6-yl)phenyl)-9,9′-spirobi[fluoren]-1-amine 1a 3a

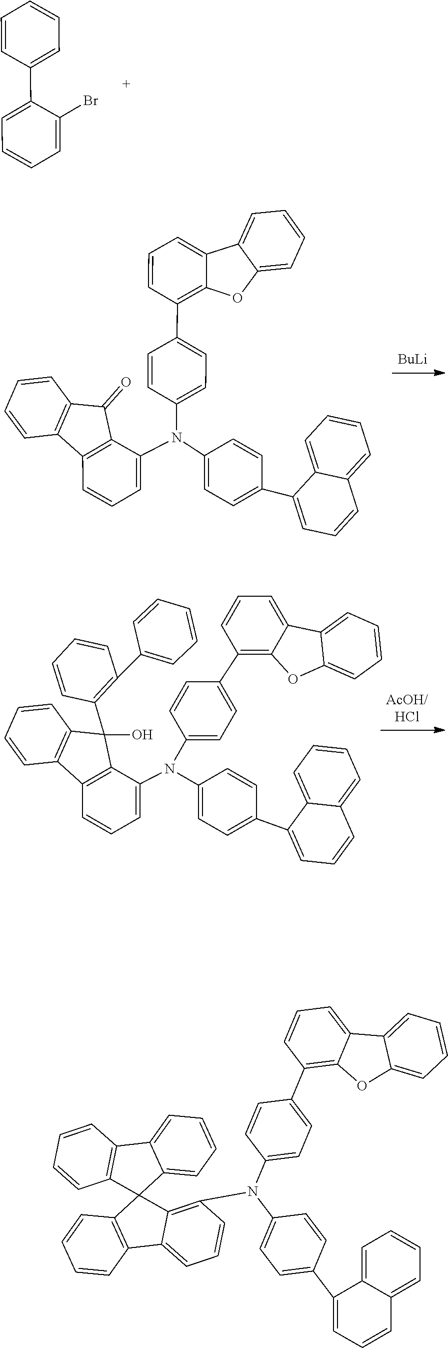

25 g (105.2 mmol) of 2-bromo-1,1′-biphenyl are dissolved in a baked-out flask in 300 ml of dried THF. The reaction mixture is cooled to −78° C. At this temperature, 39.3 ml of a 2.5 M solution of n-BuLi in hexane (98.2 mmol) are slowly added dropwise. The mixture is stirred at −70° C. for a further 1 hour. Subsequently 49.2 g of 1-{[4-(naphthalen-1-yl)phenyl](4-{8-oxatricyclo[7.4.0.0^{2,7}]trideca-1 (13),2,4,6,9,11-hexaen-6-yl}phenyl)amino}-9H-fluoren-9-one (70.1 mmol) is dissolved in 300 mL of THF and added dropwise at −70° C. After the addition has ended, the reaction mixture is left to warm up gradually to room temperature, the reaction is stopped with NH₄Cl, and then the mixture is concentrated on a rotary evaporator. The solid material is dissolved in 500 ml of toluene, and then 720 mg (3.8 mmol) of p-toluenesulfonic acid are added. The mixture is heated under reflux for 6 hours, then allowed to cool down to room temperature and admixed with water. The precipitated solid is filtered off with suction and washed with heptane (40.10 g, 68% yield). The remaining residue is recrystallized from heptane/toluene. The substance is finally sublimed under high vacuum, purity is 99.9% determined by HPLC. The yield is 19.2 g (48% of theory).

The following compounds are prepared in an analogous manner:

| | Reactant: | | |
|---|---|---|---|
| Ex. | 1-Aminofluorenone | Br-Biphenyl | Product |
| 3b | 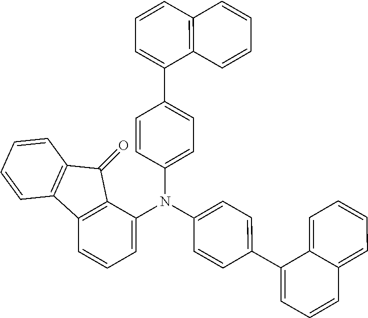 [SMILES: O=C1c2ccccc2-c2cccc(N(c3ccc(-c4cccc5ccccc45)cc3)c3ccc(-c4cccc5ccccc45)cc3)c21] | 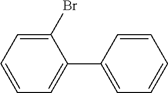 [SMILES: Brc1ccccc1-c1ccccc1] | 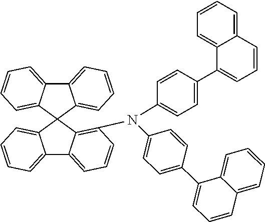 |
| 3c | 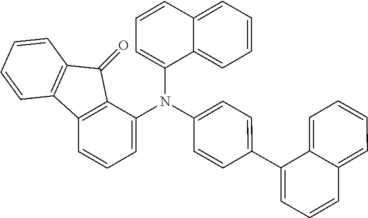 [SMILES: O=C1c2ccccc2-c2cccc(N(c3ccc(-c4cccc5ccccc45)cc3)c3cccc4ccccc34)c21] | 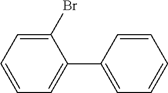 [SMILES: Brc1ccccc1-c1ccccc1] | 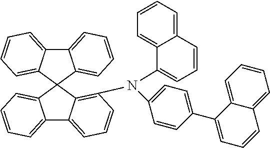 [SMILES: c1ccc2c(c1)-c1ccccc1C21c2ccccc2-c2cccc(N(c3ccc(-c4cccc5ccccc45)cc3)c3cccc4ccccc34)c21] |
| 3d | 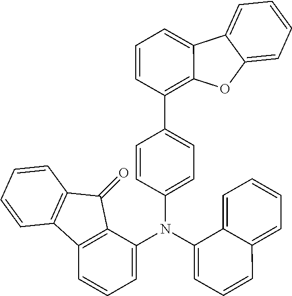 [SMILES: O=C1c2ccccc2-c2cccc(N(c3ccc(-c4cccc5c4oc4ccccc45)cc3)c3cccc4ccccc34)c21] | 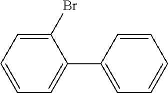 [SMILES: Brc1ccccc1-c1ccccc1] | 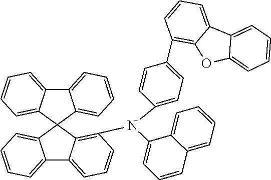 [SMILES: c1ccc2c(c1)-c1ccccc1C21c2ccccc2-c2cccc(N(c3ccc(-c4cccc5c4oc4ccccc45)cc3)c3cccc4ccccc34)c21] |
| 3e | 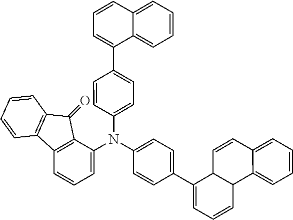 [SMILES: O=C1c2ccccc2-c2cccc(N(c3ccc(C4=CC=CC5c6ccccc6C=CC45)cc3)c3ccc(-c4cccc5ccccc45)cc3)c21] | 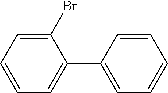 [SMILES: Brc1ccccc1-c1ccccc1] | 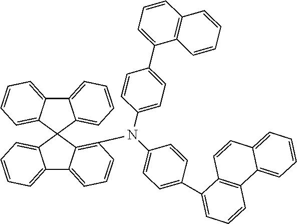 [SMILES: c1ccc2c(c1)-c1ccccc1C21c2ccccc2-c2cccc(N(c3ccc(-c4cccc5ccccc45)cc3)c3ccc(-c4cccc5c4ccc4ccccc45)cc3)c21] |
| 3f | 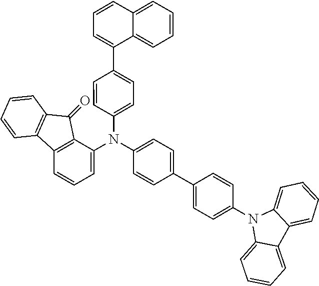 [SMILES: O=C1c2ccccc2-c2cccc(N(c3ccc(-c4ccc(-n5c6ccccc6c6ccccc65)cc4)cc3)c3ccc(-c4cccc5ccccc45)cc3)c21] | 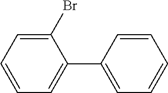 [SMILES: Brc1ccccc1-c1ccccc1] | 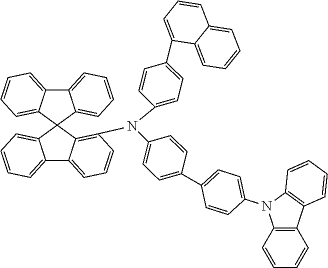 [SMILES: c1ccc2c(c1)-c1ccccc1C21c2ccccc2-c2cccc(N(c3ccc(-c4ccc(-n5c6ccccc6c6ccccc65)cc4)cc3)c3ccc(-c4cccc5ccccc45)cc3)c21] |
| 3g | 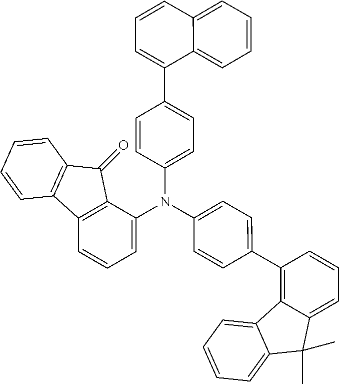 [SMILES: CC1(C)c2ccccc2-c2c(-c3ccc(N(c4ccc(-c5cccc6ccccc56)cc4)c4cccc5c4C(=O)c4ccccc4-5)cc3)cccc21] | 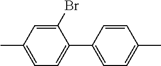 | 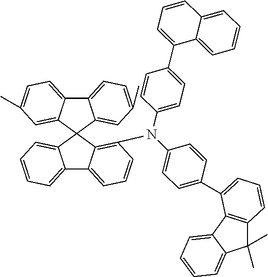 |

### 4) Synthesis of N-[4-(naphthalen-1-yl)phenyl]-N-(4-{8-oxatricyclo[7.4.0.0^{2,7}]trideca-1(13),2,4,6,9,11-hexaen-6-yl}phenyl)-4-{9,9′-spirobi[fluoren]-8-yl}aniline 4a

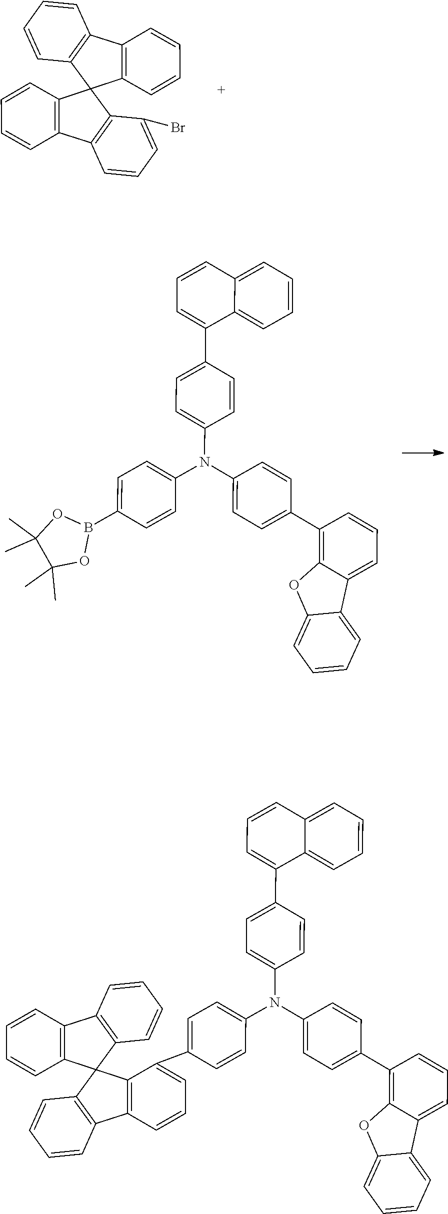 [SMILES: Brc1cccc2c1C1(c3ccccc3-c3ccccc31)c1ccccc1-2 ; CC1(C)OB(c2ccc(N(c3ccc(-c4cccc5ccccc45)cc3)c3ccc(-c4cccc5c4oc4ccccc45)cc3)cc2)OC1(C)C ; c1ccc2c(c1)-c1ccccc1C21c2ccccc2-c2cccc(-c3ccc(N(c4ccc(-c5cccc6ccccc56)cc4)c4ccc(-c5cccc6c5oc5ccccc56)cc4)cc3)c21]

25.9 g (39 mmol) of 4-(naphthalen-1-yl)-N-(4-{8-oxatricyclo[7.4.0.0^{2,7}]trideca-1(13),2,4,6,9,11-hexaen-6-yl}phenyl)-N-[4-(4,4,5,5-tetramethyl-1,3,2-dioxaborolan-2-yl)phenyl]aniline and 16.6 g (42 mmol) of 8-bromo-9,9′-spirobi[fluorene] are suspended in 400 ml of dioxane and 13.7 g of caesium fluoride (90 mmol). 4.0 g (5.4 mmol) of bis(tricyclohexylphosphine)palladium dichloride are added to this suspension, and the reaction mixture is heated under reflux for 18 h. After cooling, the organic phase is removed, filtered through silica gel, washed three times with 80 ml of water and then concentrated to dryness. After the crude product has been filtered through silica gel with toluene, the remaining residue is recrystallized from heptane/toluene and finally sublimed under high vacuum, purity is 99.9% determined by HPLC. The yield is 11 g (33% of theory).

The following compounds are prepared in an analogous manner:

| Ex. | Spiro | Amine | Product |
|---|---|---|---|
| 4b | 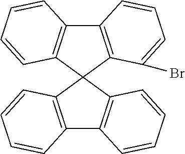 [SMILES: Brc1cccc2c1C1(c3ccccc3-c3ccccc31)c1ccccc1-2] | 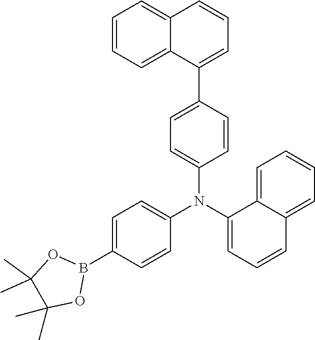 [SMILES: CC1(C)OB(c2ccc(N(c3ccc(-c4cccc5ccccc45)cc3)c3cccc4ccccc34)cc2)OC1(C)C] | 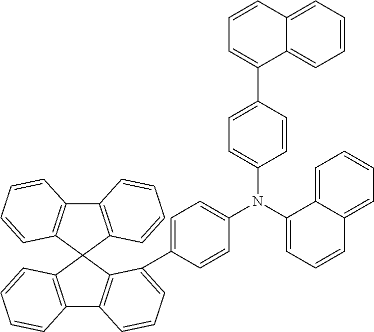 [SMILES: c1ccc2c(c1)-c1ccccc1C21c2ccccc2-c2cccc(-c3ccc(N(c4ccc(-c5cccc6ccccc56)cc4)c4cccc5ccccc45)cc3)c21] |
| 4c | 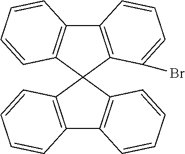 [SMILES: Brc1cccc2c1C1(c3ccccc3-c3ccccc31)c1ccccc1-2] | 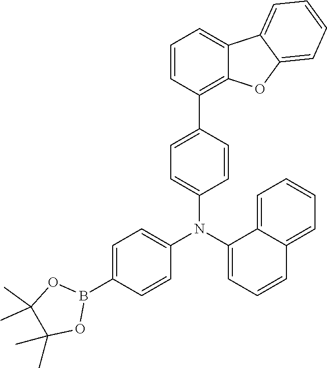 [SMILES: CC1(C)OB(c2ccc(N(c3ccc(-c4cccc5c4oc4ccccc45)cc3)c3cccc4ccccc34)cc2)OC1(C)C] | 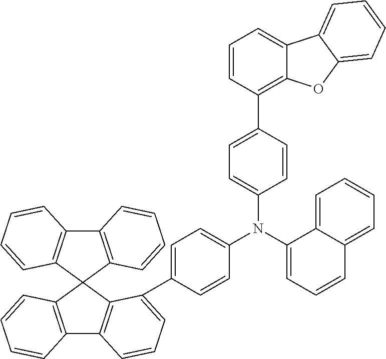 [SMILES: c1ccc2c(c1)-c1ccccc1C21c2ccccc2-c2cccc(-c3ccc(N(c4ccc(-c5cccc6c5oc5ccccc56)cc4)c4cccc5ccccc45)cc3)c21] |
| 4d | 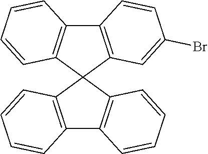 [SMILES: Brc1ccc2c(c1)C1(c3ccccc3-c3ccccc31)c1ccccc1-2] | 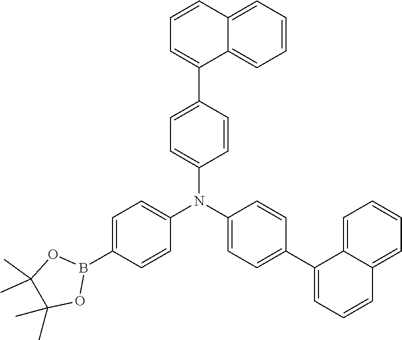 [SMILES: CC1(C)OB(c2ccc(N(c3ccc(-c4cccc5ccccc45)cc3)c3ccc(-c4cccc5ccccc45)cc3)cc2)OC1(C)C] | 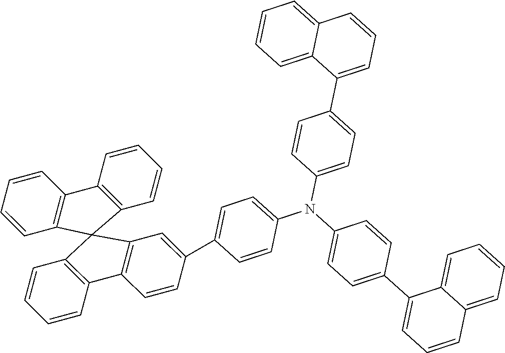 [SMILES: c1ccc2c(c1)-c1ccccc1C21c2ccccc2-c2ccc(-c3ccc(N(c4ccc(-c5cccc6ccccc56)cc4)c4ccc(-c5cccc6ccccc56)cc4)cc3)cc21] |
| 4e | 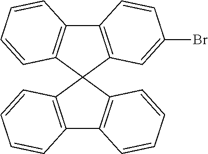 [SMILES: Brc1ccc2c(c1)C1(c3ccccc3-c3ccccc31)c1ccccc1-2] | 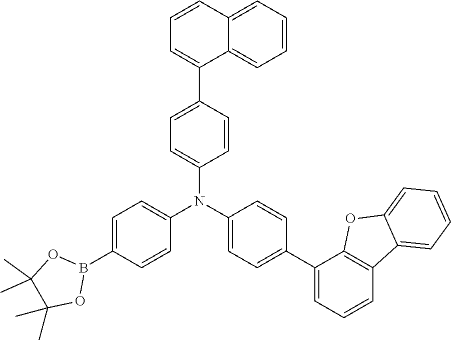 [SMILES: CC1(C)OB(c2ccc(N(c3ccc(-c4cccc5ccccc45)cc3)c3ccc(-c4cccc5c4oc4ccccc45)cc3)cc2)OC1(C)C] | 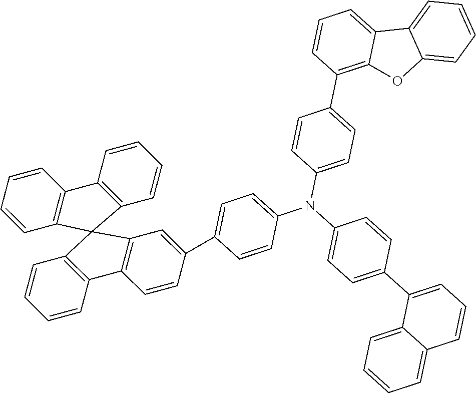 [SMILES: c1ccc2c(c1)-c1ccccc1C21c2ccccc2-c2ccc(-c3ccc(N(c4ccc(-c5cccc6ccccc56)cc4)c4ccc(-c5cccc6c5oc5ccccc56)cc4)cc3)cc21] |
| 4f | 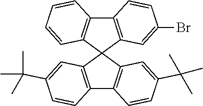 [SMILES: CC(C)(C)c1ccc2c(c1)C1(c3ccccc3-c3ccc(Br)cc31)c1cc(C(C)(C)C)ccc1-2] | 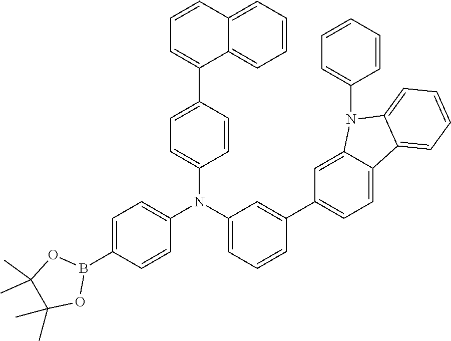 [SMILES: CC1(C)OB(c2ccc(N(c3ccc(-c4cccc5ccccc45)cc3)c3cccc(-c4ccc5c6ccccc6n(-c6ccccc6)c5c4)c3)cc2)OC1(C)C] | 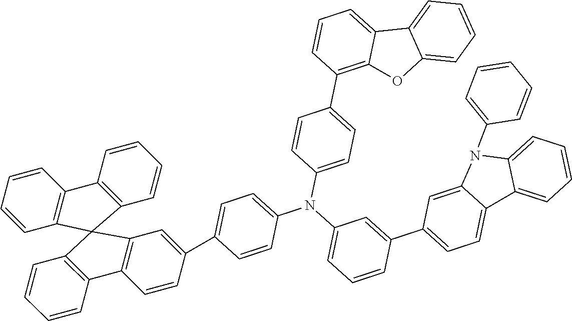 [SMILES: c1ccc(-n2c3ccccc3c3ccc(-c4cccc(N(c5ccc(-c6ccc7c(c6)C6(c8ccccc8-c8ccccc86)c6ccccc6-7)cc5)c5ccc(-c6cccc7c6oc6ccccc67)cc5)c4)cc32)cc1] |

### 5) Synthesis of 1-(4-chloro-phenyl)-fluoren-9-one 5a

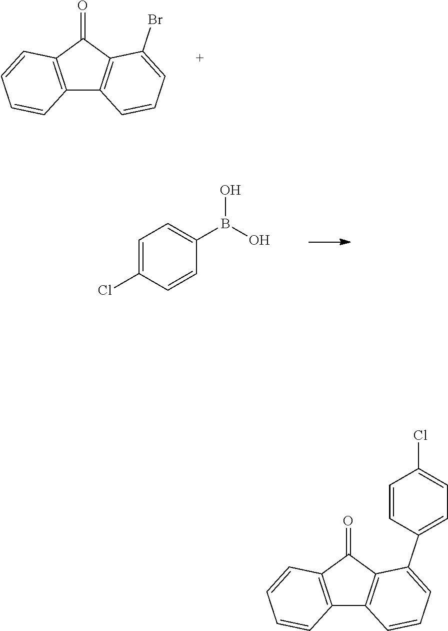 [SMILES: O=C1c2ccccc2-c2cccc(Br)c21 ; OB(O)c1ccc(Cl)cc1 ; O=C1c2ccccc2-c2cccc(-c3ccc(Cl)cc3)c21]

76 g (486 mmol) of 4-chlorophenylboronic acid, 120 g (463 mmol) of 1-bromofluorene and 16 g (14 mmol) of Pd(Ph₃P)₄ are dissolved in 1.9 L of THF. The solution is degassed and saturated with N₂ and 463 ml of 2 M potassium carbonate solution are slowly added to this suspension. The reaction mixture is heated to boiling under a protective atmosphere overnight. The mixture is subsequently partitioned between toluene and water, and the organic phase is washed three times with water and dried over Na₂SO₄ and concentrated by rotary evaporation. The residue is purified by crystallization with MeOH. Yield: 125 g (93% of theory), purity by HPLC >98%.

The following compounds are prepared in an analogous manner:

| Ex. | 1-Br-Fluorenone | Boronic acid | Product |
|---|---|---|---|
| 5b | 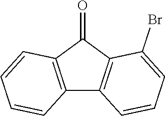 [SMILES: O=C1c2ccccc2-c2cccc(Br)c21] | 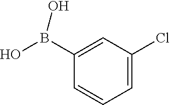 | 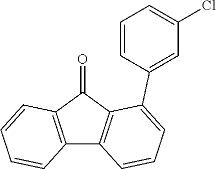 [SMILES: O=C1c2ccccc2-c2cccc(-c3cccc(Cl)c3)c21] |
| 5c | 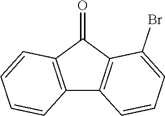 [SMILES: O=C1c2ccccc2-c2cccc(Br)c21] | 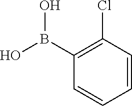 | 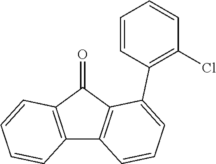 [SMILES: O=C1c2ccccc2-c2cccc(-c3ccccc3Cl)c21] |
| 5d | 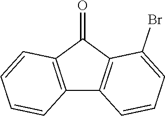 [SMILES: O=C1c2ccccc2-c2cccc(Br)c21] | 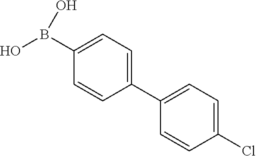 [SMILES: OB(O)c1ccc(-c2ccc(Cl)cc2)cc1] | 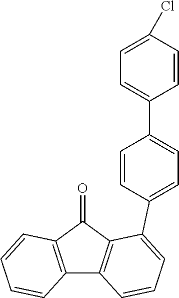 [SMILES: O=C1c2ccccc2-c2cccc(-c3ccc(-c4ccc(Cl)cc4)cc3)c21] |
| 5e | 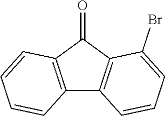 [SMILES: O=C1c2ccccc2-c2cccc(Br)c21] | 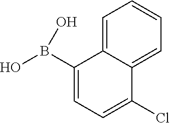 [SMILES: OB(O)c1ccc(Cl)c2ccccc12] | 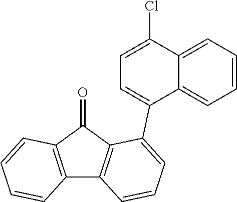 [SMILES: O=C1c2ccccc2-c2cccc(-c3ccc(Cl)c4ccccc34)c21] |

### 6) Synthesis of 1-(4-{[4-(naphthalen-1-yl)phenyl](4-{8-oxatricyclo[7.4.0.0^{2,7}]trideca-1(13),2,4,6,9,11-hexaen-6-yl}phenyl)amino}phenyl)-9H-fluoren-9-one 6a

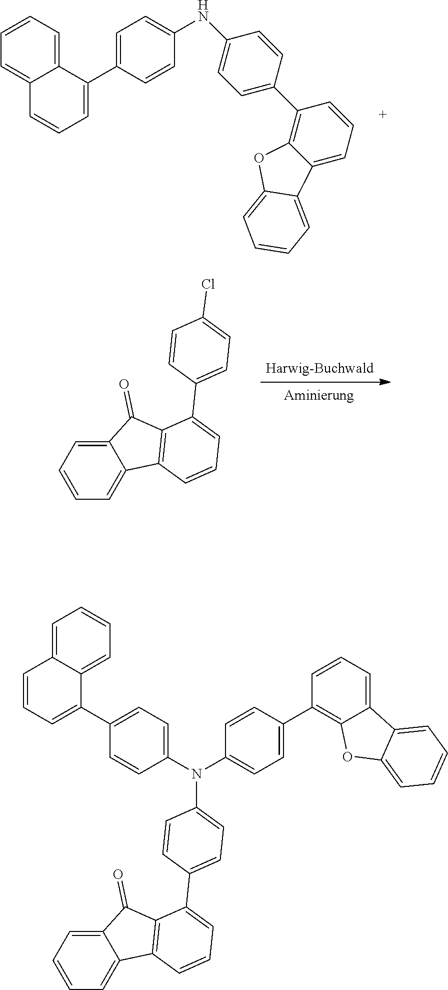 [SMILES: c1ccc2c(-c3ccc(Nc4ccc(-c5cccc6c5oc5ccccc56)cc4)cc3)cccc2c1 ; O=C1c2ccccc2-c2cccc(-c3ccc(Cl)cc3)c21 ; O=C1c2ccccc2-c2cccc(-c3ccc(N(c4ccc(-c5cccc6ccccc56)cc4)c4ccc(-c5cccc6c5oc5ccccc56)cc4)cc3)c21]

N-[4-(naphthalen-1-yl)phenyl]-4-{8-oxatricyclo[7.4.0.0^{2,7}]trideca-1(9),2(7),3,5,10,12-hexaen-6-yl}aniline (32 g, 69 mmol),1-1-(4-chlorophenyl)-fluoren-9-one, (20 g, 69 mmol) and sodium tert-butoxide (9.5 g, 138 mmol) are dissolved in 300 mL of toluene. The solution is degassed and saturated with N₂. It is subsequently admixed with tri-tert-butylphosphine (2 ml, 2 mmol, 1 M in toluene) and 0.98 g (1 mmol) of Pd₂(dba)₃. The reaction mixture is heated to boiling under a protective atmosphere overnight. The mixture is cooled and partitioned between toluene and water and the organic phase is washed three times with water and dried over Na₂SO₄ and concentrated by rotary evaporation. After the crude product has been filtered through silica gel with toluene, the remaining residue is recrystallized from heptane/toluene. The yield is 43 g (87% of theory).

The following compounds are prepared in an analogous manner:

| Ex. | Fluorenone | Amine | Product |
|---|---|---|---|
| 6b | 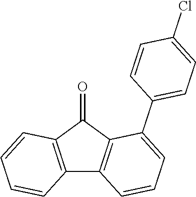 [SMILES: O=C1c2ccccc2-c2cccc(-c3ccc(Cl)cc3)c21] | 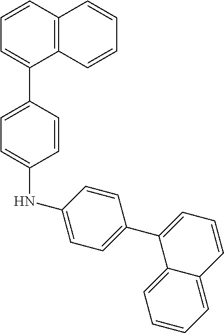 [SMILES: c1ccc2c(-c3ccc(Nc4ccc(-c5cccc6ccccc56)cc4)cc3)cccc2c1] | 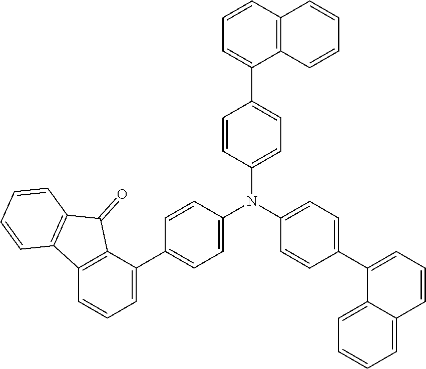 [SMILES: O=C1c2ccccc2-c2cccc(-c3ccc(N(c4ccc(-c5cccc6ccccc56)cc4)c4ccc(-c5cccc6ccccc56)cc4)cc3)c21] |
| 6c | 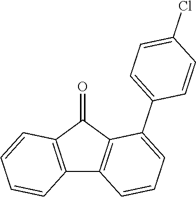 [SMILES: O=C1c2ccccc2-c2cccc(-c3ccc(Cl)cc3)c21] | 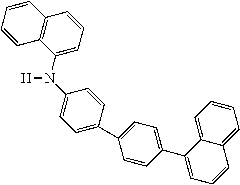 [SMILES: c1ccc2c(Nc3ccc(-c4ccc(-c5cccc6ccccc56)cc4)cc3)cccc2c1] | 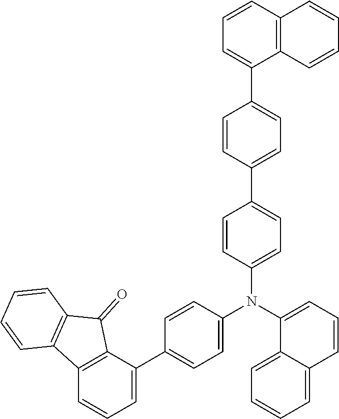 [SMILES: O=C1c2ccccc2-c2cccc(-c3ccc(N(c4ccc(-c5ccc(-c6cccc7ccccc67)cc5)cc4)c4cccc5ccccc45)cc3)c21] |
| 6d | 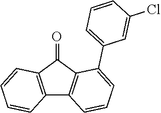 [SMILES: O=C1c2ccccc2-c2cccc(-c3cccc(Cl)c3)c21] | 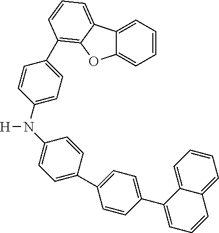 [SMILES: c1ccc2c(-c3ccc(-c4ccc(Nc5ccc(-c6cccc7c6oc6ccccc67)cc5)cc4)cc3)cccc2c1] | 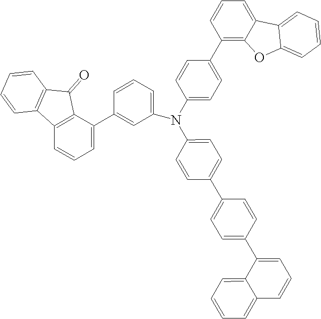 [SMILES: O=C1c2ccccc2-c2cccc(-c3cccc(N(c4ccc(-c5ccc(-c6cccc7ccccc67)cc5)cc4)c4ccc(-c5cccc6c5oc5ccccc56)cc4)c3)c21] |
| 6f | 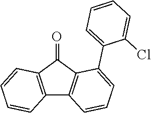 [SMILES: O=C1c2ccccc2-c2cccc(-c3ccccc3Cl)c21] | 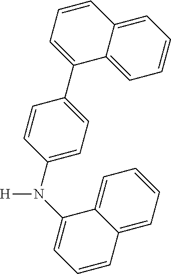 [SMILES: c1ccc2c(Nc3ccc(-c4cccc5ccccc45)cc3)cccc2c1] | 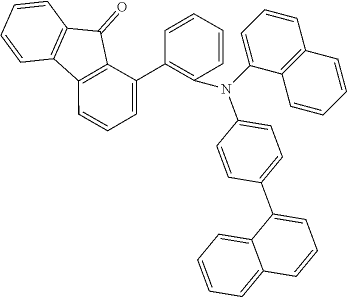 [SMILES: O=C1c2ccccc2-c2cccc(-c3ccccc3N(c3ccc(-c4cccc5ccccc45)cc3)c3cccc4ccccc34)c21] |
| 6g | 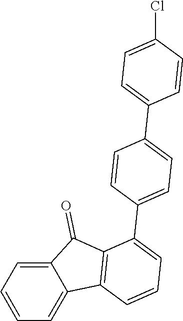 [SMILES: O=C1c2ccccc2-c2cccc(-c3ccc(-c4ccc(Cl)cc4)cc3)c21] | 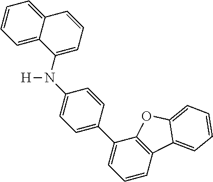 [SMILES: c1ccc2c(Nc3ccc(-c4cccc5c4oc4ccccc45)cc3)cccc2c1] | 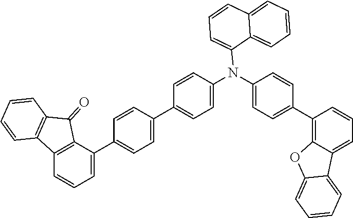 [SMILES: O=C1c2ccccc2-c2cccc(-c3ccc(-c4ccc(N(c5ccc(-c6cccc7c6oc6ccccc67)cc5)c5cccc6ccccc56)cc4)cc3)c21] |

### 7) Synthesis of N-[4-(naphthalen-1-yl)phenyl]-N-(4-{8-oxatricyclo[7.4.0.0^{2,7}]trideca-1(13),2,4,6,9,11-hexaen-6-yl}phenyl)-4-{9,9′-spirobi[fluoren]-8-yl}aniline 7a

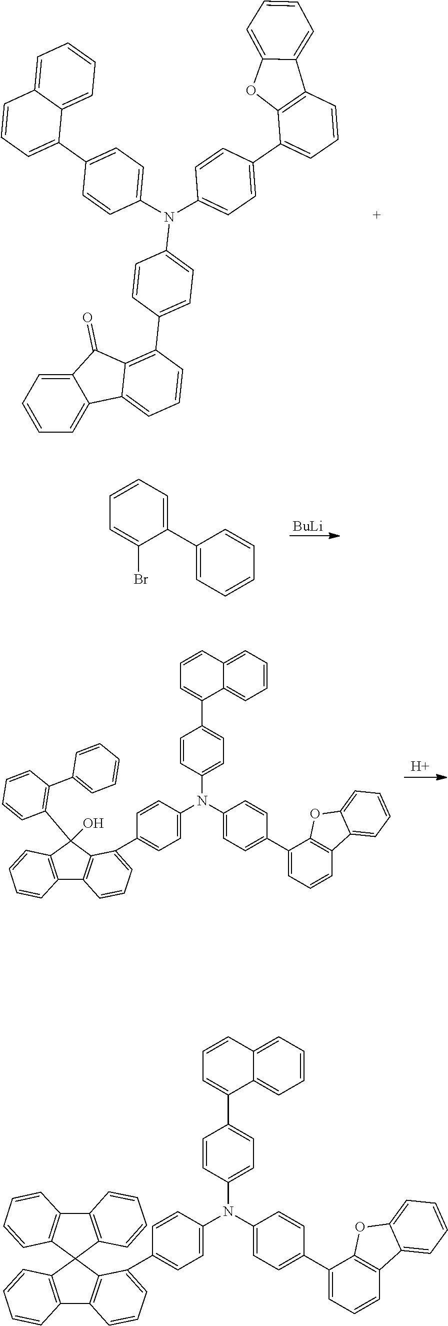

17 g (73 mmol) of 2-bromo-1,1′-biphenyl are dissolved in a baked-out flask in 90 ml of dried THF. The reaction mixture is cooled to −78° C. At this temperature, 165 ml of a 2.5 M solution of n-BuLi in hexane (66 mmol) are slowly added dropwise. The mixture is stirred at −70° C. for a further 1 hour. Subsequently 50 g of 1-(4-{[4-(naphthalen-1-yl)phenyl](4-{8-oxatricyclo[7.4.0.0^{2,7}]trideca-1(13),2,4,6,9,11-hexaen-6-yl}phenyl)amino}phenyl)-9H-fluoren-9-one (70.1 mmol) are dissolved in 300 ml of THF and added dropwise at −70° C. After the addition has ended, the reaction mixture is left to warm up gradually to room temperature, the reaction is stopped with NH₄Cl, and then the mixture is concentrated on a rotary evaporator. The solid material is dissolved in 500 ml of toluene, and then 720 mg (3.8 mmol) of p-toluenesulfonic acid are added. The mixture is heated under reflux for 6 hours, then allowed to cool down to room temperature and admixed with water. The precipitated solid is filtered off with suction and washed with heptane (40.10 g, 66% yield).

The remaining residue is recrystallized from heptane/toluene. The substance is finally sublimed under high vacuum, purity is 99.9% determined by HPLC. The yield is 28 g (48% of theory).

The following compounds are prepared in an analogous manner:

| Ex. | 1-Aminefluorenone | Br-Biphenyl | Product |
|---|---|---|---|
| 7b | 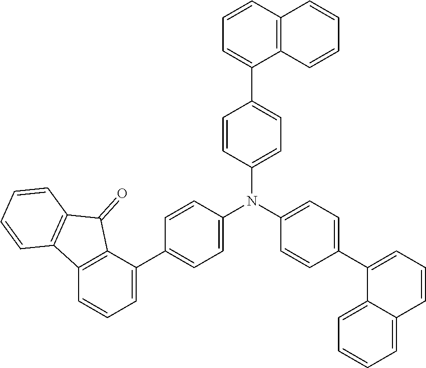 [SMILES: O=C1c2ccccc2-c2cccc(-c3ccc(N(c4ccc(-c5cccc6ccccc56)cc4)c4ccc(-c5cccc6ccccc56)cc4)cc3)c21] | 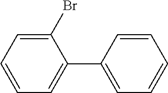 [SMILES: Brc1ccccc1-c1ccccc1] | 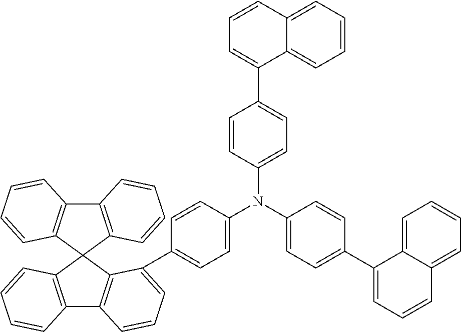 [SMILES: c1ccc2c(c1)-c1ccccc1C21c2ccccc2-c2cccc(-c3ccc(N(c4ccc(-c5cccc6ccccc56)cc4)c4ccc(-c5cccc6ccccc56)cc4)cc3)c21] |
| 7c | 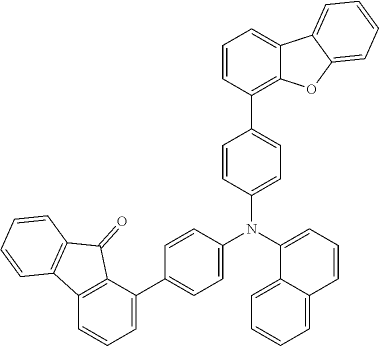 [SMILES: O=C1c2ccccc2-c2cccc(-c3ccc(N(c4ccc(-c5cccc6c5oc5ccccc56)cc4)c4cccc5ccccc45)cc3)c21] | 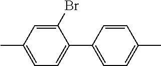 | 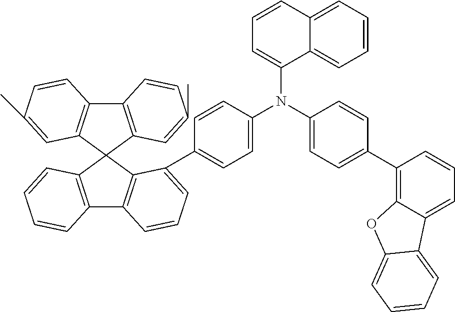 [SMILES: Cc1ccc2c(c1)C1(c3cc(C)ccc3-2)c2ccccc2-c2cccc(-c3ccc(N(c4ccc(-c5cccc6c5oc5ccccc56)cc4)c4cccc5ccccc45)cc3)c21] |
| 7d | 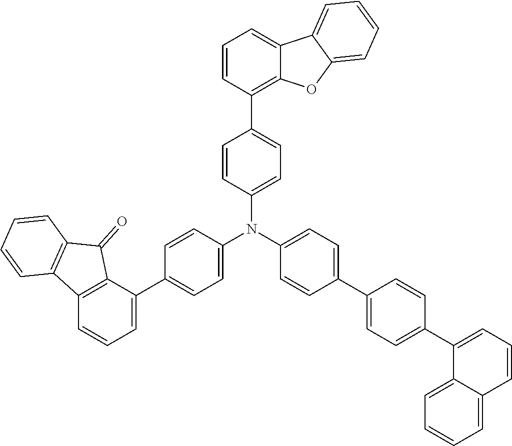 [SMILES: O=C1c2ccccc2-c2cccc(-c3ccc(N(c4ccc(-c5ccc(-c6cccc7ccccc67)cc5)cc4)c4ccc(-c5cccc6c5oc5ccccc56)cc4)cc3)c21] | 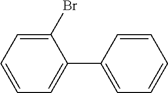 [SMILES: Brc1ccccc1-c1ccccc1] | 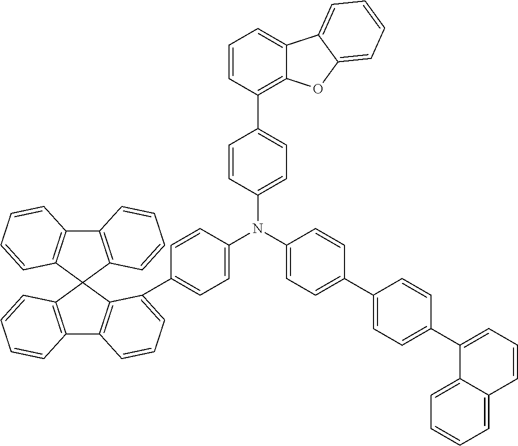 [SMILES: c1ccc2c(c1)-c1ccccc1C21c2ccccc2-c2cccc(-c3ccc(N(c4ccc(-c5ccc(-c6cccc7ccccc67)cc5)cc4)c4ccc(-c5cccc6c5oc5ccccc56)cc4)cc3)c21] |
| 7e | 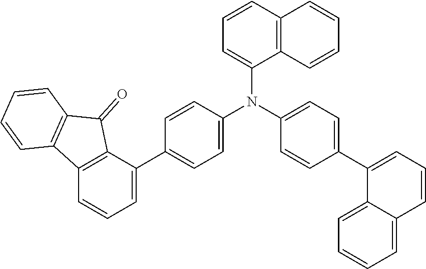 [SMILES: O=C1c2ccccc2-c2cccc(-c3ccc(N(c4ccc(-c5cccc6ccccc56)cc4)c4cccc5ccccc45)cc3)c21] | 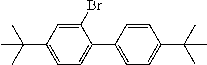 [SMILES: CC(C)(C)c1ccc(-c2ccc(C(C)(C)C)cc2Br)cc1] | 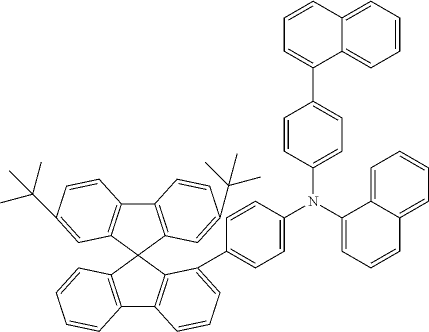 [SMILES: CC(C)(C)c1ccc2c(c1)C1(c3cc(C(C)(C)C)ccc3-2)c2ccccc2-c2cccc(-c3ccc(N(c4ccc(-c5cccc6ccccc56)cc4)c4cccc5ccccc45)cc3)c21] |

### 8) Synthesis of 8′-(4-chlorophenyl)-9,9′-spirobifluorene 8a

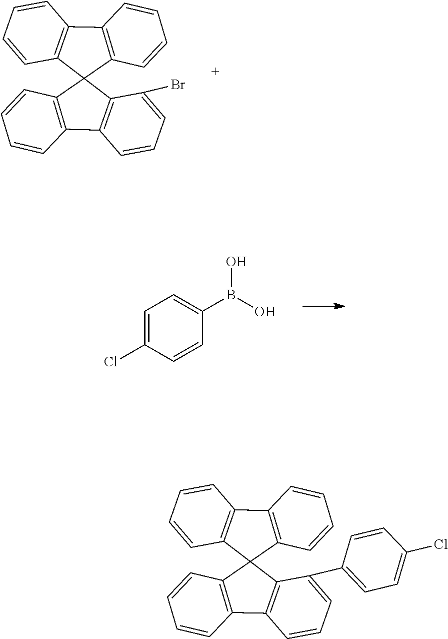 [SMILES: Brc1cccc2c1C1(c3ccccc3-c3ccccc31)c1ccccc1-2 ; OB(O)c1ccc(Cl)cc1 ; Clc1ccc(-c2cccc3c2C2(c4ccccc4-c4ccccc42)c2ccccc2-3)cc1]

10.7 g (69 mmol) of 4-chlorophenylboronic acid, 27.2 g (69 mmol) of 1-bromospiro and 5.4 g (5 mmol) of Pd(Ph₃P)₄ are dissolved in 600 ml of THF. The solution is degassed and saturated with N₂ and 155 ml of 2 M potassium carbonate solution are slowly added to this suspension. The reaction mixture is heated to boiling under a protective atmosphere overnight. The mixture is subsequently partitioned between toluene and water, and the organic phase is washed three times with water and dried over Na₂SO₄ and concentrated by rotary evaporation. The residue is purified by crystallization with MeOH. Yield: 25 g (85% of theory). Purity by HPLC >98%.

The following compounds are prepared in an analogous manner:

| Ex. | Reactant 1 | Reactant 2 | Product |
|---|---|---|---|
| 8b | 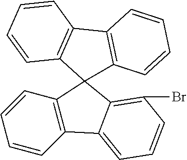 [SMILES: Brc1cccc2c1C1(c3ccccc3-c3ccccc31)c1ccccc1-2] | 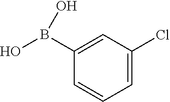 | 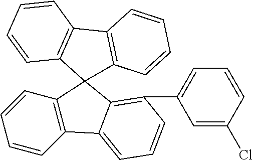 [SMILES: Clc1cccc(-c2cccc3c2C2(c4ccccc4-c4ccccc42)c2ccccc2-3)c1] |
| 8c | 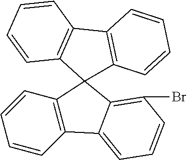 [SMILES: Brc1cccc2c1C1(c3ccccc3-c3ccccc31)c1ccccc1-2] | 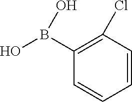 | 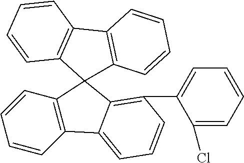 [SMILES: Clc1ccccc1-c1cccc2c1C1(c3ccccc3-c3ccccc31)c1ccccc1-2] |
| 8d | 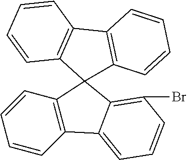 [SMILES: Brc1cccc2c1C1(c3ccccc3-c3ccccc31)c1ccccc1-2] | 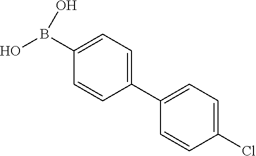 [SMILES: OB(O)c1ccc(-c2ccc(Cl)cc2)cc1] | 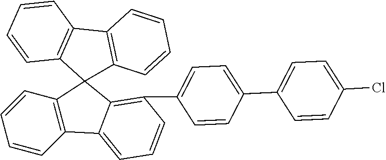 [SMILES: Clc1ccc(-c2ccc(-c3cccc4c3C3(c5ccccc5-c5ccccc53)c3ccccc3-4)cc2)cc1] |
| 8e | 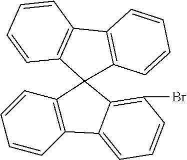 [SMILES: Brc1cccc2c1C1(c3ccccc3-c3ccccc31)c1ccccc1-2] | 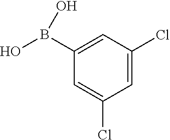 [SMILES: OB(O)c1cc(Cl)cc(Cl)c1] | 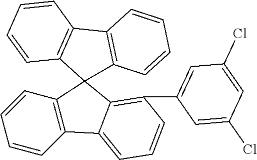 [SMILES: Clc1cc(Cl)cc(-c2cccc3c2C2(c4ccccc4-c4ccccc42)c2ccccc2-3)c1] |

### 9) Synthesis of N-((1,1′-biphenyl)-4-yl)N-(4-(2′,7′-di-tert-butyl-9,9′-spirobi(fluorene)-1-yl)phenyl)-9,9-dimethylfluoren-2-amine 9a

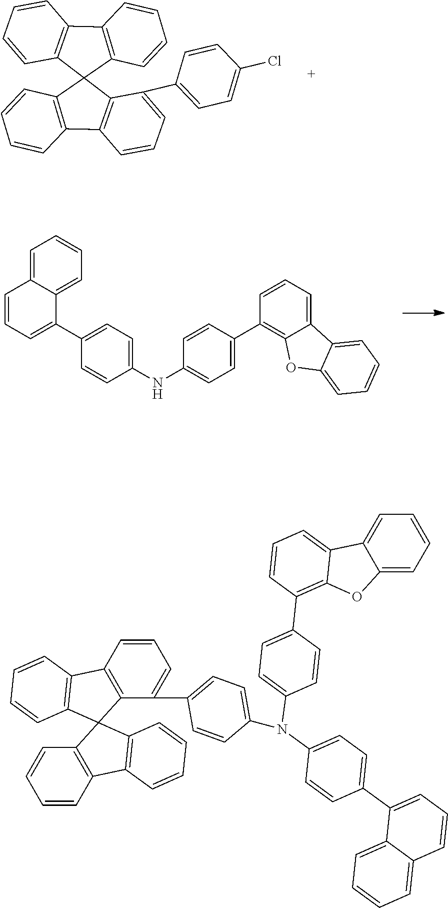 [SMILES: Clc1ccc(-c2cccc3c2C2(c4ccccc4-c4ccccc42)c2ccccc2-3)cc1 ; c1ccc2c(-c3ccc(Nc4ccc(-c5cccc6c5oc5ccccc56)cc4)cc3)cccc2c1 ; c1ccc2c(c1)-c1ccccc1C21c2ccccc2-c2cccc(-c3ccc(N(c4ccc(-c5cccc6ccccc56)cc4)c4ccc(-c5cccc6c5oc5ccccc56)cc4)cc3)c21]

10.1 g (28 mmol) of N-[4-(naphthalen-1-yl)phenyl]-4-8-oxatricyclo[7.4.0.0^{2,7}]trideca-1 (9),2(7),3,5,10,12-hexaen-6-yl-aniline and 14.5 g (27 mol) of the 8′-(4-chlorophenyl)-9,9′-spirobifluorene are dissolved in 225 ml of toluene. The solution is degassed and saturated with N₂. It is subsequently admixed with 2.1 ml (2.1 mmol) of tri-tert-butylphosphine solution (1 M in toluene) and 0.98 g (1 mmol) of Pd₂(dba)₃ and then 5.1 g of sodium tert-butoxide (53 mmol) are added. The reaction mixture is heated to boiling under a protective atmosphere overnight. The mixture is subsequently partitioned between toluene and water, and the organic phase is washed three times with water and dried over Na₂SO₄ and concentrated by rotary evaporation. After the crude product has been filtered through silica gel with toluene, the remaining residue is recrystallized from heptane/toluene. The substance is finally sublimed under high vacuum; purity is 99.9% determined by HPLC. The yield is 6 g (26% of theory).

The following compounds are prepared in an analogous manner:

| Ex. | Spirofluorene | Amine | Product |
|---|---|---|---|
| 9b | 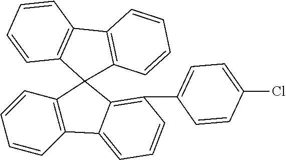 [SMILES: Clc1ccc(-c2cccc3c2C2(c4ccccc4-c4ccccc42)c2ccccc2-3)cc1] | 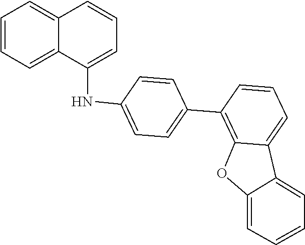 [SMILES: c1ccc2c(Nc3ccc(-c4cccc5c4oc4ccccc45)cc3)cccc2c1] | 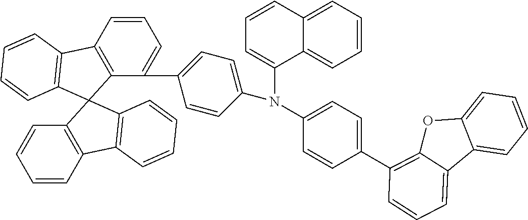 [SMILES: c1ccc2c(c1)-c1ccccc1C21c2ccccc2-c2cccc(-c3ccc(N(c4ccc(-c5cccc6c5oc5ccccc56)cc4)c4cccc5ccccc45)cc3)c21] |
| 9c | 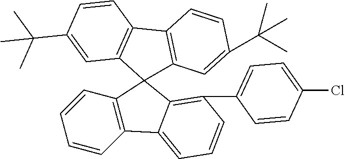 [SMILES: CC(C)(C)c1ccc2c(c1)C1(c3cc(C(C)(C)C)ccc3-2)c2ccccc2-c2cccc(-c3ccc(Cl)cc3)c21] | 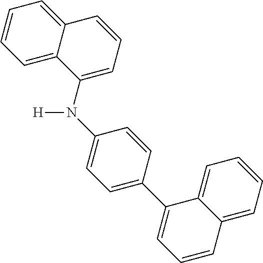 [SMILES: c1ccc2c(Nc3ccc(-c4cccc5ccccc45)cc3)cccc2c1] | 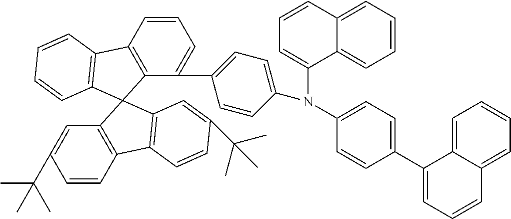 [SMILES: CC(C)(C)c1ccc2c(c1)C1(c3cc(C(C)(C)C)ccc3-2)c2ccccc2-c2cccc(-c3ccc(N(c4ccc(-c5cccc6ccccc56)cc4)c4cccc5ccccc45)cc3)c21] |
| 9d | 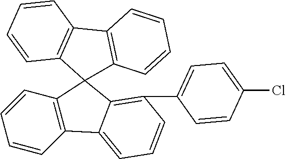 [SMILES: Clc1ccc(-c2cccc3c2C2(c4ccccc4-c4ccccc42)c2ccccc2-3)cc1] | 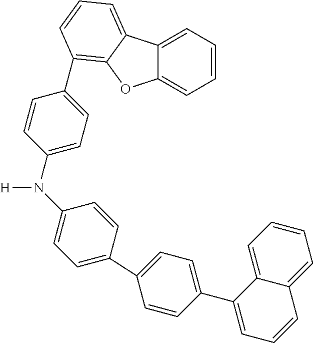 [SMILES: c1ccc2c(-c3ccc(-c4ccc(Nc5ccc(-c6cccc7c6oc6ccccc67)cc5)cc4)cc3)cccc2c1] | 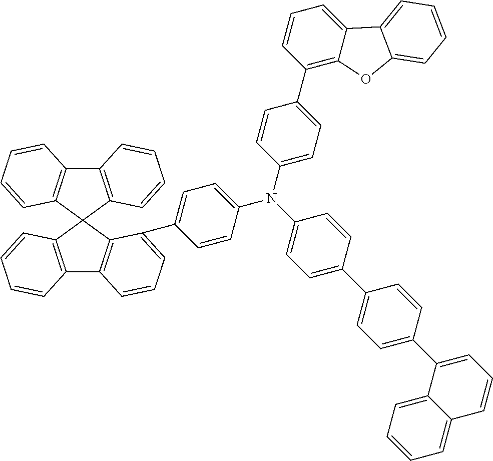 [SMILES: c1ccc2c(c1)-c1ccccc1C21c2ccccc2-c2cccc(-c3ccc(N(c4ccc(-c5ccc(-c6cccc7ccccc67)cc5)cc4)c4ccc(-c5cccc6c5oc5ccccc56)cc4)cc3)c21] |
| 9e | 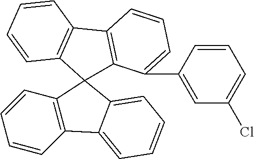 [SMILES: Clc1cccc(-c2cccc3c2C2(c4ccccc4-c4ccccc42)c2ccccc2-3)c1] | 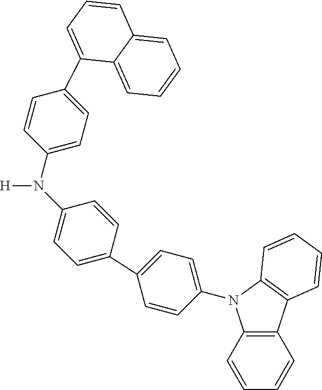 [SMILES: c1ccc2c(-c3ccc(Nc4ccc(-c5ccc(-n6c7ccccc7c7ccccc76)cc5)cc4)cc3)cccc2c1] | 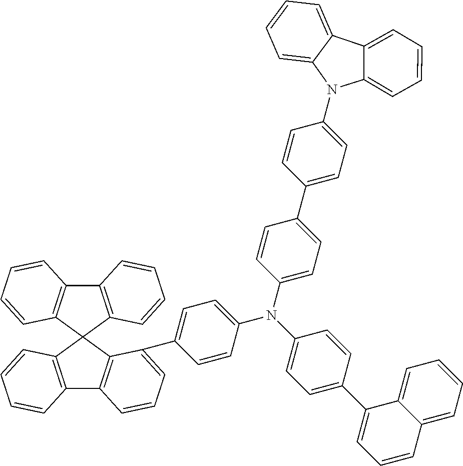 [SMILES: c1ccc2c(c1)-c1ccccc1C21c2ccccc2-c2cccc(-c3ccc(N(c4ccc(-c5ccc(-n6c7ccccc7c7ccccc76)cc5)cc4)c4ccc(-c5cccc6ccccc56)cc4)cc3)c21] |
| 9f | 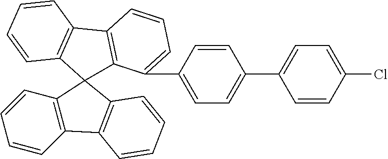 [SMILES: Clc1ccc(-c2ccc(-c3cccc4c3C3(c5ccccc5-c5ccccc53)c3ccccc3-4)cc2)cc1] | 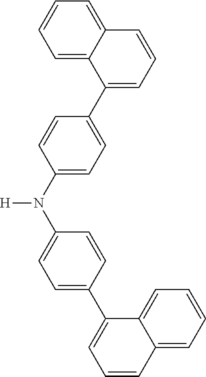 [SMILES: c1ccc2c(-c3ccc(Nc4ccc(-c5cccc6ccccc56)cc4)cc3)cccc2c1] | 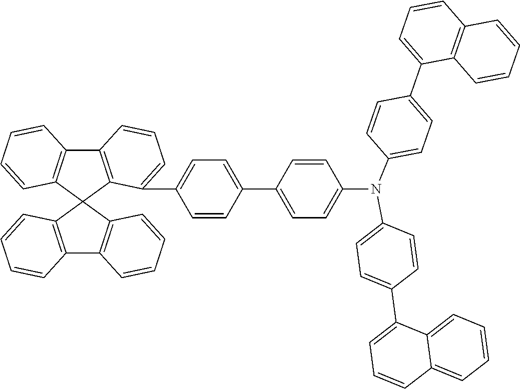 [SMILES: c1ccc2c(c1)-c1ccccc1C21c2ccccc2-c2cccc(-c3ccc(-c4ccc(N(c5ccc(-c6cccc7ccccc67)cc5)c5ccc(-c6cccc7ccccc67)cc5)cc4)cc3)c21] |
| 9g | 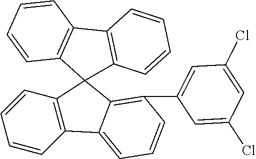 [SMILES: Clc1cc(Cl)cc(-c2cccc3c2C2(c4ccccc4-c4ccccc42)c2ccccc2-3)c1] | 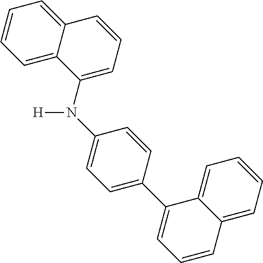 [SMILES: c1ccc2c(Nc3ccc(-c4cccc5ccccc45)cc3)cccc2c1] | 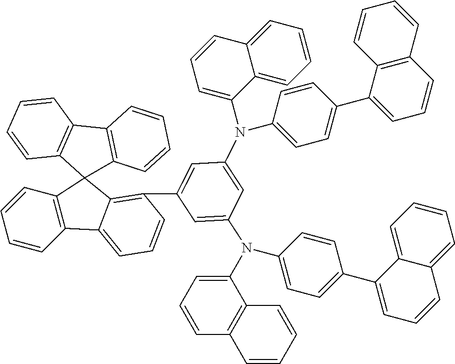 [SMILES: c1ccc2c(c1)-c1ccccc1C21c2ccccc2-c2cccc(-c3cc(N(c4ccc(-c5cccc6ccccc56)cc4)c4cccc5ccccc45)cc(N(c4ccc(-c5cccc6ccccc56)cc4)c4cccc5ccccc45)c3)c21] |

### 10) Synthesis of N-[4-(naphthalen-1-yl)phenyl]-N-(4-{8-oxatricyclo[7.4.0.0^{2,7}]trideca-1(13),2,4,6,9,11-hexaen-6-yl}phenyl)-4′-{9,9′-spirobi[fluoren]-8-yl}-[1,1′-biphenyl]-4-amine 10a

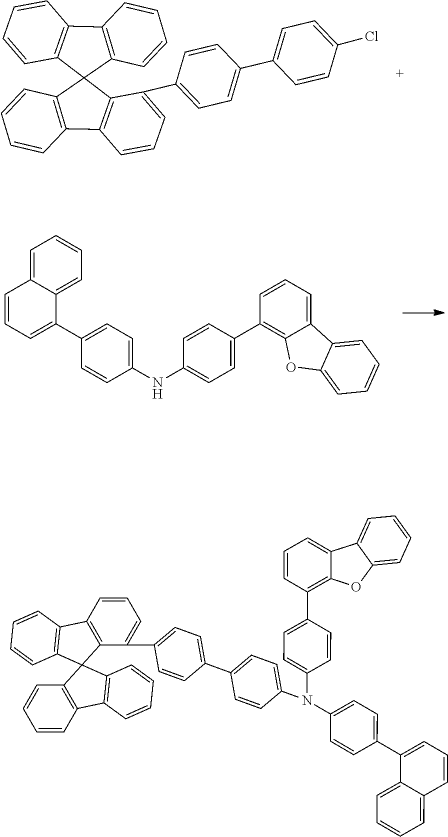 [SMILES: Clc1ccc(-c2ccc(-c3cccc4c3C3(c5ccccc5-c5ccccc53)c3ccccc3-4)cc2)cc1 ; c1ccc2c(-c3ccc(Nc4ccc(-c5cccc6c5oc5ccccc56)cc4)cc3)cccc2c1 ; c1ccc2c(c1)-c1ccccc1C21c2ccccc2-c2cccc(-c3ccc(-c4ccc(N(c5ccc(-c6cccc7ccccc67)cc5)c5ccc(-c6cccc7c6oc6ccccc67)cc5)cc4)cc3)c21]

1510.1 g (28 mmol) of N-[4-(naphthalen-1-yl)phenyl]-4-8-oxatricyclo[7.4.0.0²⁷]trideca-1(9),2(7)3,5,10,12-hexaen-6-yl-aniline and 14.5 g (29 mmol) of the 1-{4′-chloro-[1,1′-biphenyl]-4-yl}-9,9′-spirobi[fluorene] are dissolved in 225 ml of toluene. The solution is degassed and saturated with N₂. It is subsequently admixed with 2.1 ml (2.1 mmol) of tri-tert-butylphosphine solution (1 M in toluene) and 0.98 g (1 mmol) of Pd₂(dba)₃ and then 5.1 g of sodium tert-butoxide (53 mmol) are added. The reaction mixture is heated to boiling under a protective atmosphere overnight. The mixture is subsequently partitioned between toluene and water, and the organic phase is washed three times with water and dried over Na₂SO₄ and concentrated by rotary evaporation. After the crude product has been filtered through silica gel with toluene, the remaining residue is recrystallized from heptane/toluene. The substance is finally sublimed under high vacuum; purity is 99.9%. The yield is 8 g (30% of theory).

The following compounds are prepared in an analogous manner:

| Ex. | Spirofluorene | Amine | Product |
|---|---|---|---|
| 10b | 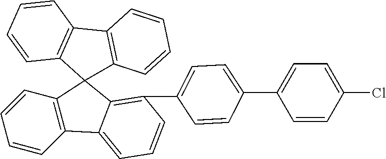 [SMILES: Clc1ccc(-c2ccc(-c3cccc4c3C3(c5ccccc5-c5ccccc53)c3ccccc3-4)cc2)cc1] | 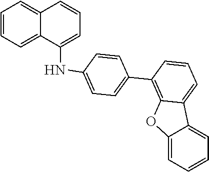 [SMILES: c1ccc2c(Nc3ccc(-c4cccc5c4oc4ccccc45)cc3)cccc2c1] | 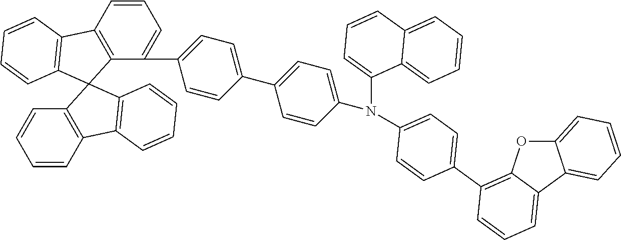 [SMILES: c1ccc2c(c1)-c1ccccc1C21c2ccccc2-c2cccc(-c3ccc(-c4ccc(N(c5ccc(-c6cccc7c6oc6ccccc67)cc5)c5cccc6ccccc56)cc4)cc3)c21] |
| 10c | 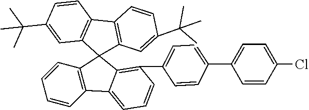 [SMILES: CC(C)(C)c1ccc2c(c1)C1(c3cc(C(C)(C)C)ccc3-2)c2ccccc2-c2cccc(-c3ccc(-c4ccc(Cl)cc4)cc3)c21] | 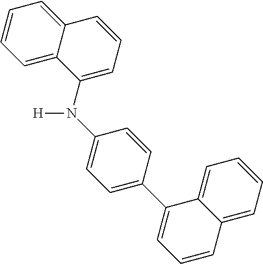 [SMILES: c1ccc2c(Nc3ccc(-c4cccc5ccccc45)cc3)cccc2c1] | 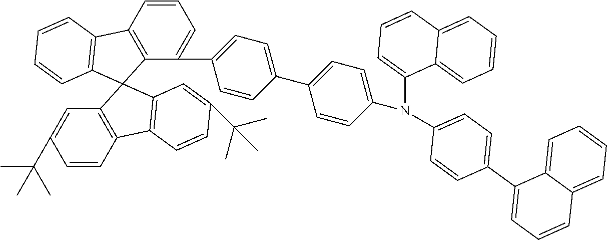 [SMILES: CC(C)(C)c1ccc2c(c1)C1(c3cc(C(C)(C)C)ccc3-2)c2ccccc2-c2cccc(-c3ccc(-c4ccc(N(c5ccc(-c6cccc7ccccc67)cc5)c5cccc6ccccc56)cc4)cc3)c21] |
| 10d | 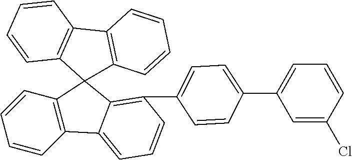 [SMILES: Clc1cccc(-c2ccc(-c3cccc4c3C3(c5ccccc5-c5ccccc53)c3ccccc3-4)cc2)c1] | 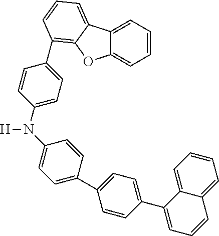 [SMILES: c1ccc2c(-c3ccc(-c4ccc(Nc5ccc(-c6cccc7c6oc6ccccc67)cc5)cc4)cc3)cccc2c1] | 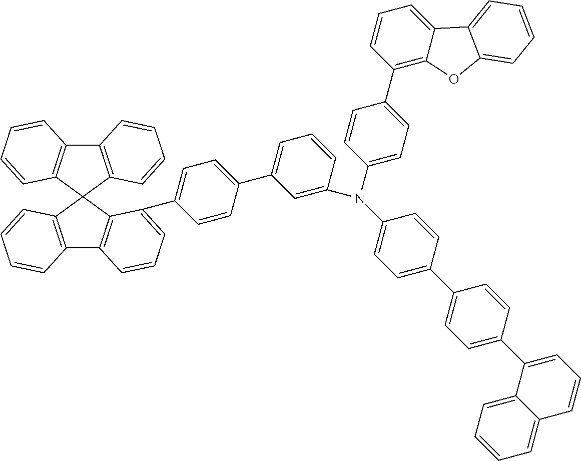 [SMILES: c1cc(-c2ccc(-c3cccc4c3C3(c5ccccc5-c5ccccc53)c3ccccc3-4)cc2)cc(N(c2ccc(-c3ccc(-c4cccc5ccccc45)cc3)cc2)c2ccc(-c3cccc4c3oc3ccccc34)cc2)c1] |
| 10e | 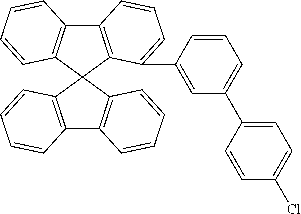 [SMILES: Clc1ccc(-c2cccc(-c3cccc4c3C3(c5ccccc5-c5ccccc53)c3ccccc3-4)c2)cc1] | 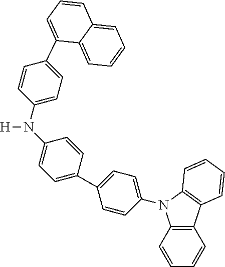 [SMILES: c1ccc2c(-c3ccc(Nc4ccc(-c5ccc(-n6c7ccccc7c7ccccc76)cc5)cc4)cc3)cccc2c1] | 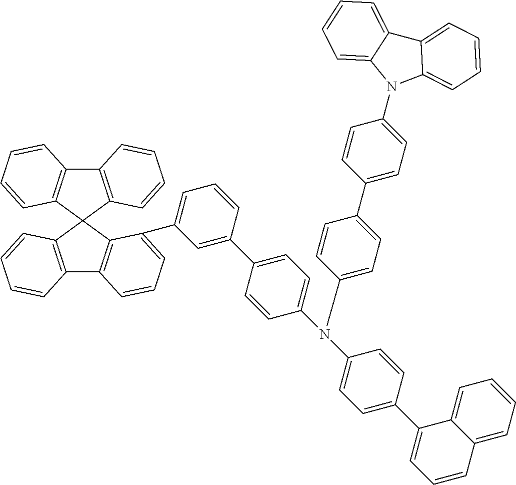 [SMILES: c1cc(-c2ccc(N(c3ccc(-c4ccc(-n5c6ccccc6c6ccccc65)cc4)cc3)c3ccc(-c4cccc5ccccc45)cc3)cc2)cc(-c2cccc3c2C2(c4ccccc4-c4ccccc42)c2ccccc2-3)c1] |
| 10f | 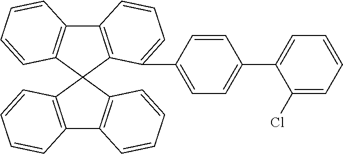 [SMILES: Clc1ccccc1-c1ccc(-c2cccc3c2C2(c4ccccc4-c4ccccc42)c2ccccc2-3)cc1] | 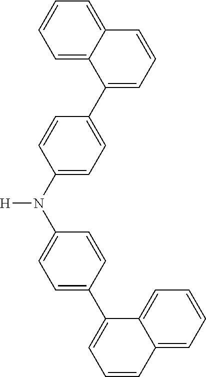 [SMILES: c1ccc2c(-c3ccc(Nc4ccc(-c5cccc6ccccc56)cc4)cc3)cccc2c1] | 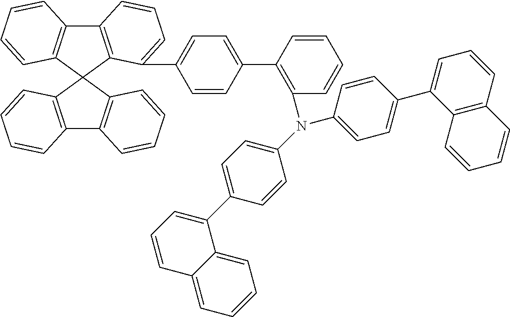 [SMILES: c1ccc(N(c2ccc(-c3cccc4ccccc34)cc2)c2ccc(-c3cccc4ccccc34)cc2)c(-c2ccc(-c3cccc4c3C3(c5ccccc5-c5ccccc53)c3ccccc3-4)cc2)c1] |
| 10g | 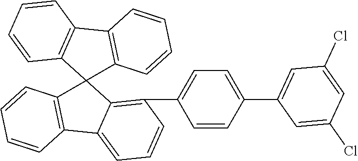 [SMILES: Clc1cc(Cl)cc(-c2ccc(-c3cccc4c3C3(c5ccccc5-c5ccccc53)c3ccccc3-4)cc2)c1] | 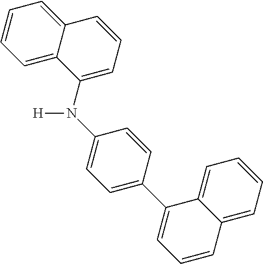 [SMILES: c1ccc2c(Nc3ccc(-c4cccc5ccccc45)cc3)cccc2c1] | 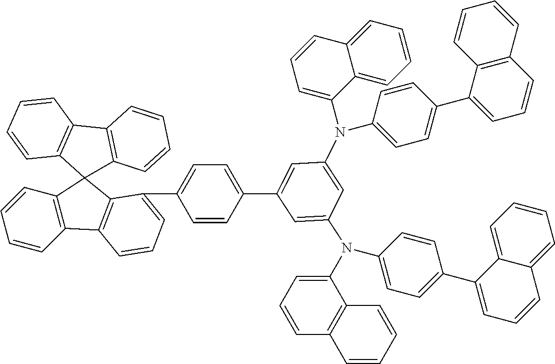 [SMILES: c1ccc2c(c1)-c1ccccc1C21c2ccccc2-c2cccc(-c3ccc(-c4cc(N(c5ccc(-c6cccc7ccccc67)cc5)c5cccc6ccccc56)cc(N(c5ccc(-c6cccc7ccccc67)cc5)c5cccc6ccccc56)c4)cc3)c21] |

### B) Device Examples

1) General Production Process for the OLEDs and Characterization of the OLEDs

Glass plaques which have been coated with structured ITO (indium tin oxide) in a thickness of 50 nm form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/electron injection layer (EIL) and finally a cathode. The cathode is formed by an aluminium layer of thickness 100 nm. The exact structure of the OLEDs can be found in the tables which follow. The materials used for production of the OLEDs are shown in a table below. The material H used here is an anthracene derivative, and the material SEB used is a spirobifluorenediamine. The p-dopant used is NDP-9 from Novaled-AG, Dresden.

All materials are applied by thermal vapour deposition in a vacuum chamber. In this case, the emission layer consists of at least one matrix material (host material) and an emitting dopant which is added to the matrix material(s) in a particular proportion by volume by co-evaporation. Details given in such a form as H:SEB (95%:5%) mean here that the material H is present in the layer in a proportion by volume of 95% and SEB in a proportion of 5%. Analogously, the electron transport layer and the hole injection layer also consist of a mixture of two materials. The structures of the materials that are used in the OLEDs are shown in Table 3.

The OLEDs are characterized in a standard manner. For this purpose, the electroluminescence spectra, the external quantum efficiency (EQE, measured in %) as a function of the luminance, calculated from current-voltage-luminance characteristics assuming Lambertian radiation characteristics, and the lifetime are determined. The parameter EQE @ 10 mA/cm² refers to the external quantum efficiency which is attained at 10 mA/cm². The parameter U @ 10 mA/cm² refers to the operating voltage at 10 mA/cm². The lifetime LT is defined as the time after which the luminance drops from the starting luminance to a certain proportion in the course of operation with constant current density. An LT90 figure means here that the lifetime reported corresponds to the time after which the luminance has dropped to 90% of its starting value. The figure @80 mA/cm² means here that the lifetime in question is measured at 80 mA/cm².

2) Inventive OLEDs Containing a Compound of the Formula (I) in the EBL of Blue-Fluorescing OLEDs: Comparison of the Inventive Substitution Positions 1 and 2 with the Substitution Positions 3 and 4

Devices as shown in the following table are produced:

| | HIL | HTL | EBL | EML | HBL | ETL | EIL |
|---|---|---|---|---|---|---|---|
| | Thickness/ | Thickness/ | Thickness/ | Thickness/ | Thickness/ | Thickness/ | Thickness/ |
| Ex. | nm | nm | nm | nm | nm | nm | nm |
| E1 | HTM: p-dopant | HTM | HTM-1 | H: SEB(3%) | HBM 10 nm | ETM: LIQ(50%) | LIQ |
| | (5%) | 180 nm | 10 nm | 20 nm | | 30 nm | 1 nm |
| | 10 nm | | | | | | |
| E2 | HTM: p-dopant | HTM | HTM-2 | H: SEB(3%) | HBM 10 nm | ETM: LIQ(50%) | LiQ |
| | (5%) | 180 nm | 10 nm | 20 nm | | 30 nm | 1 nm |
| | 10 nm | | | | | | |
| E-C1 | HTM: p-dopant | HTM | HTM-C1 | H: SEB(3%) | HBM 10 nm | ETM: LIQ(50%) | LiQ |
| | (5%) | 180 nm | 10 nm | 20 nm | | 30 nm | 1 nm |
| | 10 nm | | | | | | |
| E-C2 | HTM: p-dopant | HTM | HTM-C2 | H: SEB(3%) | HBM 10 nm | ETM: LIQ(50%) | LiQ |
| | (5%) | 180 nm | 10 nm | 20 nm | | 30 nm | 1 nm |
| | 10 nm | | | | | | |

Compounds according to the present application are employed in the OLEDs E1 and E2. OLEDs E-C1 and E-C2 serve as a reference and contain compounds substituted by the amino group at positions 3 or 4 of the spiro.

According to measurements the inventive OLEDs (substitution position 1 or 2 at the spirobifluorene) exhibit an improved lifetime compared to the OLEDs E-C1 and E-C2 with substitution positions 3 or 4 for the amino group at the spirobifluorene:

| | LT90 @ 60 |
|---|---|
| | mA/cm² (h) |
| E1 | 210 |
| E2 | 240 |
| E-C1 | 180 |
| E-C2 | 180 |

3) Use of the Inventive Compounds in the EBL of Green-Phosphorescing OLEDs

The following OLEDs are produced:

| | HIL | HTL | EBL | EML | HBL | ETL | EIL |
|---|---|---|---|---|---|---|---|
| | Thickness/ | Thickness/ | Thickness/ | Thickness/ | Thickness/ | Thickness/ | Thickness/ |
| Ex. | nm | nm | nm | nm | nm | nm | nm |
| E-3 | HTM: p- | HTM | HTM-1 | TMM-1(32%) TMM- | HBM 10 nm | ETM: LIQ(50%) | LiQ |
| | dopant (5%) | 90 nm | 20 nm | 2(60%) TEG (8%) | | 30 nm | 1 nm |
| | 20 nm | | | 30 nm | | | |
| E-4 | HTM: p- | HTM | HTM-2 | TMM-1(32%) TMM- | HBM 10 nm | ETM: LIQ(50%) | LiQ |
| | dopant (5%) | 90 nm | 20 nm | 2(60%) TEG (8%) | | 30 nm | 1 nm |
| | 20 nm | | | 30 nm | | | |
| E-5 | HTM: p- | HTM | HTM-3 | TMM-1(32%) TMM- | HBM 10 nm | ETM: LIQ(50%) | LiQ |
| | dopant (5%) | 90 nm | 20 nm | 2(60%) TEG (8%) | | 30 nm | 1 nm |
| | 20 nm | | | 30 nm | | | |

The inventive compounds HTM-1 to HTM-3 achieve good performance data in respect of lifetime (90-120 h LT90@80 mA), operating voltage (3.5V-3.9V U@10 mA) and efficiency (22.2-24.4% EQE@10 mA).

4) Use of the Inventive Compounds in the HIL and HTL of Blue-Fluorescing OLEDs

The following OLEDs are produced:

| | HIL | HTL | EBL | EML | HBL | ETL | EIL |
|---|---|---|---|---|---|---|---|
| | Thickness/ | Thickness/ | Thickness/ | Thickness/ | Thickness/ | Thickness/ | Thickness/ |
| Ex. | nm | nm | nm | nm | nm | nm | nm |
| E-6 | HTM-1: p-dopant | HTM-1 | EBM | H: SEB(3%) | HBM 10 nm | ETM: LIQ(50%) | LiQ |
| | (5%) | 180 nm | 10 nm | 20 nm | | 30 nm | 1 nm |
| | 10 nm | | | | | | |
| E-7 | HTM-2: p-dopant | HTM-2 | EBM | H: SEB(3%) | HBM 10 nm | ETM: LIQ(50%) | LiQ |
| | (5%) | 180 nm | 10 nm | 20 nm | | 30 nm | 1 nm |
| | 10 nm | | | | | | |

The inventive compounds achieve good performance data in particular in respect of efficiency (8.0-9.0% EQE@10 mA) at an operating voltage of 4.0-8.0 V U@10 mA.

5) Use of the Inventive Compounds in the EBL of Blue-Fluorescing OLEDs

The following OLED is produced:

| Ex. | HIL | HTL1 | HTL2 | EBL | EML | ETL | EIL |
|---|---|---|---|---|---|---|---|
| E-8 | HTM-A: p- | HTM-A | HTM-4: p- | HTM-4 | H-1: SEB- | ETM-1: | LiQ |
| | dopant (5%) | 160 nm | dopant (5%) | 10nm | 1(5%) | LiQ(50%) | 1 nm |
| | 20 nm | | 20nm | | 20 nm | 30 nm | |

The inventive compound HTM-4 achieves good results. The lifetime LT90@60 mA is 210 h, the efficiency EQE@10 mA is about 8% and the voltage U@10 mA is about 4 V.

TABLE 3
Structures of the compounds

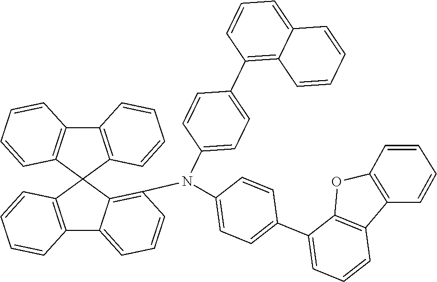 [SMILES: c1ccc2c(c1)-c1ccccc1C21c2ccccc2-c2cccc(N(c3ccc(-c4cccc5ccccc45)cc3)c3ccc(-c4cccc5c4oc4ccccc45)cc3)c21]

HTM-1

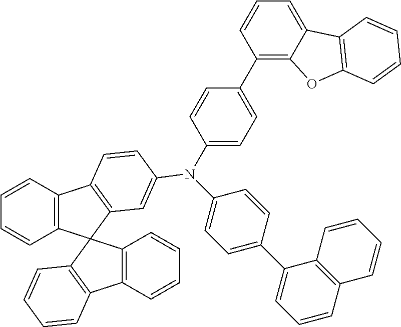 [SMILES: c1ccc2c(c1)-c1ccccc1C21c2ccccc2-c2ccc(N(c3ccc(-c4cccc5ccccc45)cc3)c3ccc(-c4cccc5c4oc4ccccc45)cc3)cc21]

HTM-2

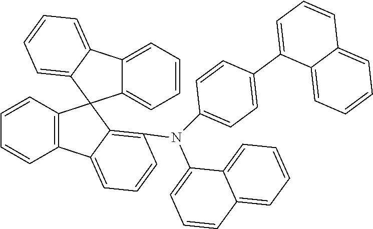 [SMILES: c1ccc2c(c1)-c1ccccc1C21c2ccccc2-c2cccc(N(c3ccc(-c4cccc5ccccc45)cc3)c3cccc4ccccc34)c21]

HTM-3

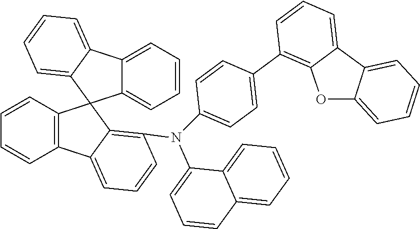 [SMILES: c1ccc2c(c1)-c1ccccc1C21c2ccccc2-c2cccc(N(c3ccc(-c4cccc5c4oc4ccccc45)cc3)c3cccc4ccccc34)c21]

HTM-4

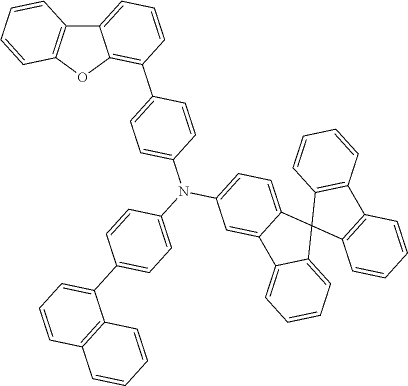 [SMILES: c1ccc2c(c1)-c1ccccc1C21c2ccccc2-c2cc(N(c3ccc(-c4cccc5ccccc45)cc3)c3ccc(-c4cccc5c4oc4ccccc45)cc3)ccc21]

HTM-C1

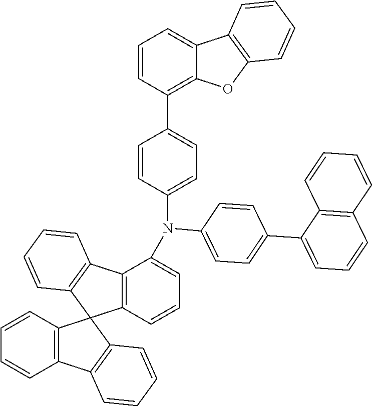 [SMILES: c1ccc2c(c1)-c1ccccc1C21c2ccccc2-c2c(N(c3ccc(-c4cccc5ccccc45)cc3)c3ccc(-c4cccc5c4oc4ccccc45)cc3)cccc21]

HTM-C2

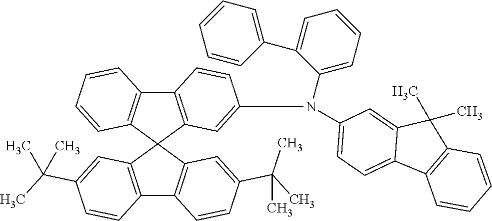 [SMILES: CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2]

HTM

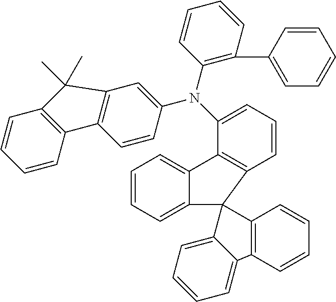 [SMILES: CC1(C)c2ccccc2-c2ccc(N(c3ccccc3-c3ccccc3)c3cccc4c3-c3ccccc3C43c4ccccc4-c4ccccc43)cc21]

EBM

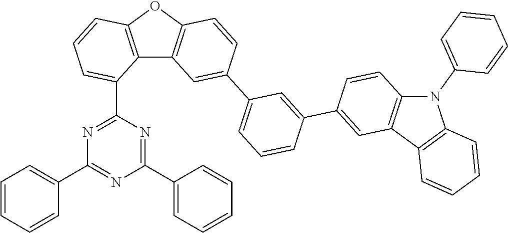 [SMILES: c1ccc(-c2nc(-c3ccccc3)nc(-c3cccc4oc5ccc(-c6cccc(-c7ccc8c(c7)c7ccccc7n8-c7ccccc7)c6)cc5c34)n2)cc1]

TMM-1

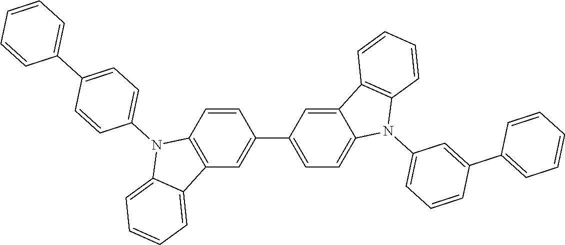 [SMILES: c1ccc(-c2ccc(-n3c4ccccc4c4cc(-c5ccc6c(c5)c5ccccc5n6-c5cccc(-c6ccccc6)c5)ccc43)cc2)cc1]

TMM-2

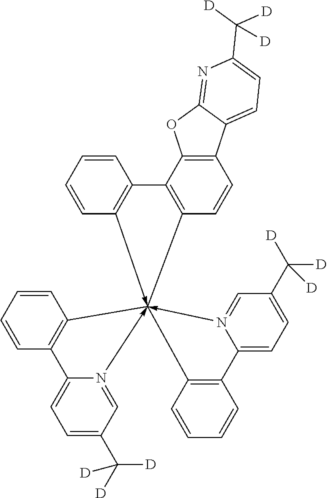

TEG

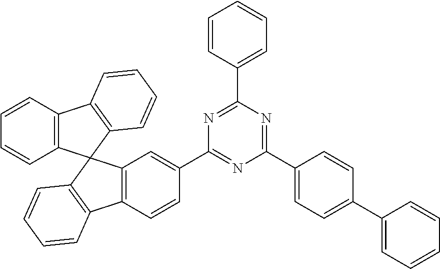 [SMILES: c1ccc(-c2ccc(-c3nc(-c4ccccc4)nc(-c4ccc5c(c4)C4(c6ccccc6-c6ccccc64)c4ccccc4-5)n3)cc2)cc1]

ETM

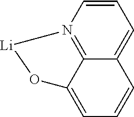

LiQ

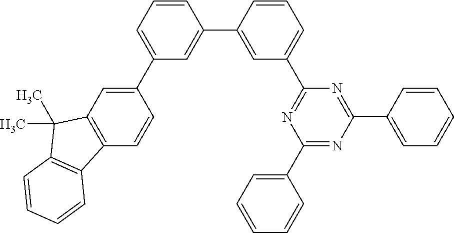 [SMILES: CC1(C)c2ccccc2-c2ccc(-c3cccc(-c4cccc(-c5nc(-c6ccccc6)nc(-c6ccccc6)n5)c4)c3)cc21]

HBM

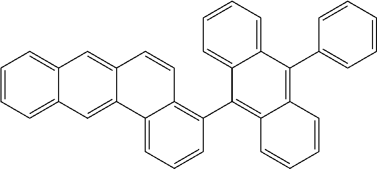 [SMILES: c1ccc(-c2c3ccccc3c(-c3cccc4c3ccc3cc5ccccc5cc34)c3ccccc23)cc1]

H-1

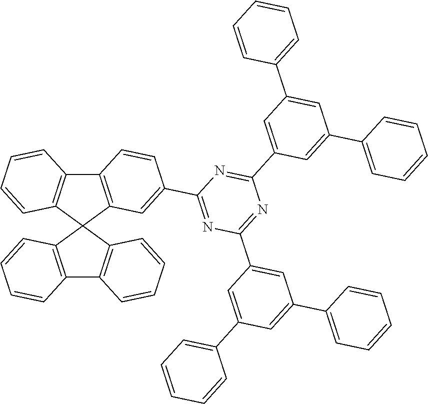 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ETM-1

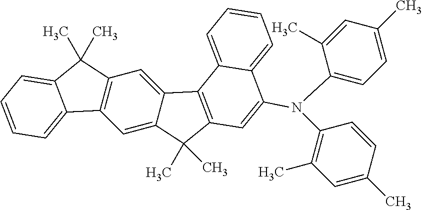 [SMILES: Cc1ccc(N(c2ccc(C)cc2C)c2cc3c(c4ccccc24)-c2cc4c(cc2C3(C)C)-c2ccccc2C4(C)C)c(C)c1]

SEB-1

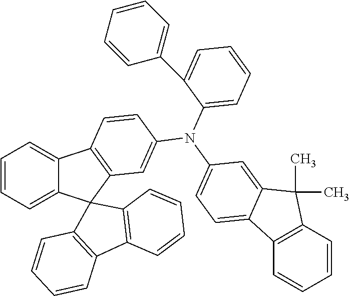 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

HTM-A

1.-19. (canceled)

20. A compound of a formula (I) 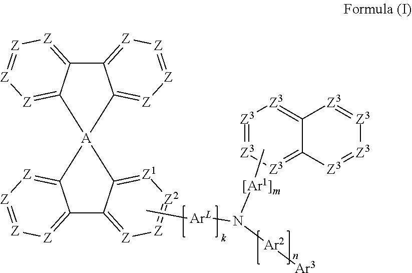
wherein the amino group shown with a variable bond position at the spirobifluorene in formula (I) is bonded either to Z¹ or to Z²; and
wherein the following applies for the groups and indices that occur:
A is C or Si;
Ar^{L} is the same or different at each instance and is selected from aromatic ring systems which have 6 to 40 aromatic ring atoms and are each substituted by R² radicals, and heteroaromatic ring systems which have 5 to 40 aromatic ring atoms and are each substituted by R² radicals;
Ar¹ is the same or different at each instance and is selected from aromatic ring systems which have 6 to 24 aromatic ring atoms and are substituted by R³ radicals, and heteroaromatic ring systems which have 5 to 24 aromatic ring atoms and are substituted by R³ radicals;
Ar² is the same or different at each instance and is selected from phenyl substituted by R⁵ radicals, naphthyl substituted by R⁵ radicals and heteroaromatic ring systems which have 6 to 10 aromatic ring atoms and are each substituted by R⁵ radicals;
Ar³ is selected from groups of the formulae (Ar³-1) to (Ar³-7) which are bonded to the formula (I) via the bond marked with * 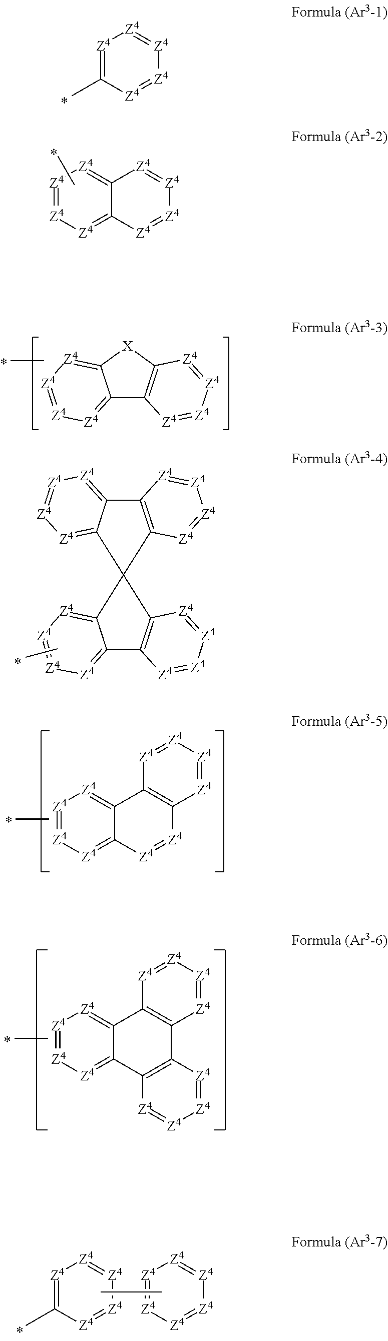
wherein Z⁴ is the same or different at each instance and is selected from CR⁶ and N, and wherein in formulae (Ar³-2) to (Ar³-7) the group Z⁴ which bonds to a freely drawn bond is C; wherein in formula (Ar³-3) X representing C(R⁶)₂ may also be bonded to the rest of formula (I), wherein a group R⁶ in this group X is then replaced by the bond to the rest of formula (I);
and wherein X is selected from O, S, NR⁶ and C(R⁶)₂; and wherein in formula (Ar³-3) one or more units Z⁴═Z⁴ may be replaced by a unit selected from the units 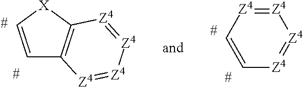
wherein the atoms marked with # in each case replace the atoms Z⁴;
and wherein in formula (Ar³-3) one or more units Z⁴—Z⁴ may be replaced by a unit selected from the units 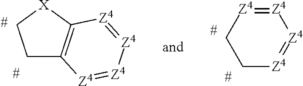
wherein the atoms marked with # in each case replace the atoms Z⁴;
Z is the same or different at each instance and is selected from CR¹ and N;
Z¹ and Z² are each C when the amino group shown with a variable bond position is bonded thereto and selected from CR¹ and N;
Z³ is the same or different at each instance and is selected from CR⁴ and N;
R¹ is the same or different at each instance and is selected from H, D, F, Cl, Br, I, C(═O)R⁷, CN, Si(R⁷)₃, N(R⁷)₂, —(Ar^{L})ₖ—N(Ar⁴)₂, P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals R¹, R², R³, R⁴, R⁵ and R⁶ may be joined to one another and may form a ring; where the alkyl, alkoxy, alkenyl and alkynyl groups mentioned and the aromatic ring systems and heteroaromatic ring systems mentioned are each substituted by R⁷ radicals; and where one or more CH₂ groups in the alkyl, alkoxy, alkenyl and alkynyl groups mentioned may be replaced by —R⁷C═CR⁷—, —C≡C—, Si(R⁷)₂, C═O, C═NR⁷, —C(═O)O—, —C(═O)NR⁷—, NR⁷, P(═O)(R⁷), —O—, —S—, SO or SO₂;
R², R³, R⁴, R⁵ and R⁶ are each the same or different at each instance and are selected from H, D, F, Cl, Br, I, C(═O)R⁷, CN, Si(R⁷)₃, N(R⁷)₂, P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals R², R³, R⁴, R⁵ and R⁶ may be joined to one another and may form a ring; where the alkyl, alkoxy, alkenyl and alkynyl groups mentioned and the aromatic ring systems and heteroaromatic ring systems mentioned are each substituted by R⁷ radicals; and where one or more CH₂ groups in the alkyl, alkoxy, alkenyl and alkynyl groups mentioned may be replaced by —R⁷C═CR⁷—, —C≡C—, Si(R⁷)₂, C═O, C═NR⁷, —C(═O)O—, —C(═O)NR⁷—, NR⁷, P(═O)(R⁷), —O—, —S—, SO or SO₂;
Ar⁴ is the same or different at each instance and is selected from aromatic ring systems which have 6 to 40 aromatic ring atoms and are substituted by one or more R⁷ radicals, and heteroaromatic ring systems which have 5 to 40 aromatic ring atoms and are substituted by one or more R⁷ radicals;
R⁷ is the same or different at each instance and is selected from H, D, F, Cl, Br, I, C(═O)R⁸, CN, Si(R⁸)₃, N(R⁸)₂, P(═O)(R⁸)₂, OR⁸, S(═O)R⁸, S(═O)₂R⁸, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹, R², R³, R⁴, R⁵ and R⁶ radicals may be joined to one another and may form a ring; where the alkyl, alkoxy, alkenyl and alkynyl groups mentioned and the aromatic ring systems and heteroaromatic ring systems mentioned are each substituted by R⁸ radicals; and where one or more CH₂ groups in the alkyl, alkoxy, alkenyl and alkynyl groups mentioned may be replaced by —R⁸C═CR⁸—, —C≡C—, Si(R⁸)₂, C═O, C═NR⁸, —C(═O)O—, —C(═O)NR⁸—, NR⁸, P(═O)(R⁸), —O—, —S—, SO or SO₂;
R¹ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁸ radicals may be joined to one another and may form a ring; and where the alkyl, alkoxy, alkenyl and alkynyl groups, aromatic ring systems and heteroaromatic ring systems mentioned may be substituted by one or more radicals selected from F and CN;
k is 0, 1, 2, 3 or 4;
m is 0, 1, 2, 3 or 4; and
n is 1, 2, 3 or 4.

21. The compound according to claim 20, wherein Z¹ and Z² are each C when the amino group shown with a variable bond position is bonded thereto and otherwise are CR¹, Z is CR¹, Z³ is CR⁴ and Z⁴ is CR⁶.

22. The compound according to claim 20, wherein R¹ is the same or different at each instance and is selected from H, —[Ar^{L}]ₖ—N(Ar⁴)₂, straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms; where the alkyl groups mentioned, the aromatic ring systems mentioned and the heteroaromatic ring systems mentioned are each substituted by R⁷ radicals.

23. The compound according to claim 20, wherein R², R³, R⁴, R⁵ and R⁶ are the same or different at each instance and are selected from H, D, F, CN, Si(R⁷)₃, N(R⁷)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁷ radicals; and where one or more CH₂ groups in the alkyl or alkoxy groups mentioned may be replaced by —C≡C—, —R⁷C═CR⁷—, Si(R⁷)₂, C═O, C═NR⁷, —NR⁷—, —O—, —S—, —C(═O)O— or —C(═O)NR⁷—.

24. The compound according to claim 20, wherein the compound contains at least one group R⁴ which is selected from aromatic ring systems having 6 to 40 aromatic ring atoms and heteroaromatic ring systems having 5 to 40 aromatic ring atoms, each of which are substituted by R⁷ radicals.

25. The compound according to claim 20, wherein index k is 0.

26. The compound according to claim 20, wherein Ar¹ is the same or different at each instance and is selected from phenyl, naphthyl, fluorenyl and biphenyl, each substituted by R³ radicals.

27. The compound according to claim 20, wherein the compound conforms to a formula selected from the following formulae: 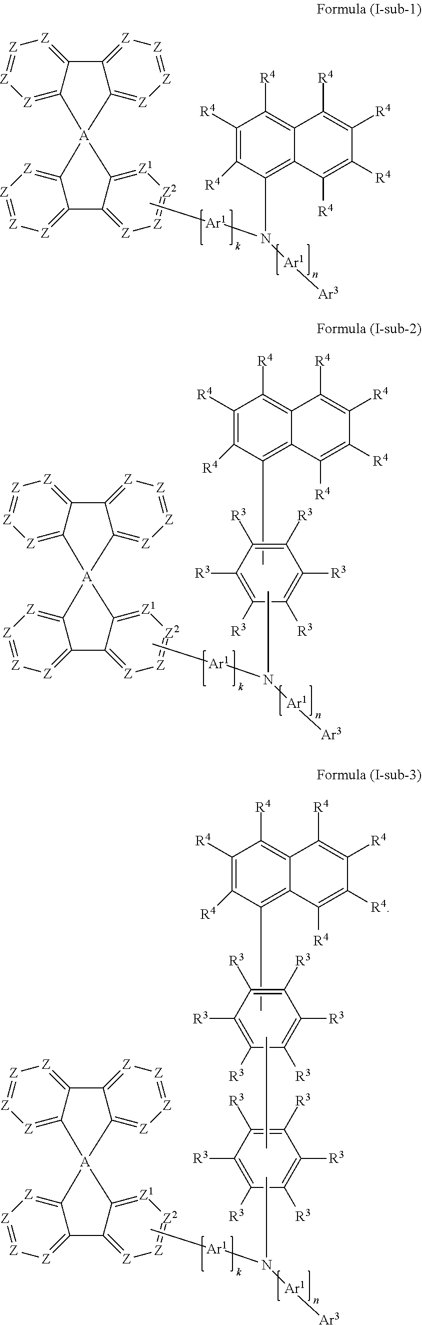
wherein the groups and indices that occur are as defined in claim 20.

28. The compound according to claim 20, wherein the compound conforms to one of the following formulae: 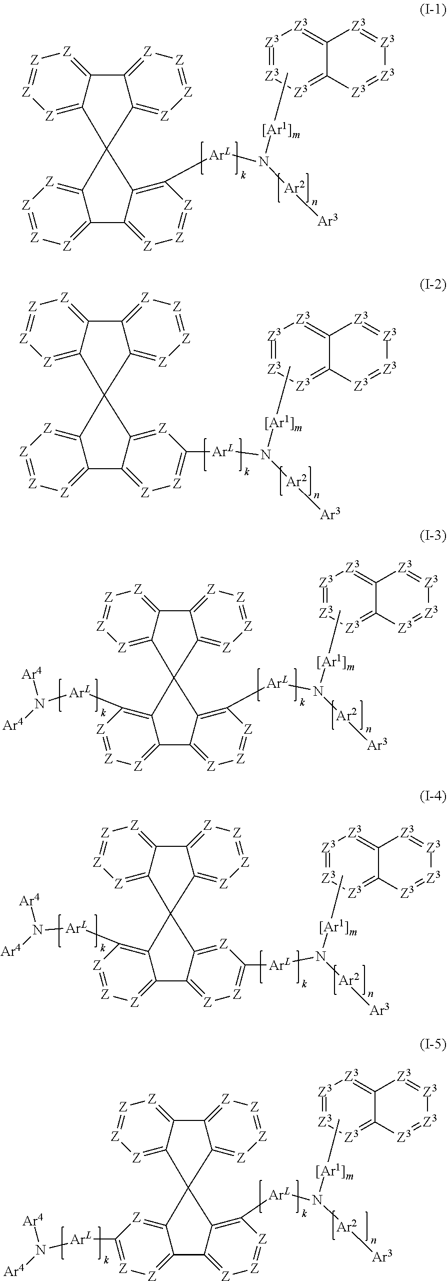 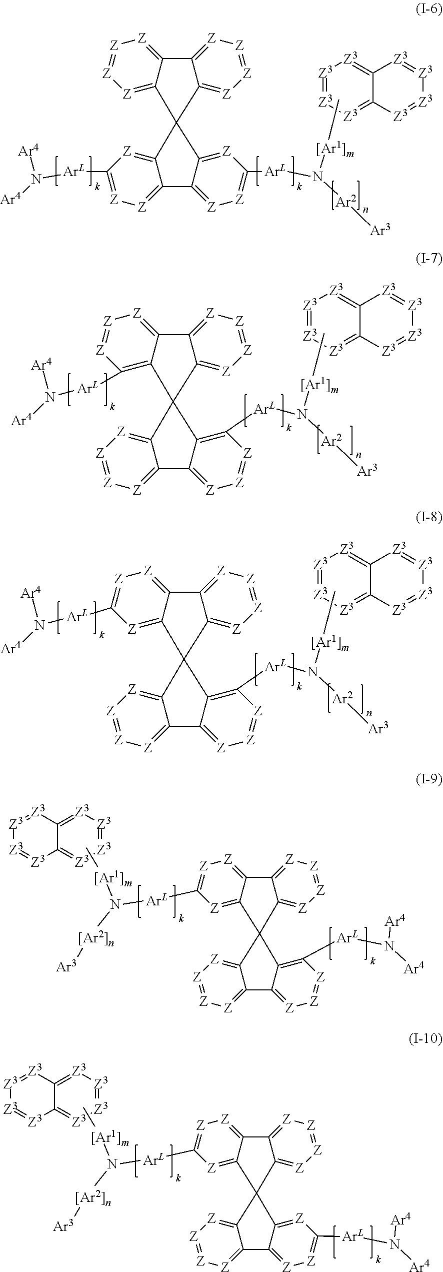
wherein the indices that occur are as defined in claim 20.

29. The compound according to claim 20, wherein Ar² is the same or different at each instance and is selected from phenyl and naphthyl, each substituted by R⁵ radicals.

30. The compound according to claim 20, wherein index n is 1 or 2.

31. The compound according to claim 20, wherein Ar³ is selected from groups of the formulae (Ar³-2) and (Ar³-3):

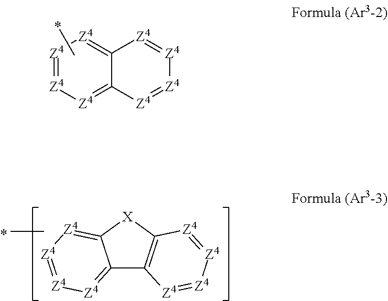

32. The compound according to claim 20, wherein A is C.

33. A process for preparing the compound according to claim 20, wherein an amino group is introduced using an organometallic coupling reaction.

34. An oligomer, polymer or dendrimer containing one or more compounds according to claim 20, wherein the bond(s) to the polymer, oligomer or dendrimer may be localized at any desired positions substituted by R¹, R², R³, R⁴, R⁵ or R⁶ in formula (I).

35. A formulation comprising at least one compound according to claim 20 and at least one solvent.

36. An electronic device comprising at least one compound according to claim 20.

37. The electronic device according to claim 36, wherein the device is an organic electroluminescent device and comprises an anode, cathode and at least one emitting layer, and in that the compound is present in a hole-transporting layer or in an emitting layer of the device.

38. A method comprising utilizing the compound as claimed in claim 20 in an electronic device.
